(19) United States
(12) Patent Application Publication
(10) Pub. No.: US 2016/0016986 A1
(43) Pub. Date: Jan. 21, 2016

(54) STABILIZED NUCLEOTIDES FOR MEDICAL TREATMENT

(71) Applicants: Milind Deshpande, Madison, CT (US); Jason Allan Wiles, Madison, CT (US); Akihiro Hashimoto, Branford, CT (US); Avinash Phadke, Branford, CT (US)

(72) Inventors: Milind Deshpande, Madison, CT (US); Jason Allan Wiles, Madison, CT (US); Akihiro Hashimoto, Branford, CT (US); Avinash Phadke, Branford, CT (US)

(21) Appl. No.: 14/805,421

(22) Filed: Jul. 21, 2015

Related U.S. Application Data
(60) Provisional application No. 62/027,061, filed on Jul. 21, 2014.

(51) Int. Cl. C07H 19/10; C07H 19/056; A61K 31/7056; A61K 31/7072

(52) U.S. Cl. CPC C07H 19/10; A61K 31/7072; C07H 19/056; A61K 31/7056

(57) ABSTRACT

5′-Deuterated nucleosides and nucleotides and modifications thereof are provided for use in medical therapies, including as antiviral, anti-tumor and anti-neoplastic agents. In one embodiment, compounds, methods and uses are provided for the treatment of hepatitis C, RSV, HSV and other viral diseases in a host, including a human.

### CROSS-REFERENCE TO RELATED APPLICATION

This application claims the benefit of U.S. Provisional Application No. 62/027,061, filed Jul. 21, 2014, which is incorporated herein by reference in its entirety.

### BACKGROUND

Nucleosides and nucleotides are used in a wide variety of biochemical pathways, including DNA and RNA synthesis, cell signaling, enzyme regulation and metabolism. Modified nucleosides and nucleotides are correspondingly used in a range of medical therapies, including as antiviral, anti-tumor, anti-neoplastic and even anti-methylating agents. Other less widely used indications include hyperuricaemia (allopurinol), immunosuppression (azathioprine and cladribine), phosphodiesterase inhibitors (theophylline), epigenetic modulators (decitabine and azacitabine) and potentially in neuroprotection and cardioprotection. In many of these therapies, the active form of the drug is the nucleotide 5′-triphosphate. See generally, Jordheim, L. P. et al. “Advances in the development of nucleoside and nucleotide analogues for cancer and viral diseases” Nat. Rev. Drug Discov. 2013 June; 12(6): 447-461.

A challenge in such therapy is that a significant proportion of administered nucleosides and nucleotides can be eliminated in the 5′-hydroxyl form and are not phosphorylated by the appropriate kinase to the corresponding 5′-monophosphate which is then converted to the active 5′-triphosphate. This leads to the result that some nucleosides and nucleotides are inactive in vivo or require a dose that compensates for the significant amount of drug that is not converted to the active 5′-triphosphorylated metabolite.

It has been observed that, even if the 5′-monophosphorylation of a 5′-hydroxyl nucleoside is accomplished in vivo by the relevant kinase, the rate can be significantly slower than the rate of mono-phosphorylation of a naturally occurring nucleoside. This reduced rate of 5′-monophosphorylation decreases the overall rate of conversion to the active 5′-triphosphate, and affects drug therapy.

As one example, this is a known problem in the case of nucleosides that are highly derivatized, including nucleosides with activity against hepatitis C, for example, including but not limited to 2′-methyl-2′-hydroxyl or -2′-fluoro-nucleosides. A common response to this problem for antiviral and anti-neoplastic nucleoside-based drugs has been to provide them as stabilized 5′-phosphate derivatives, such as phosphoramidates. However, even stabilized nucleotide 5′-phosphate prodrugs, including phosphoramidates, can be metabolized in vivo to the inactive 5′-hydroxyl nucleoside, which is passed without therapeutic effect.

Sofosbuvir (see structure below) is a nucleoside phosphoramidate NS5B inhibitor approved in December 2013 for the treatment of HCV. The approved labeling recommends the following regimens: (i) for genotypes 2 and 3 a 400 mg once a day oral tablet in combination with ribavirin and (ii) for genotypes 1 and 4 a 400 mg once a day oral tablet (triple combination therapy) with ribavirin and pegylated interferon. The sofosbuvir treatment lasts 12 weeks for genotypes 1, 2 and 4 and 24 weeks for genotype 3. Sofosbuvir can also be used with ribavirin for the treatment of chronic hepatitis C patients with hepatocellular carcinoma awaiting liver transplantation for up to 48 weeks or until liver transplantation to prevent post-transplant HCV infection. The FDA granted Sofosbuvir Priority Review and Breakthrough Therapy designation based on data from several large clinical trials that indicated a sustained viral response (SVR) of twelve weeks in 50-90% of the trial participants. Patients who achieve “SVR12” are often considered cured.

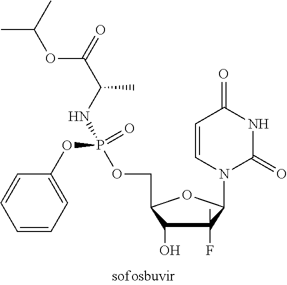

Alios BioPharma, Inc. licensed ALS-2200 to Vertex Pharmaceuticals Inc. for hepatitis C treatment development in June 2011. ALS-2200 is a mixture of diastereomers at a chiral phosphorus stereocenter. A single diastereomer, VX-135, was being developed by Vertex, and in Phase II clinical trials. While the companies have not disclosed the chemical structure of VX-135, they have said that it is a uridine nucleotide analog prodrug, and an NS5B inhibitor. In 2013, the FDA placed VX-135 on partial clinical hold after three patients receiving high dosages of VX-135 showed liver toxicity. Lowering the dose of a nucleotide inhibitor to avoid toxicity can sometimes also compromise or lower efficacy. Vertex announced in January 2014 that VX-135 in combination with daclatasvir (Bristol-Myers Squibb NS5A inhibitor) had completed a Phase 2a trial. In an intent-to-treat analysis, the sustained viral response rate four weeks after completion of treatment (SVR4) was 83% (10 of 12) in treatment-naïve genotype 1 infected individuals who received 200 mg VX-135 in combination with daclatasvir. One patient exhibited a serious adverse event of vomiting/nausea. The eleven remaining patients completed 12 weeks of treatment, for a completion of treatment rate (SVR12) of 91%. Vertex announced in May 2014 that it would no longer invest in the development of VX-135.

Idenix Pharmaceuticals Inc., recently acquired by Merck & Co., is developing IDX21437 for the treatment of hepatitis C, which is a uridine nucleotide prodrug NS5B inhibitor. Other details of the chemical structure have not been released to date. In April 2014, Idenix announced that once-daily 300 mg IDX21437 for seven days led to a mean maximum reduction in viral load of 4.2-4.3 log 10 IU/mL in 18 treatment naïve patients with genotype 1, 2 or 3.

Despite some progress in the area of hepatitis C treatment, there have also been a number of difficult setbacks. BMS-986094, a guanosine-based phosphoramidate for hepatitis C was pulled from clinical trials after the death of a patient due to heart failure in August 2012. BMS thereafter announced in 2013 that it was exiting the hepatitis C research area. Following the BMS drug withdrawal, Idenix Pharmaceuticals' similar NS5B inhibitor, IDX 19368, which shares the same active metabolite, BMS-986094, was placed on clinical hold and ultimately discontinued. This followed the previous clinical hold and discontinuation of development of the nucleotide prodrug IDX184 for the same indication.

There is a need to improve modified nucleotide and nucleoside therapies to increase the active 5′-triphosphate pool and decrease the amount of drug that is inactive in vivo. There is also a need to improve aspects of the pharmacokinetics of therapeutic modified nucleotides and nucleosides.

It is also known that effective treatment against certain viruses, including hepatitis C, includes combination therapy, due to the onset of viral resistance during monotherapy. Given the documented challenges of developing optimal antiviral and in particular hepatitis C agents, and the fact that multiple therapeutic agents are required for optimal therapy, there is a strong need for additional active agents.

### SUMMARY OF THE INVENTION

Improved modified nucleosides and nucleotides are provided for use in a range of medical therapies, including as antiviral, anti-tumor and anti-neoplastic agents. In many of these therapies, the active form of the drug is the nucleotide 5′-triphosphate. In one embodiment, improved compounds, compositions, and methods are provided for the treatment of hepatitis C and other viral diseases and other disorders that can be treated with modified nucleotides.

It has been surprisingly discovered that having deuterium in the 5′-position of the phosphate of a nucleoside stabilizes the corresponding nucleotide in cells from metabolism to the corresponding 5′-hydroxyl nucleoside. This elegant solution was not straightforward. It is surprising because the deuterium atom(s) are not cleaved during enzymatic dephosphorylation and are not bound to an atom that is cleaved during enzymatic dephosphorylation. The deuterium-carbon-oxygen bonds are not broken during 5′-dephosphorylation. It is known that 5′-monophosphates are dephosphorylated by an enzyme-catalyzed hydroxyl ion attack on the phosphate, which then eliminates the 5′-hydroxyl nucleoside. See for example, D. Koshland and S. Stein, Mechanism of Action of 5-Nucleotidase, J. Biol. Chem. 1956, 221:469-476.

The disclosure includes the use of 5′-deuterium to produce a significant effect on metabolism and efficacy through a remote and unexpectedly important secondary deuterium isotope effect. Such an important secondary deuterium isotope effect on de-monophosphorylation at the 5′-position has not been previously reported prior to reporting by the present inventors. By increasing the stability of the 5′-monophosphate of the nucleoside against dephosphorylation, an increase in the active 5′-triphosphate pool of the nucleoside can be achieved, which can result in increased efficacy at a given oral dosage or equal efficacy using a lower dose of the nucleoside. 5′-Deuteration may also have a significant effect on the half-life of a nucleoside 5′-monophosphate, and thus pharmacokinetics, of the drug. This invention thus provides the means to increase the effectiveness of a nucleotide or nucleoside, without introducing additional toxicity, through complex derivatization.

As described above, a number of strategies have been reported in the literature to stabilize 5′-phosphate nucleotides in vivo. See literature reviews that describe nonlimiting examples: A. Ray and K. Hostetler, “Application of kinase bypass strategies to nucleoside antivirals,” Antiviral Res. 2011 November; 92(2): 277-291 and M. Sofia; “Nucleotide prodrugs for HCV therapy,” Antivir. Chem. and Chemother. 2011 August; 22(1): 23-49. The stabilized phosphate prodrug strategies described in these articles, among others, can be enhanced and made more efficient with the addition of one or two deuterium(s) to the 5′-position of the nucleoside, as described in more detail below. Examples of stabilized phosphate nucleotide prodrugs that can benefit from this strategy include, but are not limited to phosphoramidates, 3,5-cyclic phosphoramidates, phosphate esters, diesters, and triesters, nucleotide derivatives of monophosphates, diphosphates, and triphosphates, 3′,5′-cyclic phosphates (including CycloSAL), phospholipids (including acylphospholipids and etherphospholipids), HepDirect prodrugs, SATE derivatives (S-acyl-2-thioester)s, DTE (dithiodiethyl) prodrugs and protein conjugates. The invention also includes administration of a 5′-deuterated analogue of the parent nucleoside or the therapeutic use of the simple 5′-deuterated mono, di or triphosphate of any of the active compounds described herein, to improve the in vivo performance of the compound.

In an embodiment, the 5′-deuterated stabilized nucleotide prodrug is a phosphoramidate which is administered as a phosphorus R or S stereoisomer, wherein the stereoisomer is at least in 90% pure form, and typically, 95%, 98%, or 99% pure form. In an alternative embodiment, the phosphoramidate is racemic.

Improved nucleoside HCV therapy can be achieved using, for example, 2′-β-methyl-uridine; 2′-deoxy-2′-α-fluoro-2′-β-methyl-uridine (the parent nucleoside of Sofosbuvir) and/or ribavirin, administered or used as the 5′-deuterated phosphate (including the mono, di or tri phosphate), 5′-deuterated-hydroxyl, a 5′-deuterated stabilized nucleotide phosphate prodrug (including a phosphoramidate), or a 5′-deuterated acyclic nucleoside prodrug as further described herein. In another embodiment, an effective amount of the uridine nucleotide analog NS5B inhibitor IDX21437 or IDX21459 or mericitabine can be administered as a 5′-mono or dideuterated derivative to a host in need thereof.

In one particular embodiment a method for the treatment of a host infected with hepatitis C, or another disorder that can be treated with a therapeutic nucleoside that is activated to the phosphate, is provided that includes the administration of an effective amount of one, two or more 5′-deuterated phosphate, 5′-deuterated-hydroxyl, a 5′-deuterated stabilized nucleotide phosphate prodrug (such as a phosphoramidate), or a 5′-deuterated acyclic nucleoside prodrug as further described herein, or a pharmaceutically acceptable salt thereof, optionally in a pharmaceutically acceptable carrier.

In another embodiment, 5′-deuterated ribavirin, in any form described herein, including in Table 4, is used to treat a flavivirus, for example West Nile virus, or a respiratory virus, such as respiratory syncytial virus (RSV), an adenovirus, or any other disorder that is treatable with ribavirin.

In one embodiment, a 5′-deuterated-2′-methyl-2′-(hydroxyl or fluoro) uridine nucleotide phosphoramidate is administered alone or in combination with an NS5A inhibitor and/or a protease inhibitor, with or without optionally 5′-deuterated ribavirin (or a 5′-deuterated stabilized phosphate prodrug of ribavirin), with or without interferon.

In another embodiment, any nucleoside drug and/or nucleotide drug used to treat a medical disorder can be modified to a 5′-deuterated analogue to achieve the desired medicinal effect. In a particular embodiment, the 5′-deuterated triphosphate is the active species for the antiviral, anti-tumor or anti-cancer indication described herein or other known indication.

In other embodiments, a 5′-deuterated nucleoside or nucleotide analog as described herein with known or discovered activity for the treatment of a viral disease is used alone or in combination with other active agents. Examples include Flaviviridae (such as flavivirus, hepacivirus (HCV), and pestivirus); respiratory viruses (such as adenovirus, avian influenza, Influenza virus type A and B, respiratory syncytial virus, rhinovirus, and SARS); gastro-enteric viruses (such as coxsackie, enterovirus, poliovirus, and rotavirus); herpes simplex 1 and 2; cytomegalovirus; varicella; Caliciviridae (such as norovirus); and retroviruses (such as HIV-1 and HIV-2). Nonlimiting examples of such drugs that are useful for HIV and hepatitis B include Emtricitabine (β-L-5-fluorocytidinyl-1′,3′-oxathiolane) and Lamivudine (β-L-cytidinyl-1′,3′-oxathiolane). Abacavir and entecavir can be modified as described herein for improved methods and compounds to treat HIV. Telbuvudine can likewise be modified as described herein to achieve improved methods and compounds to treat HBV.

In another embodiment, an effective amount of the 5′-deuterated phosphate, 5′-deuterated-hydroxyl, 5′-deuterated stabilized nucleotide phosphate prodrug, or 5′-deuterated acyclic nucleoside prodrug as further described herein or it's pharmaceutically acceptable salt, optionally in a pharmaceutically acceptable carrier, is provided to a host in need of anti-neoplastic therapy, including anti-cancer therapy.

In another embodiment, a method of treating a host afflicted with any disorder described herein, for example but not limited to HCV, is provided comprising administering to the host an effective amount of a nucleoside or nucleotide that has deuterium with at least 50% enrichment at the 5′-position of the nucleoside or nucleotide. The 5′-deuterated phosphate, 5′-deuterated-hydroxyl, 5′-deuterated stabilized nucleotide phosphate prodrug, or 5′-deuterated acyclic nucleoside prodrug is not a thiophosphate or thiophosphate prodrug unless specifically referred to. In other embodiments, the enrichment is at least 90%. In another embodiment, there are two deuteriums at the 5′-position and each has at least 50%, 60%, 70%, 80%, 90%, or 95% enrichment. In another embodiment, there are two deuteriums at the 5′-position and each has at least 96%, 97%, 98%, or 99% enrichment. In the absence of indication to the contrary, the enrichment of deuterium in the specified position of the compound described herein is at least 90%.

The invention includes additional optional deuteration or other isotopic substitution where desired to achieve the intended result, or to improve the properties of the molecule. For example, deuterium can be included in the nucleos(t)ide base, the sugar or modified sugar moiety or on other 5′-substituents. In particular, deuterium can be used in any R group that is or contains hydrogen, including R¹, R², R³, R⁴, R⁵, R⁶, R^{7a}, R^{7b}, R⁸, R^{9a}, R^{9b}, R¹⁰, R¹¹, R¹², R¹³, R¹⁴, R¹⁵, R¹⁶, R^{17a}, R^{17b}, R¹⁸, R¹⁹, R²⁰, R^{21a}, R^{21b}, R³¹, R³², R³³, R³⁴, R³⁵, R³⁶, R³⁷, R³⁸, R³⁸, R⁴⁰ and/or R⁴¹.

### BRIEF DESCRIPTION OF THE DRAWINGS

FIG. 1 is a Table showing the concentration of Formula IX (the undeuterated 2′-methyluridine) and Formula VI (5′-deuterated 2′-methyluridine) in human hepatocyte media and cell extract after incubation with 20 μM of Formula VII or Formula II, respectively. Specifically, as described in Example 33, using 20 μM Formula II or its undeuterated Formula VII counterpart (12 well plate (1 ml) with hepatocytes seeded at 0.67 million cells per well for 24 hours) results in a 1.9 fold (media, i.e., extracellular concentration) and 2.9 fold (cell extract, i.e., intracellular) higher concentration of undeuterated dephosphorylated 2′-methyl uridine (Formula IX) compared to that resulting from the 5′-deuterated form (Formula VI).

FIG. 2 is a Table showing the concentration of Formula IX (the undeuterated 2′-methyluridine) and Formula VI (5′-deuterated 2′-methyluridine) in human hepatocyte media and cell extract after incubation with 20 μM of Formula VII or Formula II, respectively. As described in Example 33, results of incubation of 20 μM Formula II or its undeuterated Formula VII counterpart (6 well plate (2 ml) with hepatocytes seeded at 1.7 million cells per well for 24 hours) indicate a 1.5 fold (cell extract, i.e., intracellular) and 2.8 fold (cell extract, i.e., intracellular) higher concentration of undeuterated dephosphorylated 2′-methyl uridine (Formula IX) compared to that resulting from the 5′-deuterated form (Formula VI).

FIG. 3 is a Table showing the concentrations of Formula IV (the active deuterated triphosphate metabolite of Formula II) produced in human liver hepatocytes (pmol Formula IV/million cells) at 2, 4, 8, 25, or 48 hours of incubation with 5 μM Formula II. The results of three experiments are shown, along with the mean and standard deviation determined for each time point. Peak levels of Formula IV were obtained at >48 hours in human hepatocytes. The concentration of VX-135-TP (the active triphosphate metabolite) generated from VX-135 is shown at 24 hours. As discussed in Example 34, the levels of triphosphate (Formula IV) generated from Formula II are 4-fold higher than levels of triphosphate (VX-135-TP) generated from VX-135, suggesting that VX-135 will be less potent than Formula II.

FIG. 4 is a graph of the concentration of Formula IV (ng/ml) vs. concentration of Formula II (μM) in human liver hepatocytes. Formula IV concentrations in human hepatocytes were determined after 24 hour incubations with 0.15, 0.45, and 1.35 μM Formula II. This Figure is described in Example 35.

FIG. 5 is a Table showing the half-lives of the active triphosphate (Formula IV or GS-7977-TP) in human, dog, monkey, and rat hepatocytes. Formula II or GS-7977 (Sofosbuvir) were added at selected concentrations to hepatocytes (human, dog, monkey and rat) and incubated at 37° C. Supernatant cell extracts of Formula IV or GS-7977-TP (the active triphosphate metabolites) were measured by high performance liquid chromatography with tandem mass spectrometric detection (LC-MS/MS). As discussed in Example 36, the triphosphate half-life values ranged from 8-30 hours and Formula IV generally had a longer half-life than GS-7977-TP across all species tested. (a−Values in parenthesis=95% confidence interval).

FIG. 6 is graph of the concentration of Formula IV (the active deuterated triphosphate metabolite of Formula II) produced in human liver hepatocytes (pmol Formula IV/million cells) during 48 hours of incubation with 5 μM Formula II. Concentrations were measured at the indicated times and the AUC was calculated with Graphpad Prism 5 software. As discussed in Example 37, peak levels of Formula IV were obtained at >48 hours in human hepatocytes.

FIG. 7 is graph of the concentration of GS-7977-TP (the active triphosphate metabolite of Sofosbuvir) produced in human liver hepatocytes (pmol GS-7977-TP/million cells) during 48 hours of incubation with 5 μM GS-7977 (Sofosbuvir). Concentrations were measured at the indicated times and the AUC was calculated with Graphpad Prism 5 software. As discussed in Example 37, peak levels of GS-7977-TP (the active triphosphate metabolite of Sofosbuvir) were obtained at 24 hours in human hepatocytes.

### DETAILED DESCRIPTION

### 5′-Deuterated Stabilized Nucleotides

It has been surprisingly discovered that having deuterium in the 5′-position of a nucleos(t)ide, for example, a 5′-deuterated phosphate (including a mono, di or triphosphate), 5′-deuterated-hydroxyl, a 5′-deuterated stabilized nucleotide prodrug (including those shown in Tables 1-5), or a 5′-deuterated acyclic nucleoside prodrug (Table 6) stabilizes the corresponding 5′-phosphate in cells from metabolizing to the corresponding 5′-hydroxyl nucleoside. This is surprising because the deuterium atom(s) are not cleaved during enzymatic dephosphorylation and are not bound to an atom that is cleaved during enzymatic dephosphorylation. The disclosure includes the use of 5′-deuterium to produce a significant effect on metabolism and efficacy through a remote and unexpectedly important secondary deuterium isotope effect. Such an important secondary deuterium isotope effect on de-monophosphorylation at the 5′-position has not been previously reported prior to reporting by the present inventors. By increasing the stability of the 5′-monophosphate of the nucleoside against dephosphorylation, an increase in the active 5′-triphosphate pool of the nucleoside can be achieved, which can result in increased efficacy at a given oral dosage or equal efficacy using a lower dose of the nucleoside. 5′-Deuteration may also have a significant effect on the half-life, and thus pharmacokinetics, of the drug. This invention thus provides the means to increase the effectiveness of a nucleotide or nucleoside without introducing additional toxicity.

Nonlimiting examples of 5′-deuterated nucleotide prodrug structures according to the present invention include, but are not limited to, those in Tables 1, 2, 3, 4, 5, and 6 below.

TABLE 1

| Structure | 5′-Deuterated Stabilized Uridine Phosphate Structures |
|---|---|
| 1-1 | 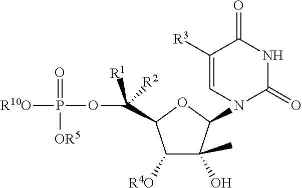 |
| 1-2 | 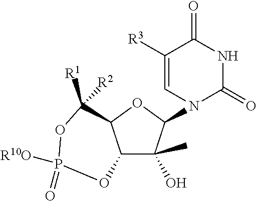 |
| 1-3 | 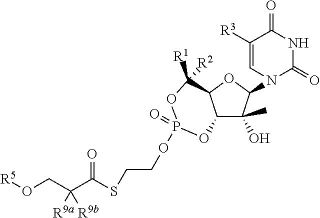 |
| 1-4 | 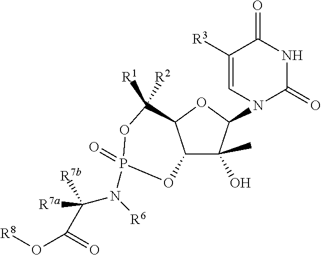 |
| 1-5 | 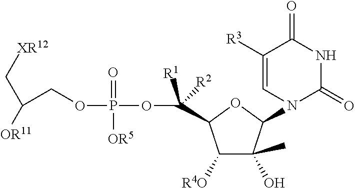 |
| 1-6 | 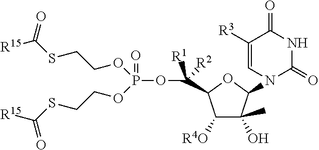 |
| 1-7 | 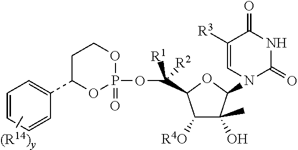 |
| 1-8 | 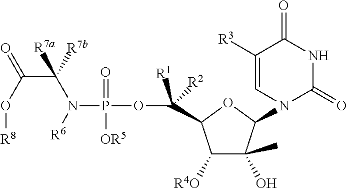 |
| 1-9 | 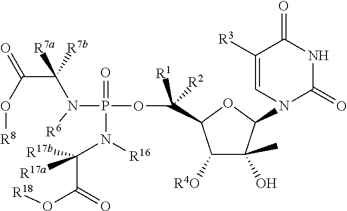 |
| 1-10 | 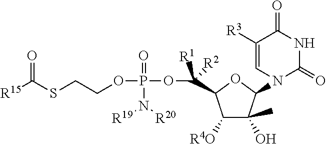 |
| 1-11 | 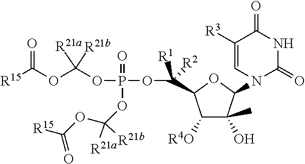 |
| 1-12 | 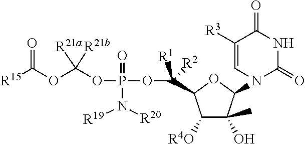 |

TABLE 2

| Structure | 5′-Deuterated Stabilized Cytidine Phosphate Structures |
|---|---|
| 2-1 | 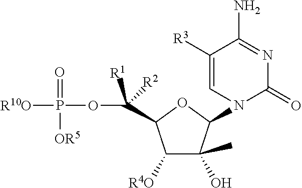 |
| 2-2 | 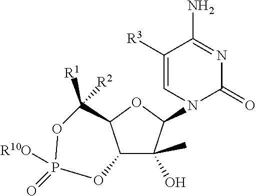 |
| 2-3 | 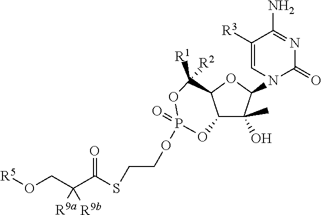 |
| 2-4 | 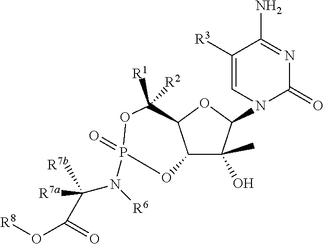 |
| 2-5 | 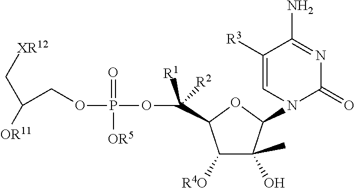 |
| 2-6 | 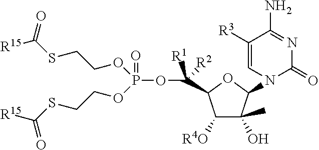 |
| 2-7 | 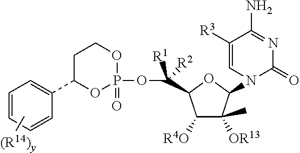 |
| 2-8 | 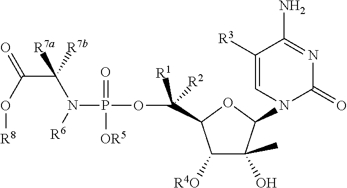 |
| 2-9 | 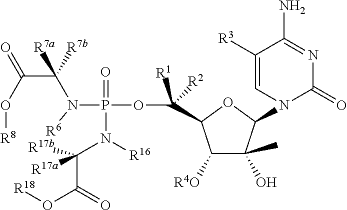 |
| 2-10 | 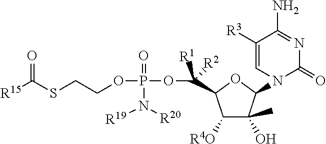 |
| 2-11 | 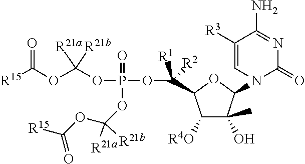 |
| 2-12 | 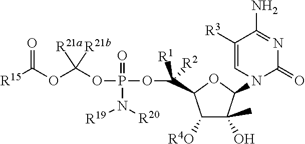 |

TABLE 3

| Structure | 5′-Deuterated Stabilized Uridine Phosphate Structures |
|---|---|
| 3-1 | 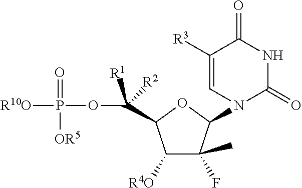 |
| 3-2 | 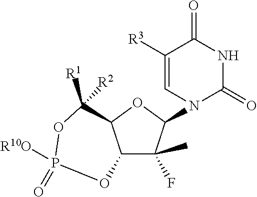 |
| 3-3 | 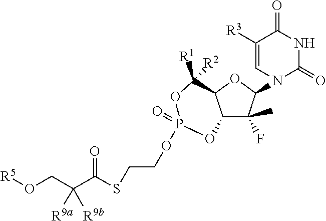 |
| 3-4 | 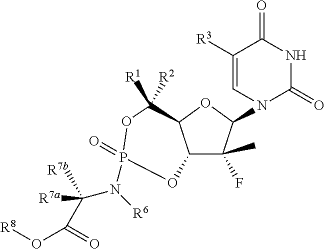 |
| 3-5 | 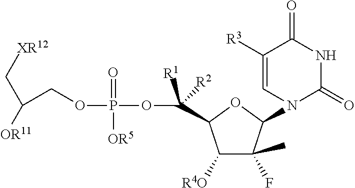 |
| 3-6 | 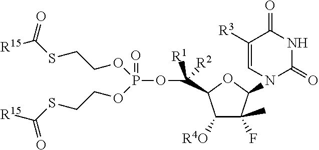 |
| 3-7 | 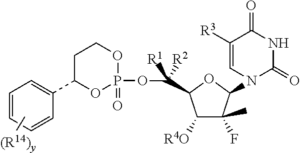 |
| 3-8 | 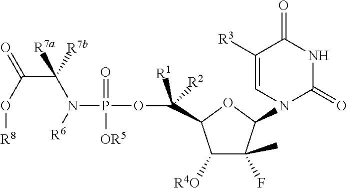 |
| 3-9 | 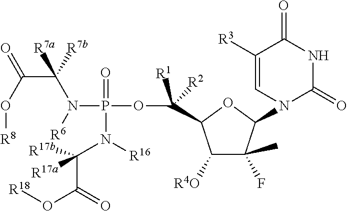 |
| 3-10 | 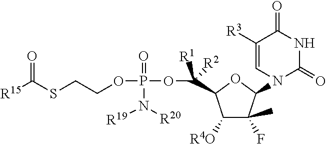 |
| 3-11 | 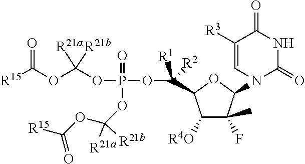 |
| 3-12 | 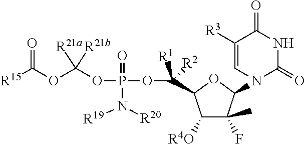 |

TABLE 4

| Structure | 5′-Deuterated Ribavirin Structures |
|---|---|
| 4-1 | 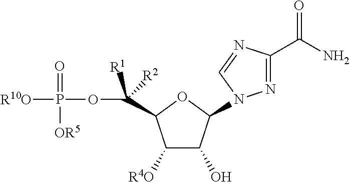 |
| 4-2 | 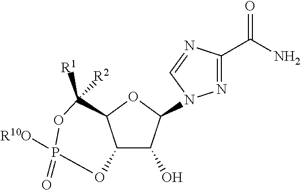 |
| 4-3 | 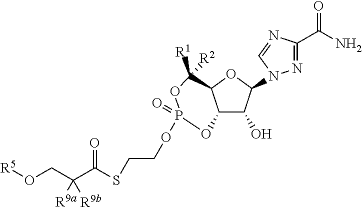 |
| 4-4 | 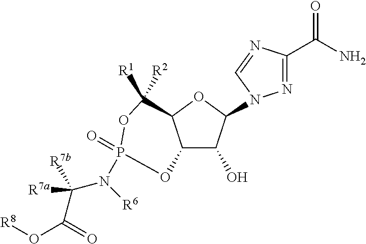 |
| 4-5 | 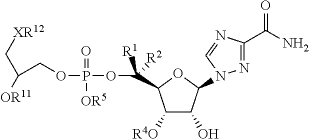 |
| 4-6 | 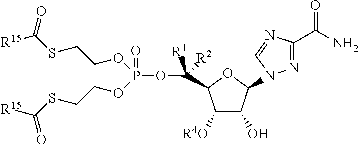 |
| 4-7 | 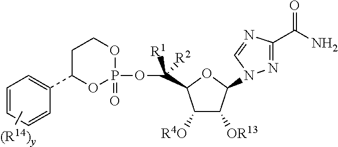 |
| 4-8 | 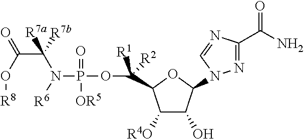 |
| 4-9 | 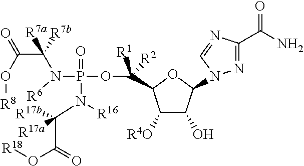 |
| 4-10 | 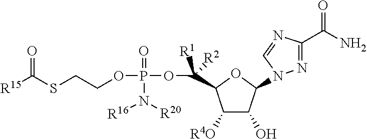 |
| 4-11 | 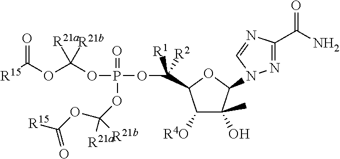 |
| 4-12 | 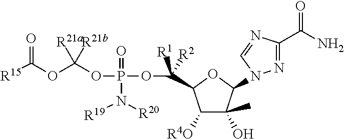 |

TABLE 5

| | Other 5′-Deuterated Stabilized Nucleotide Phosphate |
|---|---|
| Structure | Prodrug Structures |
| 5-1 | 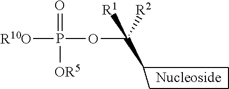 |
| 5-2 | 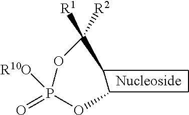 |
| 5-3 | 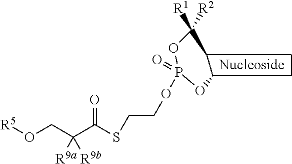 |
| 5-4 | 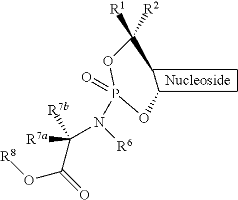 |
| 5-5 | 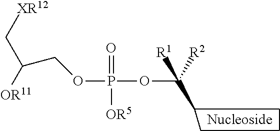 |
| 5-6 | 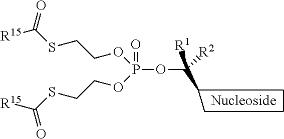 |
| 5-7 | 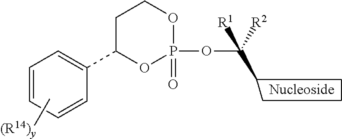 |
| 5-8 | 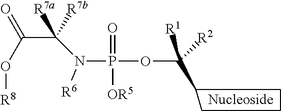 |
| 5-9 | 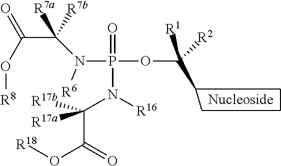 |
| 5-10 | 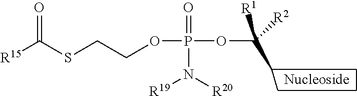 |
| 5-11 | 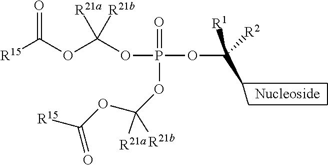 |
| 5-12 | 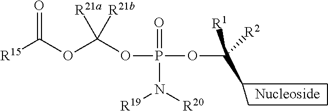 |

TABLE 6

| | 5′-Deuterated Stabilized Acyclic Nucleoside Phosphate |
|---|---|
| Structure | Prodrug Structures |
| 6-1 | 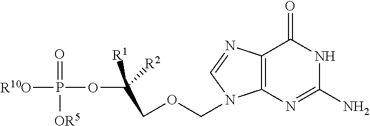 |
| 6-5 | 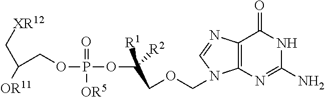 |
| 6-6 | 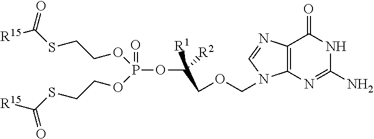 |
| 6-7 | 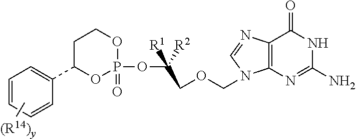 |
| 6-8 | 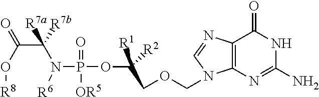 |
| 6-9 | 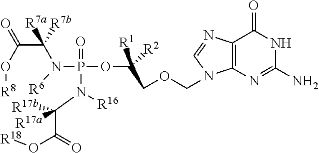 |
| 6-10 | 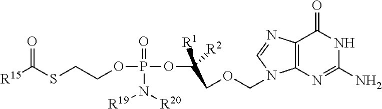 |
| 6-11 | 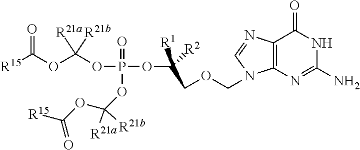 |
| 6-12 | 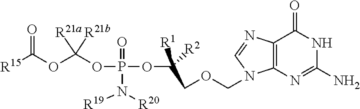 |

R¹ and R² are independently deuterium, hydrogen, or C(H)ₘ(D)ₙ; and at least one of R¹ and R² is deuterium; and typically both R¹ and R² are deuterium.

R³ is hydrogen, deuterium, halogen (F, Cl, Br, or I), C(H)ₘ(D)ₙ; or, alternatively, alkyne, wherein the R³ alkyne and the C⁴-oxygen of the pyrimidine can combine to form a heterocyclic ring.

R⁴ is hydrogen, deuterium, C(H)ₘ(D)ₙ, acyl, including an amino acid, or phosphate (including mono, di or triphosphate).

R⁵ is hydrogen, deuterium, alkyl, cycloalkyl, aryl, heteroaryl, heterocyclic, -alkyl(heterocycle), -alkyl(heteroaryl), -alkylaryl (including benzyl); which includes, but is not limited to, phenyl (including but not limited to halophenyl, cyanophenyl, and alkylphenyl) or naphthyl (for example, 1 or 2-naphthyl).

R⁶ is hydrogen, deuterium, C₁₋₃alkyl (for example, methyl) or C₃₋₅ cycloalkyl.

R^{7a} and R^{7b} are independently hydrogen, deuterium, C₁-C₆alkyl (including C₁-C₃ alkyl), halogen, C₃-C₆ cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid (i.e., a moiety which is found on the carbon linking the amino group and the carboxyl group in an amino acid) or its isomer; each of which is optionally substituted. The R^{7a} and R^{7b} substituents independently include but are not limited to any corresponding to the R^{7a} and R^{7b} positions found in natural amino acids (or their D-counterpart) and non-proteogenic amino acids, such as serine, threonine, asparagine, glutamine, cysteine, selenocysteine, glycine (e.g., hydrogen), alanine, valine, isoleucine, methionine, phenylalanine, tyrosine, tryptophan, ornithine, glutamine, arginine, histidine, proline, hydroxyproline, selenomethionine, lanthionine, 2-aminoisobutyric acid or dehydroalanine (i.e., R^{7a} or R^{7b} is an exo-double bond), with optional protection of functional groups such as hydroxyl, amino, thiol, etc. In an alternative embodiment, R^{7a} and R^{7b} can together be a cycloalkyl or heterocyclic group, which may be optionally substituted. Where the substituents in the amino acid require cyclization, the formula envisions such modifications, such as that required for proline (where R⁶ and R^{7a} or R^{7b} can come together to form the proline moiety). Alternatively, for example, R^{7a} and R^{7b} are taken together to form a 3- to 6-membered cycloalkyl ring or 3- to 6-membered heterocycloalkyl ring containing one heteroatom chosen from N, O, and S; each of which is optionally substituted. Where R^{7a} is hydrogen and R^{7b} is non-hydrogen, the amino acid moiety has L-stereochemistry similar to a naturally occurring amino acid. Where R^{7a} is non-hydrogen and R^{7b} is hydrogen, the amino acid moiety has D-stereochemistry which is not typically a naturally occurring amino acid. In this invention, the amino acid in the phosphoramidate of any of the disclosed structures can be either in the D or L configuration. If an L amino acid is depicted, it is envisioned that the D amino acid moiety can also be used.

R⁸ is C₁-C₆alkyl, C₃-C₆ cycloalkyl, aryl, C₂-C₆alkenyl, C₂-C₆alkynyl, (C₃-C₇cycloalkyl)C₀-C₄alkyl-, (aryl)C₀-C₄alkyl-, (3- to 6-membered heterocycloalkyl)C₀-C₄alkyl-, or (heteroaryl)C₀-C₄alkyl-; each of which is optionally substituted.

R^{9a} and R^{9b} are each independently hydrogen, deuterium or C₁-C₃alkyl, C₃-C₄ cycloalkyl, or wherein R^{9a} and R^{9b} together form a cycloalkyl group of C₃-C₅.

R¹⁰ is hydrogen, deuterium, alkyl, cycloalkyl, aryl, heteroaryl, heterocyclic, -alkyl(heterocycle), -alkyl(heteroaryl), -alkylaryl (including benzyl); which includes, but is not limited to, phenyl (including but not limited to halophenyl, cyanophenyl, and alkylphenyl) or naphthyl (for example, 1 or 2-naphthyl).

R¹¹ is C₁-C₂₂alkyl, cycloalkyl, C₃-C₂₂alkenyl or C₃-C₂₂alkynyl or R¹¹ is —C(O)—C₆-C₂₂alkyl, —C(O)—C₆-C₂₂alkenyl or —C(O)—C₆-C₂₂alkynyl.

R¹² is C₁-C₂₂alkyl, C₃-C₂₂alkenyl or C₃-C₂₂alkynyl of R¹² is —C(O)—C₆-C₂₂alkyl, —C(O)—C₆-C₂₂alkenyl or —C(O)—C₆-C₂₂alkynyl.

R¹³ is hydrogen, deuterium, C(H)ₘ(D)ₙ, acyl, including an amino acid, or phosphate.

R¹⁴ is independently deuterium, halogen, C₁-C₂haloalkyl, C₁-C₂haloalkoxy, C₁-C₆alkyl, cycloalkyl, allenyl, C₂-C₆alkenyl, C₂-C₆alkynyl, C₁-C₆alkoxy, (C₃-C₆cycloalkyl)C₀-C₄alkyl-, (aryl)C₀-C₂alkyl-, and y is 0, 1, 2, 3, 4, or 5.

R¹⁵ is C₁-C₆alkyl, C₃-C₆ cycloalkyl, —C₁-C₃alkyl-O—C₁-C₅alkoxy, or —C(R^{9a})(R^{9b})CH₂OR⁵.

R¹⁶ is hydrogen, deuterium, C₁₋₃alkyl (for example, methyl) or C₃₋₅ cycloalkyl.

R^{17a} and R^{17b} are independently hydrogen, deuterium, C₁-C₆alkyl (including C₁-C₃ alkyl), halogen, C₃-C₆ cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid, or its isomer; each of which is optionally substituted. The R^{17a} and R^{17b} substituents independently include but are not limited to those corresponding to the R^{17a} and R^{17b} positions found in natural amino acids (or their D-counterpart) and non-proteogenic amino acids, such as serine, threonine, asparagine, glutamine, cysteine, selenocysteine, glycine (e.g., hydrogen), alanine, valine, isoleucine, methionine, phenylalanine, tyrosine, tryptophan, ornithine, glutamine, arginine, histidine, proline, hydroxyproline, selenomethionine, lanthionine, 2-aminoisobutyric acid or dehydroalanine (i.e., R^{17a} or R^{17b} is an exo-double bond), with optional protection of functional groups such as hydroxyl, amino, thiol, etc. In an alternative embodiment, R^{17a} and R^{17b} can together be a cycloalkyl or heterocyclic group, which may be optionally substituted. Where the substituents in the amino acid require cyclization, the formula envisions such modifications, such as that required for proline (where R⁶ and R^{17a} or R^{17b} can come together to form the proline moiety). Alternatively, for example, R^{17a} and R^{17b} are taken together to form a 3- to 6-membered cycloalkyl ring or 3- to 6-membered heterocycloalkyl ring containing one heteroatom chosen from N, O, and S; each of which is optionally substituted. Where R^{17a} is hydrogen and R^{17b} is non-hydrogen, the amino acid moiety has L-stereochemistry similar to a naturally occurring amino acid. Where R^{17a} is non-hydrogen and R^{17b} is hydrogen, the amino acid moiety has D-stereochemistry which is not typically a naturally occurring amino acid. In this invention, the amino acid in the phosphoramidate of any of the disclosed structures can be either in the D or L configuration. If an L amino acid is depicted, it is envisioned that the D amino acid moiety can also be used.

R¹⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (3- to 6-membered heterocycloalkyl)C₀-C₄alkyl-, or (heteroaryl)C₀-C₄alkyl- which is optionally substituted.

R¹⁹ and R²⁰ are independently hydrogen, deuterium, alkyl, cycloalkyl, aryl, heteroaryl, heterocyclic, -alkylaryl (including benzyl); which includes, but is not limited to, phenyl (including but not limited to halophenyl, cyanophenyl, and alkylphenyl) or naphthyl (for example, 1 or 2-naphthyl); or a side chain of an amino acid ester. In an alternative embodiment, R¹⁹ and R²⁰ can together be a cycloalkyl or heterocyclic group, which may be optionally substituted.

R^{21a} and R^{21b} are independently hydrogen, deuterium, C₁₋₆alkyl, C₃-C₆ cycloalkyl, and in one embodiment, when taken together, can form a 3 to 6 membered ring.

m is 0, 1, 2, or 3.

n is 0, 1, 2, or 3.

m+n=3; and

X is S or O

“Nucleoside” refers to a pyrimidine or purine nucleoside, or in an alternative embodiment a nucleoside with a nonnaturally occurring heteroaryl or heteroaromatic moiety used in place of the purine or pyrimidine, that can have a natural or modified sugar, including as described herein, which achieves the desired therapeutic effect. The 2′-position of the nucleoside can optionally have one or two non-hydrogen substituents which are selected to provide the desired activity to the 5′-deuterated nucleos(t)ide for the target indication. For example, the 2′-position of the nucleoside can have, for example, one or two non-hydrogen substituents selected from, for example, an alkyl, including for example CH₃, CD₃, C(H)ₘ(D)ₙ a halogen, for example F, Cl, I, or Br, a hydroxy, or other 2′ substituent or combinations of substituents that provide activity for the target indication. In one embodiment, the 2-α position (the “down” group) is halogen (F, Cl, I, or Br), hydroxyl, cyano or hydrogen. In one embodiment, the 2-β position (the “up” group) is CH₃, CD₃, C(H)ₘ(D)ₙ, hydrogen, or F. The pyrimidine or purine base of the nucleoside can be a natural base or a synthetic base. Examples of such purine, pyrimidine, and heteroaryl or heterocyclic bases include, but are not limited to, adenine, N⁶-alkylpurine, N⁶-acylpurine (wherein acyl is C(O)(alkyl, aryl, alkylaryl-, or arylalkyl-), N⁶-benzylpurine, N⁶-halopurine, N⁶-vinylpurine, N⁶-acetylenic purine, N⁶-acyl purine, N⁶-hydroxyalkyl purine, N⁶-alkylaminopurine, N⁶-thioalkyl purine, N²-alkylpurine, N²-alkyl-6-thiopurine, thymine, cytosine, 5-fluorocytosine, 5-methylcytosine, 6-azapyrimidine, including 6-azacytosine, 2- and/or 4-mercaptopyrimidine, uracil, 5-halouracil, including 5-fluorouracil, C⁵-alkylpyrimidine, C⁵-benzylpyrimidine, C⁵-halopyrimidine, C⁵-vinylpyrimidine, C⁵-acetylenic pyrimidine, C⁵-acyl pyrimidine, C⁵ hydroxyalkyl purine, C⁵-amidopyrimidine; C⁵-cyanopyrimidine, C⁵-iodopyrimidine, C⁶-iodo-pyrimidine, C⁵— Br-vinyl pyrimidine, C⁶—Br-vinyl pyrimidine, C⁵-nitropyrimidine, C⁵-amino-pyrimidine, N²-alkylpurine, N²-alkyl-6-thiopurine, 5-azacytidinyl, 5-azaumcilyl, triazolopyridinyl, imidazolopyridinyl, pyrrolopyrimidinyl, guanine, hypoxanthine, 2,6-diaminopurine, and 6-chloropurine, oxazolyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzo[d]oxazolyl, benzofuranyl, benzothiazolyl, benzothiophenyl, benzoxadiazolyl, dihydrobenzodioxynyl, furanyl, imidazolyl, indolyl, and isoxazolyl. Functional oxygen and nitrogen groups on the base can be protected as necessary or desired. Suitable protecting groups are well known to those skilled in the art, and include trimethylsilyl, dimethylhexylsilyl, t-butyldimethylsilyl, and t-butyldiphenylsilyl, trityl, alkyl groups, and acyl groups such as acetyl and propionyl, methanesulfonyl, and p-toluenesulfonyl. In one embodiment, the “nucleoside” as that term is used in Table 5 is:

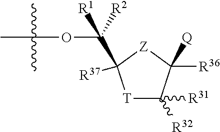

wherein;

Z is O, S or CH═CH₂;

T is O, S or CR³³R³⁴;

R¹ and R² are as defined above;

R³¹ is H, OH, C₂₋₄ alkenyl, C₂₋₄ alkynyl, C₁₋₄ alkyl-O—, C₁₋₄ alkyl, C₁₋₄ haloalkyl, or C₁₋₄ alkyl substituted with from 1 to 4 substituents each of which is independently OH or amino;

R³² is H, OH, amino, cyano, azido, halogen, C₁₋₄ alkyl-O—, C₁₋₄ alkyl, C₁₋₄ haloalkyl, or C₁₋₄ alkyl substituted with from 1 to 4 substituents each of which is independently OH or amino;

R³³ is OR⁴, H, OH, cyano, azido, halogen, amino, C₁₋₄ alkyl-O—, C₂₋₄ alkenyl, C₂₋₄ alkynyl, C₁₋₄ alkyl, C₁₋₄ haloalkyl, or C₁₋₄ alkyl substituted with from 1 to 4 substituents each of which is independently OH or amino;

R³⁴ is H, OH, cyano, azido, halogen, amino, C₁₋₄ alkyl-O—, C₂₋₄ alkenyl, C₂₋₄ alkynyl, C₁₋₄ alkyl, C₁₋₄ haloalkyl, or C₁₋₄ alkyl substituted with from 1 to 4 substituents each of which is independently OH or amino;

R³⁶ is H, methyl, hydroxymethyl, or fluoromethyl;

R³⁷ is H, halogen, azido, heteroaryl or cyano;

Q is:

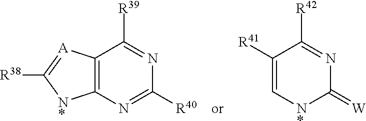

wherein:

* denotes the point of attachment of Q to the C-1 carbon of the furanose ring;

A is N or C—R^{w};
W is O or S;

R³⁸ is H, C₁₋₄ alkyl, C₂₋₄ alkenyl, C₂₋₄ alkynyl, halogen, cyano, carboxy, C₁₋₄ alkyloxycarbonyl, azido, amino, C₁₋₄alkylamino, di(C₁₋₄ alkyl)amino, OH, C₁₋₆ alkyl-O—, C₁₋₄ alkyl-S—, C₁₋₆ alkyl-SO₂—, aminomethyl, or (C₁₋₄alkyl)₁₋₂aminomethyl;

R³⁹ and R⁴² are each independently H, OH, mercapto, halogen, C₁₋₄ alkyl-O—, C₁₋₄ alkyl-S—, amino, C₁₋₄ alkylamino, di(C₁₋₄ alkyl)amino, C₃₋₆ cycloalkylamino, di(C₃₋₆ cycloalkyl)amino, or an amino acyl residue of formula:

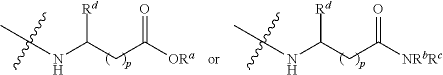

wherein p is an integer equal to zero, 1, 2, 3 or 4;

R⁴⁰ is H, OH, mercapto, halogen, C₁₋₄ alkyl-O—, C₁₋₄ alkyl-S—, amino, C₁₋₄ alkylamino, di(C₁₋₄ alkyl)amino, C₃₋₆ cycloalkylamino, di(C₃₋₆ cycloalkyl)amino, phenyl-C₁₋₂ alkylamino, or C₁₋₄ alkylC(═O)NH—;

R⁴¹ is H, C₁₋₆ alkyl, C₂₋₆ alkenyl optionally substituted with halogen, C₂₋₆ alkynyl, CF₃, or halogen;

R^{a}, R^{b}, and R^{c} are each independently H or C₁₋₆ alkyl;

R^{d} is H, C₁₋₄ alkyl, phenyl-C₁₋₂ alkyl, or phenyl;

R^{w} is H, cyano, nitro, NHC(═O)NH₂, C(═O)NR^{x}R^{x}, CSNR^{x}R^{x}, C(═O)OR^{x}, C(═NH)NH₂, OH, C₁₋₃ alkoxy, amino, C₁₋₄ alkylamino, di(C₁₋₄ alkyl)amino, halogen, C₂-C₆ alkenyl, C₂-C₆ alkynyl, C₁₋₃ alkyl, or C₁₋₃ alkyl substituted with from one to three groups independently selected from aryl, halogen, amino, OH, carboxy, and C₁₋₃ alkyl-O—; and each R^{x} is independently H or C₁₋₆alkyl.

In one embodiment, the parent nucleoside/nucleotide is a known compound with activity against a target disease such as hepatitis C or B, HIV, RSV, flavivirus, or another viral or other disorders, for example neoplastic disorders, disclosed herein.

In some embodiments, structures are provided including 1-1, 2-1, and 3-1, wherein R¹ and R² are independently D or H, wherein at least one of R¹ and R² is D, and typically both R¹ and R² are D, R³ is H or D, R⁴ is hydrogen, and R⁵ and R¹⁰ are independently alkyl, cycloalkyl, aryl, -alkylaryl, -alkyl(heterocycle), or heterocycle, any of which can be optionally substituted.

In some embodiments, structures are provided including 1-1, 2-1, and 3-1, wherein R¹ and R² are independently D or H, wherein at least one of R¹ and R² is D, and typically both R¹ and R² are D, R³ is H or D, R⁴ is hydrogen, and R⁵ and R¹⁰ are independently aryl or -alkylaryl, either of which can be optionally substituted.

In some embodiments, structures are provided including 1-2, 2-2, and 3-2, wherein R¹ and R² are independently D or H, wherein at least one of R¹ and R² is D, and typically both R¹ and R² are D, R³ is H or D, and R¹⁰ is alkyl, cycloalkyl, aryl, -alkylaryl, -alkyl(heterocycle), or heterocycle, any of which can be optionally substituted.

In further embodiments, structures are provided including 1-2, 2-2, and 3-2, wherein R¹ and R² are independently D or H, wherein at least one of R¹ and R² is D, and typically both R¹ and R² are D, R³ is H or D, and R¹⁰ is C₁-C₆alkyl.

In further embodiments, structures are provided including 1-2, 2-2, and 3-2, wherein R¹ and R² are independently D or H, wherein at least one of R¹ and R² is D, and typically both R¹ and R² are D, R³ is H or D, and R¹⁰ is isopropyl.

In other embodiments, structures are provided including 1-3, 2-3, and 3-3, wherein R¹ and R² are independently D or H, wherein at least one of R¹ and R² is D, and typically both R¹ and R² are D, R³ is H or D, R⁵ is alkyl, cycloalkyl, aryl, -alkylaryl, -alkyl(heterocycle), or heterocycle, any of which can be optionally substituted, and R^{9a} and R^{9b} are C₁-C₃alkyl.

In some embodiments, structures are provided including 1-3, 2-3, and 3-3, wherein R¹ and R² are independently D or H, wherein at least one of R¹ and R² is D, and typically both R¹ and R² are D, R³ is H or D, R⁵ is C₁-C₆alkyl, and R^{9a} and R^{9b} are C₁-C₃alkyl.

In some embodiments, structures are provided including 1-3, 2-3, and 3-3, wherein R¹ and R² are independently D or H, wherein at least one of R¹ and R² is D, and typically both R¹ and R² are D, R³ is H or D, R⁵ is n-propyl, and R^{9a} and R^{9b} are C₁-C₃alkyl.

In other embodiments, structures are provided including 1-3, 2-3, and 3-3, wherein R¹ and R² are independently D or H, wherein at least one of R¹ and R² is D, and typically both R¹ and R² are D, R³ is H or D, R⁵ is alkyl, cycloalkyl, aryl, -alkyl(aryl), -alkyl(heterocycle), or heterocycle, any of which can be optionally substituted, and R^{9a} and R^{9b} are methyl.

In other embodiments, structures are provided including 1-3, 2-3, and 3-3, wherein R¹ and R² are independently D or H, wherein at least one of R¹ and R² is D, and typically both R¹ and R² are D, R³ is H or D, R⁵ is C₁-C₆alkyl, and R^{9a} and R^{9b} are methyl.

In some embodiments, structures are provided including 1-3, 2-3, and 3-3, wherein R¹ and R² are independently D or H, wherein at least one of R¹ and R² is D, and typically both R¹ and R² are D, R³ is H or D, R⁵ is n-propyl, and R^{9a} and R^{9b} are methyl.

In other embodiments, structures are provided including 1-4, 2-4, and 3-4, wherein R¹ and R² are independently D or H, wherein at least one of R¹ and R² is D, and typically both R¹ and R² are D, R³ is H or D, R⁸ is C₁-C₆alkyl or C₃-C₆cycloalkyl, R⁶ and R^{7a} are H, and R^{7b} is an amino acid side chain.

In some embodiments, structures are provided including 1-4, 2-4, and 3-4, wherein R¹ and R² are independently D or H, wherein at least one of R¹ and R² is D, and typically both R¹ and R² are D, R³ is H or D, R⁸ is C₁-C₆alkyl or C₃-C₆cycloalkyl, R⁶ and R^{7a} are H, and R^{7b} is C₁-C₆alkyl.

In some embodiments, structures are provided including 1-4, 2-4, and 3-4, wherein R¹ and R² are independently D or H, wherein at least one of R¹ and R² is D, and typically both R¹ and R² are D, R³ is H or D, R⁸ is -alkylaryl, R⁶ and R^{7a} are H, and R^{7b} is an amino acid side chain.

In further embodiments, structures are provided including 1-4, 2-4, and 3-4, wherein R¹ and R² are independently D or H, wherein at least one of R¹ and R² is D, and typically both R¹ and R² are D, R³ is H or D, R⁸ is -alkylaryl, R⁶ and R^{7a} are H, and R^{7b} is C₁-C₆alkyl.

In other embodiments, structures are provided including 1-4, 2-4, and 3-4, wherein R¹ and R² are independently D or H, wherein at least one of R¹ and R² is D, and typically both R¹ and R² are D, R³ is H or D, R⁸ is isopropyl, R⁶ and R^{7a} are H, and R^{7b} is an amino acid side chain.

In some embodiments, structures are provided including 1-4, 2-4, and 3-4, wherein R¹ and R² are independently D or H, wherein at least one of R¹ and R² is D, and typically both R¹ and R² are D, R³ is H or D, R⁸ is isopropyl, R⁶ and R^{7a} are H, and R^{7b} is methyl.

In some embodiments, structures are provided including 1-4, 2-4, and 3-4, wherein R¹ and R² are independently D or H, wherein at least one of R¹ and R² is D, and typically both R¹ and R² are D, R³ is H or D, R⁸ is benzyl, R⁶ and R^{7a} are H, and R^{7b} is an amino acid side chain.

In further embodiments, structures are provided including 1-4, 2-4, and 3-4, wherein R¹ and R² are independently D or H, wherein at least one of R¹ and R² is D, and typically both R¹ and R² are D, R³ is H or D, R⁸ is benzyl, R⁶ and R^{7a} are H, and R^{7b} is methyl.

In other embodiments, structures are provided including 1-4, 2-4, and 3-4, wherein R¹ and R² are independently D or H, wherein at least one of R¹ and R² is D, and typically both R¹ and R² are D, R³ is H or D, R⁸ is C₁-C₆alkyl or C₃-C₆cycloalkyl, R⁶ and R^{7b} are H, and R^{7a} is an amino acid side chain.

In some embodiments, structures are provided including 1-4, 2-4, and 3-4, wherein R¹ and R² are independently D or H, wherein at least one of R¹ and R² is D, and typically both R¹ and R² are D, R³ is H or D, R⁸ is C₁-C₆alkyl or C₃-C₆cycloalkyl, R⁶ and R^{7b} are H, and R^{7a} is C₁-C₆alkyl.

In some embodiments, structures are provided including 1-4, 2-4, and 3-4, wherein R¹ and R² are independently D or H, wherein at least one of R¹ and R² is D, and typically both R¹ and R² are D, R³ is H or D, R⁸ is -alkylaryl, R⁶ and R^{7b} are H, and R^{7a} is an amino acid side chain.

In further embodiments, structures are provided including 1-4, 2-4, and 3-4, wherein R¹ and R² are independently D or H, wherein at least one of R¹ and R² is D, and typically both R¹ and R² are D, R³ is H or D, R⁸ is -alkylaryl, R⁶ and R^{7b} are H, and R^{7a} is C₁-C₆alkyl.

In other embodiments, structures are provided including 1-4, 2-4, and 3-4, wherein R¹ and R² are independently D or H, wherein at least one of R¹ and R² is D, and typically both R¹ and R² are D, R³ is H or D, R⁸ is isopropyl, R⁶ and R^{7b} are H, and R^{7a} is an amino acid side chain.

In some embodiments, structures are provided including 1-4, 2-4, and 3-4, wherein R¹ and R² are independently D or H, wherein at least one of R¹ and R² is D, and typically both R¹ and R² are D, R³ is H or D, R⁸ is isopropyl, R⁶ and R^{7b} are H, and R^{7a} is methyl.

In some embodiments, structures are provided including 1-4, 2-4, and 3-4, wherein R¹ and R² are independently D or H, wherein at least one of R¹ and R² is D, and typically both R¹ and R² are D, R³ is H or D, R⁸ is benzyl, R⁶ and R^{7b} are H, and R^{7a} is an amino acid side chain.

In further embodiments, structures are provided including 1-4, 2-4, and 3-4, wherein R¹ and R² are independently D or H, wherein at least one of R¹ and R² is D, and typically both R¹ and R² are D, R³ is H or D, R⁸ is benzyl, R⁶ and R^{7b} are H, and R^{7a} is methyl.

In some embodiments, structures are provided including 1-5, 2-5, and 3-5, wherein R¹ and R² are independently D or H, wherein at least one of R¹ and R² is D, and typically both R¹ and R² are D, R³ is H or D, R⁴ is hydrogen, R⁵ is alkyl, cycloalkyl, aryl, -alkylaryl, -alkyl(heterocycle), or heterocycle, any of which can be optionally substituted, R¹¹ and R¹² are C₁-C₂₂alkyl, and X is S.

In some embodiments, structures are provided including 1-5, 2-5, and 3-5, wherein R¹ and R² are independently D or H, wherein at least one of R¹ and R² is D, and typically both R¹ and R² are D, R³ is H or D, R⁴ is hydrogen, R⁵ is aryl, R¹¹ and R¹² are C₁-C₂₂alkyl, and X is S.

In some embodiments, structures are provided including 1-5, 2-5, and 3-5, wherein R¹ and R² are independently D or H, wherein at least one of R¹ and R² is D, and typically both R¹ and R² are D, R³ is H or D, R⁴ is hydrogen, R⁵ is alkyl, cycloalkyl, aryl, -alkylaryl, -alkyl(heterocycle), or heterocycle, any of which can be optionally substituted, R¹¹ is C₁₀H₂₁, R¹² is C₁₂H₂₅, and X is S.

In some embodiments, structures are provided including 1-5, 2-5, and 3-5, wherein R¹ and R² are independently D or H, wherein at least one of R¹ and R² is D, and typically both R¹ and R² are D, R³ is H or D, R⁴ is hydrogen, R⁵ is phenyl, R¹¹ is C₁₀H₂₁, R¹² is C₁₂H₂₅ and X is S.

In some embodiments, structures are provided including 1-6, 2-6, and 3-6, wherein R¹ and R² are independently D or H, wherein at least one of R¹ and R² is D, and typically both R¹ and R² are D, R³ is H or D, R⁴ is hydrogen, and R¹⁵ is C₁-C₆alkyl.

In some embodiments, structures are provided including 1-6, 2-6, and 3-6, wherein R¹ and R² are independently D or H, wherein at least one of R¹ and R² is D, and typically both R¹ and R² are D, R³ is H or D, R⁴ is hydrogen, and R¹⁵ is —C₁-C₃alkyl-O—C₁-C₅alkoxy.

In some embodiments, structures are provided including 1-6, 2-6, and 3-6, wherein R¹ and R² are independently D or H, wherein at least one of R¹ and R² is D, and typically both R¹ and R² are D, R³ is H or D, R⁴ is hydrogen, and R¹⁵ is t-butyl.

In other embodiments, structures are provided including 1-7, 2-7, and 3-7, wherein R¹ and R² are independently D or H, wherein at least one of R¹ and R² is D, and typically both R¹ and R² are D, R³ is H or D, R⁴ is acyl, R¹³ is hydrogen, R¹⁴ is halogen, and y=1, 2, 3, 4, or 5.

In further embodiments, structures are provided including 1-7, 2-7, and 3-7, wherein R¹ and R² are independently D or H, wherein at least one of R¹ and R² is D, and typically both R¹ and R² are D, R³ is H or D, R⁴ and R¹³ are hydrogen, R¹⁴ is halogen, and y=1, 2, 3, 4, or 5.

In other embodiments, structures are provided including 1-7, 2-7, and 3-7, wherein R¹ and R² are independently D or H, wherein at least one of R¹ and R² is D, and typically both R¹ and R² are D, R³ is H or D, R⁴ is acyl, R¹³ is hydrogen, R¹⁴ is Cl, and y=1.

In further embodiments, structures are provided including 1-7, 2-7, and 3-7, wherein R¹ and R² are independently D or H, wherein at least one of R¹ and R² is D, and typically both R¹ and R² are D, R³ is H or D, R⁴ and R¹³ are hydrogen, R¹⁴ is Cl, and y=1.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In further embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In other embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7a} are H, R^{7b} is an amino acid side chain, and R⁵ is aryl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7a} are H, R^{7b} is C₁-C₆alkyl, and R⁵ is aryl.

In other embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7a} are H, R^{7b} is an amino acid side chain, and R⁵ is aryl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7a} are H, R^{7b} is C₁-C₆alkyl, and R⁵ is aryl.

In some embodiments, structures are provided including 1-8, 2-8, and 3-8, wherein R¹, R², and R³ are D, R⁴ is hydrogen, R⁸ is C₁-C₆alkyl or C₃-C₆cycloalkyl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 1-8, 2-8, and 3-8, wherein R¹, R², and R³ are D, R⁴ is hydrogen, R⁸ is C₁-C₆alkyl or C₃-C₆cycloalkyl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In further embodiments, structures are provided including 1-8, 2-8, and 3-8, wherein R¹, R², and R³ are D, R⁴ is hydrogen, R⁸ is -alkylaryl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 1-8, 2-8, and 3-8, wherein R¹, R², and R³ are D, R⁴ is hydrogen, R⁸ is -alkylaryl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In other embodiments, structures are provided including 1-8, 2-8, and 3-8, wherein R¹, R², and R³ are D, R⁴ is hydrogen, R⁸ is -alkylaryl, R⁶ and R^{7a} are H, R^{7b} is an amino acid side chain, and R⁵ is aryl.

In some embodiments, structures are provided including 1-8, 2-8, and 3-8, wherein R¹, R², and R³ are D, R⁴ is hydrogen, R⁸ is -alkylaryl, R⁶ and R^{7a} are H, R^{7b} is C₁-C₆alkyl, and R⁵ is aryl.

In other embodiments, structures are provided including 1-8, 2-8, and 3-8, wherein R¹, R², and R³ are D, R⁴ is hydrogen, R⁸ is C₁-C₆alkyl or C₃-C₆cycloalkyl, R⁶ and R^{7a} are H, R^{7b} is an amino acid side chain, and R⁵ is aryl.

In some embodiments, structures are provided including 1-8, 2-8, and 3-8, wherein R¹, R², and R³ are D, R⁴ is hydrogen, R⁸ is C₁-C₆alkyl or C₃-C₆cycloalkyl, R⁶ and R^{7a} are H, R^{7b} is C₁-C₆alkyl, and R⁵ is aryl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7a} are H, R^{7b} is an amino acid side chain, and R⁵ is phenyl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7a} are H, R^{7b} is methyl, and R⁵ is phenyl.

In further embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7a} are H, R^{7b} is an amino acid side chain, and R⁵ is phenyl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7a} are H, R^{7b} is methyl, and R⁵ is phenyl.

In other embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7a} are H, R^{7b} is an amino acid side chain, and R⁵ is 1-naphthyl or 2-naphthyl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7a} are H, R^{7b} is methyl, and R⁵ is 1-naphthyl or 2-naphthyl.

In other embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7a} are H, R^{7b} is an amino acid side chain, and R⁵ is 1-naphthyl or 2-naphthyl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7a} are H, R^{7b} is methyl, and R⁵ is 1-naphthyl or 2-naphthyl.

In some embodiments, structures are provided including 1-8, 2-8, and 3-8, wherein R¹, R², and R³ are D, R⁴ is hydrogen, R⁸ is isopropyl, R⁶ and R^{7a} are H, R^{7b} is an amino acid side chain, and R⁵ is phenyl.

In some embodiments, structures are provided including 1-8, 2-8, and 3-8, wherein R¹, R², and R³ are D, R⁴ is hydrogen, R⁸ is isopropyl, R⁶ and R^{7a} are H, R^{7b} is methyl, and R⁵ is phenyl.

In further embodiments, structures are provided including 1-8, 2-8, and 3-8, wherein R¹, R², and R³ are D, R⁴ is hydrogen, R⁸ is benzyl, R⁶ and R^{7a} are H, R^{7b} is an amino acid side chain, and R⁵ is phenyl.

In some embodiments, structures are provided including 1-8, 2-8, and 3-8, wherein R¹, R², and R³ are D, R⁴ is hydrogen, R⁸ is benzyl, R⁶ and R^{7a} are H, R^{7b} is methyl, and R⁵ is phenyl.

In other embodiments, structures are provided including 1-8, 2-8, and 3-8, wherein R¹, R², and R³ are D, R⁴ is hydrogen, R⁸ is benzyl, R⁶ and R^{7a} are H, R^{7b} is an amino acid side chain, and R⁵ is 1-naphthyl or 2-naphthyl.

In some embodiments, structures are provided including 1-8, 2-8, and 3-8, wherein R¹, R², and R³ are D, R⁴ is hydrogen, R⁸ is benzyl, R⁶ and R^{7a} are H, R^{7b} is methyl, and R⁵ is 1-naphthyl or 2-naphthyl.

In other embodiments, structures are provided including 1-8, 2-8, and 3-8, wherein R¹, R², and R³ are D, R⁴ is hydrogen, R⁸ is isopropyl, R⁶ and R^{7a} are H, R^{7b} is an amino acid side chain, and R⁵ is 1-naphthyl or 2-naphthyl.

In some embodiments, structures are provided including 1-8, 2-8, and 3-8, wherein R¹, R², and R³ are D, R⁴ is hydrogen, R⁸ is isopropyl, R⁶ and R^{7a} are H, R^{7b} is methyl, and R⁵ is 1-naphthyl or 2-naphthyl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In further embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In other embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7b} are H, R^{7a} is an amino acid side chain, and R⁵ is aryl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7b} are H, R^{7a} is C₁-C₆alkyl, and R⁵ is aryl.

In other embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7b} are H, R^{7a} is an amino acid side chain, and R⁵ is aryl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7b} are H, R^{7a} is C₁-C₆alkyl, and R⁵ is aryl.

In some embodiments, structures are provided including 1-8, 2-8, and 3-8, wherein R¹, R², and R³ are D, R⁴ is hydrogen, R⁸ is C₁-C₆alkyl or C₃-C₆cycloalkyl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 1-8, 2-8, and 3-8, wherein R¹, R², and R³ are D, R⁴ is hydrogen, R⁸ is C₁-C₆alkyl or C₃-C₆cycloalkyl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In further embodiments, structures are provided including 1-8, 2-8, and 3-8, wherein R¹, R², and R³ are D, R⁴ is hydrogen, R⁸ is -alkylaryl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 1-8, 2-8, and 3-8, wherein R¹, R², and R³ are D, R⁴ is hydrogen, R⁸ is -alkylaryl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In other embodiments, structures are provided including 1-8, 2-8, and 3-8, wherein R¹, R², and R³ are D, R⁴ is hydrogen, R⁸ is -alkylaryl, R⁶ and R^{7b} are H, R^{7a} is an amino acid side chain, and R⁵ is aryl.

In some embodiments, structures are provided including 1-8, 2-8, and 3-8, wherein R¹, R², and R³ are D, R⁴ is hydrogen, R⁸ is -alkylaryl, R⁶ and R^{7b} are H, R^{7a} is C₁-C₆alkyl, and R⁵ is aryl.

In other embodiments, structures are provided including 1-8, 2-8, and 3-8, wherein R¹, R², and R³ are D, R⁴ is hydrogen, R⁸ is C₁-C₆alkyl or C₃-C₆cycloalkyl, R⁶ and R^{7b} are H, R^{7a} is an amino acid side chain, and R⁵ is aryl.

In some embodiments, structures are provided including 1-8, 2-8, and 3-8, wherein R¹, R², and R³ are D, R⁴ is hydrogen, R⁸ is C₁-C₆alkyl or C₃-C₆cycloalkyl, R⁶ and R^{7b} are H, R^{7a} is C₁-C₆alkyl, and R⁵ is aryl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7b} are H, R^{7a} is an amino acid side chain, and R⁵ is phenyl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7b} are H, R^{7a} is methyl, and R⁵ is phenyl.

In further embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7b} are H, R^{7a} is an amino acid side chain, and R⁵ is phenyl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7b} are H, R^{7a} is methyl, and R⁵ is phenyl.

In other embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7b} are H, R^{7a} is an amino acid side chain, and R⁵ is 1-naphthyl or 2-naphthyl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7b} are H, R^{7a} is methyl, and R⁵ is 1-naphthyl or 2-naphthyl.

In other embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7b} are H, R^{7a} is an amino acid side chain, and R⁵ is 1-naphthyl or 2-naphthyl.

In some embodiments, structures are provided including 1-8, 2-8, and 3-8, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7b} are H, R^{7a} is methyl, and R⁵ is 1-naphthyl or 2-naphthyl.

In some embodiments, structures are provided including 1-8, 2-8, and 3-8, wherein R¹, R², and R³ are D, R⁴ is hydrogen, R⁸ is isopropyl, R⁶ and R^{7b} are H, R^{7a} is an amino acid side chain, and R⁵ is phenyl.

In some embodiments, structures are provided including 1-8, 2-8, and 3-8, wherein R¹, R², and R³ are D, R⁴ is hydrogen, R⁸ is isopropyl, R⁶ and R^{7b} are H, R^{7a} is methyl, and R⁵ is phenyl.

In further embodiments, structures are provided including 1-8, 2-8, and 3-8, wherein R¹, R², and R³ are D, R⁴ is hydrogen, R⁸ is benzyl, R⁶ and R^{7b} are H, R^{7a} is an amino acid side chain, and R⁵ is phenyl.

In some embodiments, structures are provided including 1-8, 2-8, and 3-8, wherein R¹, R², and R³ are D, R⁴ is hydrogen, R⁸ is benzyl, R⁶ and R^{7b} are H, R^{7a} is methyl, and R⁵ is phenyl.

In other embodiments, structures are provided including 1-8, 2-8, and 3-8, wherein R¹, R², and R³ are D, R⁴ is hydrogen, R⁸ is benzyl, R⁶ and R^{7b} are H, R^{7a} is an amino acid side chain, and R⁵ is 1-naphthyl or 2-naphthyl.

In some embodiments, structures are provided including 1-8, 2-8, and 3-8, wherein R¹, R², and R³ are D, R⁴ is hydrogen, R⁸ is benzyl, R⁶ and R^{7b} are H, R^{7a} is methyl, and R⁵ is 1-naphthyl or 2-naphthyl.

In other embodiments, structures are provided including 1-8, 2-8, and 3-8, wherein R¹, R², and R³ are D, R⁴ is hydrogen, R⁸ is isopropyl, R⁶ and R^{7b} are H, R^{7a} is an amino acid side chain, and R⁵ is 1-naphthyl or 2-naphthyl.

In some embodiments, structures are provided including 1-8, 2-8, and 3-8, wherein R¹, R², and R³ are D, R⁴ is hydrogen, R⁸ is isopropyl, R⁶ and R^{7b} are H, R^{7a} is methyl, and R⁵ is 1-naphthyl or 2-naphthyl.

In other embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7a} are H, R^{7b} is an amino acid side chain, R¹⁸ is C₁-C₆alkyl or C₃-C₆cycloalkyl, R¹⁶ and R^{17a} are H, and R^{17b} is an amino acid side chain.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7a} are H, R^{7b} is C₁-C₆alkyl, R¹⁸ is C₁-C₆alkyl or C₃-C₆cycloalkyl, R¹⁶ and R^{17a} are H, and R^{17b} is C₁-C₆alkyl.

In other embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7a} are H, R^{7b} is an amino acid side chain, R¹⁸ is -alkylaryl, R¹⁶ and R^{17a} are H, and R^{17b} is an amino acid side chain.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7a} are H, R^{17b} is C₁-C₆alkyl, R¹⁸ is -alkylaryl, R¹⁶ and R^{17a} are H, and R^{17b} is C₁-C₆alkyl.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7a} are H, R^{7b} is an amino acid side chain, R¹⁸ is -alkylaryl, R¹⁶ and R^{17a} are H, and R^{17b} is an amino acid side chain.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7a} are H, R^{7b} is C₁-C₆alkyl, R¹⁸ is -alkylaryl, R¹⁶ and R^{17a} are H, and R^{17b} is C₁-C₆alkyl.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7a} are H, R^{7b} is an amino acid side chain, R¹⁸ is C₁-C₆alkyl or C₃-C₆cycloalkyl, R¹⁶ and R^{17a} are H, and R^{17b} is an amino acid side chain.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7a} are H, R^{7b} is C₁-C₆alkyl, R¹⁸ is C₁-C₆alkyl or C₃-C₆cycloalkyl, R¹⁶ and R^{17a} are H, and R^{17b} is C₁-C₆alkyl.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7a} are H, R^{7b} is an amino acid side chain, R¹⁸ is isopropyl, R¹⁶ and R^{17a} are H, and R^{17b} is an amino acid side chain.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7a} are H, R^{7b} is methyl, R¹⁸ is isopropyl, R¹⁶ and R^{17a} are H, and R^{17b} is methyl.

In other embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7a} are H, R^{7b} is an amino acid side chain, R¹⁸ is benzyl, R¹⁶ and R^{17a} are H, and R^{17b} is an amino acid side chain.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7a} are H, R^{7b} is methyl, R¹⁸ is benzyl, R¹⁶ and R^{17a} are H, and R^{17b} is methyl.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7a} are H, R^{7b} is an amino acid side chain, R¹⁸ is benzyl, R¹⁶ and R^{17a} are H, and R^{17b} is an amino acid side chain.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7a} are H, R^{7b} is methyl, R¹⁸ is benzyl, R¹⁶ and R^{17a} are H, and R^{17b} is methyl.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7a} are H, R^{7b} is an amino acid side chain R¹⁸ is isopropyl, R¹⁶ and R^{17a} are H, and R^{17b} is an amino acid side chain.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7a} are H, R^{7b} is methyl, R¹⁸ is isopropyl, R¹⁶ and R^{17a} are H, and R^{17b} is methyl.

In other embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7b} are H, R^{7a} is an amino acid side chain, R¹⁸ is C₁-C₆alkyl or C₃-C₆cycloalkyl, R¹⁶ and R^{17b} are H, and R^{17a} is an amino acid side chain.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7b} are H, R^{7a} is C₁-C₆alkyl, R¹⁸ is C₁-C₆alkyl or C₃-C₆cycloalkyl, R¹⁶ and R^{17b} are H, and R^{17a} is C₁-C₆alkyl.

In other embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7b} are H, R^{7a} is an amino acid side chain, R¹⁸ is -alkylaryl, R¹⁶ and R^{17b} are H, and R^{17a} is an amino acid side chain.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7b} are H, R^{7a} is C₁-C₆alkyl, R¹⁸ is -alkylaryl, R¹⁶ and R^{17b} are H, and R^{17a} is C₁-C₆alkyl.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7b} are H, R^{7a} is an amino acid side chain, R¹⁸ is -alkylaryl, R¹⁶ and R^{17b} are H, and R^{17a} is an amino acid side chain.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is -alkylaryl, R⁶ and R^{7b} are H, R^{7a} is C₁-C₆alkyl, R¹⁸ is -alkylaryl, R¹⁶ and R^{17b} are H, and R^{17a} is C₁-C₆alkyl.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7b} are H, R^{7a} is an amino acid side chain, R¹⁸ is C₁-C₆alkyl or C₃-C₆cycloalkyl, R¹⁶ and R^{17b} are H, and R^{17a} is an amino acid side chain.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is C₁-C₆alkyl or C₃-C₆cycloalkyl, R⁶ and R^{7b} are H, R^{7a} is C₁-C₆alkyl, R¹⁸ is C₁-C₆alkyl or C₃-C₆cycloalkyl, R¹⁶ and R^{17b} are H, and R^{17a} is C₁-C₆alkyl.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7b} are H, R^{7a} is an amino acid side chain, R¹⁸ is isopropyl, R¹⁶ and R^{17b} are H, and R^{17a} is an amino acid side chain.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7b} are H, R^{7a} is methyl, R¹⁸ is isopropyl, R¹⁶ and R^{17b} are H, and R^{17a} is methyl.

In other embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7b} are H, R^{7a} is an amino acid side chain, R¹⁸ is benzyl, R¹⁶ and R^{17b} are H, and R^{17a} is an amino acid side chain.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7b} are H, R^{7a} is methyl, R¹⁸ is benzyl, R¹⁶ and R^{17b} are H, and R^{17a} is methyl.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7b} are H, R^{7a} is an amino acid side chain, R¹⁸ is benzyl, R¹⁶ and R^{17b} are H, and R^{17a} is an amino acid side chain.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is benzyl, R⁶ and R^{7b} are H, R^{7a} is methyl, R¹⁸ is benzyl, R¹⁶ and R^{17b} are H, and R^{17a} is methyl.

In further embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7b} are H, R^{7a} is an amino acid side chain, R¹⁸ is isopropyl, R¹⁶ and R^{17b} are H, and R^{17a} is an amino acid side chain.

In some embodiments, structures are provided including 1-9, 2-9, and 3-9, wherein R¹ and R² are independently D or H, wherein at least one of R¹ and R² is D, and typically both R¹ and R² are D, R³ is H or D, R⁴ is hydrogen, R⁸ is isopropyl, R⁶ and R^{7b} are H, R^{7a} is methyl, R¹⁸ is isopropyl, R¹⁶ and R^{17b} are H, and R^{17a} is methyl.

In further embodiments, structures are provided including 1-10, 2-10, and 3-10, wherein R¹ and R² are independently D or H, wherein at least one of R¹ and R² is D, and typically both R¹ and R² are D, R³ is H or D, R⁴ is hydrogen, R¹⁵ is —C₁-C₃alkyl-O—C₁-C₅alkoxy, R¹⁹ is H, and R²⁰ is -alkylaryl.

In further embodiments, structures are provided including 1-10, 2-10, and 3-10, wherein R¹ and R² are independently D or H, wherein at least one of R¹ and R² is D, and typically both R¹ and R² are D, R³ is H or D, R⁴ is hydrogen, R¹⁵ is C₁-C₆alkyl, R¹⁹ is H, and R²⁰ is -alkylaryl.

In further embodiments, structures are provided including 1-10, 2-10, and 3-10, wherein R¹ and R² are independently D or H, wherein at least one of R¹ and R² is D, and typically both R¹ and R² are D, R³ is H or D, R⁴ is hydrogen, R¹⁵ is isopropoxyl, R¹⁹ is H, and R²⁰ is -alkylaryl.

In further embodiments, structures are provided including 1-10, 2-10, and 3-10, wherein R¹ and R² are independently D or H, wherein at least one of R¹ and R² is D, and typically both R¹ and R² are D, R³ is H or D, R⁴ is hydrogen, R¹⁵ is —C₁-C₃alkyl-O—C₁-C₅alkoxy, R¹⁹ is H, and R²⁰ is benzyl.

In further embodiments, structures are provided including 1-10, 2-10, and 3-10, wherein R¹ and R² are independently D or H, wherein at least one of R¹ and R² is D, and typically both R¹ and R² are D, R³ is H or D, R⁴ is hydrogen, R¹⁵ is C₁-C₆alkyl, R¹⁹ is H, and R²⁰ is benzyl.

In further embodiments, structures are provided including 1-10, 2-10, and 3-10, wherein R¹ and R² are independently D or H, wherein at least one of R¹ and R² is D, and typically both R¹ and R² are D, R³ is H or D, R⁴ is hydrogen, R¹⁵ is isopropoxyl, R¹⁹ is H, and R²⁰ is benzyl.

In some embodiments, structures are provided including 1-11, 2-11, and 3-11, R¹ and R² are independently D or H, wherein at least one of R¹ and R² is D, and typically both R¹ and R² are D, R³ is H or D, R⁴, R^{21a}, and R^{21b} are hydrogen, and R¹⁵ is —C₁-C₃alkyl-O—C₁-C₅alkoxy.

In some embodiments, structures are provided including 1-11, 2-11, and 3-11, R¹ and R² are independently D or H, wherein at least one of R¹ and R² is D, and typically both R¹ and R² are D, R³ is H or D, R⁴, R^{21a}, and R^{21b} are hydrogen, and R¹⁵ is C₁-C₆alkyl.

In some embodiments, structures are provided including 1-11, 2-11, and 3-11, R¹ and R² are independently D or H, wherein at least one of R¹ and R² is D, and typically both R¹ and R² are D, R³ is H or D, R⁴, R^{21a}, and R^{21b} are hydrogen, and R¹⁵ is isopropoxyl.

In some embodiments, structures are provided including 1-11, 2-11, and 3-11, R¹ and R² are independently D or H, wherein at least one of R¹ and R² is D, and typically both R¹ and R² are D, R³ is H or D, R⁴, R^{21a}, and R^{21b} are hydrogen, and R¹⁵ is t-butyl.

In some embodiments, structures are provided including 1-12, 2-12, and 3-12, R¹ and R² are independently D or H, wherein at least one of R¹ and R² is D, and typically both R¹ and R² are D, R³ is H or D, R⁴, R^{21a}, and R^{21b} are hydrogen, and R¹⁵ is —C₁-C₃alkyl-O—C₁-C₅alkoxy, R¹⁹ is H, and R²⁰ is -alkylaryl.

In some embodiments, structures are provided including 1-12, 2-12, and 3-12, R¹ and R² are independently D or H, wherein at least one of R¹ and R² is D, and typically both R¹ and R², 21a are D, R³ is H or D, R⁴, R^{21a}, and R^{21b} are hydrogen, and R¹⁵ is C₁-C₆alkyl, R¹⁹ is H, and R²⁰ is -alkylaryl.

In some embodiments, structures are provided including 1-12, 2-12, and 3-12, R¹ and R² are independently D or H, wherein at least one of R¹ and R² is D, and typically both R¹ and R² are D, R³ is H or D, R⁴, R^{21a}, and R^{21b} are hydrogen, and R¹⁵ is isopropoxyl, R¹⁹ is H, and R²⁰ is benzyl.

In some embodiments, structures are provided including 1-12, 2-12, and 3-12, R¹ and R² are independently D or H, wherein at least one of R¹ and R² is D, and typically both R¹ and R², 21a are D, R³ is H or D, R⁴, R^{21a}, and R^{21b} are hydrogen, and R¹⁵ is t-butyl, R¹⁹ is H, and R²⁰ is benzyl.

In some embodiments, structures are provided including 1-12, 2-12, and 3-12, R¹ and R² are independently D or H, wherein at least one of R¹ and R² is D, and typically both R¹ and R² are D, R³ is H or D, R⁴, R^{21a}, and R^{21b} are hydrogen, and R¹⁵ is isopropoxyl, R¹⁹ is H, and R²⁰ is -alkylaryl.

In some embodiments, structures are provided including 1-12, 2-12, and 3-12, R¹ and R² are independently D or H, wherein at least one of R¹ and R² is D, and typically both R¹ and R², 21a are D, R³ is H or D, R⁴, R^{21a}, and R^{21b} are hydrogen, and R¹⁵ is t-butyl, R¹⁹ is H, and R²⁰ is -alkylaryl.

In some embodiments, structures are provided including 1-12, 2-12, and 3-12, R¹ and R² are independently D or H, wherein at least one of R¹ and R² is D, and typically both R¹ and R² are D, R³ is H or D, R⁴, R^{21a}, and R^{21b} are hydrogen, and R¹⁵ is —C₁-C₃alkyl-O—C₁-C₅alkoxy, R¹⁹ is H, and R²⁰ is benzyl.

In some embodiments, structures are provided including 1-12, 2-12, and 3-12, R¹ and R² are independently D or H, wherein at least one of R¹ and R² is D, and typically both R¹ and R², 21a are D, R³ is H or D, R⁴, R^{21a}, and R^{21b} are hydrogen, and R¹⁵ is C₁-C₆alkyl, R¹⁹ is H, and R²⁰ is benzyl.

In some embodiments, structures are provided including 4-1 and 5-1, wherein R¹ and R² are independently D or H, wherein at least one of R¹ and R² is D, and typically both R¹ and R² are D, R⁴ is hydrogen, and R⁵ and R¹⁰ are independently alkyl, cycloalkyl, aryl, -alkylaryl, -alkyl(heterocycle), or heterocycle, any of which can be optionally substituted.

In some embodiments, structures are provided including 4-1 and 5-1, wherein R¹ and R² are independently D or H, wherein at least one of R¹ and R² is D, and typically both R¹ and R² are D, R⁴ is hydrogen, and R⁵ and R¹⁰ are independently aryl or -alkylaryl, either of which can be optionally substituted.

In some embodiments, structures are provided including 4-2 and 5-2, wherein R¹ and R² are independently D or H, wherein at least one of R¹ and R² is D, and typically both R¹ and R² are D, and R¹⁰ is alkyl, cycloalkyl, aryl, -alkylaryl, -alkyl(heterocycle), or heterocycle, any of which can be optionally substituted.

In some embodiments, structures are provided including 4-2 and 5-2, wherein R¹ and R² are independently D or H, wherein at least one of R¹ and R² is D, and typically both R¹ and R² are D, and R¹⁰ is C₁-C₆alkyl.

In some embodiments, structures are provided including 4-2 and 5-2, wherein R¹ and R² are independently D or H, wherein at least one of R¹ and R² is D, and typically both R¹ and R² are D, and R¹⁰ is isopropyl.

In some embodiments, structures are provided including 4-3 and 5-3, wherein R¹ and R² are independently D or H, wherein at least one of R¹ and R² is D, and typically both R¹ and R² are D, R⁵ is alkyl, cycloalkyl, aryl, -alkylaryl, -alkyl(heterocycle), or heterocycle, any of which can be optionally substituted, and R^{9a} and R^{9b} are C₁-C₃alkyl.

In some embodiments, structures are provided including 4-3 and 5-3, wherein R¹ and R² are independently D or H, wherein at least one of R¹ and R² is D, and typically both R¹ and R² are D, R⁵ is C₁-C₆alkyl, and R^{9a} and R^{9b} are C₁-C₃alkyl.

In some embodiments, structures are provided including 4-3 and 5-3, wherein R¹ and R² are independently D or H, wherein at least one of R¹ and R² is D, and typically both R¹ and R² are D, R⁵ is n-propyl, and R^{9a} and R^{9b} are C₁-C₃alkyl.

In some embodiments, structures are provided including 4-3 and 5-3, wherein R¹ and R² are independently D or H, wherein at least one of R¹ and R² is D, and typically both R¹ and R² are D, R⁵ is alkyl, cycloalkyl, aryl, -alkylaryl, -alkyl(heterocycle), or heterocycle, any of which can be optionally substituted, and R^{9a} and R^{9b} are methyl.

In some embodiments, structures are provided including 4-3 and 5-3, wherein R¹ and R² are independently D or H, wherein at least one of R¹ and R² is D, and typically both R¹ and R² are D, R⁵ is C₁-C₆alkyl, and R^{9a} and R^{9b} are methyl.

In some embodiments, structures are provided including 4-3 and 5-3, wherein R¹ and R² are independently D or H, wherein at least one of R¹ and R² is D, and typically both R¹ and R² are D, R⁵ is n-propyl, and R^{9a} and R^{9b} are methyl.

In some embodiments, structures are provided including 4-4 and 5-4, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7a} are H, and R^{7b} is an amino acid side chain.

In some embodiments, structures are provided including 4-4 and 5-4, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7a} are H, and R^{7b} is C₁-C₆alkyl.

In some embodiments, structures are provided including 4-4 and 5-4, wherein R¹ and R² are independently D or H, wherein at least one of R¹ and R² is D, and typically both R¹ and R² are D, R⁸ is -alkylaryl, R⁶ and R^{7a} are H, and R^{7b} is an amino acid side chain.

In some embodiments, structures are provided including 4-4 and 5-4, wherein R¹ and R² are independently D or H, wherein at least one of R¹ and R² is D, and typically both R¹ and R² are D, R⁸ is -alkylaryl, R⁶ and R^{7a} are H, and R^{7b} is C₁-C₆alkyl.

In some embodiments, structures are provided including 4-4 and 5-4, wherein R¹ and R² are independently D or H, wherein at least one of R¹ and R² is D, and typically both R¹ and R² are D, R⁸ is isopropyl, R⁶ and R^{7a} are H, and R^{7b} is an amino acid side chain.

In some embodiments, structures are provided including 4-4 and 5-4, wherein R¹ and R² are independently D or H, wherein at least one of R¹ and R² is D, and typically both R¹ and R² are D, R⁸ is isopropyl, R⁶ and R^{7a} are H, and R^{7b} is methyl.

In some embodiments, structures are provided including 4-4 and 5-4, wherein R¹ and R² are independently D or H, wherein at least one of R¹ and R² is D, and typically both R¹ and R² are D, R⁸ is benzyl, R⁶ and R^{7a} are H, and R^{7b} is an amino acid side chain.

In some embodiments, structures are provided including 4-4 and 5-4, wherein R¹ and R² are independently D or H, wherein at least one of R¹ and R² is D, and typically both R¹ and R² are D, R⁸ is benzyl, R⁶ and R^{7a} are H, and R^{7b} is methyl.

In some embodiments, structures are provided including 4-4 and 5-4, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7b} are H, and R^{7a} is an amino acid side chain.

In some embodiments, structures are provided including 4-4 and 5-4, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7b} are H, and R^{7a} is C₁-C₆alkyl.

In some embodiments, structures are provided including 4-4 and 5-4, wherein R¹ and R² are independently D or H, wherein at least one of R¹ and R² is D, and typically both R¹ and R² are D, R⁸ is -alkylaryl, R⁶ and R^{7b} are H, and R^{7a} is an amino acid side chain.

In some embodiments, structures are provided including 4-4 and 5-4, wherein R¹ and R² are independently D or H, wherein at least one of R¹ and R² is D, and typically both R¹ and R² are D, R⁸ is -alkylaryl, R⁶ and R^{7b} are H, and R^{7a} is C₁-C₆alkyl.

In some embodiments, structures are provided including 4-4 and 5-4, wherein R¹ and R² are independently D or H, wherein at least one of R¹ and R² is D, and typically both R¹ and R² are D, R⁸ is isopropyl, R⁶ and R^{7b} are H, and R^{7a} is an amino acid side chain.

In some embodiments, structures are provided including 4-4 and 5-4, wherein R¹ and R² are independently D or H, wherein at least one of R¹ and R² is D, and typically both R¹ and R² are D, R⁸ is isopropyl, R⁶ and R^{7b} are H, and R^{7a} is methyl.

In some embodiments, structures are provided including 4-4 and 5-4, wherein R¹ and R² are independently D or H, wherein at least one of R¹ and R² is D, and typically both R¹ and R² are D, R⁸ is benzyl, R⁶ and R^{7b} are H, and R^{7a} is an amino acid side chain.

In some embodiments, structures are provided including 4-4 and 5-4, wherein R¹ and R² are independently D or H, wherein at least one of R¹ and R² is D, and typically both R¹ and R² are D, R⁸ is benzyl, R⁶ and R^{7b} are H, and R^{7a} is methyl.

In some embodiments, structures are provided including 4-5 and 5-5, wherein R¹ and R² are independently D or H, wherein at least one of R¹ and R² is D, and typically both R¹ and R² are D, R⁴ is hydrogen, R⁵ is alkyl, cycloalkyl, aryl, -alkylaryl, -alkyl(heterocycle), or heterocycle, any of which can be optionally substituted, R¹¹ and R¹² are C₁-C₂₂alkyl, and X is S.

In some embodiments, structures are provided including 4-5 and 5-5, wherein R¹ and R² are independently D or H, wherein at least one of R¹ and R² is D, and typically both R¹ and R² are D, R⁴ is hydrogen, R⁵ is aryl, R¹¹ and R¹² are C₁-C₂₂alkyl, and X is S.

In some embodiments, structures are provided including 4-5 and 5-5, wherein R¹ and R² are independently D or H, wherein at least one of R¹ and R² is D, and typically both R¹ and R² are D, R⁴ is hydrogen, R⁵ is alkyl, cycloalkyl, aryl, -alkylaryl, -alkyl(heterocycle), or heterocycle, any of which can be optionally substituted, R¹¹ is C₁₀H₂₁, R¹² is C₁₂H₂₅, and X is S.

In some embodiments, structures are provided including 4-5 and 5-5, wherein R¹ and R² are independently D or H, wherein at least one of R¹ and R² is D, and typically both R¹ and R² are D, R⁴ is hydrogen, R⁵ is phenyl, R¹¹ is C₁₀H₂₁, R¹² is C₁₂H₂₅ and X is S.

In some embodiments, structures are provided including 4-6 and 5-6, wherein R¹ and R² are independently D or H, wherein at least one of R¹ and R² is D, and typically both R¹ and R² are D, R⁴ is hydrogen, and R¹⁵ is C₁-C₆alkyl.

In some embodiments, structures are provided including 4-6 and 5-6, wherein R¹ and R² are independently D or H, wherein at least one of R¹ and R² is D, and typically both R¹ and R² are D, R⁴ is hydrogen, and R¹⁵ is —C₁-C₃alkyl-O—C₁-C₅alkoxy.

In some embodiments, structures are provided including 4-6 and 5-6, wherein R¹ and R² are independently D or H, wherein at least one of R¹ and R² is D, and typically both R¹ and R² are D, R⁴ is hydrogen, and R¹⁵ is t-butyl.

In some embodiments, structures are provided including 4-7, wherein R¹ and R² are independently D or H, wherein at least one of R¹ and R² is D, and typically both R¹ and R² are D, R¹³ is hydrogen, R⁴ is acyl, R¹⁴ is halogen, and y=1, 2, 3, 4, or 5.

In some embodiments, structures are provided including 4-7, wherein R¹ and R² are independently D or H, wherein at least one of R¹ and R² is D, and typically both R¹ and R² are D, R⁴ and R¹³ are hydrogen, R¹⁴ is halogen, and y=1, 2, 3, 4, or 5.

In some embodiments, structures are provided including 4-7, wherein R¹ and R² are independently D or H, wherein at least one of R¹ and R² is D, and typically both R¹ and R² are D, R¹³ is hydrogen, R⁴ is acyl, R¹⁴ is Cl, and y=1.

In some embodiments, structures are provided including 4-7, wherein R¹ and R² are independently D or H, wherein at least one of R¹ and R² is D, and typically both R¹ and R² are D, R⁴ and R¹³ are hydrogen, R¹⁴ is Cl, and y=1.

In some embodiments, structures are provided including 5-7, wherein R¹ and R² are independently D or H, wherein at least one of R¹ and R² is D, and typically both R¹ and R² are D, R⁴ is acyl, R¹⁴ is halogen, and y=1, 2, 3, 4, or 5.

In some embodiments, structures are provided including 5-7, wherein R¹ and R² are independently D or H, wherein at least one of R¹ and R² is D, and typically both R¹ and R² are D, R⁴ is hydrogen, R¹⁴ is halogen, and y=1, 2, 3, 4, or 5.

In some embodiments, structures are provided including 5-7, wherein R¹ and R² are independently D or H, wherein at least one of R¹ and R² is D, and typically both R¹ and R² are D, R⁴ is acyl, R¹⁴ is Cl, and y=1.

In some embodiments, structures are provided including 5-7, wherein R¹ and R² are independently D or H, wherein at least one of R¹ and R² is D, and typically both R¹ and R² are D, R⁴ is hydrogen, R¹⁴ is Cl, and y=1.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is -alkylaryl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is -alkylaryl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is -alkylaryl, R⁶ and R^{7a} are H, R^{7b} is an amino acid side chain, and R⁵ is aryl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is -alkylaryl, R⁶ and R^{7a} are H, R^{7b} is C₁-C₆alkyl, and R⁵ is aryl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7a} are H, R^{7b} is an amino acid side chain, and R⁵ is aryl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7a} are H, R^{7b} is C₁-C₆alkyl, and R⁵ is aryl.

In some embodiments, structures are provided including 4-8 and 5-8, wherein R¹ and R² are deuterium, R⁴ is hydrogen, R⁸ is C₁-C₆alkyl or C₃-C₆cycloalkyl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are deuterium, R⁴ is hydrogen, R⁸ is C₁-C₆alkyl or C₃-C₆cycloalkyl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are deuterium, R⁴ is hydrogen, R⁸ is -alkylaryl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are deuterium, R⁴ is hydrogen, R⁸ is -alkylaryl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are deuterium, R⁴ is hydrogen, R⁸ is -alkylaryl, R⁶ and R^{7a} are H, R^{7b} is an amino acid side chain, and R⁵ is aryl.

In some embodiments, structures are provided including 4-8 and 5-8, wherein R¹ and R² and are deuterium, R⁴ is hydrogen, R⁸ is -alkylaryl, R⁶ and R^{7a} are H, R^{7b} is C₁-C₆alkyl, and R⁵ is aryl.

In some embodiments, structures are provided including 4-8 and 5-8, wherein R¹ and R² are deuterium, R⁴ is hydrogen, R⁸ is C₁-C₆alkyl or C₃-C₆cycloalkyl, R⁶ and R^{7a} are H, R^{7b} is an amino acid side chain, and R⁵ is aryl.

In some embodiments, structures are provided including 4-8 and 5-8, wherein R¹ and R² are deuterium, R⁴ is hydrogen, R⁸ is C₁-C₆alkyl or C₃-C₆cycloalkyl, R⁶ and R^{7a} are H, R^{7b} is C₁-C₆alkyl, and R⁵ is aryl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is isopropyl, R⁶ and R^{7a} are H, R^{7b} is an amino acid side chain, and R⁵ is phenyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is isopropyl, R⁶ and R^{7a} are H, R^{7b} is methyl, and R⁵ is phenyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is benzyl, R⁶ and R^{7a} are H, R^{7b} is an amino acid side chain, and R⁵ is phenyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is benzyl, R⁶ and R^{7a} are H, R^{7b} is methyl, and R⁵ is phenyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is benzyl, R⁶ and R^{7a} are H, R^{7b} is an amino acid side chain, and R⁵ is 1-naphthyl or 2-naphthyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is benzyl, R⁶ and R^{7a} are H, R^{7b} is methyl, and R⁵ is 1-naphthyl or 2-naphthyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is isopropyl, R⁶ and R^{7a} are H, R^{7b} is an amino acid side chain, and R⁵ is 1-naphthyl or 2-naphthyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is isopropyl, R⁶ and R^{7a} are H, R^{7b} is methyl, and R⁵ is 1-naphthyl or 2-naphthyl.

In some embodiments, structures are provided including 4-8 and 5-8, wherein R¹ and R² are deuterium, R⁴ is hydrogen, R⁸ is isopropyl, R⁶ and R^{7a} are H, R^{7b} is an amino acid side chain, and R⁵ is phenyl.

In some embodiments, structures are provided including 4-8 and 5-8, wherein R¹ and R² are deuterium, R⁴ is hydrogen, R⁸ is isopropyl, R⁶ and R^{7a} are H, R^{7b} is methyl, and R⁵ is phenyl.

In some embodiments, structures are provided including 4-8 and 5-8, wherein R¹ and R² are deuterium, R⁴ is hydrogen, R⁸ is benzyl, R⁶ and R^{7a} are H, R^{7b} is an amino acid side chain, and R⁵ is phenyl.

In some embodiments, structures are provided including 4-8 and 5-8, wherein R¹ and R² are deuterium, R⁴ is hydrogen, R⁸ is benzyl, R⁶ and R^{7a} are H, R^{7b} is methyl, and R⁵ is phenyl.

In some embodiments, structures are provided including 4-8 and 5-8, wherein R¹ and R² are deuterium, R⁴ is hydrogen, R⁸ is benzyl, R⁶ and R^{7a} are H, R^{7b} is an amino acid side chain, and R⁵ is 1-naphthyl or 2-naphthyl.

In some embodiments, structures are provided including 4-8 and 5-8, wherein R¹ and R² are deuterium, R⁴ is hydrogen, R⁸ is benzyl, R⁶ and R^{7a} are H, R^{7b} is methyl, and R⁵ is 1-naphthyl or 2-naphthyl.

In some embodiments, structures are provided including 4-8 and 5-8, wherein R¹ and R² are deuterium, R⁴ is hydrogen, R⁸ is isopropyl, R⁶ and R^{7a} are H, R^{7b} is an amino acid side chain, and R⁵ is 1-naphthyl or 2-naphthyl.

In some embodiments, structures are provided including 4-8 and 5-8, wherein R¹ and R² are deuterium, R⁴ is hydrogen, R⁸ is isopropyl, R⁶ and R^{7b} are H, R^{7b} is methyl, and R⁵ is 1-naphthyl or 2-naphthyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is -alkylaryl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is -alkylaryl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is -alkylaryl, R⁶ and R^{7b} are H, R^{7a} is an amino acid side chain, and R⁵ is aryl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is -alkylaryl, R⁶ and R^{7b} are H, R^{7a} is C₁-C₆alkyl, and R⁵ is aryl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7b} are H, R^{7a} is an amino acid side chain, and R⁵ is aryl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7b} are H, R^{7a} is C₁-C₆alkyl, and R⁵ is aryl.

In some embodiments, structures are provided including 4-8 and 5-8, wherein R¹ and R² are deuterium, R⁴ is hydrogen, R⁸ is C₁-C₆alkyl or C₃-C₆cycloalkyl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are deuterium, R⁴ is hydrogen, R⁸ is C₁-C₆alkyl or C₃-C₆cycloalkyl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are deuterium, R⁴ is hydrogen, R⁸ is -alkylaryl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are deuterium, R⁴ is hydrogen, R⁸ is -alkylaryl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 4-8 and 5-8, wherein R¹ and R² are deuterium, R⁴ is hydrogen, R⁸ is -alkylaryl, R⁶ and R^{7b} are H, R^{7a} is an amino acid side chain, and R⁵ is aryl.

In some embodiments, structures are provided including 4-8 and 5-8, wherein R¹ and R² are deuterium, R⁴ is hydrogen, R⁸ is -alkylaryl, R⁶ and R^{7b} are H, R^{7a} is C₁-C₆alkyl, and R⁵ is aryl.

In some embodiments, structures are provided including 4-8 and 5-8, wherein R¹ and R² are deuterium, R⁴ is hydrogen, R⁸ is C₁-C₆alkyl or C₃-C₆cycloalkyl, R⁶ and R^{7b} are H, R^{7a} is an amino acid side chain, and R⁵ is aryl.

In some embodiments, structures are provided including 4-8 and 5-8, wherein R¹ and R² are deuterium, R⁴ is hydrogen, R⁸ is C₁-C₆alkyl or C₃-C₆cycloalkyl, R⁶ and R^{7b} are H, R^{7a} is C₁-C₆alkyl, and R⁵ is aryl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is isopropyl, R⁶ and R^{7b} are H, R^{7a} is an amino acid side chain, and R⁵ is phenyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is isopropyl, R⁶ and R^{7b} are H, R^{7a} is methyl, and R⁵ is phenyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is benzyl, R⁶ and R^{7b} are H, R^{7a} is an amino acid side chain, and R⁵ is phenyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is benzyl, R⁶ and R^{7b} are H, R^{7a} is methyl, and R⁵ is phenyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is benzyl, R⁶ and R^{7b} are H, R^{7a} is an amino acid side chain, and R⁵ is 1-naphthyl or 2-naphthyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is benzyl, R⁶ and R^{7b} are H, R^{7a} is methyl, and R⁵ is 1-naphthyl or 2-naphthyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is isopropyl, R⁶ and R^{7b} are H, R^{7a} is an amino acid side chain, and R⁵ is 1-naphthyl or 2-naphthyl.

In some embodiments, structures are provided including 4-8 and 5-8, wherein R¹ and R² are independently D or H, wherein at least one of R¹ and R² is D, and typically both R¹ and R² are D, R⁴ is hydrogen, R⁸ is isopropyl, R⁶ and R^{7b} are H, R^{7a} is methyl, and R⁵ is 1-naphthyl or 2-naphthyl.

In some embodiments, structures are provided including 4-8 and 5-8, wherein R¹ and R² are deuterium, R⁴ is hydrogen, R⁸ is isopropyl, R⁶ and R^{7b} are H, R^{7a} is an amino acid side chain, and R⁵ is phenyl.

In some embodiments, structures are provided including 4-8 and 5-8, wherein R¹ and R² are deuterium, R⁴ is hydrogen, R⁸ is isopropyl, R⁶ and R^{7b} are H, R^{7a} is methyl, and R⁵ is phenyl.

In some embodiments, structures are provided including 4-8 and 5-8, wherein R¹ and R² are deuterium, R⁴ is hydrogen, R⁸ is benzyl, R⁶ and R^{7b} are H, R^{7a} is an amino acid side chain, and R⁵ is phenyl.

In some embodiments, structures are provided including 4-8 and 5-8, wherein R¹ and R² are deuterium, R⁴ is hydrogen, R⁸ is benzyl, R⁶ and R^{7b} are H, R^{7a} is methyl, and R⁵ is phenyl.

In some embodiments, structures are provided including 4-8 and 5-8, wherein R¹ and R² are deuterium, R⁴ is hydrogen, R⁸ is benzyl, R⁶ and R^{7b} are H, R^{7a} is an amino acid side chain, and R⁵ is 1-naphthyl or 2-naphthyl.

In some embodiments, structures are provided including 4-8 and 5-8, wherein R¹ and R² are deuterium, R⁴ is hydrogen, R⁸ is benzyl, R⁶ and R^{7b} are H, R^{7a} is methyl, and R⁵ is 1-naphthyl or 2-naphthyl.

In some embodiments, structures are provided including 4-8 and 5-8, wherein R¹ and R² are deuterium, R⁴ is hydrogen, R⁸ is isopropyl, R⁶ and R^{7b} are H, R^{7a} is an amino acid side chain, and R⁵ is 1-naphthyl or 2-naphthyl.

In some embodiments, structures are provided including 4-8 and 5-8, wherein R¹ and R² are deuterium, R⁴ is hydrogen, R⁸ is isopropyl, R⁶ and R^{7b} are H, R^{7a} is methyl, and R⁵ is 1-naphthyl or 2-naphthyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7a} are H, R^{7b} is an amino acid side chain, R¹⁸ is C₁-C₆alkyl or C₃-C₆cycloalkyl, R¹⁶ and R^{17a} are H, and R^{17b} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7a} are H, R^{7b} is C₁-C₆alkyl, R¹⁸ is C₁-C₆alkyl or C₃-C₆cycloalkyl, R¹⁶ and R^{17a} are H, and R^{17b} is C₁-C₆alkyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is -alkylaryl, R⁶ and R^{7a} are H, R^{7b} is an amino acid side chain, R¹⁸ is -alkylaryl, R¹⁶ and R^{17a} are H, and R^{17b} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is -alkylaryl, R⁶ and R^{7a} are H, R^{7b} is C₁-C₆alkyl, R¹⁸ is -alkylaryl, R¹⁶ and R^{17a} are H, and R^{17b} is C₁-C₆alkyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is -alkylaryl, R⁶ and R^{7a} are H, R^{7b} is an amino acid side chain, R¹⁸ is -alkylaryl, R¹⁶ and R^{17a} are H, and R^{17b} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is -alkylaryl, R⁶ and R^{7a} are H, R^{7b} is C₁-C₆alkyl, R¹⁸ is -alkylaryl, R¹⁶ and R^{17a} are H, and R^{17b} is C₁-C₆alkyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7a} are H, R^{7b} is an amino acid side chain, R¹⁸ is C₁-C₆alkyl or C₃-C₆cycloalkyl, R¹⁶ and R^{17a} are H, and R^{17b} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7a} are H, R^{7b} is C₁-C₆alkyl, R¹⁸ is C₁-C₆alkyl or C₃-C₆cycloalkyl, R¹⁶ and R^{17a} are H, and R^{17b} is C₁-C₆alkyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is isopropyl, R⁶ and R^{7a} are H, R^{7b} is an amino acid side chain, R¹⁸ is isopropyl, R¹⁶ and R^{17a} are H, and R^{17b} is an amino acid side chain

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is isopropyl, R⁶ and R^{7a} are H, R^{7b} is methyl, R¹⁸ is isopropyl, R¹⁶ and R^{17a} are H, and R^{17b} is methyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is benzyl, R⁶ and R^{7a} are H, R^{7b} is an amino acid side chain, R¹⁸ is benzyl, R¹⁶ and R^{17a} are H, and R^{17b} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is benzyl, R⁶ and R^{7a} are H, R^{7b} is methyl, R¹⁸ is benzyl, R¹⁶ and R^{17a} are H, and R^{17b} is methyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is benzyl, R⁶ and R^{7a} are H, R^{7b} is an amino acid side chain, R¹⁸ is benzyl, R¹⁶ and R^{17a} are H, and R^{17b} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is benzyl, R⁶ and R^{7a} are H, R^{7b} is methyl, R¹⁸ is benzyl, R¹⁶ and R^{17a} are H, and R^{17b} is methyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is isopropyl, R⁶ and R^{7a} are H, R^{7b} is an amino acid side chain, R¹⁸ is isopropyl, R¹⁶ and R^{17a} are H, and R^{17b} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is isopropyl, R⁶ and R^{7a} are H, R^{7b} is methyl, R¹⁸ is isopropyl, R¹⁶ and R^{17a} are H, and R^{17b} is methyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7b} are H, R^{7a} is an amino acid side chain, R¹⁸ is C₁-C₆alkyl or C₃-C₆cycloalkyl, R¹⁶ and R^{17b} are H, and R^{17a} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7b} are H, R^{7a} is C₁-C₆alkyl, R¹⁸ is C₁-C₆alkyl or C₃-C₆cycloalkyl, R¹⁶ and R^{17b} are H, and R^{17a} is C₁-C₆alkyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is -alkylaryl, R⁶ and R^{7b} are H, R^{7a} is an amino acid side chain, R¹⁸ is -alkylaryl, R¹⁶ and R^{17b} are H, and R^{17a} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is -alkylaryl, R⁶ and R^{7b} are H, R^{7a} is C₁-C₆alkyl, R¹⁸ is -alkylaryl, R¹⁶ and R^{17b} are H, and R^{17a} is C₁-C₆alkyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is -alkylaryl, R⁶ and R^{7b} are H, R^{7a} is an amino acid side chain, R¹⁸ is -alkylaryl, R¹⁶ and R^{17b} are H, and R^{17a} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is -alkylaryl, R⁶ and R^{7b} are H, R^{7a} is C₁-C₆alkyl, R¹⁸ is -alkylaryl, R¹⁶ and R^{17b} are H, and R^{17a} is C₁-C₆alkyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7b} are H, R^{7a} is an amino acid side chain, R¹⁸ is C₁-C₆alkyl or C₃-C₆cycloalkyl, R¹⁶ and R^{17b} are H, and R^{17a} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is C₁-C₆alkyl or C₃-C₆cycloalkyl, R⁶ and R^{7b} are H, R^{7a} is C₁-C₆alkyl, R¹⁸ is C₁-C₆alkyl or C₃-C₆cycloalkyl, R¹⁶ and R^{17b} are H, and R^{17a} is C₁-C₆alkyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is isopropyl, R⁶ and R^{7b} are H, R^{7a} is an amino acid side chain, R¹⁸ is isopropyl, R¹⁶ and R^{17b} are H, and R^{17a} is an amino acid side chain

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is isopropyl, R⁶ and R^{7b} are H, R^{7a} is methyl, R¹⁸ is isopropyl, R¹⁶ and R^{17b} are H, and R^{17a} is methyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is benzyl, R⁶ and R^{7b} are H, R^{7a} is an amino acid side chain, R¹⁸ is benzyl, R¹⁶ and R^{17b} are H, and R^{17a} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is benzyl, R⁶ and R^{7b} are H, R^{7a} is methyl, R¹⁸ is benzyl, R¹⁶ and R^{17b} are H, and R^{17a} is methyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is benzyl, R⁶ and R^{7b} are H, R^{7a} is an amino acid side chain, R¹⁸ is benzyl, R¹⁶ and R^{17b} are H, and R^{17a} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is benzyl, R⁶ and R^{7b} are H, R^{7a} is methyl, R¹⁸ is benzyl, R¹⁶ and R^{17b} are H, and R^{17a} is methyl.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is isopropyl, R⁶ and R^{7b} are H, R^{7a} is an amino acid side chain, R¹⁸ is isopropyl, R¹⁶ and R^{17b} are H, and R^{17a} is an amino acid side chain.

In some embodiments, structures are provided including 4-9 and 5-9, wherein R¹ and R² are independently D or H, wherein at least one of R¹ and R² is D, and typically both R¹ and R² are D, R⁴ is hydrogen, R⁸ is isopropyl, R⁶ and R^{7b} are H, R^{7a} is methyl, R¹⁸ is isopropyl, R¹⁶ and R^{17b} are H, and R^{17a} is methyl.

In some embodiments, structures are provided including 4-10 and 5-10, wherein R¹ and R² are independently D or H, wherein at least one of R¹ and R² is D, and typically both R¹ and R² are D, R⁴ is hydrogen, R¹⁵ is —C₁-C₃alkyl-O—C₁-C₅alkoxy, R¹⁹ is H, and R²⁰ is -alkylaryl.

In some embodiments, structures are provided including 4-10 and 5-10, wherein R¹ and R² are independently D or H, wherein at least one of R¹ and R² is D, and typically both R¹ and R² are D, R⁴ is hydrogen, R¹⁵ is C₁-C₆alkyl, R¹⁹ is H, and R²⁰ is -alkylaryl.

In some embodiments, structures are provided including 4-10 and 5-10, wherein R¹ and R² are independently D or H, wherein at least one of R¹ and R² is D, and typically both R¹ and R² are D, R⁴ is hydrogen, R¹⁵ is isopropoxyl, R¹⁹ is H and R²⁰ is -alkylaryl.

In some embodiments, structures are provided including 4-10 and 5-10, wherein R¹ and R² are independently D or H, wherein at least one of R¹ and R² is D, and typically both R¹ and R² are D, R⁴ is hydrogen, R¹⁵ is —C₁-C₃alkyl-O—C₁-C₅alkoxy, R¹⁹ is H, and R²⁰ is benzyl.

In some embodiments, structures are provided including 4-10 and 5-10, wherein R¹ and R² are independently D or H, wherein at least one of R¹ and R² is D, and typically both R¹ and R² are D, R⁴ is hydrogen, R¹⁵ is C₁-C₆alkyl, R¹⁹ is H, and R²⁰ is benzyl.

In some embodiments, structures are provided including 4-10 and 5-10, wherein R¹ and R² are independently D or H, wherein at least one of R¹ and R² is D, and typically both R¹ and R² are D, R⁴ is hydrogen, R¹⁵ is isopropoxyl, R¹⁹ is H and R²⁰ is benzyl.

In some embodiments, structures are provided including 4-11 and 5-11, R¹ and R² are independently D or H, wherein at least one of R¹ and R² is D, and typically both R¹ and R² are D, R⁴, R^{21a}, and R^{21b} are hydrogen, and R¹⁵ is —C₁-C₃alkyl-O—C₁-C₅alkoxy.

In some embodiments, structures are provided including 4-11 and 5-11, R¹ and R² are independently D or H, wherein at least one of R¹ and R² is D, and typically both R¹ and R² are D, R⁴, R^{21a}, and R^{21b} are hydrogen, and R¹⁵ is C₁-C₆alkyl.

In some embodiments, structures are provided including 4-11 and 5-11, R¹ and R² are independently D or H, wherein at least one of R¹ and R² is D, and typically both R¹ and R² are D, R⁴, R^{21a}, and R^{21b} are hydrogen, and R¹⁵ is isopropoxyl.

In some embodiments, structures are provided including 4-11 and 5-11, R¹ and R² are independently D or H, wherein at least one of R¹ and R² is D, and typically both R¹ and R² are D, R⁴, R^{21a}, and R^{21b} are hydrogen, and R¹⁵ is t-butyl.

In some embodiments, structures are provided including 4-12 and 5-12, R¹ and R² are independently D or H, wherein at least one of R¹ and R² is D, and typically both R¹ and R² are D, R⁴, R^{21a}, and R^{21b} are hydrogen, R¹⁵ is —C₁-C₃alkyl-O—C₁-C₅alkoxy, R¹⁹ is H, and R²⁰ is -alkylaryl.

In some embodiments, structures are provided including 4-12 and 5-12, R¹ and R² are independently D or H, wherein at least one of R¹ and R² is D, and typically both R¹ and R² are D, R⁴, R^{21a}, and R^{21b} are hydrogen, R¹⁵ is C₁-C₆alkyl, R¹⁹ is H, and R²⁰ is -alkylaryl.

In some embodiments, structures are provided including 4-12 and 5-12, R¹ and R² are independently D or H, wherein at least one of R¹ and R² is D, and typically both R¹ and R² are D, R⁴, R^{21a}, and R^{21b} are hydrogen, R¹⁵ is isopropoxyl, R¹⁹ is H, and R²⁰ is benzyl.

In some embodiments, structures are provided including 4-12 and 5-12, R¹ and R² are independently D or H, wherein at least one of R¹ and R² is D, and typically both R¹ and R² are D, R⁴, R^{21a}, and R^{21b} are hydrogen, R¹⁵ is t-butyl, R¹⁹ is H, and R²⁰ is benzyl.

In some embodiments, structures are provided including 4-12 and 5-12, R¹ and R² are independently D or H, wherein at least one of R¹ and R² is D, and typically both R¹ and R² are D, R⁴, R^{21a}, and R^{21b} are hydrogen, R¹⁵ is isopropoxyl, R¹⁹ is H, and R²⁰ is -alkylaryl.

In some embodiments, structures are provided including 4-12 and 5-12, R¹ and R² are independently D or H, wherein at least one of R¹ and R² is D, and typically both R¹ and R² are D, R⁴, R^{21a}, and R^{21b} are hydrogen, R¹⁵ is t-butyl, R¹⁹ is H, and R²⁰ is -alkylaryl.

In some embodiments, structures are provided including 4-12 and 5-12, R¹ and R² are independently D or H, wherein at least one of R¹ and R² is D, and typically both R¹ and R² are D, R⁴, R^{21a}, and R^{21b} are hydrogen, R¹⁵ is —C₁-C₃alkyl-O—C₁-C₅alkoxy, R¹⁹ is H, and R²⁰ is benzyl.

In some embodiments, structures are provided including 4-12 and 5-12, R¹ and R² are independently D or H, wherein at least one of R¹ and R² is D, and typically both R¹ and R² are D, R⁴, R^{21a}, and R^{21b} are hydrogen, R¹⁵ is C₁-C₆alkyl, R¹⁹ is H, and R²⁰ is benzyl.

In some embodiments, structures are provided including 6-1, wherein R¹ and R² are independently D or H, wherein at least one of R¹ and R² is D, and typically both R¹ and R² are D, and R⁵ and R¹⁰ are independently alkyl, cycloalkyl, aryl, -alkylaryl, -alkyl(heterocycle), or heterocycle, any of which can be optionally substituted.

In some embodiments, structures are provided including 6-1, wherein R¹ and R² are independently D or H, wherein at least one of R¹ and R² is D, and typically both R¹ and R² are D, and R⁵ and R¹⁰ are independently aryl or -alkylaryl, either of which can be optionally substituted.

In some embodiments, structures are provided including 6-5, wherein R¹ and R² are independently D or H, wherein at least one of R¹ and R² is D, and typically both R¹ and R² are D, R⁵ is alkyl, cycloalkyl, aryl, -alkylaryl, -alkyl(heterocycle), or heterocycle, any of which can be optionally substituted, R¹¹ and R¹² are C₁-C₂₂alkyl, and X is S.

In some embodiments, structures are provided including 6-5, wherein R¹ and R² are independently D or H, wherein at least one of R¹ and R² is D, and typically both R¹ and R² are D, R⁵ is alkyl, cycloalkyl, aryl, -alkylaryl, -alkyl(heterocycle), or heterocycle, any of which can be optionally substituted, R¹¹ and R¹² are C₁-C₂₂alkyl, and X is S.

In some embodiments, structures are provided including 6-5, wherein R¹ and R² are independently D or H, wherein at least one of R¹ and R² is D, and typically both R¹ and R² are D, R⁵ is alkyl, cycloalkyl, aryl, -alkylaryl, -alkyl(heterocycle), or heterocycle, any of which can be optionally substituted, R¹¹ is C₁₀H₂₁, R¹² is C₁₂H₂₅, and X is S.

In some embodiments, structures are provided including 6-5, wherein R¹ and R² are independently D or H, wherein at least one of R¹ and R² is D, and typically both R¹ and R² are D, R⁵ is phenyl, R¹¹ is C₁₀H₁₂, R¹² is C₁₂H₂₅, and X is S.

In some embodiments, structures are provided including 6-6, wherein R¹ and R² are independently D or H, wherein at least one of R¹ and R² is D, and typically both R¹ and R² are D, and R¹⁵ is C₁-C₆alkyl.

In some embodiments, structures are provided including 6-6, wherein R¹ and R² are independently D or H, wherein at least one of R¹ and R² is D, and typically both R¹ and R² are D, and R¹⁵ is —C₁-C₃alkyl-O—C₁-C₅alkoxy.

In some embodiments, structures are provided including 6-6, wherein R¹ and R² are independently D or H, wherein at least one of R¹ and R² is D, and typically both R¹ and R² are D, and R¹⁵ is t-butyl.

In some embodiments, structures are provided including 6-7, wherein R¹ and R² are independently D or H, wherein at least one of R¹ and R² is D, and typically both R¹ and R² are D, R¹⁴ is halogen, and y=1, 2, 3, 4, or 5.

In some embodiments, structures are provided including 6-7, wherein R¹ and R² are independently D or H, wherein at least one of R¹ and R² is D, and typically both R¹ and R² are D, R¹⁴ is Cl, and y=1.

In some embodiments, structures are provided including 6-8, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are independently D or H, wherein at least one of R¹ and R² is D, and typically both R¹ and R² are D, R⁸ is -alkylaryl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are independently D or H, wherein at least one of R¹ and R² is D, and typically both R¹ and R² are D, R⁸ is -alkylaryl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are independently D or H, wherein at least one of R¹ and R² is D, and typically both R¹ and R² are D, R⁸ is -alkylaryl, R⁶ and R^{7a} are H, R^{7b} is an amino acid side chain, and R⁵ is aryl.

In some embodiments, structures are provided including 6-8, wherein R¹ and R² are independently D or H, wherein at least one of R¹ and R² is D, and typically both R¹ and R² are D, R⁸ is -alkylaryl, R⁶ and R^{7a} are H, R^{7b} is C₁-C₆alkyl, and R⁵ is aryl.

In some embodiments, structures are provided including 6-8, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7a} are H, R^{7b} is an amino acid side chain, and R⁵ is aryl.

In some embodiments, structures are provided including 6-8, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7a} are H, R^{7b} is C₁-C₆alkyl, and R⁵ is aryl.

In some embodiments, structures are provided including 6-8, wherein R¹ and R² are deuterium, R⁸ is C₁-C₆alkyl or C₃-C₆cycloalkyl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are deuterium, R⁸ is C₁-C₆alkyl or C₃-C₆cycloalkyl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are deuterium, R⁸ is -alkylaryl, R⁶ and R^{7a} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are deuterium, R⁸ is -alkylaryl, R⁶ and R^{7a} are H, R^{7b} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are deuterium, R⁸ is -alkylaryl, R⁶ and R^{7a} are H, R^{7b} is an amino acid side chain, and R⁵ is aryl.

In some embodiments, structures are provided including 6-8, wherein R¹ and R² are deuterium, R⁸ is -alkylaryl, R⁶ and R^{7a} are H, R^{7b} is C₁-C₆alkyl, and R⁵ is aryl.

In some embodiments, structures are provided including 6-8, wherein R¹ and R² are deuterium, R⁸ is C₁-C₆alkyl or C₃-C₆cycloalkyl, R⁶ and R^{7a} are H, R^{7b} is an amino acid side chain, and R⁵ is aryl.

In some embodiments, structures are provided including 6-8, wherein R¹ and R² are deuterium, R⁸ is C₁-C₆alkyl or C₃-C₆cycloalkyl, R⁶ and R^{7a} are H, R^{7b} is C₁-C₆alkyl, and R⁵ is aryl.

In some embodiments, structures are provided including 6-8, wherein R¹ and R² are independently D or H, wherein at least one of R¹ and R² is D, and typically both R¹ and R² are D, R⁸ is isopropyl, R⁶ and R^{7a} are H, R^{7b} is an amino acid side chain, and R⁵ is phenyl.

In some embodiments, structures are provided including 6-8, wherein R¹ and R² are independently D or H, wherein at least one of R¹ and R² is D, and typically both R¹ and R² are D, R⁸ is isopropyl, R⁶ and R^{7a} are H, R^{7b} is methyl, and R⁵ is phenyl.

In some embodiments, structures are provided including 6-8, wherein R¹ and R² are independently D or H, wherein at least one of R¹ and R² is D, and typically both R¹ and R² are D, R⁸ is benzyl, R⁶ and R^{7a} are H, R^{7b} is an amino acid side chain, and R⁵ is phenyl.

In some embodiments, structures are provided including 6-8, wherein R¹ and R² are independently D or H, wherein at least one of R¹ and R² is D, and typically both R¹ and R² are D, R⁸ is benzyl, R⁶ and R^{7a} are H, R^{7b} is methyl, and R⁵ is phenyl.

In some embodiments, structures are provided including 6-8, wherein R¹ and R² are independently D or H, wherein at least one of R¹ and R² is D, and typically both R¹ and R² are D, R⁸ is benzyl, R⁶ and R^{7a} are H, R^{7b} is an amino acid side chain, and R⁵ is 1-naphthyl or 2-naphthyl.

In some embodiments, structures are provided including 6-8, wherein R¹ and R² are independently D or H, wherein at least one of R¹ and R² is D, and typically both R¹ and R² are D, R⁸ is benzyl, R⁶ and R^{7a} are H, R^{7b} is methyl, and R⁵ is 1-naphthyl or 2-naphthyl.

In some embodiments, structures are provided including 6-8, wherein R¹ and R² are independently D or H, wherein at least one of R¹ and R² is D, and typically both R¹ and R² are D, R⁸ is isopropyl, R⁶ and R^{7a} are H, R^{7b} is an amino acid side chain, and R⁵ is 1-naphthyl or 2-naphthyl.

In some embodiments, structures are provided including 6-8, wherein R¹ and R² are independently D or H, wherein at least one of R¹ and R² is D, and typically both R¹ and R² are D, R⁸ is isopropyl, R⁶ and R^{7a} are H, R^{7b} is methyl, and R⁵ is 1-naphthyl or 2-naphthyl.

In some embodiments, structures are provided including 6-8, wherein R¹ and R² are deuterium, R⁸ is isopropyl, R⁶ and R^{7a} are H, R^{7b} is an amino acid side chain, and R⁵ is phenyl.

In some embodiments, structures are provided including 6-8, wherein R¹ and R² are deuterium, R⁸ is isopropyl, R⁶ and R^{7a} are H, R^{7b} is methyl, and R⁵ is phenyl.

In some embodiments, structures are provided including 6-8, wherein R¹ and R² are deuterium, R⁸ is benzyl, R⁶ and R^{7a} are H, R^{7b} is an amino acid side chain, and R⁵ is phenyl.

In some embodiments, structures are provided including 6-8, wherein R¹ and R² are deuterium, R⁸ is benzyl, R⁶ and R^{7a} are H, R^{7b} is methyl, and R⁵ is phenyl.

In some embodiments, structures are provided including 6-8, wherein R¹ and R² are deuterium, R⁸ is benzyl, R⁶ and R^{7a} are H, R^{7b} is an amino acid side chain, and R⁵ is 1-naphthyl or 2-naphthyl.

In some embodiments, structures are provided including 6-8, wherein R¹ and R² are deuterium, R⁸ is benzyl, R⁶ and R^{7a} are H, R^{7b} is methyl, and R⁵ is 1-naphthyl or 2-naphthyl.

In some embodiments, structures are provided including 6-8, wherein R¹ and R² are deuterium, R⁸ is isopropyl, R⁶ and R^{7a} are H, R^{7b} is an amino acid side chain, and R⁵ is 1-naphthyl or 2-naphthyl.

In some embodiments, structures are provided including 6-8, wherein R¹ and R² are deuterium, R⁸ is isopropyl, R⁶ and R^{7a} are H, R^{7b} is methyl, and R⁵ is 1-naphthyl or 2-naphthyl.

In some embodiments, structures are provided including 6-8, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are independently D or H, wherein at least one of R¹ and R² is D, and typically both R¹ and R² are D, R⁸ is -alkylaryl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are independently D or H, wherein at least one of R¹ and R² is D, and typically both R¹ and R² are D, R⁸ is -alkylaryl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are independently D or H, wherein at least one of R¹ and R² is D, and typically both R¹ and R² are D, R⁸ is -alkylaryl, R⁶ and R^{7b} are H, R^{7a} is an amino acid side chain, and R⁵ is aryl.

In some embodiments, structures are provided including 6-8, wherein R¹ and R² are independently D or H, wherein at least one of R¹ and R² is D, and typically both R¹ and R² are D, R⁸ is -alkylaryl, R⁶ and R^{7b} are H, R^{7a} is C₁-C₆alkyl, and R⁵ is aryl.

In some embodiments, structures are provided including 6-8, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7b} are H, R^{7a} is an amino acid side chain, and R⁵ is aryl.

In some embodiments, structures are provided including 6-8, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7b} are H, R^{7a} is C₁-C₆alkyl, and R⁵ is aryl.

In some embodiments, structures are provided including 6-8, wherein R¹ and R² are deuterium, R⁸ is C₁-C₆alkyl or C₃-C₆cycloalkyl, R⁶ and R^{7b} are H, R^{7b} is an amino acid side chain, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are deuterium, R⁸ is C₁-C₆alkyl or C₃-C₆cycloalkyl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are deuterium, R⁸ is -alkylaryl, R⁶ and R^{7b} are H, R^{7a} is an amino acid side chain, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are deuterium, R⁸ is -alkylaryl, R⁶ and R^{7b} are H, R^{7a} is C₁-C₆alkyl, and R⁵ is alkyl, cycloalkyl, aryl, -alkylaryl, -alkyl(heterocycle), or heterocycle, any of which can be optionally substituted.

In some embodiments, structures are provided including 6-8, wherein R¹ and R² are deuterium, R⁸ is -alkylaryl, R⁶ and R^{7b} are H, R^{7a} is an amino acid side chain, and R⁵ is aryl.

In some embodiments, structures are provided including 6-8, wherein R¹ and R² are deuterium, R⁸ is -alkylaryl, R⁶ and R^{7b} are H, R^{7a} is C₁-C₆alkyl, and R⁵ is aryl.

In some embodiments, structures are provided including 6-8, wherein R¹ and R² are deuterium, R⁸ is C₁-C₆alkyl or C₃-C₆cycloalkyl, R⁶ and R^{7b} are H, R^{7a} is an amino acid side chain, and R⁵ is aryl.

In some embodiments, structures are provided including 6-8, wherein R¹ and R² are deuterium, R⁸ is C₁-C₆alkyl or C₃-C₆cycloalkyl, R⁶ and R^{7b} are H, R^{7a} is C₁-C₆alkyl, and R⁵ is aryl.

In some embodiments, structures are provided including 6-8, wherein R¹ and R² are independently D or H, wherein at least one of R¹ and R² is D, and typically both R¹ and R² are D, R⁸ is isopropyl, R⁶ and R^{7b} are H, R^{7a} is an amino acid side chain, and R⁵ is phenyl.

In some embodiments, structures are provided including 6-8, wherein R¹ and R² are independently D or H, wherein at least one of R¹ and R² is D, and typically both R¹ and R² are D, R⁸ is isopropyl, R⁶ and R^{7b} are H, R^{7a} is methyl, and R⁵ is phenyl.

In some embodiments, structures are provided including 6-8, wherein R¹ and R² are independently D or H, wherein at least one of R¹ and R² is D, and typically both R¹ and R² are D, R⁸ is benzyl, R⁶ and R^{7b} are H, R^{7a} is an amino acid side chain, and R⁵ is phenyl.

In some embodiments, structures are provided including 6-8, wherein R¹ and R² are independently D or H, wherein at least one of R¹ and R² is D, and typically both R¹ and R² are D, R⁸ is benzyl, R⁶ and R^{7b} are H, R^{7a} is methyl, and R⁵ is phenyl.

In some embodiments, structures are provided including 6-8, wherein R¹ and R² are independently D or H, wherein at least one of R¹ and R² is D, and typically both R¹ and R² are D, R⁸ is benzyl, R⁶ and R^{7b} are H, R^{7a} is an amino acid side chain, and R⁵ is 1-naphthyl or 2-naphthyl.

In some embodiments, structures are provided including 6-8, wherein R¹ and R² are independently D or H, wherein at least one of R¹ and R² is D, and typically both R¹ and R² are D, R⁸ is benzyl, R⁶ and R^{7b} are H, R^{7a} is methyl, and R⁵ is 1-naphthyl or 2-naphthyl.

In some embodiments, structures are provided including 6-8, wherein R¹ and R² are independently D or H, wherein at least one of R¹ and R² is D, and typically both R¹ and R² are D, R⁸ is isopropyl, R⁶ and R^{7b} are H, R^{7a} is an amino acid side chain, and R⁵ is 1-naphthyl or 2-naphthyl.

In some embodiments, structures are provided including 6-8, wherein R¹ and R² are independently D or H, wherein at least one of R¹ and R² is D, and typically both R¹ and R² are D, R⁸ is isopropyl, R⁶ and R^{7b} are H, R^{7a} is methyl, and R⁵ is 1-naphthyl or 2-naphthyl.

In some embodiments, structures are provided including 6-8, wherein R¹ and R² are deuterium, R⁸ is isopropyl, R⁶ and R^{7b} are H, R^{7a} is an amino acid side chain, and R⁵ is phenyl.

In some embodiments, structures are provided including 6-8, wherein R¹ and R² are deuterium, R⁸ is isopropyl, R⁶ and R^{7b} are H, R^{7a} is methyl, and R⁵ is phenyl.

In some embodiments, structures are provided including 6-8, wherein R¹ and R² are deuterium, R⁸ is benzyl, R⁶ and R^{7b} are H, R^{7a} is an amino acid side chain, and R⁵ is phenyl.

In some embodiments, structures are provided including 6-8, wherein R¹ and R² are deuterium, R⁸ is benzyl, R⁶ and R^{7b} are H, R^{7a} is methyl, and R⁵ is phenyl.

In some embodiments, structures are provided including 6-8, wherein R¹ and R² are deuterium, R⁸ is benzyl, R⁶ and R^{7b} are H, R^{7a} is an amino acid side chain, and R⁵ is 1-naphthyl or 2-naphthyl.

In some embodiments, structures are provided including 6-8, wherein R¹ and R² are deuterium, R⁸ is benzyl, R⁶ and R^{7b} are H, R^{7a} is methyl, and R⁵ is 1-naphthyl or 2-naphthyl.

In some embodiments, structures are provided including 6-8, wherein R¹ and R² are deuterium, R⁸ is isopropyl, R⁶ and R^{7b} are H, R^{7a} is an amino acid side chain, and R⁵ is 1-naphthyl or 2-naphthyl.

In some embodiments, structures are provided including 6-8, wherein R¹ and R² are deuterium, R⁸ is isopropyl, R⁶ and R^{7b} are H, R^{7a} is methyl, and R⁵ is 1-naphthyl or 2-naphthyl.

In some embodiments, structures are provided including 6-9, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7a} are H, R^{7b} is an amino acid side chain, R¹⁸ is C₁-C₆alkyl or C₃-C₆cycloalkyl, R¹⁶ and R^{17a} are H, and R^{17b} is an amino acid side chain.

In some embodiments, structures are provided including 6-9, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7a} are H, R^{7b} is C₁-C₆alkyl, R¹⁸ is C₁-C₆alkyl or C₃-C₆cycloalkyl, R¹⁶ and R^{17a} are H, and R^{17b} is C₁-C₆alkyl.

In some embodiments, structures are provided including 6-9, wherein R¹ and R² are independently D or H, wherein at least one of R¹ and R² is D, and typically both R¹ and R² are D, R⁸ is -alkylaryl, R⁶ and R^{7a} are H, R^{7b} is an amino acid side chain, R¹⁸ is -alkylaryl, R¹⁶ and R^{17a} are H, and R^{17b} is an amino acid side chain.

In some embodiments, structures are provided including 6-9, wherein R¹ and R² are independently D or H, wherein at least one of R¹ and R² is D, and typically both R¹ and R² are D, R⁸ is -alkylaryl, R⁶ and R^{7a} are H, R^{7b} is C₁-C₆alkyl, R¹⁸ is -alkylaryl, R¹⁶ and R^{17a} are H, and R^{17b} is C₁-C₆alkyl.

In some embodiments, structures are provided including 6-9, wherein R¹ and R² are independently D or H, wherein at least one of R¹ and R² is D, and typically both R¹ and R² are D, R⁸ is -alkylaryl, R⁶ and R^{7a} are H, R^{7b} is an amino acid side chain, R¹⁸ is -alkylaryl, R¹⁶ and R^{17a} are H, and R^{17b} is an amino acid side chain.

In some embodiments, structures are provided including 6-9, wherein R¹ and R² are independently D or H, wherein at least one of R¹ and R² is D, and typically both R¹ and R² are D, R⁸ is -alkylaryl, R⁶ and R^{7a} are H, R^{7b} is C₁-C₆alkyl, R¹⁸ is -alkylaryl, R¹⁶ and R^{17a} are H, and R^{17b} is C₁-C₆alkyl.

In some embodiments, structures are provided including 6-9, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7a} are H, R^{7b} is an amino acid side chain, R¹⁸ is C₁-C₆alkyl or C₃-C₆cycloalkyl, R¹⁶ and R^{17a} are H, and R^{17b} is an amino acid side chain.

In some embodiments, structures are provided including 6-9, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7a} are H, R^{7b} is C₁-C₆alkyl, R¹⁸ is C₁-C₆alkyl or C₃-C₆cycloalkyl, R¹⁶ and R^{17a} are H, and R^{17b} is C₁-C₆alkyl.

In some embodiments, structures are provided including 6-9, wherein R¹ and R² are independently D or H, wherein at least one of R¹ and R² is D, and typically both R¹ and R² are D, R⁸ is isopropyl, R⁶ and R^{7a} are H, R^{7b} is an amino acid side chain, R¹⁸ is isopropyl, R¹⁶ and R^{17a} are H, and R^{17b} is an amino acid side chain.

In some embodiments, structures are provided including 6-9, wherein R¹ and R² are independently D or H, wherein at least one of R¹ and R² is D, and typically both R¹ and R² are D, R⁸ is isopropyl, R⁶ and R^{7a} are H, R^{7b} is methyl, R¹⁸ is isopropyl, R¹⁶ and R^{17a} are H, and R^{17b} is methyl.

In some embodiments, structures are provided including 6-9, wherein R¹ and R² are independently D or H, wherein at least one of R¹ and R² is D, and typically both R¹ and R² are D, R⁸ is benzyl, R⁶ and R^{7a} are H, R^{7b} is an amino acid side chain, R¹⁸ is benzyl, R¹⁶ and R^{17a} are H, and R^{17b} is an amino acid side chain.

In some embodiments, structures are provided including 6-9, wherein R¹ and R² are independently D or H, wherein at least one of R¹ and R² is D, and typically both R¹ and R² are D, R⁸ is benzyl, R⁶ and R^{7a} are H, R^{7b} is methyl, R¹⁸ is benzyl, R¹⁶ and R^{17a} are H, and R^{17b} is methyl.

In some embodiments, structures are provided including 6-9, wherein R¹ and R² are independently D or H, wherein at least one of R¹ and R² is D, and typically both R¹ and R² are D, R⁸ is benzyl, R⁶ and R^{7a} are H, R^{7b} is an amino acid side chain, R¹⁸ is benzyl, R¹⁶ and R^{17a} are H, and R^{17b} is an amino acid side chain.

In some embodiments, structures are provided including 6-9, wherein R¹ and R² are independently D or H, wherein at least one of R¹ and R² is D, and typically both R¹ and R² are D, R⁸ is benzyl, R⁶ and R^{7a} are H, R^{7b} is methyl, R¹⁸ is benzyl, R¹⁶ and R^{17a} are H, and R^{17b} is methyl.

In some embodiments, structures are provided including 6-9, wherein R¹ and R² are independently D or H, wherein at least one of R¹ and R² is D, and typically both R¹ and R² are D, R⁸ is isopropyl, R⁶ and R^{7a} are H, R^{7b} is an amino acid side chain, R¹⁸ is isopropyl, R¹⁶ and R^{17a} are H, and R^{17b} is an amino acid side chain.

In some embodiments, structures are provided including 6-9, wherein R¹ and R² are independently D or H, wherein at least one of R¹ and R² is D, and typically both R¹ and R² are D, R⁸ is isopropyl, R⁶ and R^{7a} are H, R^{7b} is methyl, R¹⁸ is isopropyl, R¹⁶ and R^{17a} are H, and R^{17b} is methyl.

In some embodiments, structures are provided including 6-9, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7b} are H, R^{7a} is an amino acid side chain, R¹⁸ is C₁-C₆alkyl or C₃-C₆cycloalkyl, R¹⁶ and R^{17b} are H, and R^{17a} is an amino acid side chain.

In some embodiments, structures are provided including 6-9, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7b} are H, R^{7a} is C₁-C₆alkyl, R¹⁸ is C₁-C₆alkyl or C₃-C₆cycloalkyl, R¹⁶ and R^{17b} are H, and R^{17a} is C₁-C₆alkyl.

In some embodiments, structures are provided including 6-9, wherein R¹ and R² are independently D or H, wherein at least one of R¹ and R² is D, and typically both R¹ and R² are D, R⁸ is -alkylaryl, R⁶ and R^{7b} are H, R^{7a} is an amino acid side chain, R¹⁸ is -alkylaryl, R¹⁶ and R^{17b} are H, and R^{17a} is an amino acid side chain.

In some embodiments, structures are provided including 6-9, wherein R¹ and R² are independently D or H, wherein at least one of R¹ and R² is D, and typically both R¹ and R² are D, R⁸ is -alkylaryl, R⁶ and R^{7b} are H, R^{7a} is C₁-C₆alkyl, R¹⁸ is -alkylaryl, R¹⁶ and R^{17b} are H, and R^{17a} is C₁-C₆alkyl.

In some embodiments, structures are provided including 6-9, wherein R¹ and R² are independently D or H, wherein at least one of R¹ and R² is D, and typically both R¹ and R² are D, R⁸ is -alkylaryl, R⁶ and R^{7b} are H, R^{7a} is an amino acid side chain, R¹⁸ is -alkylaryl, R¹⁶ and R^{17b} are H, and R^{17a} is an amino acid side chain.

In some embodiments, structures are provided including 6-9, wherein R¹ and R² are independently D or H, wherein at least one of R¹ and R² is D, and typically both R¹ and R² are D, R⁸ is -alkylaryl, R⁶ and R^{7b} are H, R^{7a} is C₁-C₆alkyl, R¹⁸ is -alkylaryl, R¹⁶ and R^{17b} are H, and R^{17a} is C₁-C₆alkyl.

In some embodiments, structures are provided including 6-9, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7b} are H, R^{7a} is an amino acid side chain, R¹⁸ is C₁-C₆alkyl or C₃-C₆cycloalkyl, R¹⁶ and R^{17b} are H, and R^{17a} is an amino acid side chain.

In some embodiments, structures are provided including 6-9, wherein R¹ and R² are independently D or H, wherein at least one of R¹ and R² is D, and typically both R¹ and R² are D, R⁸ is C₁-C₆alkyl or C₃-C₆cycloalkyl, R⁶ and R^{7b} are H, R^{7a} is C₁-C₆alkyl, R¹⁸ is C₁-C₆alkyl or C₃-C₆cycloalkyl, R¹⁶ and R^{17b} are H, and R^{17a} is C₁-C₆alkyl.

In some embodiments, structures are provided including 6-9, wherein R¹ and R² are independently D or H, wherein at least one of R¹ and R² is D, and typically both R¹ and R² are D, R⁸ is isopropyl, R⁶ and R^{7b} are H, R^{7a} is an amino acid side chain, R¹⁸ is isopropyl, R¹⁶ and R^{17b} are H, and R^{17a} is an amino acid side chain.

In some embodiments, structures are provided including 6-9, wherein R¹ and R² are independently D or H, wherein at least one of R¹ and R² is D, and typically both R¹ and R² are D, R⁸ is isopropyl, R⁶ and R^{7b} are H, R^{7a} is methyl, R¹⁸ is isopropyl, R¹⁶ and R^{17b} are H, and R^{17a} is methyl.

In some embodiments, structures are provided including 6-9, wherein R¹ and R² are independently D or H, wherein at least one of R¹ and R² is D, and typically both R¹ and R² are D, R⁸ is benzyl, R⁶ and R^{7b} are H, R^{7a} is an amino acid side chain, R¹⁸ is benzyl, R¹⁶ and R^{17b} are H, and R^{17a} is an amino acid side chain.

In some embodiments, structures are provided including 6-9, wherein R¹ and R² are independently D or H, wherein at least one of R¹ and R² is D, and typically both R¹ and R² are D, R⁸ is benzyl, R⁶ and R^{7b} are H, R^{7a} is methyl, R¹⁸ is benzyl, R¹⁶ and R^{17b} are H, and R^{17a} is methyl.

In some embodiments, structures are provided including 6-9, wherein R¹ and R² are independently D or H, wherein at least one of R¹ and R² is D, and typically both R¹ and R² are D, R⁸ is benzyl, R⁶ and R^{7b} are H, R^{7a} is an amino acid side chain, R¹⁸ is benzyl, R¹⁶ and R^{17b} are H, and R^{17a} is an amino acid side chain.

In some embodiments, structures are provided including 6-9, wherein R¹ and R² are independently D or H, wherein at least one of R¹ and R² is D, and typically both R¹ and R² are D, R⁸ is benzyl, R⁶ and R^{7b} are H, R^{7a} is methyl, R¹⁸ is benzyl, R¹⁶ and R^{17b} are H, and R^{17a} is methyl.

In some embodiments, structures are provided including 6-9, wherein R¹ and R² are independently D or H, wherein at least one of R¹ and R² is D, and typically both R¹ and R² are D, R⁸ is isopropyl, R⁶ and R^{7b} are H, R^{7a} is an amino acid side chain, R¹⁸ is isopropyl, R¹⁶ and R^{17b} are H, and R^{17a} is an amino acid side chain.

In some embodiments, structures are provided including 6-9, wherein R¹ and R² are independently D or H, wherein at least one of R¹ and R² is D, and typically both R¹ and R² are D, R⁸ is isopropyl, R⁶ and R^{7b} are H, R^{7a} is methyl, R¹⁸ is isopropyl, R¹⁶ and R^{17b} are H, and R^{17a} is methyl.

In some embodiments, structures are provided including 6-10, wherein R¹ and R² are independently D or H, wherein at least one of R¹ and R² is D, and typically both R¹ and R² are D, R¹⁵ is —C₁-C₃alkyl-O—C₁-C₅alkoxy, R¹⁹ is H, and R²⁰ is -alkylaryl.

In some embodiments, structures are provided including 6-10, wherein R¹ and R² are independently D or H, wherein at least one of R¹ and R² is D, and typically both R¹ and R² are D, R¹⁵ is C₁-C₆alkyl, R¹⁹ is H, and R²⁰ is -alkylaryl.

In some embodiments, structures are provided including 6-10, wherein R¹ and R² are independently D or H, wherein at least one of R¹ and R² is D, and typically both R¹ and R² are D, R¹⁵ is isopropoxyl, R¹⁹ is H, and R²⁰ is -alkylaryl.

In some embodiments, structures are provided including 6-10, wherein R¹ and R² are independently D or H, wherein at least one of R¹ and R² is D, and typically both R¹ and R² are D, R¹⁵ is —C₁-C₃alkyl-O—C₁-C₅alkoxy, R¹⁹ is H, and R²⁰ is benzyl.

In some embodiments, structures are provided including 6-10, wherein R¹ and R² are independently D or H, wherein at least one of R¹ and R² is D, and typically both R¹ and R² are D, R¹⁵ is C₁-C₆alkyl, R¹⁹ is H, and R²⁰ is benzyl.

In some embodiments, structures are provided including 6-10, wherein R¹ and R² are independently D or H, wherein at least one of R¹ and R² is D, and typically both R¹ and R² are D, R¹⁵ is isopropoxyl, R¹⁹ is H, and R²⁰ is benzyl.

In some embodiments, structures are provided including 6-11, R¹ and R² are independently D or H, wherein at least one of R¹ and R² is D, and typically both R¹ and R² are D, R^{21a} and R^{21b} are hydrogen, and R¹⁵ is —C₁-C₃alkyl-O—C₁-C₅alkoxy.

In some embodiments, structures are provided including 6-11, R¹ and R² are independently D or H, wherein at least one of R¹ and R² is D, and typically both R¹ and R² are D, R^{21a} and R^{21b} are hydrogen, and R¹⁵ is C₁-C₆alkyl.

In some embodiments, structures are provided including 6-11, R¹ and R² are independently D or H, wherein at least one of R¹ and R² is D, and typically both R¹ and R² are D, R^{21a} and R^{21b} are hydrogen, and R¹⁵ is isopropoxyl.

In some embodiments, structures are provided including 6-11, R¹ and R² are independently D or H, wherein at least one of R¹ and R² is D, and typically both R¹ and R² are D, R^{21a} and R^{21b} are hydrogen, and R¹⁵ is t-butyl.

In some embodiments, structures are provided including 6-12, R¹ and R² are independently D or H, wherein at least one of R¹ and R² is D, and typically both R¹ and R² are D, R^{21a} and R^{21b} are hydrogen, R¹⁵ is —C₁-C₃alkyl-O—C₁-C₅alkoxy, R¹⁹ is H, and R²⁰ is -alkylaryl.

In some embodiments, structures are provided including 6-12, R¹ and R² are independently D or H, wherein at least one of R¹ and R² is D, and typically both R¹ and R² are D, R^{21a} and R^{21b} are hydrogen, R¹⁵ is C₁-C₆alkyl, R¹⁹ is H, and R²⁰ is -alkylaryl.

In some embodiments, structures are provided including 6-12, R¹ and R² are independently D or H, wherein at least one of R¹ and R² is D, and typically both R¹ and R² are D, R^{21a} and R^{21b} are hydrogen, R¹⁵ is isopropoxyl, R¹⁹ is H, and R²⁰ is benzyl.

In some embodiments, structures are provided including 6-12, R¹ and R² are independently D or H, wherein at least one of R¹ and R² is D, and typically both R¹ and R² are D, R^{21a} and R^{21b} are hydrogen, R¹⁵ is t-butyl, R¹⁹ is H, and R²⁰ is benzyl.

In some embodiments, structures are provided including 6-12, R¹ and R² are independently D or H, wherein at least one of R¹ and R² is D, and typically both R¹ and R² are D, R^{21a} and R^{21b} are hydrogen, R¹⁵ is isopropoxyl, R¹⁹ is H, and R²⁰ is -alkylaryl.

In some embodiments, structures are provided including 6-12, R¹ and R² are independently D or H, wherein at least one of R¹ and R² is D, and typically both R¹ and R² are D, R^{21a} and R^{21b} are hydrogen, R¹⁵ is t-butyl, R¹⁹ is H, and R²⁰ is -alkylaryl.

In some embodiments, structures are provided including 6-12, R¹ and R² are independently D or H, wherein at least one of R¹ and R² is D, and typically both R¹ and R² are D, R^{21a} and R^{21b} are hydrogen, R¹⁵ is —C₁-C₃alkyl-O—C₁-C₅alkoxy, R¹⁹ is H, and R²⁰ is benzyl.

In some embodiments, structures are provided including 6-12, R¹ and R² are independently D or H, wherein at least one of R¹ and R² is D, and typically both R¹ and R² are D, R^{21a} and R^{21b} are hydrogen, R¹⁵ is C₁-C₆alkyl, R¹⁹ is H, and R²⁰ is benzyl.

In one embodiment, T is oxygen.

In one embodiment, T is sulfur.

In one embodiment, T is CR³³R³⁴.

In one embodiment, the 5′-deuterated structures as described herein do not have a phosphate or phosphorus atom attached to the 3′-position. In another embodiment, the 5′-deuterated structures as described herein have enriched deuterium only in the 5′-position of the molecule. In yet another embodiment, the 5′-deuterated structures as described herein only have enriched deuterium in the 5′-position of the molecule and in the base (i.e., the pyrimidine or purine base or other base as described herein). In another embodiment, any of the hydrogens of the nucleoside can be substituted for enriched deuterium as long as there is at least one, and typically two, enriched deuterium in the 5′-position. In yet another embodiment, the 5′-oxygen is not bound to a carbon to create a 5′-ether group. In none of the embodiments is the 5′-oxygen bound to a synthetic oxygen protecting group, as is used to make oligonucleotides.

In one embodiment, an effective amount of the 5′-deuterated phosphate, 5′-deuterated-hydroxyl, 5′-deuterated stabilized nucleotide prodrug, or 5′-deuterated stabilized acyclic nucleoside prodrug as further described herein or its pharmaceutically acceptable salt, optionally in a pharmaceutically acceptable carrier, is provided to a host in need of anti-viral therapy, including anti-HCV therapy.

For example, in one embodiment, a nucleos(t)ide selected from those listed below can be administered in the form of a 5′-deuterated prodrug, for example as described in Table 5: Edoxudine (EdU, Aedurid) developed by Upjohn and approved for use in the treatment of herpes simplex virus (HSV); Vidarabine (Ara-A, Vira-A) developed by Parkedale Pharmaceuticals and approved for use in the treatment of HSV and varicella-zoster virus (VZV); Brivudine (BVDU, Helpin) developed by Berlin Chemie and approved for use in the treatment of HSV and VZV; Idoxuridine (IdU, Herplex) developed by GSK and approved for use in the treatment of HSV and VZV; Trifluridine (F3T, Viroptic) developed by King Pharmaceuticals and approved for use in the treatment of HSV; Stavudine (d4T, Zerit) developed by BMS and approved for use in the treatment of human immunodeficiency virus (HIV); Lamivudine (3TC, Epivir) developed by GSK and approved for use in the treatment of HIV and hepatitis B virus (HBV); Abacavir (ABV, Ziagen) developed by GSK and approved for use in the treatment of HIV; and Emtricitabine (FTC, Emtriva) developed by Gilead Sciences and approved for use in the treatment of HIV and HBV. Other nucleosides that can be modified as described are Entecavir (BMS, Baraclude), Telbivudine (Novartis, Tyzeka), Clevudine (Bukwang, Levovir) for the treatment of hepatitis B.

In another embodiment, an acyclic nucleoside selected from those listed below can be administered in the form of a 5′-deuterated stabilized acyclic nucleoside prodrug, for example as described in Table 6: Acyclovir (ACV, Aciclovir) developed by many manufacturers and approved for use in the treatment of HSV and VZV; Valacyclovir (Valtrex) developed by GSK for use in the treatment of HSV, herpes labialis, and VZV; Ganciclovir (GCV, Cytovene) developed by Hoffmann-La Roche and approved for use in the treatment of CMV; Valganciclovir (VGCV, Valcyte) developed by Hoffmann-La Roche and approved for use in the treatment of CMV; Penciclovir (PE2, Denavir) developed by Novartis and approved for use in the treatment of HSV, and Famciclovir, developed by Novartis for the treatment of HSV.

In one embodiment, an effective amount of the 5′-deuterated phosphate, 5′-deuterated-hydroxyl, 5′-deuterated stabilized nucleotide prodrug, or 5′-deuterated acyclic nucleoside prodrug as further described herein or it's pharmaceutically acceptable salt, optionally in a pharmaceutically acceptable carrier, is provided to a host in need of anti-neoplastic therapy, including anti-cancer therapy.

For example, in one embodiment, a nucleos(t)ide selected from those listed below can be administered in the form of a 5′-deuterated prodrug, for example as described in Table 5: Cytarabine (AraC, Aracytine) developed by Pfizer and approved for use in the treatment of acute myeloid leukemia, acute lymphocytic leukemia, and lymphomas; Fludarabine (FaraAMP, Fludara) developed by Genzyme and approved for use in the treatment of chronic lymphocytic leukemia, Non-Hodgkins lymphoma, acute myeloid leukemia; Cladribine (2CdA, Leustatine) developed by Janssen-Cilag and approved for use in the treatment of Hairy cell leukemia; Gemcitabine (dFdC, Gemzar) developed by Eli Lilly and Co. and approved for use in the treatment of non-small lung cancer, pancreatic cancer, bladder cancer, and breast cancer; Clofarabine (CAFdA, Evoltra, Clolar) developed by Genzyme and approved for use in the treatment of acute lymphocytic leukemia; Nelarabine (AraG, Atriance, Arranon) developed by SmithKline Beecham and approved for use in the treatment of T-cell acute lymphoblastic leukemia; Capecitabine (Xeloda) developed by Hoffmann-La Roche and approved for use in the treatment of metastatic breast cancer, metastatic colorectal cancer; Floxuridine (FUDR) developed by Hospira and approved for use in the treatment of advanced colon cancer, kidney cancer, and stomach cancer; Deoxycoformycin (pentostatin, Nipent) developed by Hospira Inc., for use in the treatment of hairy cell leukemia, chronic lymphocytic leukemia; Azacitidine (Vidaza) developed by Celgene for use in the treatment of myelodysplastic syndrome; Decitabine (Dacogen) developed by Eisai Inc. and Janssen-Cilag for use in the treatment of myelodysplastic syndrome and acute myeloid leukemia.

5′-Deuterated Stabilized Nucleotide Phosphoramidates

A number of publications and patents describe a range of nucleotide phosphoramidates. According to the present invention, any of such disclosed phosphoramidates can be stabilized via 5′-deuteration. For example, see McGuigan C, et al. Synthesis, anti-human immunodeficiency virus activity and esterase lability of some novel carboxylic ester-modified phosphoramidate derivatives of stavudine (d4T). Antiviral Chemistry and Chemotherapy. 1998, Volume 9:473-9; McGuigan C, et al. Phosphoramidate derivatives of d4T as inhibitors of HIV: the effect of amino acid variation. Antiviral Res. 1997 August; 35(3):195-204) and Siddiqui, A Q, et al. The presence of substituents on the aryl moiety of the aryl phosphoramidate derivative of d4T enhances anti-HIV efficacy in cell culture: A structure-activity relationship. J. Med. Chem. 1999, Volume 42: 393-399. See also, U.S. Pat. No. 7,951,787 (Phosphoramidate compounds and methods of use), U.S. Pat. No. 7,115,590 (Phosphoramidate, and mono-, di-, and tri-phosphate esters of (1R, cis)-4-(6-amino-9H-purin-9-yl)-2-cyclopentene-1-methanol as antiviral agents), U.S. Pat. No. 8,263,575 (Phosphoramidate derivatives of nucleoside compounds for use in the treatment of cancer), and U.S. Pat. No. 8,658,616 (Nucleoside aryl phosphoramidates and their use as anti-viral agents for the treatment of hepatitis C virus), U.S. Pat. No. 8,119,779 (Phosphoramidate Derivatives). Other publications include: McGuigan C, et al. Phosphorodiamidates as a promising new phosphate prodrug motif for antiviral drug discovery: application to anti-HCV agents. J. Med. Chem. 2011 Dec. 22; 54(24):8632-45; McGuigan C, et al. Dual pro-drugs of 2′-C-methyl guanosine monophosphate as potent and selective inhibitors of hepatitis C virus. Bioorg Med Chem Lett. 2011 Oct. 1; 21(19):6007-12; Quintiliani M, et al. Design, synthesis and biological evaluation of 2′-deoxy-2′,2′-difluoro-5-halouridine phosphoramidate ProTides. Bioorg Med Chem. 2011 Jul. 15; 19(14):4338-45; and McGuigan C, et al. Phosphoramidate ProTides of 2′-C-methylguanosine as highly potent inhibitors of hepatitis C virus. Study of their in vitro and in vivo properties. J Med Chem. 2010 Jul. 8; 53(13):4949-57. The phosphoramidate can include an amino acid residue that has either a L or D stereoconfiguration. See for example, McGuigan C, et al. The phosphoramidate ProTide approach greatly enhances the activity of beta-2′-C-methylguanosine against hepatitis C virus. Bioorg Med Chem Lett. 2009 Aug. 1; 19(15):4316-20) and WO 2013/177219 filed by Idenix Pharmaceuticals, et al.

In one embodiment, the invention is 5′-deuterated-2′-methyluridine phosphoramidate of the structure below, and its compositions and medical uses, including to treat hepatitis C,

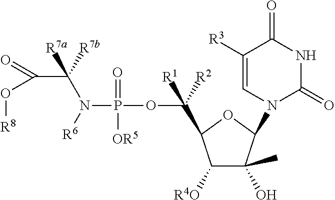

wherein R¹ and R² are both deuterium or at least one of R¹ and R² is deuterium and the other is hydrogen, R³ is hydrogen or deuterium. In one embodiment, R⁴ and R⁶ are hydrogen, R⁵, R^{7a}, R^{7b}, and R⁸ are as provided below in Table 7. In an alternative embodiment, R^{7a} and R^{7b} are reversed to form an amino acid residue with D-stereoconfiguration.

TABLE 7
5′-Deuterated-2′-β-methyl-uridine phosphoramidates

| Structure | R⁵ | R^{7a} | R^{7b} | R⁸ |
|---|---|---|---|---|
| 1-8-1 | Ph | H | H | iPr |
| 1-8-2 | Ph | H | CH₃ | iPr |
| 1-8-3 | Ph | H | CH(CH₃)₂ | iPr |
| 1-8-4 | Ph | H | CH₂CH(CH₃)₂ | iPr |
| 1-8-5 | Ph | H | CH(CH₃)CH₂CH₃ | iPr |
| 1-8-6 | Ph | H | CH₂Ph | iPr |
| 1-8-7 | Ph | H | CH₂-indol-3-yl | iPr |
| 1-8-8 | Ph | H | CH₂CH₂SCH₃ | iPr |
| 1-8-9* | Ph | H | * | iPr |
| 1-8-10 | Ph | H | H | nBu |
| 1-8-11 | Ph | H | CH₃ | nBu |
| 1-8-12 | Ph | H | CH(CH₃)₂ | nBu |
| 1-8-13 | Ph | H | CH₂CH(CH₃)₂ | nBu |
| 1-8-14 | Ph | H | CH(CH₃)CH₂CH₃ | nBu |
| 1-8-15 | Ph | H | CH₂Ph | nBu |
| 1-8-16 | Ph | H | CH₂-indol-3-yl | nBu |
| 1-8-17 | Ph | H | CH₂CH₂SCH₃ | nBu |
| 1-8-18* | Ph | H | * | nBu |
| 1-8-19 | Ph | H | H | Ph |
| 1-8-20 | Ph | H | CH₃ | Ph |
| 1-8-21 | Ph | H | CH(CH₃)₂ | Ph |
| 1-8-22 | Ph | H | CH₂CH(CH₃)₂ | Ph |
| 1-8-23 | Ph | H | CH(CH₃)CH₂CH₃ | Ph |
| 1-8-24 | Ph | H | CH₂Ph | Ph |
| 1-8-25 | Ph | H | CH₂-indol-3-yl | Ph |
| 1-8-26 | Ph | H | CH₂CH₂SCH₃ | Ph |
| 1-8-27* | Ph | H | * | Ph |
| 1-8-28 | Ph | H | H | Bn |
| 1-8-29 | Ph | H | CH₃ | Bn |
| 1-8-30 | Ph | H | CH(CH₃)₂ | Bn |
| 1-8-31 | Ph | H | CH₂CH(CH₃)₂ | Bn |
| 1-8-32 | Ph | H | CH(CH₃)CH₂CH₃ | Bn |
| 1-8-33 | Ph | H | CH₂Ph | Bn |
| 1-8-34 | Ph | H | CH₂-indol-3-yl | Bn |
| 1-8-35 | Ph | H | CH₂CH₂SCH₃ | Bn |
| 1-8-36* | Ph | H | * | Bn |
| 1-8-37 | Ph | H | H | CH₃ |
| 1-8-38 | Ph | H | CH₃ | CH₃ |
| 1-8-39 | Ph | H | CH(CH₃)₂ | CH₃ |
| 1-8-40 | Ph | H | CH₂CH(CH₃)₂ | CH₃ |
| 1-8-41 | Ph | H | CH(CH₃)CH₂CH₃ | CH₃ |
| 1-8-42 | Ph | H | CH₂Ph | CH₃ |
| 1-8-43 | Ph | H | CH₂-indol-3-yl | CH₃ |
| 1-8-44 | Ph | H | CH₂CH₂SCH₃ | CH₃ |
| 1-8-45* | Ph | H | * | CH₃ |
| 1-8-46 | Ph | H | H | Et |
| 1-8-47 | Ph | H | CH₃ | Et |
| 1-8-48 | Ph | H | CH(CH₃)₂ | Et |
| 1-8-49 | Ph | H | CH₂CH(CH₃)₂ | Et |
| 1-8-50 | Ph | H | CH(CH₃)CH₂CH₃ | Et |
| 1-8-51 | Ph | H | CH₂Ph | Et |
| 1-8-52 | Ph | H | CH₂-indol-3-yl | Et |
| 1-8-53 | Ph | H | CH₂CH₂SCH₃ | Et |
| 1-8-54* | Ph | H | * | Et |
| 1-8-55 | 1-Nap | H | H | iPr |
| 1-8-56 | 1-Nap | H | CH₃ | iPr |
| 1-8-57 | 1-Nap | H | CH(CH₃)₂ | iPr |
| 1-8-58 | 1-Nap | H | CH₂CH(CH₃)₂ | iPr |
| 1-8-59 | 1-Nap | H | CH(CH₃)CH₂CH₃ | iPr |
| 1-8-60 | 1-Nap | H | CH₂Ph | iPr |
| 1-8-61 | 1-Nap | H | CH₂-indol-3-yl | iPr |
| 1-8-62 | 1-Nap | H | CH₂CH₂SCH₃ | iPr |
| 1-8-63* | 1-Nap | H | * | iPr |
| 1-8-64 | 1-Nap | H | H | nBu |
| 1-8-65 | 1-Nap | H | CH₃ | nBu |
| 1-8-66 | 1-Nap | H | CH(CH₃)₂ | nBu |
| 1-8-67 | 1-Nap | H | CH₂CH(CH₃)₂ | nBu |
| 1-8-68 | 1-Nap | H | CH(CH₃)CH₂CH₃ | nBu |
| 1-8-69 | 1-Nap | H | CH₂Ph | nBu |
| 1-8-70 | 1-Nap | H | CH₂-indol-3-yl | nBu |
| 1-8-71 | 1-Nap | H | CH₂CH₂SCH₃ | nBu |
| 1-8-72* | 1-Nap | H | * | nBu |
| 1-8-73 | 1-Nap | H | H | Ph |
| 1-8-74 | 1-Nap | H | CH₃ | Ph |
| 1-8-75 | 1-Nap | H | CH(CH₃)₂ | Ph |
| 1-8-76 | 1-Nap | H | CH₂CH(CH₃)₂ | Ph |
| 1-8-77 | 1-Nap | H | CH(CH₃)CH₂CH₃ | Ph |
| 1-8-78 | 1-Nap | H | CH₂Ph | Ph |
| 1-8-79 | 1-Nap | H | CH₂-indol-3-yl | Ph |
| 1-8-80 | 1-Nap | H | CH₂CH₂SCH₃ | Ph |
| 1-8-81* | 1-Nap | H | * | Ph |
| 1-8-82 | 1-Nap | H | H | Bn |
| 1-8-83 | 1-Nap | H | CH₃ | Bn |
| 1-8-84 | 1-Nap | H | CH(CH₃)₂ | Bn |
| 1-8-85 | 1-Nap | H | CH₂CH(CH₃)₂ | Bn |
| 1-8-86 | 1-Nap | H | CH(CH₃)CH₂CH₃ | Bn |
| 1-8-87 | 1-Nap | H | CH₂Ph | Bn |
| 1-8-88 | 1-Nap | H | CH₂-indol-3-yl | Bn |
| 1-8-89 | 1-Nap | H | CH₂CH₂SCH₃ | Bn |
| 1-8-90* | 1-Nap | H | * | Bn |
| 1-8-91 | 1-Nap | H | H | CH₃ |
| 1-8-92 | 1-Nap | H | CH₃ | CH₃ |
| 1-8-93 | 1-Nap | H | CH(CH₃)₂ | CH₃ |
| 1-8-94 | 1-Nap | H | CH₂CH(CH₃)₂ | CH₃ |
| 1-8-95 | 1-Nap | H | CH(CH₃)CH₂CH₃ | CH₃ |
| 1-8-96 | 1-Nap | H | CH₂Ph | CH₃ |
| 1-8-97 | 1-Nap | H | CH₂-indol-3-yl | CH₃ |
| 1-8-98 | 1-Nap | H | CH₂CH₂SCH₃ | CH₃ |
| 1-8-99* | 1-Nap | H | * | CH₃ |
| 1-8-100 | 1-Nap | H | H | Et |
| 1-8-101 | 1-Nap | H | CH₃ | Et |
| 1-8-102 | 1-Nap | H | CH(CH₃)₂ | Et |
| 1-8-103 | 1-Nap | H | CH₂CH(CH₃)₂ | Et |
| 1-8-104 | 1-Nap | H | CH(CH₃)CH₂CH₃ | Et |
| 1-8-105 | 1-Nap | H | CH₂Ph | Et |
| 1-8-106 | 1-Nap | H | CH₂-indol-3-yl | Et |
| 1-8-107 | 1-Nap | H | CH₂CH₂SCH₃ | Et |
| 1-8-108* | 1-Nap | H | * | Et |
| 1-8-109 | 2-Nap | H | H | iPr |
| 1-8-110 | 2-Nap | H | CH₃ | iPr |
| 1-8-111 | 2-Nap | H | CH(CH₃)₂ | iPr |
| 1-8-112 | 2-Nap | H | CH₂CH(CH₃)₂ | iPr |
| 1-8-113 | 2-Nap | H | CH(CH₃)CH₂CH₃ | iPr |
| 1-8-114 | 2-Nap | H | CH₂Ph | iPr |
| 1-8-115 | 2-Nap | H | CH₂-indol-3-yl | iPr |
| 1-8-116 | 2-Nap | H | CH₂CH₂SCH₃ | iPr |
| 1-8-117* | 2-Nap | H | * | iPr |
| 1-8-118 | 2-Nap | H | H | nBu |
| 1-8-119 | 2-Nap | H | CH₃ | nBu |
| 1-8-120 | 2-Nap | H | CH(CH₃)₂ | nBu |
| 1-8-121 | 2-Nap | H | CH₂CH(CH₃)₂ | nBu |
| 1-8-122 | 2-Nap | H | CH(CH₃)CH₂CH₃ | nBu |
| 1-8-123 | 2-Nap | H | CH₂Ph | nBu |
| 1-8-124 | 2-Nap | H | CH₂-indol-3-yl | nBu |
| 1-8-125 | 2-Nap | H | CH₂CH₂SCH₃ | nBu |
| 1-8-126* | 2-Nap | H | * | nBu |
| 1-8-127 | 2-Nap | H | H | Ph |
| 1-8-128 | 2-Nap | H | CH₃ | Ph |
| 1-8-129 | 2-Nap | H | CH(CH₃)₂ | Ph |
| 1-8-130 | 2-Nap | H | CH₂CH(CH₃)₂ | Ph |
| 1-8-131 | 2-Nap | H | CH(CH₃)CH₂CH₃ | Ph |
| 1-8-132 | 2-Nap | H | CH₂Ph | Ph |
| 1-8-133 | 2-Nap | H | CH₂-indol-3-yl | Ph |
| 1-8-134 | 2-Nap | H | CH₂CH₂SCH₃ | Ph |
| 1-8-135* | 2-Nap | H | * | Ph |
| 1-8-136 | 2-Nap | H | H | Bn |
| 1-8-137 | 2-Nap | H | CH₃ | Bn |
| 1-8-138 | 2-Nap | H | CH(CH₃)₂ | Bn |
| 1-8-139 | 2-Nap | H | CH₂CH(CH₃)₂ | Bn |
| 1-8-140 | 2-Nap | H | CH(CH₃)CH₂CH₃ | Bn |
| 1-8-141 | 2-Nap | H | CH₂Ph | Bn |
| 1-8-142 | 2-Nap | H | CH₂-indol-3-yl | Bn |
| 1-8-143 | 2-Nap | H | CH₂CH₂SCH₃ | Bn |
| 1-8-144* | 2-Nap | H | * | Bn |
| 1-8-145 | 2-Nap | H | H | CH₃ |
| 1-8-146 | 2-Nap | H | CH₃ | CH₃ |
| 1-8-147 | 2-Nap | H | CH(CH₃)₂ | CH₃ |
| 1-8-148 | 2-Nap | H | CH₂CH(CH₃)₂ | CH₃ |
| 1-8-149 | 2-Nap | H | CH(CH₃)CH₂CH₃ | CH₃ |
| 1-8-150 | 2-Nap | H | CH₂Ph | CH₃ |
| 1-8-151 | 2-Nap | H | CH₂-indol-3-yl | CH₃ |
| 1-8-152 | 2-Nap | H | CH₂CH₂SCH₃ | CH₃ |
| 1-8-153* | 2-Nap | H | * | CH₃ |
| 1-8-154 | 2-Nap | H | H | Et |
| 1-8-155 | 2-Nap | H | CH₃ | Et |
| 1-8-156 | 2-Nap | H | CH(CH₃)₂ | Et |
| 1-8-157 | 2-Nap | H | CH₂CH(CH₃)₂ | Et |
| 1-8-158 | 2-Nap | H | CH(CH₃)CH₂CH₃ | Et |
| 1-8-159 | 2-Nap | H | CH₂Ph | Et |
| 1-8-160 | 2-Nap | H | CH₂-indol-3-yl | Et |
| 1-8-161 | 2-Nap | H | CH₂CH₂SCH₃ | Et |
| 1-8-162* | 2-Nap | H | * | Et |
| 1-8-163 | p-Br—Ph | H | H | iPr |
| 1-8-164 | p-Br—Ph | H | CH₃ | iPr |
| 1-8-165 | p-Br—Ph | H | CH(CH₃)₂ | iPr |
| 1-8-166 | p-Br—Ph | H | CH₂CH(CH₃)₂ | iPr |
| 1-8-167 | p-Br—Ph | H | CH(CH₃)CH₂CH₃ | iPr |
| 1-8-168 | p-Br—Ph | H | CH₂Ph | iPr |
| 1-8-169 | p-Br—Ph | H | CH₂-indol-3-yl | iPr |
| 1-8-170 | p-Br—Ph | H | CH₂CH₂SCH₃ | iPr |
| 1-8-171* | p-Br—Ph | H | * | iPr |
| 1-8-172 | p-Br—Ph | H | H | nBu |
| 1-8-173 | p-Br—Ph | H | CH₃ | nBu |
| 1-8-174 | p-Br—Ph | H | CH(CH₃)₂ | nBu |
| 1-8-175 | p-Br—Ph | H | CH₂CH(CH₃)₂ | nBu |
| 1-8-176 | p-Br—Ph | H | CH(CH₃)CH₂CH₃ | nBu |
| 1-8-177 | p-Br—Ph | H | CH₂Ph | nBu |
| 1-8-178 | p-Br—Ph | H | CH₂-indol-3-yl | nBu |
| 1-8-179 | p-Br—Ph | H | CH₂CH₂SCH₃ | nBu |
| 1-8-180* | p-Br—Ph | H | * | nBu |
| 1-8-181 | p-Br—Ph | H | H | Ph |
| 1-8-182 | p-Br—Ph | H | CH₃ | Ph |
| 1-8-183 | p-Br—Ph | H | CH(CH₃)₂ | Ph |
| 1-8-184 | p-Br—Ph | H | CH₂CH(CH₃)₂ | Ph |
| 1-8-185 | p-Br—Ph | H | CH(CH₃)CH₂CH₃ | Ph |
| 1-8-186 | p-Br—Ph | H | CH₂Ph | Ph |
| 1-8-187 | p-Br—Ph | H | CH₂-indol-3-yl | Ph |
| 1-8-188 | p-Br—Ph | H | CH₂CH₂SCH₃ | Ph |
| 1-8-189* | p-Br—Ph | H | * | Ph |
| 1-8-190 | p-Br—Ph | H | H | Bn |
| 1-8-191 | p-Br—Ph | H | CH₃ | Bn |
| 1-8-192 | p-Br—Ph | H | CH(CH₃)₂ | Bn |
| 1-8-193 | p-Br—Ph | H | CH₂CH(CH₃)₂ | Bn |
| 1-8-194 | p-Br—Ph | H | CH(CH₃)CH₂CH₃ | Bn |
| 1-8-195 | p-Br—Ph | H | CH₂Ph | Bn |
| 1-8-196 | p-Br—Ph | H | CH₂-indol-3-yl | Bn |
| 1-8-197 | p-Br—Ph | H | CH₂CH₂SCH₃ | Bn |
| 1-8-198* | p-Br—Ph | H | * | Bn |
| 1-8-199 | p-Br—Ph | H | H | CH₃ |
| 1-8-200 | p-Br—Ph | H | CH₃ | CH₃ |
| 1-8-201 | p-Br—Ph | H | CH(CH₃)₂ | CH₃ |
| 1-8-202 | p-Br—Ph | H | CH₂CH(CH₃)₂ | CH₃ |
| 1-8-203 | p-Br—Ph | H | CH(CH₃)CH₂CH₃ | CH₃ |
| 1-8-204 | p-Br—Ph | H | CH₂Ph | CH₃ |
| 1-8-205 | p-Br—Ph | H | CH₂-indol-3-yl | CH₃ |
| 1-8-206 | p-Br—Ph | H | CH₂CH₂SCH₃ | CH₃ |
| 1-8-207* | p-Br—Ph | H | * | CH₃ |
| 1-8-208 | p-Br—Ph | H | H | Et |
| 1-8-209 | p-Br—Ph | H | CH₃ | Et |
| 1-8-210 | p-Br—Ph | H | CH(CH₃)₂ | Et |
| 1-8-211 | p-Br—Ph | H | CH₂CH(CH₃)₂ | Et |
| 1-8-212 | p-Br—Ph | H | CH(CH₃)CH₂CH₃ | Et |
| 1-8-213 | p-Br—Ph | H | CH₂Ph | Et |
| 1-8-214 | p-Br—Ph | H | CH₂-indol-3-yl | Et |
| 1-8-215 | p-Br—Ph | H | CH₂CH₂SCH₃ | Et |
| 1-8-216* | p-Br—Ph | H | * | Et |
| 1-8-217 | p-Cl—Ph | H | H | iPr |
| 1-8-218 | p-Cl—Ph | H | CH₃ | iPr |
| 1-8-219 | p-Cl—Ph | H | CH(CH₃)₂ | iPr |
| 1-8-220 | p-Cl—Ph | H | CH₂CH(CH₃)₂ | iPr |
| 1-8-221 | p-Cl—Ph | H | CH(CH₃)CH₂CH₃ | iPr |
| 1-8-222 | p-Cl—Ph | H | CH₂Ph | iPr |
| 1-8-223 | p-Cl—Ph | H | CH₂-indol-3-yl | iPr |
| 1-8-224 | p-Cl—Ph | H | CH₂CH₂SCH₃ | iPr |
| 1-8-225* | p-Cl—Ph | H | * | iPr |
| 1-8-226 | p-Cl—Ph | H | H | nBu |
| 1-8-227 | p-Cl—Ph | H | CH₃ | nBu |
| 1-8-228 | p-Cl—Ph | H | CH(CH₃)₂ | nBu |
| 1-8-229 | p-Cl—Ph | H | CH₂CH(CH₃)₂ | nBu |
| 1-8-230 | p-Cl—Ph | H | CH(CH₃)CH₂CH₃ | nBu |
| 1-8-231 | p-Cl—Ph | H | CH₂Ph | nBu |
| 1-8-232 | p-Cl—Ph | H | CH₂-indol-3-yl | nBu |
| 1-8-233 | p-Cl—Ph | H | CH₂CH₂SCH₃ | nBu |
| 1-8-234* | p-Cl—Ph | H | * | nBu |
| 1-8-235 | p-Cl—Ph | H | H | Ph |
| 1-8-236 | p-Cl—Ph | H | CH₃ | Ph |
| 1-8-237 | p-Cl—Ph | H | CH(CH₃)₂ | Ph |
| 1-8-238 | p-Cl—Ph | H | CH₂CH(CH₃)₂ | Ph |
| 1-8-239 | p-Cl—Ph | H | CH(CH₃)CH₂CH₃ | Ph |
| 1-8-240 | p-Cl—Ph | H | CH₂Ph | Ph |
| 1-8-241 | p-Cl—Ph | H | CH₂-indol-3-yl | Ph |
| 1-8-242 | p-Cl—Ph | H | CH₂CH₂SCH₃ | Ph |
| 1-8-243* | p-Cl—Ph | H | * | Ph |
| 1-8-244 | p-Cl—Ph | H | H | Bn |
| 1-8-245 | p-Cl—Ph | H | CH₃ | Bn |
| 1-8-246 | p-Cl—Ph | H | CH(CH₃)₂ | Bn |
| 1-8-247 | p-Cl—Ph | H | CH₂CH(CH₃)₂ | Bn |
| 1-8-248 | p-Cl—Ph | H | CH(CH₃)CH₂CH₃ | Bn |
| 1-8-249 | p-Cl—Ph | H | CH₂Ph | Bn |
| 1-8-250 | p-Cl—Ph | H | CH₂-indol-3-yl | Bn |
| 1-8-251 | p-Cl—Ph | H | CH₂CH₂SCH₃ | Bn |
| 1-8-252* | p-Cl—Ph | H | * | Bn |
| 1-8-253 | p-Cl—Ph | H | H | CH₃ |
| 1-8-254 | p-Cl—Ph | H | CH₃ | CH₃ |
| 1-8-255 | p-Cl—Ph | H | CH(CH₃)₂ | CH₃ |
| 1-8-256 | p-Cl—Ph | H | CH₂CH(CH₃)₂ | CH₃ |
| 1-8-257 | p-Cl—Ph | H | CH(CH₃)CH₂CH₃ | CH₃ |
| 1-8-258 | p-Cl—Ph | H | CH₂Ph | CH₃ |
| 1-8-259 | p-Cl—Ph | H | CH₂-indol-3-yl | CH₃ |
| 1-8-260 | p-Cl—Ph | H | CH₂CH₂SCH₃ | CH₃ |
| 1-8-261* | p-Cl—Ph | H | * | CH₃ |
| 1-8-262 | p-Cl—Ph | H | H | Et |
| 1-8-263 | p-Cl—Ph | H | CH₃ | Et |
| 1-8-264 | p-Cl—Ph | H | CH(CH₃)₂ | Et |
| 1-8-265 | p-Cl—Ph | H | CH₂CH(CH₃)₂ | Et |
| 1-8-266 | p-Cl—Ph | H | CH(CH₃)CH₂CH₃ | Et |
| 1-8-267 | p-Cl—Ph | H | CH₂Ph | Et |
| 1-8-268 | p-Cl—Ph | H | CH₂-indol-3-yl | Et |
| 1-8-269 | p-Cl—Ph | H | CH₂CH₂SCH₃ | Et |
| 1-8-270* | p-Cl—Ph | H | * | Et |
| 1-8-271 | p-F—Ph | H | H | iPr |
| 1-8-272 | p-F—Ph | H | CH₃ | iPr |
| 1-8-273 | p-F—Ph | H | CH(CH₃)₂ | iPr |
| 1-8-274 | p-F—Ph | H | CH₂CH(CH₃)₂ | iPr |
| 1-8-275 | p-F—Ph | H | CH(CH₃)CH₂CH₃ | iPr |
| 1-8-276 | p-F—Ph | H | CH₂Ph | iPr |
| 1-8-277 | p-F—Ph | H | CH₂-indol-3-yl | iPr |
| 1-8-278 | p-F—Ph | H | CH₂CH₂SCH₃ | iPr |
| 1-8-279* | p-F—Ph | H | * | iPr |
| 1-8-280 | p-F—Ph | H | H | nBu |
| 1-8-281 | p-F—Ph | H | CH₃ | nBu |
| 1-8-282 | p-F—Ph | H | CH(CH₃)₂ | nBu |
| 1-8-283 | p-F—Ph | H | CH₂CH(CH₃)₂ | nBu |
| 1-8-284 | p-F—Ph | H | CH(CH₃)CH₂CH₃ | nBu |
| 1-8-285 | p-F—Ph | H | CH₂Ph | nBu |
| 1-8-286 | p-F—Ph | H | CH₂-indol-3-yl | nBu |
| 1-8-287 | p-F—Ph | H | CH₂CH₂SCH₃ | nBu |
| 1-8-288* | p-F—Ph | H | * | nBu |
| 1-8-289 | p-F—Ph | H | H | Ph |
| 1-8-290 | p-F—Ph | H | CH₃ | Ph |
| 1-8-291 | p-F—Ph | H | CH(CH₃)₂ | Ph |
| 1-8-292 | p-F—Ph | H | CH₂CH(CH₃)₂ | Ph |
| 1-8-293 | p-F—Ph | H | CH(CH₃)CH₂CH₃ | Ph |
| 1-8-294 | p-F—Ph | H | CH₂Ph | Ph |
| 1-8-295 | p-F—Ph | H | CH₂-indol-3-yl | Ph |
| 1-8-296 | p-F—Ph | H | CH₂CH₂SCH₃ | Ph |
| 1-8-297* | p-F—Ph | H | * | Ph |
| 1-8-298 | p-F—Ph | H | H | Bn |
| 1-8-299 | p-F—Ph | H | CH₃ | Bn |
| 1-8-300 | p-F—Ph | H | CH(CH₃)₂ | Bn |
| 1-8-301 | p-F—Ph | H | CH₂CH(CH₃)₂ | Bn |
| 1-8-302 | p-F—Ph | H | CH(CH₃)CH₂CH₃ | Bn |
| 1-8-303 | p-F—Ph | H | CH₂Ph | Bn |
| 1-8-304 | p-F—Ph | H | CH₂-indol-3-yl | Bn |
| 1-8-305 | p-F—Ph | H | CH₂CH₂SCH₃ | Bn |
| 1-8-306* | p-F—Ph | H | * | Bn |
| 1-8-307 | p-F—Ph | H | H | CH₃ |
| 1-8-308 | p-F—Ph | H | CH₃ | CH₃ |
| 1-8-309 | p-F—Ph | H | CH(CH₃)₂ | CH₃ |
| 1-8-310 | p-F—Ph | H | CH₂CH(CH₃)₂ | CH₃ |
| 1-8-311 | p-F—Ph | H | CH(CH₃)CH₂CH₃ | CH₃ |
| 1-8-312 | p-F—Ph | H | CH₂Ph | CH₃ |
| 1-8-313 | p-F—Ph | H | CH₂-indol-3-yl | CH₃ |
| 1-8-314 | p-F—Ph | H | CH₂CH₂SCH₃ | CH₃ |
| 1-8-315* | p-F—Ph | H | * | CH₃ |
| 1-8-316 | p-F—Ph | H | H | Et |
| 1-8-317 | p-F—Ph | H | CH₃ | Et |
| 1-8-318 | p-F—Ph | H | CH(CH₃)₂ | Et |
| 1-8-319 | p-F—Ph | H | CH₂CH(CH₃)₂ | Et |
| 1-8-320 | p-F—Ph | H | CH(CH₃)CH₂CH₃ | Et |
| 1-8-321 | p-F—Ph | H | CH₂Ph | Et |
| 1-8-322 | p-F—Ph | H | CH₂-indol-3-yl | Et |
| 1-8-323 | p-F—Ph | H | CH₂CH₂SCH₃ | Et |
| 1-8-324* | p-F—Ph | H | * | Et |
| 1-8-325 | p-I—Ph | H | H | iPr |
| 1-8-326 | p-I—Ph | H | CH₃ | iPr |
| 1-8-327 | p-I—Ph | H | CH(CH₃)₂ | iPr |
| 1-8-328 | p-I—Ph | H | CH₂CH(CH₃)₂ | iPr |
| 1-8-329 | p-I—Ph | H | CH(CH₃)CH₂CH₃ | iPr |
| 1-8-330 | p-I—Ph | H | CH₂Ph | iPr |
| 1-8-331 | p-I—Ph | H | CH₂-indol-3-yl | iPr |
| 1-8-332 | p-I—Ph | H | CH₂CH₂SCH₃ | iPr |
| 1-8-333* | p-I—Ph | H | * | iPr |
| 1-8-334 | p-I—Ph | H | H | nBu |
| 1-8-335 | p-I—Ph | H | CH₃ | nBu |
| 1-8-336 | p-I—Ph | H | CH(CH₃)₂ | nBu |
| 1-8-337 | p-I—Ph | H | CH₂CH(CH₃)₂ | nBu |
| 1-8-338 | p-I—Ph | H | CH(CH₃)CH₂CH₃ | nBu |
| 1-8-339 | p-I—Ph | H | CH₂Ph | nBu |
| 1-8-340 | p-I—Ph | H | CH₂-indol-3-yl | nBu |
| 1-8-341 | p-I—Ph | H | CH₂CH₂SCH₃ | nBu |
| 1-8-342* | p-I—Ph | H | * | nBu |
| 1-8-343 | p-I—Ph | H | H | Ph |
| 1-8-344 | p-I—Ph | H | CH₃ | Ph |
| 1-8-345 | p-I—Ph | H | CH(CH₃)₂ | Ph |
| 1-8-346 | p-I—Ph | H | CH₂CH(CH₃)₂ | Ph |
| 1-8-347 | p-I—Ph | H | CH(CH₃)CH₂CH₃ | Ph |
| 1-8-348 | p-I—Ph | H | CH₂Ph | Ph |
| 1-8-349 | p-I—Ph | H | CH₂-indol-3-yl | Ph |
| 1-8-350 | p-I—Ph | H | CH₂CH₂SCH₃ | Ph |
| 1-8-351* | p-I—Ph | H | * | Ph |
| 1-8-352 | p-I—Ph | H | H | Bn |
| 1-8-353 | p-I—Ph | H | CH₃ | Bn |
| 1-8-354 | p-I—Ph | H | CH(CH₃)₂ | Bn |
| 1-8-355 | p-I—Ph | H | CH₂CH(CH₃)₂ | Bn |
| 1-8-356 | p-I—Ph | H | CH(CH₃)CH₂CH₃ | Bn |
| 1-8-357 | p-I—Ph | H | CH₂Ph | Bn |
| 1-8-358 | p-I—Ph | H | CH₂-indol-3-yl | Bn |
| 1-8-359 | p-I—Ph | H | CH₂CH₂SCH₃ | Bn |
| 1-8-360* | p-I—Ph | H | * | Bn |
| 1-8-361 | p-I—Ph | H | H | CH₃ |
| 1-8-362 | p-I—Ph | H | CH₃ | CH₃ |
| 1-8-363 | p-I—Ph | H | CH(CH₃)₂ | CH₃ |
| 1-8-364 | p-I—Ph | H | CH₂CH(CH₃)₂ | CH₃ |
| 1-8-365 | p-I—Ph | H | CH(CH₃)CH₂CH₃ | CH₃ |
| 1-8-366 | p-I—Ph | H | CH₂Ph | CH₃ |
| 1-8-367 | p-I—Ph | H | CH₂-indol-3-yl | CH₃ |
| 1-8-368 | p-I—Ph | H | CH₂CH₂SCH₃ | CH₃ |
| 1-8-369* | p-I—Ph | H | * | CH₃ |
| 1-8-370 | p-I—Ph | H | H | Et |
| 1-8-371 | p-I—Ph | H | CH₃ | Et |
| 1-8-372 | p-I—Ph | H | CH(CH₃)₂ | Et |
| 1-8-373 | p-I—Ph | H | CH₂CH(CH₃)₂ | Et |
| 1-8-374 | p-I—Ph | H | CH(CH₃)CH₂CH₃ | Et |
| 1-8-375 | p-I—Ph | H | CH₂Ph | Et |
| 1-8-376 | p-I—Ph | H | CH₂-indol-3-yl | Et |
| 1-8-377 | p-I—Ph | H | CH₂CH₂SCH₃ | Et |
| 1-8-378* | p-I—Ph | H | * | Et |
| 1-8-379 | p-Me—Ph | H | H | iPr |
| 1-8-380 | p-Me—Ph | H | CH₃ | iPr |
| 1-8-381 | p-Me—Ph | H | CH(CH₃)₂ | iPr |
| 1-8-382 | p-Me—Ph | H | CH₂CH(CH₃)₂ | iPr |
| 1-8-383 | p-Me—Ph | H | CH(CH₃)CH₂CH₃ | iPr |
| 1-8-384 | p-Me—Ph | H | CH₂Ph | iPr |
| 1-8-385 | p-Me—Ph | H | CH₂-indol-3-yl | iPr |
| 1-8-386 | p-Me—Ph | H | CH₂CH₂SCH₃ | iPr |
| 1-8-387* | p-Me—Ph | H | * | iPr |
| 1-8-388 | p-Me—Ph | H | H | nBu |
| 1-8-389 | p-Me—Ph | H | CH₃ | nBu |
| 1-8-390 | p-Me—Ph | H | CH(CH₃)₂ | nBu |
| 1-8-391 | p-Me—Ph | H | CH₂CH(CH₃)₂ | nBu |
| 1-8-392 | p-Me—Ph | H | CH(CH₃)CH₂CH₃ | nBu |
| 1-8-393 | p-Me—Ph | H | CH₂Ph | nBu |
| 1-8-394 | p-Me—Ph | H | CH₂-indol-3-yl | nBu |
| 1-8-395 | p-Me—Ph | H | CH₂CH₂SCH₃ | nBu |
| 1-8-396* | p-Me—Ph | H | * | nBu |
| 1-8-397 | p-Me—Ph | H | H | Ph |
| 1-8-398 | p-Me—Ph | H | CH₃ | Ph |
| 1-8-399 | p-Me—Ph | H | CH(CH₃)₂ | Ph |
| 1-8-400 | p-Me—Ph | H | CH₂CH(CH₃)₂ | Ph |
| 1-8-401 | p-Me—Ph | H | CH(CH₃)CH₂CH₃ | Ph |
| 1-8-402 | p-Me—Ph | H | CH₂Ph | Ph |
| 1-8-403 | p-Me—Ph | H | CH₂-indol-3-yl | Ph |
| 1-8-404 | p-Me—Ph | H | CH₂CH₂SCH₃ | Ph |
| 1-8-405* | p-Me—Ph | H | * | Ph |
| 1-8-406 | p-Me—Ph | H | H | Bn |
| 1-8-407 | p-Me—Ph | H | CH₃ | Bn |
| 1-8-408 | p-Me—Ph | H | CH(CH₃)₂ | Bn |
| 1-8-409 | p-Me—Ph | H | CH₂CH(CH₃)₂ | Bn |
| 1-8-410 | p-Me—Ph | H | CH(CH₃)CH₂CH₃ | Bn |
| 1-8-411 | p-Me—Ph | H | CH₂Ph | Bn |
| 1-8-412 | p-Me—Ph | H | CH₂-indol-3-yl | Bn |
| 1-8-413 | p-Me—Ph | H | CH₂CH₂SCH₃ | Bn |
| 1-8-414* | p-Me—Ph | H | * | Bn |
| 1-8-415 | p-Me—Ph | H | H | CH₃ |
| 1-8-416 | p-Me—Ph | H | CH₃ | CH₃ |
| 1-8-417 | p-Me—Ph | H | CH(CH₃)₂ | CH₃ |
| 1-8-418 | p-Me—Ph | H | CH₂CH(CH₃)₂ | CH₃ |
| 1-8-419 | p-Me—Ph | H | CH(CH₃)CH₂CH₃ | CH₃ |
| 1-8-420 | p-Me—Ph | H | CH₂Ph | CH₃ |
| 1-8-421 | p-Me—Ph | H | CH₂-indol-3-yl | CH₃ |
| 1-8-422 | p-Me—Ph | H | CH₂CH₂SCH₃ | CH₃ |
| 1-8-423* | p-Me—Ph | H | * | CH₃ |
| 1-8-424 | p-Me—Ph | H | H | Et |
| 1-8-425 | p-Me—Ph | H | CH₃ | Et |
| 1-8-426 | p-Me—Ph | H | CH(CH₃)₂ | Et |
| 1-8-427 | p-Me—Ph | H | CH₂CH(CH₃)₂ | Et |
| 1-8-428 | p-Me—Ph | H | CH(CH₃)CH₂CH₃ | Et |
| 1-8-429 | p-Me—Ph | H | CH₂Ph | Et |
| 1-8-430 | p-Me—Ph | H | CH₂-indol-3-yl | Et |
| 1-8-431 | p-Me—Ph | H | CH₂CH₂SCH₃ | Et |
| 1-8-432* | p-Me—Ph | H | * | Et |
| 1-8-433 | tBu | H | H | iPr |
| 1-8-434 | tBu | H | CH₃ | iPr |
| 1-8-435 | tBu | H | CH(CH₃)₂ | iPr |
| 1-8-436 | tBu | H | CH₂CH(CH₃)₂ | iPr |
| 1-8-437 | tBu | H | CH(CH₃)CH₂CH₃ | iPr |
| 1-8-43 8 | tBu | H | CH₂Ph | iPr |
| 1-8-439 | tBu | H | CH₂-indol-3-yl | iPr |
| 1-8-440 | tBu | H | CH₂CH₂SCH₃ | iPr |
| 1-8-441* | tBu | H | * | iPr |
| 1-8-442 | tBu | H | H | nBu |
| 1-8-443 | tBu | H | CH₃ | nBu |
| 1-8-444 | tBu | H | CH(CH₃)₂ | nBu |
| 1-8-445 | tBu | H | CH₂CH(CH₃)₂ | nBu |
| 1-8-446 | tBu | H | CH(CH₃)CH₂CH₃ | nBu |
| 1-8-447 | tBu | H | CH₂Ph | nBu |
| 1-8-448 | tBu | H | CH₂-indol-3-yl | nBu |
| 1-8-449 | tBu | H | CH₂CH₂SCH₃ | nBu |
| 1-8-450* | tBu | H | * | nBu |
| 1-8-451 | tBu | H | H | Ph |
| 1-8-452 | tBu | H | CH₃ | Ph |
| 1-8-453 | tBu | H | CH(CH₃)₂ | Ph |
| 1-8-454 | tBu | H | CH₂CH(CH₃)₂ | Ph |
| 1-8-455 | tBu | H | CH(CH₃)CH₂CH₃ | Ph |
| 1-8-456 | tBu | H | CH₂Ph | Ph |
| 1-8-457 | tBu | H | CH₂-indol-3-yl | Ph |
| 1-8-458 | tBu | H | CH₂CH₂SCH₃ | Ph |
| 1-8-459* | tBu | H | * | Ph |
| 1-8-460 | tBu | H | H | Bn |
| 1-8-461 | tBu | H | CH₃ | Bn |
| 1-8-462 | tBu | H | CH(CH₃)₂ | Bn |
| 1-8-463 | tBu | H | CH₂CH(CH₃)₂ | Bn |
| 1-8-464 | tBu | H | CH(CH₃)CH₂CH₃ | Bn |
| 1-8-465 | tBu | H | CH₂Ph | Bn |
| 1-8-466 | tBu | H | CH₂-indol-3-yl | Bn |
| 1-8-467 | tBu | H | CH₂CH₂SCH₃ | Bn |
| 1-8-468* | tBu | H | * | Bn |
| 1-8-469 | tBu | H | H | CH₃ |
| 1-8-470 | tBu | H | CH₃ | CH₃ |
| 1-8-471 | tBu | H | CH(CH₃)₂ | CH₃ |
| 1-8-472 | tBu | H | CH₂CH(CH₃)₂ | CH₃ |
| 1-8-473 | tBu | H | CH(CH₃)CH₂CH₃ | CH₃ |
| 1-8-474 | tBu | H | CH₂Ph | CH₃ |
| 1-8-475 | tBu | H | CH₂-indol-3-yl | CH₃ |
| 1-8-476 | tBu | H | CH₂CH₂SCH₃ | CH₃ |
| 1-8-477* | tBu | H | * | CH₃ |
| 1-8-478 | tBu | H | H | Et |
| 1-8-479 | tBu | H | CH₃ | Et |
| 1-8-480 | tBu | H | CH(CH₃)₂ | Et |
| 1-8-481 | tBu | H | CH₂CH(CH₃)₂ | Et |
| 1-8-482 | tBu | H | CH(CH₃)CH₂CH₃ | Et |
| 1-8-483 | tBu | H | CH₂Ph | Et |
| 1-8-484 | tBu | H | CH₂-indol-3-yl | Et |
| 1-8-485 | tBu | H | CH₂CH₂SCH₃ | Et |
| 1-8-486* | tBu | H | * | Et |
| 1-8-487 | Ph | H | H | iPr |
| 1-8-488 | Ph | CH₃ | H | iPr |
| 1-8-489 | Ph | CH(CH₃)₂ | H | iPr |
| 1-8-490 | Ph | CH₂CH(CH₃)₂ | H | iPr |
| 1-8-491 | Ph | CH(CH₃)CH₂CH₃ | H | iPr |
| 1-8-492 | Ph | CH₂Ph | H | iPr |
| 1-8-493 | Ph | CH₂-indol-3-yl | H | iPr |
| 1-8-494 | Ph | CH₂CH₂SCH₃ | H | iPr |
| 1-8-495* | Ph | * | H | iPr |
| 1-8-496 | Ph | H | H | nBu |
| 1-8-497 | Ph | CH₃ | H | nBu |
| 1-8-498 | Ph | CH(CH₃)₂ | H | nBu |
| 1-8-499 | Ph | CH₂CH(CH₃)₂ | H | nBu |
| 1-8-500 | Ph | CH(CH₃)CH₂CH₃ | H | nBu |
| 1-8-501 | Ph | CH₂Ph | H | nBu |
| 1-8-502 | Ph | CH₂-indol-3-yl | H | nBu |
| 1-8-503 | Ph | CH₂CH₂SCH₃ | H | nBu |
| 1-8-504* | Ph | * | H | nBu |
| 1-8-505 | Ph | H | H | Ph |
| 1-8-506 | Ph | CH₃ | H | Ph |
| 1-8-507 | Ph | CH(CH₃)₂ | H | Ph |
| 1-8-508 | Ph | CH₂CH(CH₃)₂ | H | Ph |
| 1-8-509 | Ph | CH(CH₃)CH₂CH₃ | H | Ph |
| 1-8-510 | Ph | CH₂Ph | H | Ph |
| 1-8-511 | Ph | CH₂-indol-3-yl | H | Ph |
| 1-8-512 | Ph | CH₂CH₂SCH₃ | H | Ph |
| 1-8-513* | Ph | * | H | Ph |
| 1-8-514 | Ph | H | H | Bn |
| 1-8-515 | Ph | CH₃ | H | Bn |
| 1-8-516 | Ph | CH(CH₃)₂ | H | Bn |
| 1-8-517 | Ph | CH₂CH(CH₃)₂ | H | Bn |
| 1-8-518 | Ph | CH(CH₃)CH₂CH₃ | H | Bn |
| 1-8-519 | Ph | CH₂Ph | H | Bn |
| 1-8-520 | Ph | CH₂-indol-3-yl | H | Bn |
| 1-8-521 | Ph | CH₂CH₂SCH₃ | H | Bn |
| 1-8-522* | Ph | * | H | Bn |
| 1-8-523 | Ph | H | H | CH₃ |
| 1-8-524 | Ph | CH₃ | H | CH₃ |
| 1-8-525 | Ph | CH(CH₃)₂ | H | CH₃ |
| 1-8-526 | Ph | CH₂CH(CH₃)₂ | H | CH₃ |
| 1-8-527 | Ph | CH(CH₃)CH₂CH₃ | H | CH₃ |
| 1-8-528 | Ph | CH₂Ph | H | CH₃ |
| 1-8-529 | Ph | CH₂-indol-3-yl | H | CH₃ |
| 1-8-530 | Ph | CH₂CH₂SCH₃ | H | CH₃ |
| 1-8-531* | Ph | * | H | CH₃ |
| 1-8-532 | Ph | H | H | Et |
| 1-8-533 | Ph | CH₃ | H | Et |
| 1-8-534 | Ph | CH(CH₃)₂ | H | Et |
| 1-8-535 | Ph | CH₂CH(CH₃)₂ | H | Et |
| 1-8-536 | Ph | CH(CH₃)CH₂CH₃ | H | Et |
| 1-8-537 | Ph | CH₂Ph | H | Et |
| 1-8-538 | Ph | CH₂-indol-3-yl | H | Et |
| 1-8-539 | Ph | CH₂CH₂SCH₃ | H | Et |
| 1-8-540* | Ph | * | H | Et |
| 1-8-541 | 1-Nap | H | H | iPr |
| 1-8-542 | 1-Nap | CH₃ | H | iPr |
| 1-8-543 | 1-Nap | CH(CH₃)₂ | H | iPr |
| 1-8-544 | 1-Nap | CH₂CH(CH₃)₂ | H | iPr |
| 1-8-545 | 1-Nap | CH(CH₃)CH₂CH₃ | H | iPr |
| 1-8-546 | 1-Nap | CH₂Ph | H | iPr |
| 1-8-547 | 1-Nap | CH₂-indol-3-yl | H | iPr |
| 1-8-548 | 1-Nap | CH₂CH₂SCH₃ | H | iPr |
| 1-8-549* | 1-Nap | * | H | iPr |
| 1-8-550 | 1-Nap | H | H | nBu |
| 1-8-551 | 1-Nap | CH₃ | H | nBu |
| 1-8-552 | 1-Nap | CH(CH₃)₂ | H | nBu |
| 1-8-553 | 1-Nap | CH₂CH(CH₃)₂ | H | nBu |
| 1-8-554 | 1-Nap | CH(CH₃)CH₂CH₃ | H | nBu |
| 1-8-555 | 1-Nap | CH₂Ph | H | nBu |
| 1-8-556 | 1-Nap | CH₂-indol-3-yl | H | nBu |
| 1-8-557 | 1-Nap | CH₂CH₂SCH₃ | H | nBu |
| 1-8-558* | 1-Nap | * | H | nBu |
| 1-8-559 | 1-Nap | H | H | Ph |
| 1-8-560 | 1-Nap | CH₃ | H | Ph |
| 1-8-561 | 1-Nap | CH(CH₃)₂ | H | Ph |
| 1-8-562 | 1-Nap | CH₂CH(CH₃)₂ | H | Ph |
| 1-8-563 | 1-Nap | CH(CH₃)CH₂CH₃ | H | Ph |
| 1-8-564 | 1-Nap | CH₂Ph | H | Ph |
| 1-8-565 | 1-Nap | CH₂-indol-3-yl | H | Ph |
| 1-8-566 | 1-Nap | CH₂CH₂SCH₃ | H | Ph |
| 1-8-567* | 1-Nap | * | H | Ph |
| 1-8-568 | 1-Nap | H | H | Bn |
| 1-8-569 | 1-Nap | CH₃ | H | Bn |
| 1-8-570 | 1-Nap | CH(CH₃)₂ | H | Bn |
| 1-8-571 | 1-Nap | CH₂CH(CH₃)₂ | H | Bn |
| 1-8-572 | 1-Nap | CH(CH₃)CH₂CH₃ | H | Bn |
| 1-8-573 | 1-Nap | CH₂Ph | H | Bn |
| 1-8-574 | 1-Nap | CH₂-indol-3-yl | H | Bn |
| 1-8-575 | 1-Nap | CH₂CH₂SCH₃ | H | Bn |
| 1-8-576* | 1-Nap | * | H | Bn |
| 1-8-577 | 1-Nap | H | H | CH₃ |
| 1-8-578 | 1-Nap | CH₃ | H | CH₃ |
| 1-8-579 | 1-Nap | CH(CH₃)₂ | H | CH₃ |
| 1-8-580 | 1-Nap | CH₂CH(CH₃)₂ | H | CH₃ |
| 1-8-581 | 1-Nap | CH(CH₃)CH₂CH₃ | H | CH₃ |
| 1-8-582 | 1-Nap | CH₂Ph | H | CH₃ |
| 1-8-583 | 1-Nap | CH₂-indol-3-yl | H | CH₃ |
| 1-8-584 | 1-Nap | CH₂CH₂SCH₃ | H | CH₃ |
| 1-8-585* | 1-Nap | * | H | CH₃ |
| 1-8-586 | 1-Nap | H | H | Et |
| 1-8-587 | 1-Nap | CH₃ | H | Et |
| 1-8-588 | 1-Nap | CH(CH₃)₂ | H | Et |
| 1-8-589 | 1-Nap | CH₂CH(CH₃)₂ | H | Et |
| 1-8-590 | 1-Nap | CH(CH₃)CH₂CH₃ | H | Et |
| 1-8-591 | 1-Nap | CH₂Ph | H | Et |
| 1-8-592 | 1-Nap | CH₂-indol-3-yl | H | Et |
| 1-8-593 | 1-Nap | CH₂CH₂SCH₃ | H | Et |
| 1-8-594* | 1-Nap | * | H | Et |
| 1-8-595 | 2-Nap | H | H | iPr |
| 1-8-596 | 2-Nap | CH₃ | H | iPr |
| 1-8-597 | 2-Nap | CH(CH₃)₂ | H | iPr |
| 1-8-598 | 2-Nap | CH₂CH(CH₃)₂ | H | iPr |
| 1-8-599 | 2-Nap | CH(CH₃)CH₂CH₃ | H | iPr |
| 1-8-600 | 2-Nap | CH₂Ph | H | iPr |
| 1-8-601 | 2-Nap | CH₂-indol-3-yl | H | iPr |
| 1-8-602 | 2-Nap | CH₂CH₂SCH₃ | H | iPr |
| 1-8-603* | 2-Nap | * | H | iPr |
| 1-8-604 | 2-Nap | H | H | nBu |
| 1-8-605 | 2-Nap | CH₃ | H | nBu |
| 1-8-606 | 2-Nap | CH(CH₃)₂ | H | nBu |
| 1-8-607 | 2-Nap | CH₂CH(CH₃)₂ | H | nBu |
| 1-8-608 | 2-Nap | CH(CH₃)CH₂CH₃ | H | nBu |
| 1-8-609 | 2-Nap | CH₂Ph | H | nBu |
| 1-8-610 | 2-Nap | CH₂-indol-3-yl | H | nBu |
| 1-8-611 | 2-Nap | CH₂CH₂SCH₃ | H | nBu |
| 1-8-612* | 2-Nap | * | H | nBu |
| 1-8-613 | 2-Nap | H | H | Ph |
| 1-8-614 | 2-Nap | CH₃ | H | Ph |
| 1-8-615 | 2-Nap | CH(CH₃)₂ | H | Ph |
| 1-8-616 | 2-Nap | CH₂CH(CH₃)₂ | H | Ph |
| 1-8-617 | 2-Nap | CH(CH₃)CH₂CH₃ | H | Ph |
| 1-8-618 | 2-Nap | CH₂Ph | H | Ph |
| 1-8-619 | 2-Nap | CH₂-indol-3-yl | H | Ph |
| 1-8-620 | 2-Nap | CH₂CH₂SCH₃ | H | Ph |
| 1-8-621* | 2-Nap | * | H | Ph |
| 1-8-622 | 2-Nap | H | H | Bn |
| 1-8-623 | 2-Nap | CH₃ | H | Bn |
| 1-8-624 | 2-Nap | CH(CH₃)₂ | H | Bn |
| 1-8-625 | 2-Nap | CH₂CH(CH₃)₂ | H | Bn |
| 1-8-626 | 2-Nap | CH(CH₃)CH₂CH₃ | H | Bn |
| 1-8-627 | 2-Nap | CH₂Ph | H | Bn |
| 1-8-628 | 2-Nap | CH₂-indol-3-yl | H | Bn |
| 1-8-629 | 2-Nap | CH₂CH₂SCH₃ | H | Bn |
| 1-8-630* | 2-Nap | * | H | Bn |
| 1-8-631 | 2-Nap | H | H | CH₃ |
| 1-8-632 | 2-Nap | CH₃ | H | CH₃ |
| 1-8-633 | 2-Nap | CH(CH₃)₂ | H | CH₃ |
| 1-8-634 | 2-Nap | CH₂CH(CH₃)₂ | H | CH₃ |
| 1-8-635 | 2-Nap | CH(CH₃)CH₂CH₃ | H | CH₃ |
| 1-8-636 | 2-Nap | CH₂Ph | H | CH₃ |
| 1-8-637 | 2-Nap | CH₂-indol-3-yl | H | CH₃ |
| 1-8-638 | 2-Nap | CH₂CH₂SCH₃ | H | CH₃ |
| 1-8-639* | 2-Nap | * | H | CH₃ |
| 1-8-640 | 2-Nap | H | H | Et |
| 1-8-641 | 2-Nap | CH₃ | H | Et |
| 1-8-642 | 2-Nap | CH(CH₃)₂ | H | Et |
| 1-8-643 | 2-Nap | CH₂CH(CH₃)₂ | H | Et |
| 1-8-644 | 2-Nap | CH(CH₃)CH₂CH₃ | H | Et |
| 1-8-645 | 2-Nap | CH₂Ph | H | Et |
| 1-8-646 | 2-Nap | CH₂-indol-3-yl | H | Et |
| 1-8-647 | 2-Nap | CH₂CH₂SCH₃ | H | Et |
| 1-8-648* | 2-Nap | * | H | Et |
| 1-8-649 | p-Br—Ph | H | H | iPr |
| 1-8-650 | p-Br—Ph | CH₃ | H | iPr |
| 1-8-651 | p-Br—Ph | CH(CH₃)₂ | H | iPr |
| 1-8-652 | p-Br—Ph | CH₂CH(CH₃)₂ | H | iPr |
| 1-8-653 | p-Br—Ph | CH(CH₃)CH₂CH₃ | H | iPr |
| 1-8-654 | p-Br—Ph | CH₂Ph | H | iPr |
| 1-8-655 | p-Br—Ph | CH₂-indol-3-yl | H | iPr |
| 1-8-656 | p-Br—Ph | CH₂CH₂SCH₃ | H | iPr |
| 1-8-657* | p-Br—Ph | * | H | iPr |
| 1-8-658 | p-Br—Ph | H | H | nBu |
| 1-8-659 | p-Br—Ph | CH₃ | H | nBu |
| 1-8-660 | p-Br—Ph | CH(CH₃)₂ | H | nBu |
| 1-8-661 | p-Br—Ph | CH₂CH(CH₃)₂ | H | nBu |
| 1-8-662 | p-Br—Ph | CH(CH₃)CH₂CH₃ | H | nBu |
| 1-8-663 | p-Br—Ph | CH₂Ph | H | nBu |
| 1-8-664 | p-Br—Ph | CH₂-indol-3-yl | H | nBu |
| 1-8-665 | p-Br—Ph | CH₂CH₂SCH₃ | H | nBu |
| 1-8-666* | p-Br—Ph | * | H | nBu |
| 1-8-667 | p-Br—Ph | H | H | Ph |
| 1-8-668 | p-Br—Ph | CH₃ | H | Ph |
| 1-8-669 | p-Br—Ph | CH(CH₃)₂ | H | Ph |
| 1-8-670 | p-Br—Ph | CH₂CH(CH₃)₂ | H | Ph |
| 1-8-671 | p-Br—Ph | CH(CH₃)CH₂CH₃ | H | Ph |
| 1-8-672 | p-Br—Ph | CH₂Ph | H | Ph |
| 1-8-673 | p-Br—Ph | CH₂-indol-3-yl | H | Ph |
| 1-8-674 | p-Br—Ph | CH₂CH₂SCH₃ | H | Ph |
| 1-8-675* | p-Br—Ph | * | H | Ph |
| 1-8-676 | p-Br—Ph | H | H | Bn |
| 1-8-677 | p-Br—Ph | CH₃ | H | Bn |
| 1-8-678 | p-Br—Ph | CH(CH₃)₂ | H | Bn |
| 1-8-679 | p-Br—Ph | CH₂CH(CH₃)₂ | H | Bn |
| 1-8-680 | p-Br—Ph | CH(CH₃)CH₂CH₃ | H | Bn |
| 1-8-681 | p-Br—Ph | CH₂Ph | H | Bn |
| 1-8-682 | p-Br—Ph | CH₂-indol-3-yl | H | Bn |
| 1-8-683 | p-Br—Ph | CH₂CH₂SCH₃ | H | Bn |
| 1-8-684* | p-Br—Ph | * | H | Bn |
| 1-8-685 | p-Br—Ph | H | H | CH₃ |
| 1-8-686 | p-Br—Ph | CH₃ | H | CH₃ |
| 1-8-687 | p-Br—Ph | CH(CH₃)₂ | H | CH₃ |
| 1-8-688 | p-Br—Ph | CH₂CH(CH₃)₂ | H | CH₃ |
| 1-8-689 | p-Br—Ph | CH(CH₃)CH₂CH₃ | H | CH₃ |
| 1-8-690 | p-Br—Ph | CH₂Ph | H | CH₃ |
| 1-8-691 | p-Br—Ph | CH₂-indol-3-yl | H | CH₃ |
| 1-8-692 | p-Br—Ph | CH₂CH₂SCH₃ | H | CH₃ |
| 1-8-693* | p-Br—Ph | * | H | CH₃ |
| 1-8-694 | p-Br—Ph | H | H | Et |
| 1-8-695 | p-Br—Ph | CH₃ | H | Et |
| 1-8-696 | p-Br—Ph | CH(CH₃)₂ | H | Et |
| 1-8-697 | p-Br—Ph | CH₂CH(CH₃)₂ | H | Et |
| 1-8-698 | p-Br—Ph | CH(CH₃)CH₂CH₃ | H | Et |
| 1-8-699 | p-Br—Ph | CH₂Ph | H | Et |
| 1-8-700 | p-Br—Ph | CH₂-indol-3-yl | H | Et |
| 1-8-701 | p-Br—Ph | CH₂CH₂SCH₃ | H | Et |
| 1-8-702* | p-Br—Ph | * | H | Et |
| 1-8-703 | p-Cl—Ph | H | H | iPr |
| 1-8-704 | p-Cl—Ph | CH₃ | H | iPr |
| 1-8-705 | p-Cl—Ph | CH(CH₃)₂ | H | iPr |
| 1-8-706 | p-Cl—Ph | CH₂CH(CH₃)₂ | H | iPr |
| 1-8-707 | p-Cl—Ph | CH(CH₃)CH₂CH₃ | H | iPr |
| 1-8-708 | p-Cl—Ph | CH₂Ph | H | iPr |
| 1-8-709 | p-Cl—Ph | CH₂-indol-3-yl | H | iPr |
| 1-8-710 | p-Cl—Ph | CH₂CH₂SCH₃ | H | iPr |
| 1-8-711* | p-Cl—Ph | * | H | iPr |
| 1-8-712 | p-Cl—Ph | H | H | nBu |
| 1-8-713 | p-Cl—Ph | CH₃ | H | nBu |
| 1-8-714 | p-Cl—Ph | CH(CH₃)₂ | H | nBu |
| 1-8-715 | p-Cl—Ph | CH₂CH(CH₃)₂ | H | nBu |
| 1-8-716 | p-Cl—Ph | CH(CH₃)CH₂CH₃ | H | nBu |
| 1-8-717 | p-Cl—Ph | CH₂Ph | H | nBu |
| 1-8-718 | p-Cl—Ph | CH₂-indol-3-yl | H | nBu |
| 1-8-719 | p-Cl—Ph | CH₂CH₂SCH₃ | H | nBu |
| 1-8-720* | p-Cl—Ph | * | H | nBu |
| 1-8-721 | p-Cl—Ph | H | H | Ph |
| 1-8-722 | p-Cl—Ph | CH₃ | H | Ph |
| 1-8-723 | p-Cl—Ph | CH(CH₃)₂ | H | Ph |
| 1-8-724 | p-Cl—Ph | CH₂CH(CH₃)₂ | H | Ph |
| 1-8-725 | p-Cl—Ph | CH(CH₃)CH₂CH₃ | H | Ph |
| 1-8-726 | p-Cl—Ph | CH₂Ph | H | Ph |
| 1-8-727 | p-Cl—Ph | CH₂-indol-3-yl | H | Ph |
| 1-8-728 | p-Cl—Ph | CH₂CH₂SCH₃ | H | Ph |
| 1-8-729* | p-Cl—Ph | * | H | Ph |
| 1-8-730 | p-Cl—Ph | H | H | Bn |
| 1-8-731 | p-Cl—Ph | CH₃ | H | Bn |
| 1-8-732 | p-Cl—Ph | CH(CH₃)₂ | H | Bn |
| 1-8-733 | p-Cl—Ph | CH₂CH(CH₃)₂ | H | Bn |
| 1-8-734 | p-Cl—Ph | CH(CH₃)CH₂CH₃ | H | Bn |
| 1-8-735 | p-Cl—Ph | CH₂Ph | H | Bn |
| 1-8-736 | p-Cl—Ph | CH₂-indol-3-yl | H | Bn |
| 1-8-737 | p-Cl—Ph | CH₂CH₂SCH₃ | H | Bn |
| 1-8-738* | p-Cl—Ph | * | H | Bn |
| 1-8-739 | p-Cl—Ph | H | H | CH₃ |
| 1-8-740 | p-Cl—Ph | CH₃ | H | CH₃ |
| 1-8-741 | p-Cl—Ph | CH(CH₃)₂ | H | CH₃ |
| 1-8-742 | p-Cl—Ph | CH₂CH(CH₃)₂ | H | CH₃ |
| 1-8-743 | p-Cl—Ph | CH(CH₃)CH₂CH₃ | H | CH₃ |
| 1-8-744 | p-Cl—Ph | CH₂Ph | H | CH₃ |
| 1-8-745 | p-Cl—Ph | CH₂-indol-3-yl | H | CH₃ |
| 1-8-746 | p-Cl—Ph | CH₂CH₂SCH₃ | H | CH₃ |
| 1-8-747* | p-Cl—Ph | * | H | CH₃ |
| 1-8-748 | p-Cl—Ph | H | H | Et |
| 1-8-749 | p-Cl—Ph | CH₃ | H | Et |
| 1-8-750 | p-Cl—Ph | CH(CH₃)₂ | H | Et |
| 1-8-751 | p-Cl—Ph | CH₂CH(CH₃)₂ | H | Et |
| 1-8-752 | p-Cl—Ph | CH(CH₃)CH₂CH₃ | H | Et |
| 1-8-753 | p-Cl—Ph | CH₂Ph | H | Et |
| 1-8-754 | p-Cl—Ph | CH₂-indol-3-yl | H | Et |
| 1-8-755 | p-Cl—Ph | CH₂CH₂SCH₃ | H | Et |
| 1-8-756* | p-Cl—Ph | * | H | Et |
| 1-8-757 | p-F—Ph | H | H | iPr |
| 1-8-758 | p-F—Ph | CH₃ | H | iPr |
| 1-8-759 | p-F—Ph | CH(CH₃)₂ | H | iPr |
| 1-8-760 | p-F—Ph | CH₂CH(CH₃)₂ | H | iPr |
| 1-8-761 | p-F—Ph | CH(CH₃)CH₂CH₃ | H | iPr |
| 1-8-762 | p-F—Ph | CH₂Ph | H | iPr |
| 1-8-763 | p-F—Ph | CH₂-indol-3-ylH | iPr | |
| 1-8-764 | p-F—Ph | CH₂CH₂SCH₃ | H | iPr |
| 1-8-765* | p-F—Ph | * | H | iPr |
| 1-8-766 | p-F—Ph | H | H | nBu |
| 1-8-767 | p-F—Ph | CH₃ | H | nBu |
| 1-8-768 | p-F—Ph | CH(CH₃)₂ | H | nBu |
| 1-8-769 | p-F—Ph | CH₂CH(CH₃)₂ | H | nBu |
| 1-8-770 | p-F—Ph | CH(CH₃)CH₂CH₃ | H | nBu |
| 1-8-771 | p-F—Ph | CH₂Ph | H | nBu |
| 1-8-772 | p-F—Ph | CH₂-indol-3-yl | H | nBu |
| 1-8-773 | p-F—Ph | CH₂CH₂SCH₃ | H | nBu |
| 1-8-774* | p-F—Ph | * | H | nBu |
| 1-8-775 | p-F—Ph | H | H | Ph |
| 1-8-776 | p-F—Ph | CH₃ | H | Ph |
| 1-8-777 | p-F—Ph | CH(CH₃)₂ | H | Ph |
| 1-8-778 | p-F—Ph | CH₂CH(CH₃)₂ | H | Ph |
| 1-8-779 | p-F—Ph | CH(CH₃)CH₂CH₃ | H | Ph |
| 1-8-780 | p-F—Ph | CH₂Ph | H | Ph |
| 1-8-781 | p-F—Ph | CH₂-indol-3-yl | H | Ph |
| 1-8-782 | p-F—Ph | CH₂CH₂SCH₃ | H | Ph |
| 1-8-783* | p-F—Ph | * | H | Ph |
| 1-8-784 | p-F—Ph | H | H | Bn |
| 1-8-785 | p-F—Ph | CH₃ | H | Bn |
| 1-8-786 | p-F—Ph | CH(CH₃)₂ | H | Bn |
| 1-8-787 | p-F—Ph | CH₂CH(CH₃)₂ | H | Bn |
| 1-8-788 | p-F—Ph | CH(CH₃)CH₂CH₃ | H | Bn |
| 1-8-789 | p-F—Ph | CH₂Ph | H | Bn |
| 1-8-790 | p-F—Ph | CH₂-indol-3-yl | H | Bn |
| 1-8-791 | p-F—Ph | CH₂CH₂SCH₃ | H | Bn |
| 1-8-792* | p-F—Ph | * | H | Bn |
| 1-8-793 | p-F—Ph | H | H | CH₃ |
| 1-8-794 | p-F—Ph | CH₃ | H | CH₃ |
| 1-8-795 | p-F—Ph | CH(CH₃)₂ | H | CH₃ |
| 1-8-796 | p-F—Ph | CH₂CH(CH₃)₂ | H | CH₃ |
| 1-8-797 | p-F—Ph | CH(CH₃)CH₂CH₃ | H | CH₃ |
| 1-8-798 | p-F—Ph | CH₂Ph | H | CH₃ |
| 1-8-799 | p-F—Ph | CH₂-indol-3-yl | H | CH₃ |
| 1-8-800 | p-F—Ph | CH₂CH₂SCH₃ | H | CH₃ |
| 1-8-801* | p-F—Ph | * | H | CH₃ |
| 1-8-802 | p-F—Ph | H | H | Et |
| 1-8-803 | p-F—Ph | CH₃ | H | Et |
| 1-8-804 | p-F—Ph | CH(CH₃)₂ | H | Et |
| 1-8-805 | p-F—Ph | CH₂CH(CH₃)₂ | H | Et |
| 1-8-806 | p-F—Ph | CH(CH₃)CH₂CH₃ | H | Et |
| 1-8-807 | p-F—Ph | CH₂Ph | H | Et |
| 1-8-808 | p-F—Ph | CH₂-indol-3-yl | H | Et |
| 1-8-809 | p-F—Ph | CH₂CH₂SCH₃ | H | Et |
| 1-8-810* | p-F—Ph | * | H | Et |
| 1-8-811 | p-I—Ph | H | H | iPr |
| 1-8-812 | p-I—Ph | CH₃ | H | iPr |
| 1-8-813 | p-I—Ph | CH(CH₃)₂ | H | iPr |
| 1-8-814 | p-I—Ph | CH₂CH(CH₃)₂ | H | iPr |
| 1-8-815 | p-I—Ph | CH(CH₃)CH₂CH₃ | H | iPr |
| 1-8-816 | p-I—Ph | CH₂Ph | H | iPr |
| 1-8-817 | p-I—Ph | CH₂-indol-3-yl | H | iPr |
| 1-8-818 | p-I—Ph | CH₂CH₂SCH₃ | H | iPr |
| 1-8-819* | p-I—Ph | * | H | iPr |
| 1-8-820 | p-I—Ph | H | H | nBu |
| 1-8-821 | p-I—Ph | CH₃ | H | nBu |
| 1-8-822 | p-I—Ph | CH(CH₃)₂ | H | nBu |
| 1-8-823 | p-I—Ph | CH₂CH(CH₃)₂ | H | nBu |
| 1-8-824 | p-I—Ph | CH(CH₃)CH₂CH₃ | H | nBu |
| 1-8-825 | p-I—Ph | CH₂Ph | H | nBu |
| 1-8-826 | p-I—Ph | CH₂-indol-3-yl | H | nBu |
| 1-8-827 | p-I—Ph | CH₂CH₂SCH₃ | H | nBu |
| 1-8-828* | p-I—Ph | * | H | nBu |
| 1-8-829 | p-I—Ph | H | H | Ph |
| 1-8-830 | p-I—Ph | CH₃ | H | Ph |
| 1-8-831 | p-I—Ph | CH(CH₃)₂ | H | Ph |
| 1-8-832 | p-I—Ph | CH₂CH(CH₃)₂ | H | Ph |
| 1-8-833 | p-I—Ph | CH(CH₃)CH₂CH₃ | H | Ph |
| 1-8-834 | p-I—Ph | CH₂Ph | H | Ph |
| 1-8-835 | p-I—Ph | CH₂-indol-3-yl | H | Ph |
| 1-8-836 | p-I—Ph | CH₂CH₂SCH₃ | H | Ph |
| 1-8-837* | p-I—Ph | * | H | Ph |
| 1-8-838 | p-I—Ph | H | H | Bn |
| 1-8-839 | p-I—Ph | CH₃ | H | Bn |
| 1-8-840 | p-I—Ph | CH(CH₃)₂ | H | Bn |
| 1-8-841 | p-I—Ph | CH₂CH(CH₃)₂ | H | Bn |
| 1-8-842 | p-I—Ph | CH(CH₃)CH₂CH₃ | H | Bn |
| 1-8-843 | p-I—Ph | CH₂Ph | H | Bn |
| 1-8-844 | p-I—Ph | CH₂-indol-3-yl | H | Bn |
| 1-8-845 | p-I—Ph | CH₂CH₂SCH₃ | H | Bn |
| 1-8-846* | p-I—Ph | * | H | Bn |
| 1-8-847 | p-I—Ph | H | H | CH₃ |
| 1-8-848 | p-I—Ph | CH₃ | H | CH₃ |
| 1-8-849 | p-I—Ph | CH(CH₃)₂ | H | CH₃ |
| 1-8-850 | p-I—Ph | CH₂CH(CH₃)₂ | H | CH₃ |
| 1-8-851 | p-I—Ph | CH(CH₃)CH₂CH₃ | H | CH₃ |
| 1-8-852 | p-I—Ph | CH₂Ph | H | CH₃ |
| 1-8-853 | p-I—Ph | CH₂-indol-3-yl | H | CH₃ |
| 1-8-854 | p-I—Ph | CH₂CH₂SCH₃ | H | CH₃ |
| 1-8-855* | p-I—Ph | * | H | CH₃ |
| 1-8-856 | p-I—Ph | H | H | Et |
| 1-8-857 | p-I—Ph | CH₃ | H | Et |
| 1-8-858 | p-I—Ph | CH(CH₃)₂ | H | Et |
| 1-8-859 | p-I—Ph | CH₂CH(CH₃)₂ | H | Et |
| 1-8-860 | p-I—Ph | CH(CH₃)CH₂CH₃ | H | Et |
| 1-8-861 | p-I—Ph | CH₂Ph | H | Et |
| 1-8-862 | p-I—Ph | CH₂-indol-3-yl | H | Et |
| 1-8-863 | p-I—Ph | CH₂CH₂SCH₃ | H | Et |
| 1-8-864* | p-I—Ph | * | H | Et |
| 1-8-865 | p-Me—Ph | H | H | iPr |
| 1-8-866 | p-Me—Ph | CH₃ | H | iPr |
| 1-8-867 | p-Me—Ph | CH(CH₃)₂ | H | iPr |
| 1-8-868 | p-Me—Ph | CH₂CH(CH₃)₂ | H | iPr |
| 1-8-869 | p-Me—Ph | CH(CH₃)CH₂CH₃ | H | iPr |
| 1-8-870 | p-Me—Ph | CH₂Ph | H | iPr |
| 1-8-871 | p-Me—Ph | CH₂-indol-3-yl | H | iPr |
| 1-8-872 | p-Me—Ph | CH₂CH₂SCH₃ | H | iPr |
| 1-8-873* | p-Me—Ph | * | H | iPr |
| 1-8-874 | p-Me—Ph | H | H | nBu |
| 1-8-875 | p-Me—Ph | CH₃ | H | nBu |
| 1-8-876 | p-Me—Ph | CH(CH₃)₂ | H | nBu |
| 1-8-877 | p-Me—Ph | CH₂CH(CH₃)₂ | H | nBu |
| 1-8-878 | p-Me—Ph | CH(CH₃)CH₂CH₃ | H | nBu |
| 1-8-879 | p-Me—Ph | CH₂Ph | H | nBu |
| 1-8-880 | p-Me—Ph | CH₂-indol-3-yl | H | nBu |
| 1-8-881 | p-Me—Ph | CH₂CH₂SCH₃ | H | nBu |
| 1-8-882* | p-Me—Ph | * | H | nBu |
| 1-8-883 | p-Me—Ph | H | H | Ph |
| 1-8-884 | p-Me—Ph | CH₃ | H | Ph |
| 1-8-885 | p-Me—Ph | CH(CH₃)₂ | H | Ph |
| 1-8-886 | p-Me—Ph | CH₂CH(CH₃)₂ | H | Ph |
| 1-8-887 | p-Me—Ph | CH(CH₃)CH₂CH₃ | H | Ph |
| 1-8-888 | p-Me—Ph | CH₂Ph | H | Ph |
| 1-8-889 | p-Me—Ph | CH₂-indol-3-yl | H | Ph |
| 1-8-890 | p-Me—Ph | CH₂CH₂SCH₃ | H | Ph |
| 1-8-891* | p-Me—Ph | * | H | Ph |
| 1-8-892 | p-Me—Ph | H | H | Bn |
| 1-8-893 | p-Me—Ph | CH₃ | H | Bn |
| 1-8-894 | p-Me—Ph | CH(CH₃)₂ | H | Bn |
| 1-8-895 | p-Me—Ph | CH₂CH(CH₃)₂ | H | Bn |
| 1-8-896 | p-Me—Ph | CH(CH₃)CH₂CH₃ | H | Bn |
| 1-8-897 | p-Me—Ph | CH₂Ph | H | Bn |
| 1-8-898 | p-Me—Ph | CH₂-indol-3-yl | H | Bn |
| 1-8-899 | p-Me—Ph | CH₂CH₂SCH₃ | H | Bn |
| 1-8-900* | p-Me—Ph | * | H | Bn |
| 1-8-901 | p-Me—Ph | H | H | CH₃ |
| 1-8-902 | p-Me—Ph | CH₃ | H | CH₃ |
| 1-8-903 | p-Me—Ph | CH(CH₃)₂ | H | CH₃ |
| 1-8-904 | p-Me—Ph | CH₂CH(CH₃)₂ | H | CH₃ |
| 1-8-905 | p-Me—Ph | CH(CH₃)CH₂CH₃ | H | CH₃ |
| 1-8-906 | p-Me—Ph | CH₂Ph | H | CH₃ |
| 1-8-907 | p-Me—Ph | CH₂-indol-3-yl | H | CH₃ |
| 1-8-908 | p-Me—Ph | CH₂CH₂SCH₃ | H | CH₃ |
| 1-8-909* | p-Me—Ph | * | H | CH₃ |
| 1-8-910 | p-Me—Ph | H | H | Et |
| 1-8-911 | p-Me—Ph | CH₃ | H | Et |
| 1-8-912 | p-Me—Ph | CH(CH₃)₂ | H | Et |
| 1-8-913 | p-Me—Ph | CH₂CH(CH₃)₂ | H | Et |
| 1-8-914 | p-Me—Ph | CH(CH₃)CH₂CH₃ | H | Et |
| 1-8-915 | p-Me—Ph | CH₂Ph | H | Et |
| 1-8-916 | p-Me—Ph | CH₂-indol-3-yl | H | Et |
| 1-8-917 | p-Me—Ph | CH₂CH₂SCH₃ | H | Et |
| 1-8-918* | p-Me—Ph | * | H | Et |
| 1-8-919 | tBu | H | H | iPr |
| 1-8-920 | tBu | CH₃ | H | iPr |
| 1-8-921 | tBu | CH(CH₃)₂ | H | iPr |
| 1-8-922 | tBu | CH₂CH(CH₃)₂ | H | iPr |
| 1-8-923 | tBu | CH(CH₃)CH₂CH₃ | H | iPr |
| 1-8-924 | tBu | CH₂Ph | H | iPr |
| 1-8-925 | tBu | CH₂-indol-3-yl | H | iPr |
| 1-8-926 | tBu | CH₂CH₂SCH₃ | H | iPr |
| 1-8-927* | tBu | * | H | iPr |
| 1-8-928 | tBu | H | H | nBu |
| 1-8-929 | tBu | CH₃ | H | nBu |
| 1-8-930 | tBu | CH(CH₃)₂ | H | nBu |
| 1-8-931 | tBu | CH₂CH(CH₃)₂ | H | nBu |
| 1-8-932 | tBu | CH(CH₃)CH₂CH₃ | H | nBu |
| 1-8-933 | tBu | CH₂Ph | H | nBu |
| 1-8-934 | tBu | CH₂-indol-3-yl | H | nBu |
| 1-8-935 | tBu | CH₂CH₂SCH₃ | H | nBu |
| 1-8-936* | tBu | * | H | nBu |
| 1-8-937 | tBu | H | H | Ph |
| 1-8-938 | tBu | CH₃ | H | Ph |
| 1-8-939 | tBu | CH(CH₃)₂ | H | Ph |
| 1-8-940 | tBu | CH₂CH(CH₃)₂ | H | Ph |
| 1-8-941 | tBu | CH(CH₃)CH₂CH₃ | H | Ph |
| 1-8-942 | tBu | CH₂Ph | H | Ph |
| 1-8-943 | tBu | CH₂-indol-3-yl | H | Ph |
| 1-8-944 | tBu | CH₂CH₂SCH₃ | H | Ph |
| 1-8-945* | tBu | * | H | Ph |
| 1-8-946 | tBu | H | H | Bn |
| 1-8-947 | tBu | CH₃ | H | Bn |
| 1-8-948 | tBu | CH(CH₃)₂ | H | Bn |
| 1-8-949 | tBu | CH₂CH(CH₃)₂ | H | Bn |
| 1-8-950 | tBu | CH(CH₃)CH₂CH₃ | H | Bn |
| 1-8-951 | tBu | CH₂Ph | H | Bn |
| 1-8-952 | tBu | CH₂-indol-3-yl | H | Bn |
| 1-8-953 | tBu | CH₂CH₂SCH₃ | H | Bn |
| 1-8-954* | tBu | * | H | Bn |
| 1-8-955 | tBu | H | H | CH₃ |
| 1-8-956 | tBu | CH₃ | H | CH₃ |
| 1-8-957 | tBu | CH(CH₃)₂ | H | CH₃ |
| 1-8-958 | tBu | CH₂CH(CH₃)₂ | H | CH₃ |
| 1-8-959 | tBu | CH(CH₃)CH₂CH₃ | H | CH₃ |
| 1-8-960 | tBu | CH₂Ph | H | CH₃ |
| 1-8-961 | tBu | CH₂-indol-3-yl | H | CH₃ |
| 1-8-962 | tBu | CH₂CH₂SCH₃ | H | CH₃ |
| 1-8-963* | tBu | * | H | CH₃ |
| 1-8-964 | tBu | H | H | Et |
| 1-8-965 | tBu | CH₃ | H | Et |
| 1-8-966 | tBu | CH(CH₃)₂ | H | Et |
| 1-8-967 | tBu | CH₂CH(CH₃)₂ | H | Et |
| 1-8-968 | tBu | CH(CH₃)CH₂CH₃ | H | Et |
| 1-8-969 | tBu | CH₂Ph | H | Et |
| 1-8-970 | tBu | CH₂-indol-3-yl | H | Et |
| 1-8-971 | tBu | CH₂CH₂SCH₃ | H | Et |
| 1-8-972* | tBu | * | H | Et |

*R⁶ and R^{7a} (or R⁶ and R^{7b}) are joined together by (CH₂)₃ to form a five-membered ring.
Abbreviations used in Table 7:
Ph = Phenyl;
iPr = isopropyl;
nBu = n-butyl;
1-Nap = 1-naphthyl;
2-Nap = 2-naphthyl;
Bn = benzyl;
Et = ethyl; and
tBu = tert-butyl.

In another particular embodiment, the invention is a 5′-deuterated-2′-methyl, 2′-fluoro -uridine phosphoramidate having structure 3-8:

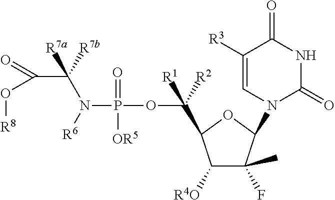

and its compositions and medical uses, including to treat hepatitis C, wherein R¹ and R² are both deuterium or at least one of R¹ and R² is deuterium and the other is hydrogen, R³ is hydrogen or deuterium. In one embodiment, R⁴ and R⁶ are hydrogen, R⁵, R^{7a}, R^{7b}, and R⁸ are as provided below in Table 8. In an alternative embodiment, R^{7a} and R^{7b} are reversed to form an amino acid residue with D-stereoconfiguration.

TABLE 8
5′-Deuterated-2′-deoxy-2′-α-fluoro-2′-
β-methyl-uridine phosphoramidates

| Structure | R⁵ | R^{7a} | R^{7b} | R⁸ |
|---|---|---|---|---|
| 3-8-1 | Ph | H | H | iPr |
| 3-8-2 | Ph | H | CH₃ | iPr |
| 3-8-3 | Ph | H | CH(CH₃)₂ | iPr |
| 3-8-4 | Ph | H | CH₂CH(CH₃)₂ | iPr |
| 3-8-5 | Ph | H | CH(CH₃)CH₂CH₃ | iPr |
| 3-8-6 | Ph | H | CH₂Ph | iPr |
| 3-8-7 | Ph | H | CH₂-indol-3-yl | iPr |
| 3-8-8 | Ph | H | CH₂CH₂SCH₃ | iPr |
| 3-8-9* | Ph | H | * | iPr |
| 3-8-10 | Ph | H | H | nBu |
| 3-8-11 | Ph | H | CH₃ | nBu |
| 3-8-12 | Ph | H | CH(CH₃)₂ | nBu |
| 3-8-13 | Ph | H | CH₂CH(CH₃)₂ | nBu |
| 3-8-14 | Ph | H | CH(CH₃)CH₂CH₃ | nBu |
| 3-8-15 | Ph | H | CH₂Ph | nBu |
| 3-8-16 | Ph | H | CH₂-indol-3-yl | nBu |
| 3-8-17 | Ph | H | CH₂CH₂SCH₃ | nBu |
| 3-8-18* | Ph | H | * | nBu |
| 3-8-19 | Ph | H | H | Ph |
| 3-8-20 | Ph | H | CH₃ | Ph |
| 3-8-21 | Ph | H | CH(CH₃)₂ | Ph |
| 3-8-22 | Ph | H | CH₂CH(CH₃)₂ | Ph |
| 3-8-23 | Ph | H | CH(CH₃)CH₂CH₃ | Ph |
| 3-8-24 | Ph | H | CH₂Ph | Ph |
| 3-8-25 | Ph | H | CH₂-indol-3-yl | Ph |
| 3-8-26 | Ph | H | CH₂CH₂SCH₃ | Ph |
| 3-8-27* | Ph | H | * | Ph |
| 3-8-28 | Ph | H | H | Bn |
| 3-8-29 | Ph | H | CH₃ | Bn |
| 3-8-30 | Ph | H | CH(CH₃)₂ | Bn |
| 3-8-31 | Ph | H | CH₂CH(CH₃)₂ | Bn |
| 3-8-32 | Ph | H | CH(CH₃)CH₂CH₃ | Bn |
| 3-8-33 | Ph | H | CH₂Ph | Bn |
| 3-8-34 | Ph | H | CH₂-indol-3-yl | Bn |
| 3-8-35 | Ph | H | CH₂CH₂SCH₃ | Bn |
| 3-8-36* | Ph | H | * | Bn |
| 3-8-37 | Ph | H | H | CH₃ |
| 3-8-38 | Ph | H | CH₃ | CH₃ |
| 3-8-39 | Ph | H | CH(CH₃)₂ | CH₃ |
| 3-8-40 | Ph | H | CH₂CH(CH₃)₂ | CH₃ |
| 3-8-41 | Ph | H | CH(CH₃)CH₂CH₃ | CH₃ |
| 3-8-42 | Ph | H | CH₂Ph | CH₃ |
| 3-8-43 | Ph | H | CH₂-indol-3-yl | CH₃ |
| 3-8-44 | Ph | H | CH₂CH₂SCH₃ | CH₃ |
| 3-8-45* | Ph | H | * | CH₃ |
| 3-8-46 | Ph | H | H | Et |
| 3-8-47 | Ph | H | CH₃ | Et |
| 3-8-48 | Ph | H | CH(CH₃)₂ | Et |
| 3-8-49 | Ph | H | CH₂CH(CH₃)₂ | Et |
| 3-8-50 | Ph | H | CH(CH₃)CH₂CH₃ | Et |
| 3-8-51 | Ph | H | CH₂Ph | Et |
| 3-8-52 | Ph | H | CH₂-indol-3-yl | Et |
| 3-8-53 | Ph | H | CH₂CH₂SCH₃ | Et |
| 3-8-54* | Ph | H | * | Et |
| 3-8-55 | 1-Nap | H | H | iPr |
| 3-8-56 | 1-Nap | H | CH₃ | iPr |
| 3-8-57 | 1-Nap | H | CH(CH₃)₂ | iPr |
| 3-8-58 | 1-Nap | H | CH₂CH(CH₃)₂ | iPr |
| 3-8-59 | 1-Nap | H | CH(CH₃)CH₂CH₃ | iPr |
| 3-8-60 | 1-Nap | H | CH₂Ph | iPr |
| 3-8-61 | 1-Nap | H | CH₂-indol-3-yl | iPr |
| 3-8-62 | 1-Nap | H | CH₂CH₂SCH₃ | iPr |
| 3-8-63* | 1-Nap | H | * | iPr |
| 3-8-64 | 1-Nap | H | H | nBu |
| 3-8-65 | 1-Nap | H | CH₃ | nBu |
| 3-8-66 | 1-Nap | H | CH(CH₃)₂ | nBu |
| 3-8-67 | 1-Nap | H | CH₂CH(CH₃)₂ | nBu |
| 3-8-68 | 1-Nap | H | CH(CH₃)CH₂CH₃ | nBu |
| 3-8-69 | 1-Nap | H | CH₂Ph | nBu |
| 3-8-70 | 1-Nap | H | CH₂-indol-3-yl | nBu |
| 3-8-71 | 1-Nap | H | CH₂CH₂SCH₃ | nBu |
| 3-8-72* | 1-Nap | H | * | nBu |
| 3-8-73 | 1-Nap | H | H | Ph |
| 3-8-74 | 1-Nap | H | CH₃ | Ph |
| 3-8-75 | 1-Nap | H | CH(CH₃)₂ | Ph |
| 3-8-76 | 1-Nap | H | CH₂CH(CH₃)₂ | Ph |
| 3-8-77 | 1-Nap | H | CH(CH₃)CH₂CH₃ | Ph |
| 3-8-78 | 1-Nap | H | CH₂Ph | Ph |
| 3-8-79 | 1-Nap | H | CH₂-indol-3-yl | Ph |
| 3-8-80 | 1-Nap | H | CH₂CH₂SCH₃ | Ph |
| 3-8-81* | 1-Nap | H | * | Ph |
| 3-8-82 | 1-Nap | H | H | Bn |
| 3-8-83 | 1-Nap | H | CH₃ | Bn |
| 3-8-84 | 1-Nap | H | CH(CH₃)₂ | Bn |
| 3-8-85 | 1-Nap | H | CH₂CH(CH₃)₂ | Bn |
| 3-8-86 | 1-Nap | H | CH(CH₃)CH₂CH₃ | Bn |
| 3-8-87 | 1-Nap | H | CH₂Ph | Bn |
| 3-8-88 | 1-Nap | H | CH₂-indol-3-yl | Bn |
| 3-8-89 | 1-Nap | H | CH₂CH₂SCH₃ | Bn |
| 3-8-90* | 1-Nap | H | * | Bn |
| 3-8-91 | 1-Nap | H | H | CH₃ |
| 3-8-92 | 1-Nap | H | CH₃ | CH₃ |
| 3-8-93 | 1-Nap | H | CH(CH₃)₂ | CH₃ |
| 3-8-94 | 1-Nap | H | CH₂CH(CH₃)₂ | CH₃ |
| 3-8-95 | 1-Nap | H | CH(CH₃)CH₂CH₃ | CH₃ |
| 3-8-96 | 1-Nap | H | CH₂Ph | CH₃ |
| 3-8-97 | 1-Nap | H | CH₂-indol-3-yl | CH₃ |
| 3-8-98 | 1-Nap | H | CH₂CH₂SCH₃ | CH₃ |
| 3-8-99* | 1-Nap | H | * | CH₃ |
| 3-8-100 | 1-Nap | H | H | Et |
| 3-8-101 | 1-Nap | H | CH₃ | Et |
| 3-8-102 | 1-Nap | H | CH(CH₃)₂ | Et |
| 3-8-103 | 1-Nap | H | CH₂CH(CH₃)₂ | Et |
| 3-8-104 | 1-Nap | H | CH(CH₃)CH₂CH₃ | Et |
| 3-8-105 | 1-Nap | H | CH₂Ph | Et |
| 3-8-106 | 1-Nap | H | CH₂-indol-3-yl | Et |
| 3-8-107 | 1-Nap | H | CH₂CH₂SCH₃ | Et |
| 3-8-108* | 1-Nap | H | * | Et |
| 3-8-109 | 2-Nap | H | H | iPr |
| 3-8-110 | 2-Nap | H | CH₃ | iPr |
| 3-8-111 | 2-Nap | H | CH(CH₃)₂ | iPr |
| 3-8-112 | 2-Nap | H | CH₂CH(CH₃)₂ | iPr |
| 3-8-113 | 2-Nap | H | CH(CH₃)CH₂CH₃ | iPr |
| 3-8-114 | 2-Nap | H | CH₂Ph | iPr |
| 3-8-115 | 2-Nap | H | CH₂-indol-3-yl | iPr |
| 3-8-116 | 2-Nap | H | CH₂CH₂SCH₃ | iPr |
| 3-8-117* | 2-Nap | H | * | iPr |
| 3-8-118 | 2-Nap | H | H | nBu |
| 3-8-119 | 2-Nap | H | CH₃ | nBu |
| 3-8-120 | 2-Nap | H | CH(CH₃)₂ | nBu |
| 3-8-121 | 2-Nap | H | CH₂CH(CH₃)₂ | nBu |
| 3-8-122 | 2-Nap | H | CH(CH₃)CH₂CH₃ | nBu |
| 3-8-123 | 2-Nap | H | CH₂Ph | nBu |
| 3-8-124 | 2-Nap | H | CH₂-indol-3-yl | nBu |
| 3-8-125 | 2-Nap | H | CH₂CH₂SCH₃ | nBu |
| 3-8-126* | 2-Nap | H | * | nBu |
| 3-8-127 | 2-Nap | H | H | Ph |
| 3-8-128 | 2-Nap | H | CH₃ | Ph |
| 3-8-129 | 2-Nap | H | CH(CH₃)₂ | Ph |
| 3-8-130 | 2-Nap | H | CH₂CH(CH₃)₂ | Ph |
| 3-8-131 | 2-Nap | H | CH(CH₃)CH₂CH₃ | Ph |
| 3-8-132 | 2-Nap | H | CH₂Ph | Ph |
| 3-8-133 | 2-Nap | H | CH₂-indol-3-yl | Ph |
| 3-8-134 | 2-Nap | H | CH₂CH₂SCH₃ | Ph |
| 3-8-135* | 2-Nap | H | * | Ph |
| 3-8-136 | 2-Nap | H | H | Bn |
| 3-8-137 | 2-Nap | H | CH₃ | Bn |
| 3-8-138 | 2-Nap | H | CH(CH₃)₂ | Bn |
| 3-8-139 | 2-Nap | H | CH₂CH(CH₃)₂ | Bn |
| 3-8-140 | 2-Nap | H | CH(CH₃)CH₂CH₃ | Bn |
| 3-8-141 | 2-Nap | H | CH₂Ph | Bn |
| 3-8-142 | 2-Nap | H | CH₂-indol-3-yl | Bn |
| 3-8-143 | 2-Nap | H | CH₂CH₂SCH₃ | Bn |
| 3-8-144* | 2-Nap | H | * | Bn |
| 3-8-145 | 2-Nap | H | H | CH₃ |
| 3-8-146 | 2-Nap | H | CH₃ | CH₃ |
| 3-8-147 | 2-Nap | H | CH(CH₃)₂ | CH₃ |
| 3-8-148 | 2-Nap | H | CH₂CH(CH₃)₂ | CH₃ |
| 3-8-149 | 2-Nap | H | CH(CH₃)CH₂CH₃ | CH₃ |
| 3-8-150 | 2-Nap | H | CH₂Ph | CH₃ |
| 3-8-151 | 2-Nap | H | CH₂-indol-3-yl | CH₃ |
| 3-8-152 | 2-Nap | H | CH₂CH₂SCH₃ | CH₃ |
| 3-8-153* | 2-Nap | H | * | CH₃ |
| 3-8-154 | 2-Nap | H | H | Et |
| 3-8-155 | 2-Nap | H | CH₃ | Et |
| 3-8-156 | 2-Nap | H | CH(CH₃)₂ | Et |
| 3-8-157 | 2-Nap | H | CH₂CH(CH₃)₂ | Et |
| 3-8-158 | 2-Nap | H | CH(CH₃)CH₂CH₃ | Et |
| 3-8-159 | 2-Nap | H | CH₂Ph | Et |
| 3-8-160 | 2-Nap | H | CH₂-indol-3-yl | Et |
| 3-8-161 | 2-Nap | H | CH₂CH₂SCH₃ | Et |
| 3-8-162* | 2-Nap | H | * | Et |
| 3-8-163 | p-Br-Ph | H | H | iPr |
| 3-8-164 | p-Br-Ph | H | CH₃ | iPr |
| 3-8-165 | p-Br-Ph | H | CH(CH₃)₂ | iPr |
| 3-8-166 | p-Br-Ph | H | CH₂CH(CH₃)₂ | iPr |
| 3-8-167 | p-Br-Ph | H | CH(CH₃)CH₂CH₃ | iPr |
| 3-8-168 | p-Br-Ph | H | CH₂Ph | iPr |
| 3-8-169 | p-Br-Ph | H | CH₂-indol-3-yl | iPr |
| 3-8-170 | p-Br-Ph | H | CH₂CH₂SCH₃ | iPr |
| 3-8-171* | p-Br-Ph | H | * | iPr |
| 3-8-172 | p-Br-Ph | H | H | nBu |
| 3-8-173 | p-Br-Ph | H | CH₃ | nBu |
| 3-8-174 | p-Br-Ph | H | CH(CH₃)₂ | nBu |
| 3-8-175 | p-Br-Ph | H | CH₂CH(CH₃)₂ | nBu |
| 3-8-176 | p-Br-Ph | H | CH(CH₃)CH₂CH₃ | nBu |
| 3-8-177 | p-Br-Ph | H | CH₂Ph | nBu |
| 3-8-178 | p-Br-Ph | H | CH₂-indol-3-yl | nBu |
| 3-8-179 | p-Br-Ph | H | CH₂CH₂SCH₃ | nBu |
| 3-8-180* | p-Br-Ph | H | * | nBu |
| 3-8-181 | p-Br-Ph | H | H | Ph |
| 3-8-182 | p-Br-Ph | H | CH₃ | Ph |
| 3-8-183 | p-Br-Ph | H | CH(CH₃)₂ | Ph |
| 3-8-184 | p-Br-Ph | H | CH₂CH(CH₃)₂ | Ph |
| 3-8-185 | p-Br-Ph | H | CH(CH₃)CH₂CH₃ | Ph |
| 3-8-186 | p-Br-Ph | H | CH₂Ph | Ph |
| 3-8-187 | p-Br-Ph | H | CH₂-indol-3-yl | Ph |
| 3-8-188 | p-Br-Ph | H | CH₂CH₂SCH₃ | Ph |
| 3-8-189* | p-Br-Ph | H | * | Ph |
| 3-8-190 | p-Br-Ph | H | H | Bn |
| 3-8-191 | p-Br-Ph | H | CH₃ | Bn |
| 3-8-192 | p-Br-Ph | H | CH(CH₃)₂ | Bn |
| 3-8-193 | p-Br-Ph | H | CH₂CH(CH₃)₂ | Bn |
| 3-8-194 | p-Br-Ph | H | CH(CH₃)CH₂CH₃ | Bn |
| 3-8-195 | p-Br-Ph | H | CH₂Ph | Bn |
| 3-8-196 | p-Br-Ph | H | CH₂-indol-3-yl | Bn |
| 3-8-197 | p-Br-Ph | H | CH₂CH₂SCH₃ | Bn |
| 3-8-198* | p-Br-Ph | H | * | Bn |
| 3-8-199 | p-Br-Ph | H | H | CH₃ |
| 3-8-200 | p-Br-Ph | H | CH₃ | CH₃ |
| 3-8-201 | p-Br-Ph | H | CH(CH₃)₂ | CH₃ |
| 3-8-202 | p-Br-Ph | H | CH₂CH(CH₃)₂ | CH₃ |
| 3-8-203 | p-Br-Ph | H | CH(CH₃)CH₂CH₃ | CH₃ |
| 3-8-204 | p-Br-Ph | H | CH₂Ph | CH₃ |
| 3-8-205 | p-Br-Ph | H | CH₂-indol-3-yl | CH₃ |
| 3-8-206 | p-Br-Ph | H | CH₂CH₂SCH₃ | CH₃ |
| 3-8-207* | p-Br-Ph | H | * | CH₃ |
| 3-8-208 | p-Br-Ph | H | H | Et |
| 3-8-209 | p-Br-Ph | H | CH₃ | Et |
| 3-8-210 | p-Br-Ph | H | CH(CH₃)₂ | Et |
| 3-8-211 | p-Br-Ph | H | CH₂CH(CH₃)₂ | Et |
| 3-8-212 | p-Br-Ph | H | CH(CH₃)CH₂CH₃ | Et |
| 3-8-213 | p-Br-Ph | H | CH₂Ph | Et |
| 3-8-214 | p-Br-Ph | H | CH₂-indol-3-yl | Et |
| 3-8-215 | p-Br-Ph | H | CH₂CH₂SCH₃ | Et |
| 3-8-216* | p-Br-Ph | H | * | Et |
| 3-8-217 | p-Cl-Ph | H | H | iPr |
| 3-8-218 | p-Cl-Ph | H | CH₃ | iPr |
| 3-8-219 | p-Cl-Ph | H | CH(CH₃)₂ | iPr |
| 3-8-220 | p-Cl-Ph | H | CH₂CH(CH₃)₂ | iPr |
| 3-8-221 | p-Cl-Ph | H | CH(CH₃)CH₂CH₃ | iPr |
| 3-8-222 | p-Cl-Ph | H | CH₂Ph | iPr |
| 3-8-223 | p-Cl-Ph | H | CH₂-indol-3-yl | iPr |
| 3-8-224 | p-Cl-Ph | H | CH₂CH₂SCH₃ | iPr |
| 3-8-225* | p-Cl-Ph | H | * | iPr |
| 3-8-226 | p-Cl-Ph | H | H | nBu |
| 3-8-227 | p-Cl-Ph | H | CH₃ | nBu |
| 3-8-228 | p-Cl-Ph | H | CH(CH₃)₂ | nBu |
| 3-8-229 | p-Cl-Ph | H | CH₂CH(CH₃)₂ | nBu |
| 3-8-230 | p-Cl-Ph | H | CH(CH₃)CH₂CH₃ | nBu |
| 3-8-231 | p-Cl-Ph | H | CH₂Ph | nBu |
| 3-8-232 | p-Cl-Ph | H | CH₂-indol-3-yl | nBu |
| 3-8-233 | p-Cl-Ph | H | CH₂CH₂SCH₃ | nBu |
| 3-8-234* | p-Cl-Ph | H | * | nBu |
| 3-8-235 | p-Cl-Ph | H | H | Ph |
| 3-8-236 | p-Cl-Ph | H | CH₃ | Ph |
| 3-8-237 | p-Cl-Ph | H | CH(CH₃)₂ | Ph |
| 3-8-238 | p-Cl-Ph | H | CH₂CH(CH₃)₂ | Ph |
| 3-8-239 | p-Cl-Ph | H | CH(CH₃)CH₂CH₃ | Ph |
| 3-8-240 | p-Cl-Ph | H | CH₂Ph | Ph |
| 3-8-241 | p-Cl-Ph | H | CH₂-indol-3-yl | Ph |
| 3-8-242 | p-Cl-Ph | H | CH₂CH₂SCH₃ | Ph |
| 3-8-243* | p-Cl-Ph | H | * | Ph |
| 3-8-244 | p-Cl-Ph | H | H | Bn |
| 3-8-245 | p-Cl-Ph | H | CH₃ | Bn |
| 3-8-246 | p-Cl-Ph | H | CH(CH₃)₂ | Bn |
| 3-8-247 | p-Cl-Ph | H | CH₂CH(CH₃)₂ | Bn |
| 3-8-248 | p-Cl-Ph | H | CH(CH₃)CH₂CH₃ | Bn |
| 3-8-249 | p-Cl-Ph | H | CH₂Ph | Bn |
| 3-8-250 | p-Cl-Ph | H | CH₂-indol-3-yl | Bn |
| 3-8-251 | p-Cl-Ph | H | CH₂CH₂SCH₃ | Bn |
| 3-8-252* | p-Cl-Ph | H | * | Bn |
| 3-8-253 | p-Cl-Ph | H | H | CH₃ |
| 3-8-254 | p-Cl-Ph | H | CH₃ | CH₃ |
| 3-8-255 | p-Cl-Ph | H | CH(CH₃)₂ | CH₃ |
| 3-8-256 | p-Cl-Ph | H | CH₂CH(CH₃)₂ | CH₃ |
| 3-8-257 | p-Cl-Ph | H | CH(CH₃)CH₂CH₃ | CH₃ |
| 3-8-258 | p-Cl-Ph | H | CH₂Ph | CH₃ |
| 3-8-259 | p-Cl-Ph | H | CH₂-indol-3-yl | CH₃ |
| 3-8-260 | p-Cl-Ph | H | CH₂CH₂SCH₃ | CH₃ |
| 3-8-261* | p-Cl-Ph | H | * | CH₃ |
| 3-8-262 | p-Cl-Ph | H | H | Et |
| 3-8-263 | p-Cl-Ph | H | CH₃ | Et |
| 3-8-264 | p-Cl-Ph | H | CH(CH₃)₂ | Et |
| 3-8-265 | p-Cl-Ph | H | CH₂CH(CH₃)₂ | Et |
| 3-8-266 | p-Cl-Ph | H | CH(CH₃)CH₂CH₃ | Et |
| 3-8-267 | p-Cl-Ph | H | CH₂Ph | Et |
| 3-8-268 | p-Cl-Ph | H | CH₂-indol-3-yl | Et |
| 3-8-269 | p-Cl-Ph | H | CH₂CH₂SCH₃ | Et |
| 3-8-270* | p-Cl-Ph | H | * | Et |
| 3-8-271 | p-F-Ph | H | H | iPr |
| 3-8-272 | p-F-Ph | H | CH₃ | iPr |
| 3-8-273 | p-F-Ph | H | CH(CH₃)₂ | iPr |
| 3-8-274 | p-F-Ph | H | CH₂CH(CH₃)₂ | iPr |
| 3-8-275 | p-F-Ph | H | CH(CH₃)CH₂CH₃ | iPr |
| 3-8-276 | p-F-Ph | H | CH₂Ph | iPr |
| 3-8-277 | p-F-Ph | H | CH₂-indol-3-yl | iPr |
| 3-8-278 | p-F-Ph | H | CH₂CH₂SCH₃ | iPr |
| 3-8-279* | p-F-Ph | H | * | iPr |
| 3-8-280 | p-F-Ph | H | H | nBu |
| 3-8-281 | p-F-Ph | H | CH₃ | nBu |
| 3-8-282 | p-F-Ph | H | CH(CH₃)₂ | nBu |
| 3-8-283 | p-F-Ph | H | CH₂CH(CH₃)₂ | nBu |
| 3-8-284 | p-F-Ph | H | CH(CH₃)CH₂CH₃ | nBu |
| 3-8-285 | p-F-Ph | H | CH₂Ph | nBu |
| 3-8-286 | p-F-Ph | H | CH₂-indol-3-yl | nBu |
| 3-8-287 | p-F-Ph | H | CH₂CH₂SCH₃ | nBu |
| 3-8-288* | p-F-Ph | H | * | nBu |
| 3-8-289 | p-F-Ph | H | H | Ph |
| 3-8-290 | p-F-Ph | H | CH₃ | Ph |
| 3-8-291 | p-F-Ph | H | CH(CH₃)₂ | Ph |
| 3-8-292 | p-F-Ph | H | CH₂CH(CH₃)₂ | Ph |
| 3-8-293 | p-F-Ph | H | CH(CH₃)CH₂CH₃ | Ph |
| 3-8-294 | p-F-Ph | H | CH₂Ph | Ph |
| 3-8-295 | p-F-Ph | H | CH₂-indol-3-yl | Ph |
| 3-8-296 | p-F-Ph | H | CH₂CH₂SCH₃ | Ph |
| 3-8-297* | p-F-Ph | H | * | Ph |
| 3-8-298 | p-F-Ph | H | H | Bn |
| 3-8-299 | p-F-Ph | H | CH₃ | Bn |
| 3-8-300 | p-F-Ph | H | CH(CH₃)₂ | Bn |
| 3-8-301 | p-F-Ph | H | CH₂CH(CH₃)₂ | Bn |
| 3-8-302 | p-F-Ph | H | CH(CH₃)CH₂CH₃ | Bn |
| 3-8-303 | p-F-Ph | H | CH₂Ph | Bn |
| 3-8-304 | p-F-Ph | H | CH₂-indol-3-yl | Bn |
| 3-8-305 | p-F-Ph | H | CH₂CH₂SCH₃ | Bn |
| 3-8-306* | p-F-Ph | H | * | Bn |
| 3-8-307 | p-F-Ph | H | H | CH₃ |
| 3-8-308 | p-F-Ph | H | CH₃ | CH₃ |
| 3-8-309 | p-F-Ph | H | CH(CH₃)₂ | CH₃ |
| 3-8-310 | p-F-Ph | H | CH₂CH(CH₃)₂ | CH₃ |
| 3-8-311 | p-F-Ph | H | CH(CH₃)CH₂CH₃ | CH₃ |
| 3-8-312 | p-F-Ph | H | CH₂Ph | CH₃ |
| 3-8-313 | p-F-Ph | H | CH₂-indol-3-yl | CH₃ |
| 3-8-314 | p-F-Ph | H | CH₂CH₂SCH₃ | CH₃ |
| 3-8-315* | p-F-Ph | H | * | CH₃ |
| 3-8-316 | p-F-Ph | H | H | Et |
| 3-8-317 | p-F-Ph | H | CH₃ | Et |
| 3-8-318 | p-F-Ph | H | CH(CH₃)₂ | Et |
| 3-8-319 | p-F-Ph | H | CH₂CH(CH₃)₂ | Et |
| 3-8-320 | p-F-Ph | H | CH(CH₃)CH₂CH₃ | Et |
| 3-8-321 | p-F-Ph | H | CH₂Ph | Et |
| 3-8-322 | p-F-Ph | H | CH₂-indol-3-yl | Et |
| 3-8-323 | p-F-Ph | H | CH₂CH₂SCH₃ | Et |
| 3-8-324* | p-F-Ph | H | * | Et |
| 3-8-325 | p-I-Ph | H | H | iPr |
| 3-8-326 | p-I-Ph | H | CH₃ | iPr |
| 3-8-327 | p-I-Ph | H | CH(CH₃)₂ | iPr |
| 3-8-328 | p-I-Ph | H | CH₂CH(CH₃)₂ | iPr |
| 3-8-329 | p-I-Ph | H | CH(CH₃)CH₂CH₃ | iPr |
| 3-8-330 | p-I-Ph | H | CH₂Ph | iPr |
| 3-8-331 | p-I-Ph | H | CH₂-indol-3-yl | iPr |
| 3-8-332 | p-I-Ph | H | CH₂CH₂SCH₃ | iPr |
| 3-8-333* | p-I-Ph | H | * | iPr |
| 3-8-334 | p-I-Ph | H | H | nBu |
| 3-8-335 | p-I-Ph | H | CH₃ | nBu |
| 3-8-336 | p-I-Ph | H | CH(CH₃)₂ | nBu |
| 3-8-337 | p-I-Ph | H | CH₂CH(CH₃)₂ | nBu |
| 3-8-338 | p-I-Ph | H | CH(CH₃)CH₂CH₃ | nBu |
| 3-8-339 | p-I-Ph | H | CH₂Ph | nBu |
| 3-8-340 | p-I-Ph | H | CH₂-indol-3-yl | nBu |
| 3-8-341 | p-I-Ph | H | CH₂CH₂SCH₃ | nBu |
| 3-8-342* | p-I-Ph | H | * | nBu |
| 3-8-343 | p-I-Ph | H | H | Ph |
| 3-8-344 | p-I-Ph | H | CH₃ | Ph |
| 3-8-345 | p-I-Ph | H | CH(CH₃)₂ | Ph |
| 3-8-346 | p-I-Ph | H | CH₂CH(CH₃)₂ | Ph |
| 3-8-347 | p-I-Ph | H | CH(CH₃)CH₂CH₃ | Ph |
| 3-8-348 | p-I-Ph | H | CH₂Ph | Ph |
| 3-8-349 | p-I-Ph | H | CH₂-indol-3-yl | Ph |
| 3-8-350 | p-I-Ph | H | CH₂CH₂SCH₃ | Ph |
| 3-8-351* | p-I-Ph | H | * | Ph |
| 3-8-352 | p-I-Ph | H | H | Bn |
| 3-8-353 | p-I-Ph | H | CH₃ | Bn |
| 3-8-354 | p-I-Ph | H | CH(CH₃)₂ | Bn |
| 3-8-355 | p-I-Ph | H | CH₂CH(CH₃)₂ | Bn |
| 3-8-356 | p-I-Ph | H | CH(CH₃)CH₂CH₃ | Bn |
| 3-8-357 | p-I-Ph | H | CH₂Ph | Bn |
| 3-8-358 | p-I-Ph | H | CH₂-indol-3-yl | Bn |
| 3-8-359 | p-I-Ph | H | CH₂CH₂SCH₃ | Bn |
| 3-8-360* | p-I-Ph | H | * | Bn |
| 3-8-361 | p-I-Ph | H | H | CH₃ |
| 3-8-362 | p-I-Ph | H | CH₃ | CH₃ |
| 3-8-363 | p-I-Ph | H | CH(CH₃)₂ | CH₃ |
| 3-8-364 | p-I-Ph | H | CH₂CH(CH₃)₂ | CH₃ |
| 3-8-365 | p-I-Ph | H | CH(CH₃)CH₂CH₃ | CH₃ |
| 3-8-366 | p-I-Ph | H | CH₂Ph | CH₃ |
| 3-8-367 | p-I-Ph | H | CH₂-indol-3-yl | CH₃ |
| 3-8-368 | p-I-Ph | H | CH₂CH₂SCH₃ | CH₃ |
| 3-8-369* | p-I-Ph | H | * | CH₃ |
| 3-8-370 | p-I-Ph | H | H | Et |
| 3-8-371 | p-I-Ph | H | CH₃ | Et |
| 3-8-372 | p-I-Ph | H | CH(CH₃)₂ | Et |
| 3-8-373 | p-I-Ph | H | CH₂CH(CH₃)₂ | Et |
| 3-8-374 | p-I-Ph | H | CH(CH₃)CH₂CH₃ | Et |
| 3-8-375 | p-I-Ph | H | CH₂Ph | Et |
| 3-8-376 | p-I-Ph | H | CH₂-indol-3-yl | Et |
| 3-8-377 | p-I-Ph | H | CH₂CH₂SCH₃ | Et |
| 3-8-378* | p-I-Ph | H | * | Et |
| 3-8-379 | p-Me-Ph | H | H | iPr |
| 3-8-380 | p-Me-Ph | H | CH₃ | iPr |
| 3-8-381 | p-Me-Ph | H | CH(CH₃)₂ | iPr |
| 3-8-382 | p-Me-Ph | H | CH₂CH(CH₃)₂ | iPr |
| 3-8-383 | p-Me-Ph | H | CH(CH₃)CH₂CH₃ | iPr |
| 3-8-384 | p-Me-Ph | H | CH₂Ph | iPr |
| 3-8-385 | p-Me-Ph | H | CH₂-indol-3-yl | iPr |
| 3-8-386 | p-Me-Ph | H | CH₂CH₂SCH₃ | iPr |
| 3-8-387* | p-Me-Ph | H | * | iPr |
| 3-8-388 | p-Me-Ph | H | H | nBu |
| 3-8-389 | p-Me-Ph | H | CH₃ | nBu |
| 3-8-390 | p-Me-Ph | H | CH(CH₃)₂ | nBu |
| 3-8-391 | p-Me-Ph | H | CH₂CH(CH₃)₂ | nBu |
| 3-8-392 | p-Me-Ph | H | CH(CH₃)CH₂CH₃ | nBu |
| 3-8-393 | p-Me-Ph | H | CH₂Ph | nBu |
| 3-8-394 | p-Me-Ph | H | CH₂-indol-3-yl | nBu |
| 3-8-395 | p-Me-Ph | H | CH₂CH₂SCH₃ | nBu |
| 3-8-396* | p-Me-Ph | H | * | nBu |
| 3-8-397 | p-Me-Ph | H | H | Ph |
| 3-8-398 | p-Me-Ph | H | CH₃ | Ph |
| 3-8-399 | p-Me-Ph | H | CH(CH₃)₂ | Ph |
| 3-8-400 | p-Me-Ph | H | CH₂CH(CH₃)₂ | Ph |
| 3-8-401 | p-Me-Ph | H | CH(CH₃)CH₂CH₃ | Ph |
| 3-8-402 | p-Me-Ph | H | CH₂Ph | Ph |
| 3-8-403 | p-Me-Ph | H | CH₂-indol-3-yl | Ph |
| 3-8-404 | p-Me-Ph | H | CH₂CH₂SCH₃ | Ph |
| 3-8-405* | p-Me-Ph | H | * | Ph |
| 3-8-406 | p-Me-Ph | H | H | Bn |
| 3-8-407 | p-Me-Ph | H | CH₃ | Bn |
| 3-8-408 | p-Me-Ph | H | CH(CH₃)₂ | Bn |
| 3-8-409 | p-Me-Ph | H | CH₂CH(CH₃)₂ | Bn |
| 3-8-410 | p-Me-Ph | H | CH(CH₃)CH₂CH₃ | Bn |
| 3-8-411 | p-Me-Ph | H | CH₂Ph | Bn |
| 3-8-412 | p-Me-Ph | H | CH₂-indol-3-yl | Bn |
| 3-8-413 | p-Me-Ph | H | CH₂CH₂SCH₃ | Bn |
| 3-8-414* | p-Me-Ph | H | * | Bn |
| 3-8-415 | p-Me-Ph | H | H | CH₃ |
| 3-8-416 | p-Me-Ph | H | CH₃ | CH₃ |
| 3-8-417 | p-Me-Ph | H | CH(CH₃)₂ | CH₃ |
| 3-8-418 | p-Me-Ph | H | CH₂CH(CH₃)₂ | CH₃ |
| 3-8-419 | p-Me-Ph | H | CH(CH₃)CH₂CH₃ | CH₃ |
| 3-8-420 | p-Me-Ph | H | CH₂Ph | CH₃ |
| 3-8-421 | p-Me-Ph | H | CH₂-indol-3-yl | CH₃ |
| 3-8-422 | p-Me-Ph | H | CH₂CH₂SCH₃ | CH₃ |
| 3-8-423* | p-Me-Ph | H | * | CH₃ |
| 3-8-424 | p-Me-Ph | H | H | Et |
| 3-8-425 | p-Me-Ph | H | CH₃ | Et |
| 3-8-426 | p-Me-Ph | H | CH(CH₃)₂ | Et |
| 3-8-427 | p-Me-Ph | H | CH₂CH(CH₃)₂ | Et |
| 3-8-428 | p-Me-Ph | H | CH(CH₃)CH₂CH₃ | Et |
| 3-8-429 | p-Me-Ph | H | CH₂Ph | Et |
| 3-8-430 | p-Me-Ph | H | CH₂-indol-3-yl | Et |
| 3-8-431 | p-Me-Ph | H | CH₂CH₂SCH₃ | Et |
| 3-8-432* | p-Me-Ph | H | * | Et |
| 3-8-433 | tBu | H | H | iPr |
| 3-8-434 | tBu | H | CH₃ | iPr |
| 3-8-435 | tBu | H | CH(CH₃)₂ | iPr |
| 3-8-436 | tBu | H | CH₂CH(CH₃)₂ | iPr |
| 3-8-437 | tBu | H | CH(CH₃)CH₂CH₃ | iPr |
| 3-8-438 | tBu | H | CH₂Ph | iPr |
| 3-8-439 | tBu | H | CH₂-indol-3-yl | iPr |
| 3-8-440 | tBu | H | CH₂CH₂SCH₃ | iPr |
| 3-8-441* | tBu | H | * | iPr |
| 3-8-442 | tBu | H | H | nBu |
| 3-8-443 | tBu | H | CH₃ | nBu |
| 3-8-444 | tBu | H | CH(CH₃)₂ | nBu |
| 3-8-445 | tBu | H | CH₂CH(CH₃)₂ | nBu |
| 3-8-446 | tBu | H | CH(CH₃)CH₂CH₃ | nBu |
| 3-8-447 | tBu | H | CH₂Ph | nBu |
| 3-8-448 | tBu | H | CH₂-indol-3-yl | nBu |
| 3-8-449 | tBu | H | CH₂CH₂SCH₃ | nBu |
| 3-8-450* | tBu | H | * | nBu |
| 3-8-451 | tBu | H | H | Ph |
| 3-8-452 | tBu | H | CH₃ | Ph |
| 3-8-453 | tBu | H | CH(CH₃)₂ | Ph |
| 3-8-454 | tBu | H | CH₂CH(CH₃)₂ | Ph |
| 3-8-455 | tBu | H | CH(CH₃)CH₂CH₃ | Ph |
| 3-8-456 | tBu | H | CH₂Ph | Ph |
| 3-8-457 | tBu | H | CH₂-indol-3-yl | Ph |
| 3-8-458 | tBu | H | CH₂CH₂SCH₃ | Ph |
| 3-8-459* | tBu | H | * | Ph |
| 3-8-460 | tBu | H | H | Bn |
| 3-8-461 | tBu | H | CH₃ | Bn |
| 3-8-462 | tBu | H | CH(CH₃)₂ | Bn |
| 3-8-463 | tBu | H | CH₂CH(CH₃)₂ | Bn |
| 3-8-464 | tBu | H | CH(CH₃)CH₂CH₃ | Bn |
| 3-8-465 | tBu | H | CH₂Ph | Bn |
| 3-8-466 | tBu | H | CH₂-indol-3-yl | Bn |
| 3-8-467 | tBu | H | CH₂CH₂SCH₃ | Bn |
| 3-8-468* | tBu | H | * | Bn |
| 3-8-469 | tBu | H | H | CH₃ |
| 3-8-470 | tBu | H | CH₃ | CH₃ |
| 3-8-471 | tBu | H | CH(CH₃)₂ | CH₃ |
| 3-8-472 | tBu | H | CH₂CH(CH₃)₂ | CH₃ |
| 3-8-473 | tBu | H | CH(CH₃)CH₂CH₃ | CH₃ |
| 3-8-474 | tBu | H | CH₂Ph | CH₃ |
| 3-8-475 | tBu | H | CH₂-indol-3-yl | CH₃ |
| 3-8-476 | tBu | H | CH₂CH₂SCH₃ | CH₃ |
| 3-8-477* | tBu | H | * | CH₃ |
| 3-8-478 | tBu | H | H | Et |
| 3-8-479 | tBu | H | CH₃ | Et |
| 3-8-480 | tBu | H | CH(CH₃)₂ | Et |
| 3-8-481 | tBu | H | CH₂CH(CH₃)₂ | Et |
| 3-8-482 | tBu | H | CH(CH₃)CH₂CH₃ | Et |
| 3-8-483 | tBu | H | CH₂Ph | Et |
| 3-8-484 | tBu | H | CH₂-indol-3-yl | Et |
| 3-8-485 | tBu | H | CH₂CH₂SCH₃ | Et |
| 3-8-486* | tBu | H | * | Et |
| 3-8-487 | Ph | H | H | iPr |
| 3-8-488 | Ph | CH₃ | H | iPr |
| 3-8-489 | Ph | CH(CH₃)₂ | H | iPr |
| 3-8-490 | Ph | CH₂CH(CH₃)₂ | H | iPr |
| 3-8-491 | Ph | CH(CH₃)CH₂CH₃ | H | iPr |
| 3-8-492 | Ph | CH₂Ph | H | iPr |
| 3-8-493 | Ph | CH₂-indol-3-yl | H | iPr |
| 3-8-494 | Ph | CH₂CH₂SCH₃ | H | iPr |
| 3-8-495* | Ph | * | H | iPr |
| 3-8-496 | Ph | H | H | nBu |
| 3-8-497 | Ph | CH₃ | H | nBu |
| 3-8-498 | Ph | CH(CH₃)₂ | H | nBu |
| 3-8-499 | Ph | CH₂CH(CH₃)₂ | H | nBu |
| 3-8-500 | Ph | CH(CH₃)CH₂CH₃ | H | nBu |
| 3-8-501 | Ph | CH₂Ph | H | nBu |
| 3-8-502 | Ph | CH₂-indol-3-yl | H | nBu |
| 3-8-503 | Ph | CH₂CH₂SCH₃ | H | nBu |
| 3-8-504* | Ph | * | H | nBu |
| 3-8-505 | Ph | H | H | Ph |
| 3-8-506 | Ph | CH₃ | H | Ph |
| 3-8-507 | Ph | CH(CH₃)₂ | H | Ph |
| 3-8-508 | Ph | CH₂CH(CH₃)₂ | H | Ph |
| 3-8-509 | Ph | CH(CH₃)CH₂CH₃ | H | Ph |
| 3-8-510 | Ph | CH₂Ph | H | Ph |
| 3-8-511 | Ph | CH₂-indol-3-yl | H | Ph |
| 3-8-512 | Ph | CH₂CH₂SCH₃ | H | Ph |
| 3-8-513* | Ph | * | H | Ph |
| 3-8-514 | Ph | H | H | Bn |
| 3-8-515 | Ph | CH₃ | H | Bn |
| 3-8-516 | Ph | CH(CH₃)₂ | H | Bn |
| 3-8-517 | Ph | CH₂CH(CH₃)₂ | H | Bn |
| 3-8-518 | Ph | CH(CH₃)CH₂CH₃ | H | Bn |
| 3-8-519 | Ph | CH₂Ph | H | Bn |
| 3-8-520 | Ph | CH₂-indol-3-yl | H | Bn |
| 3-8-521 | Ph | CH₂CH₂SCH₃ | H | Bn |
| 3-8-522* | Ph | * | H | Bn |
| 3-8-523 | Ph | H | H | CH₃ |
| 3-8-524 | Ph | CH₃ | H | CH₃ |
| 3-8-525 | Ph | CH(CH₃)₂ | H | CH₃ |
| 3-8-526 | Ph | CH₂CH(CH₃)₂ | H | CH₃ |
| 3-8-527 | Ph | CH(CH₃)CH₂CH₃ | H | CH₃ |
| 3-8-528 | Ph | CH₂Ph | H | CH₃ |
| 3-8-529 | Ph | CH₂-indol-3-yl | H | CH₃ |
| 3-8-530 | Ph | CH₂CH₂SCH₃ | H | CH₃ |
| 3-8-531* | Ph | * | H | CH₃ |
| 3-8-532 | Ph | H | H | Et |
| 3-8-533 | Ph | CH₃ | H | Et |
| 3-8-534 | Ph | CH(CH₃)₂ | H | Et |
| 3-8-535 | Ph | CH₂CH(CH₃)₂ | H | Et |
| 3-8-536 | Ph | CH(CH₃)CH₂CH₃ | H | Et |
| 3-8-537 | Ph | CH₂Ph | H | Et |
| 3-8-538 | Ph | CH₂-indol-3-yl | H | Et |
| 3-8-539 | Ph | CH₂CH₂SCH₃ | H | Et |
| 3-8-540* | Ph | * | H | Et |
| 3-8-541 | 1-Nap | H | H | iPr |
| 3-8-542 | 1-Nap | CH₃ | H | iPr |
| 3-8-543 | 1-Nap | CH(CH₃)₂ | H | iPr |
| 3-8-544 | 1-Nap | CH₂CH(CH₃)₂ | H | iPr |
| 3-8-545 | 1-Nap | CH(CH₃)CH₂CH₃ | H | iPr |
| 3-8-546 | 1-Nap | CH₂Ph | H | iPr |
| 3-8-547 | 1-Nap | CH₂-indol-3-yl | H | iPr |
| 3-8-548 | 1-Nap | CH₂CH₂SCH₃ | H | iPr |
| 3-8-549* | 1-Nap | * | H | iPr |
| 3-8-550 | 1-Nap | H | H | nBu |
| 3-8-551 | 1-Nap | CH₃ | H | nBu |
| 3-8-552 | 1-Nap | CH(CH₃)₂ | H | nBu |
| 3-8-553 | 1-Nap | CH₂CH(CH₃)₂ | H | nBu |
| 3-8-554 | 1-Nap | CH(CH₃)CH₂CH₃ | H | nBu |
| 3-8-555 | 1-Nap | CH₂Ph | H | nBu |
| 3-8-556 | 1-Nap | CH₂-indol-3-yl | H | nBu |
| 3-8-557 | 1-Nap | CH₂CH₂SCH₃ | H | nBu |
| 3-8-558* | 1-Nap | * | H | nBu |
| 3-8-559 | 1-Nap | H | H | Ph |
| 3-8-560 | 1-Nap | CH₃ | H | Ph |
| 3-8-561 | 1-Nap | CH(CH₃)₂ | H | Ph |
| 3-8-562 | 1-Nap | CH₂CH(CH₃)₂ | H | Ph |
| 3-8-563 | 1-Nap | CH(CH₃)CH₂CH₃ | H | Ph |
| 3-8-564 | 1-Nap | CH₂Ph | H | Ph |
| 3-8-565 | 1-Nap | CH₂-indol-3-yl | H | Ph |
| 3-8-566 | 1-Nap | CH₂CH₂SCH₃ | H | Ph |
| 3-8-567* | 1-Nap | * | H | Ph |
| 3-8-568 | 1-Nap | H | H | Bn |
| 3-8-569 | 1-Nap | CH₃ | H | Bn |
| 3-8-570 | 1-Nap | CH(CH₃)₂ | H | Bn |
| 3-8-571 | 1-Nap | CH₂CH(CH₃)₂ | H | Bn |
| 3-8-572 | 1-Nap | CH(CH₃)CH₂CH₃ | H | Bn |
| 3-8-573 | 1-Nap | CH₂Ph | H | Bn |
| 3-8-574 | 1-Nap | CH₂-indol-3-yl | H | Bn |
| 3-8-575 | 1-Nap | CH₂CH₂SCH₃ | H | Bn |
| 3-8-576* | 1-Nap | * | H | Bn |
| 3-8-577 | 1-Nap | H | H | CH₃ |
| 3-8-578 | 1-Nap | CH₃ | H | CH₃ |
| 3-8-579 | 1-Nap | CH(CH₃)₂ | H | CH₃ |
| 3-8-580 | 1-Nap | CH₂CH(CH₃)₂ | H | CH₃ |
| 3-8-581 | 1-Nap | CH(CH₃)CH₂CH₃ | H | CH₃ |
| 3-8-582 | 1-Nap | CH₂Ph | H | CH₃ |
| 3-8-583 | 1-Nap | CH₂-indol-3-yl | H | CH₃ |
| 3-8-584 | 1-Nap | CH₂CH₂SCH₃ | H | CH₃ |
| 3-8-585* | 1-Nap | * | H | CH₃ |
| 3-8-586 | 1-Nap | H | H | Et |
| 3-8-587 | 1-Nap | CH₃ | H | Et |
| 3-8-588 | 1-Nap | CH(CH₃)₂ | H | Et |
| 3-8-589 | 1-Nap | CH₂CH(CH₃)₂ | H | Et |
| 3-8-590 | 1-Nap | CH(CH₃)CH₂CH₃ | H | Et |
| 3-8-591 | 1-Nap | CH₂Ph | H | Et |
| 3-8-592 | 1-Nap | CH₂-indol-3-yl | H | Et |
| 3-8-593 | 1-Nap | CH₂CH₂SCH₃ | H | Et |
| 3-8-594* | 1-Nap | * | H | Et |
| 3-8-595 | 2-Nap | H | H | iPr |
| 3-8-596 | 2-Nap | CH₃ | H | iPr |
| 3-8-597 | 2-Nap | CH(CH₃)₂ | H | iPr |
| 3-8-598 | 2-Nap | CH₂CH(CH₃)₂ | H | iPr |
| 3-8-599 | 2-Nap | CH(CH₃)CH₂CH₃ | H | iPr |
| 3-8-600 | 2-Nap | CH₂Ph | H | iPr |
| 3-8-601 | 2-Nap | CH₂-indol-3-yl | H | iPr |
| 3-8-602 | 2-Nap | CH₂CH₂SCH₃ | H | iPr |
| 3-8-603* | 2-Nap | * | H | iPr |
| 3-8-604 | 2-Nap | H | H | nBu |
| 3-8-605 | 2-Nap | CH₃ | H | nBu |
| 3-8-606 | 2-Nap | CH(CH₃)₂ | H | nBu |
| 3-8-607 | 2-Nap | CH₂CH(CH₃)₂ | H | nBu |
| 3-8-608 | 2-Nap | CH(CH₃)CH₂CH₃ | H | nBu |
| 3-8-609 | 2-Nap | CH₂Ph | H | nBu |
| 3-8-610 | 2-Nap | CH₂-indol-3-yl | H | nBu |
| 3-8-611 | 2-Nap | CH₂CH₂SCH₃ | H | nBu |
| 3-8-612* | 2-Nap | * | H | nBu |
| 3-8-613 | 2-Nap | H | H | Ph |
| 3-8-614 | 2-Nap | CH₃ | H | Ph |
| 3-8-615 | 2-Nap | CH(CH₃)₂ | H | Ph |
| 3-8-616 | 2-Nap | CH₂CH(CH₃)₂ | H | Ph |
| 3-8-617 | 2-Nap | CH(CH₃)CH₂CH₃ | H | Ph |
| 3-8-618 | 2-Nap | CH₂Ph | H | Ph |
| 3-8-619 | 2-Nap | CH₂-indol-3-yl | H | Ph |
| 3-8-620 | 2-Nap | CH₂CH₂SCH₃ | H | Ph |
| 3-8-621* | 2-Nap | * | H | Ph |
| 3-8-622 | 2-Nap | H | H | Bn |
| 3-8-623 | 2-Nap | CH₃ | H | Bn |
| 3-8-624 | 2-Nap | CH(CH₃)₂ | H | Bn |
| 3-8-625 | 2-Nap | CH₂CH(CH₃)₂ | H | Bn |
| 3-8-626 | 2-Nap | CH(CH₃)CH₂CH₃ | H | Bn |
| 3-8-627 | 2-Nap | CH₂Ph | H | Bn |
| 3-8-628 | 2-Nap | CH₂-indol-3-yl | H | Bn |
| 3-8-629 | 2-Nap | CH₂CH₂SCH₃ | H | Bn |
| 3-8-630* | 2-Nap | * | H | Bn |
| 3-8-631 | 2-Nap | H | H | CH₃ |
| 3-8-632 | 2-Nap | CH₃ | H | CH₃ |
| 3-8-633 | 2-Nap | CH(CH₃)₂ | H | CH₃ |
| 3-8-634 | 2-Nap | CH₂CH(CH₃)₂ | H | CH₃ |
| 3-8-635 | 2-Nap | CH(CH₃)CH₂CH₃ | H | CH₃ |
| 3-8-636 | 2-Nap | CH₂Ph | H | CH₃ |
| 3-8-637 | 2-Nap | CH₂-indol-3-yl | H | CH₃ |
| 3-8-638 | 2-Nap | CH₂CH₂SCH₃ | H | CH₃ |
| 3-8-639* | 2-Nap | * | H | CH₃ |
| 3-8-640 | 2-Nap | H | H | Et |
| 3-8-641 | 2-Nap | CH₃ | H | Et |
| 3-8-642 | 2-Nap | CH(CH₃)₂ | H | Et |
| 3-8-643 | 2-Nap | CH₂CH(CH₃)₂ | H | Et |
| 3-8-644 | 2-Nap | CH(CH₃)CH₂CH₃ | H | Et |
| 3-8-645 | 2-Nap | CH₂Ph | H | Et |
| 3-8-646 | 2-Nap | CH₂-indol-3-yl | H | Et |
| 3-8-647 | 2-Nap | CH₂CH₂SCH₃ | H | Et |
| 3-8-648* | 2-Nap | * | H | Et |
| 3-8-649 | p-Br-Ph | H | H | iPr |
| 3-8-650 | p-Br-Ph | CH₃ | H | iPr |
| 3-8-651 | p-Br-Ph | CH(CH₃)₂ | H | iPr |
| 3-8-652 | p-Br-Ph | CH₂CH(CH₃)₂ | H | iPr |
| 3-8-653 | p-Br-Ph | CH(CH₃)CH₂CH₃ | H | iPr |
| 3-8-654 | p-Br-Ph | CH₂Ph | H | iPr |
| 3-8-655 | p-Br-Ph | CH₂-indol-3-yl | H | iPr |
| 3-8-656 | p-Br-Ph | CH₂CH₂SCH₃ | H | iPr |
| 3-8-657* | p-Br-Ph | * | H | iPr |
| 3-8-658 | p-Br-Ph | H | H | nBu |
| 3-8-659 | p-Br-Ph | CH₃ | H | nBu |
| 3-8-660 | p-Br-Ph | CH(CH₃)₂ | H | nBu |
| 3-8-661 | p-Br-Ph | CH₂CH(CH₃)₂ | H | nBu |
| 3-8-662 | p-Br-Ph | CH(CH₃)CH₂CH₃ | H | nBu |
| 3-8-663 | p-Br-Ph | CH₂Ph | H | nBu |
| 3-8-664 | p-Br-Ph | CH₂-indol-3-yl | H | nBu |
| 3-8-665 | p-Br-Ph | CH₂CH₂SCH₃ | H | nBu |
| 3-8-666* | p-Br-Ph | * | H | nBu |
| 3-8-667 | p-Br-Ph | H | H | Ph |
| 3-8-668 | p-Br-Ph | CH₃ | H | Ph |
| 3-8-669 | p-Br-Ph | CH(CH₃)₂ | H | Ph |
| 3-8-670 | p-Br-Ph | CH₂CH(CH₃)₂ | H | Ph |
| 3-8-671 | p-Br-Ph | CH(CH₃)CH₂CH₃ | H | Ph |
| 3-8-672 | p-Br-Ph | CH₂Ph | H | Ph |
| 3-8-673 | p-Br-Ph | CH₂-indol-3-yl | H | Ph |
| 3-8-674 | p-Br-Ph | CH₂CH₂SCH₃ | H | Ph |
| 3-8-675* | p-Br-Ph | * | H | Ph |
| 3-8-676 | p-Br-Ph | H | H | Bn |
| 3-8-677 | p-Br-Ph | CH₃ | H | Bn |
| 3-8-678 | p-Br-Ph | CH(CH₃)₂ | H | Bn |
| 3-8-679 | p-Br-Ph | CH₂CH(CH₃)₂ | H | Bn |
| 3-8-680 | p-Br-Ph | CH(CH₃)CH₂CH₃ | H | Bn |
| 3-8-681 | p-Br-Ph | CH₂Ph | H | Bn |
| 3-8-682 | p-Br-Ph | CH₂-indol-3-yl | H | Bn |
| 3-8-683 | p-Br-Ph | CH₂CH₂SCH₃ | H | Bn |
| 3-8-684* | p-Br-Ph | * | H | Bn |
| 3-8-685 | p-Br-Ph | H | H | CH₃ |
| 3-8-686 | p-Br-Ph | CH₃ | H | CH₃ |
| 3-8-687 | p-Br-Ph | CH(CH₃)₂ | H | CH₃ |
| 3-8-688 | p-Br-Ph | CH₂CH(CH₃)₂ | H | CH₃ |
| 3-8-689 | p-Br-Ph | CH(CH₃)CH₂CH₃ | H | CH₃ |
| 3-8-690 | p-Br-Ph | CH₂Ph | H | CH₃ |
| 3-8-691 | p-Br-Ph | CH₂-indol-3-yl | H | CH₃ |
| 3-8-692 | p-Br-Ph | CH₂CH₂SCH₃ | H | CH₃ |
| 3-8-693* | p-Br-Ph | * | H | CH₃ |
| 3-8-694 | p-Br-Ph | H | H | Et |
| 3-8-695 | p-Br-Ph | CH₃ | H | Et |
| 3-8-696 | p-Br-Ph | CH(CH₃)₂ | H | Et |
| 3-8-697 | p-Br-Ph | CH₂CH(CH₃)₂ | H | Et |
| 3-8-698 | p-Br-Ph | CH(CH₃)CH₂CH₃ | H | Et |
| 3-8-699 | p-Br-Ph | CH₂Ph | H | Et |
| 3-8-700 | p-Br-Ph | CH₂-indol-3-yl | H | Et |
| 3-8-701 | p-Br-Ph | CH₂CH₂SCH₃ | H | Et |
| 3-8-702* | p-Br-Ph | * | H | Et |
| 3-8-703 | p-Cl-Ph | H | H | iPr |
| 3-8-704 | p-Cl-Ph | CH₃ | H | iPr |
| 3-8-705 | p-Cl-Ph | CH(CH₃)₂ | H | iPr |
| 3-8-706 | p-Cl-Ph | CH₂CH(CH₃)₂ | H | iPr |
| 3-8-707 | p-Cl-Ph | CH(CH₃)CH₂CH₃ | H | iPr |
| 3-8-708 | p-Cl-Ph | CH₂Ph | H | iPr |
| 3-8-709 | p-Cl-Ph | CH₂-indol-3-yl | H | iPr |
| 3-8-710 | p-Cl-Ph | CH₂CH₂SCH₃ | H | iPr |
| 3-8-711* | p-Cl-Ph | * | H | iPr |
| 3-8-712 | p-Cl-Ph | H | H | nBu |
| 3-8-713 | p-Cl-Ph | CH₃ | H | nBu |
| 3-8-714 | p-Cl-Ph | CH(CH₃)₂ | H | nBu |
| 3-8-715 | p-Cl-Ph | CH₂CH(CH₃)₂ | H | nBu |
| 3-8-716 | p-Cl-Ph | CH(CH₃)CH₂CH₃ | H | nBu |
| 3-8-717 | p-Cl-Ph | CH₂Ph | H | nBu |
| 3-8-718 | p-Cl-Ph | CH₂-indol-3-yl | H | nBu |
| 3-8-719 | p-Cl-Ph | CH₂CH₂SCH₃ | H | nBu |
| 3-8-720* | p-Cl-Ph | * | H | nBu |
| 3-8-721 | p-Cl-Ph | H | H | Ph |
| 3-8-722 | p-Cl-Ph | CH₃ | H | Ph |
| 3-8-723 | p-Cl-Ph | CH(CH₃)₂ | H | Ph |
| 3-8-724 | p-Cl-Ph | CH₂CH(CH₃)₂ | H | Ph |
| 3-8-725 | p-Cl-Ph | CH(CH₃)CH₂CH₃ | H | Ph |
| 3-8-726 | p-Cl-Ph | CH₂Ph | H | Ph |
| 3-8-727 | p-Cl-Ph | CH₂-indol-3-yl | H | Ph |
| 3-8-728 | p-Cl-Ph | CH₂CH₂SCH₃ | H | Ph |
| 3-8-729* | p-Cl-Ph | * | H | Ph |
| 3-8-730 | p-Cl-Ph | H | H | Bn |
| 3-8-731 | p-Cl-Ph | CH₃ | H | Bn |
| 3-8-732 | p-Cl-Ph | CH(CH₃)₂ | H | Bn |
| 3-8-733 | p-Cl-Ph | CH₂CH(CH₃)₂ | H | Bn |
| 3-8-734 | p-Cl-Ph | CH(CH₃)CH₂CH₃ | H | Bn |
| 3-8-735 | p-Cl-Ph | CH₂Ph | H | Bn |
| 3-8-736 | p-Cl-Ph | CH₂-indol-3-yl | H | Bn |
| 3-8-737 | p-Cl-Ph | CH₂CH₂SCH₃ | H | Bn |
| 3-8-738* | p-Cl-Ph | * | H | Bn |
| 3-8-739 | p-Cl-Ph | H | H | CH₃ |
| 3-8-740 | p-Cl-Ph | CH₃ | H | CH₃ |
| 3-8-741 | p-Cl-Ph | CH(CH₃)₂ | H | CH₃ |
| 3-8-742 | p-Cl-Ph | CH₂CH(CH₃)₂ | H | CH₃ |
| 3-8-743 | p-Cl-Ph | CH(CH₃)CH₂CH₃ | H | CH₃ |
| 3-8-744 | p-Cl-Ph | CH₂Ph | H | CH₃ |
| 3-8-745 | p-Cl-Ph | CH₂-indol-3-yl | H | CH₃ |
| 3-8-746 | p-Cl-Ph | CH₂CH₂SCH₃ | H | CH₃ |
| 3-8-747* | p-Cl-Ph | * | H | CH₃ |
| 3-8-748 | p-Cl-Ph | H | H | Et |
| 3-8-749 | p-Cl-Ph | CH₃ | H | Et |
| 3-8-750 | p-Cl-Ph | CH(CH₃)₂ | H | Et |
| 3-8-751 | p-Cl-Ph | CH₂CH(CH₃)₂ | H | Et |
| 3-8-752 | p-Cl-Ph | CH(CH₃)CH₂CH₃ | H | Et |
| 3-8-753 | p-Cl-Ph | CH₂Ph | H | Et |
| 3-8-754 | p-Cl-Ph | CH₂-indol-3-yl | H | Et |
| 3-8-755 | p-Cl-Ph | CH₂CH₂SCH₃ | H | Et |
| 3-8-756* | p-Cl-Ph | * | H | Et |
| 3-8-757 | p-F-Ph | H | H | iPr |
| 3-8-758 | p-F-Ph | CH₃ | H | iPr |
| 3-8-759 | p-F-Ph | CH(CH₃)₂ | H | iPr |
| 3-8-760 | p-F-Ph | CH₂CH(CH₃)₂ | H | iPr |
| 3-8-761 | p-F-Ph | CH(CH₃)CH₂CH₃ | H | iPr |
| 3-8-762 | p-F-Ph | CH₂Ph | H | iPr |
| 3-8-763 | p-F-Ph | CH₂-indol-3-yl | H | iPr |
| 3-8-764 | p-F-Ph | CH₂CH₂SCH₃ | H | iPr |
| 3-8-765* | p-F-Ph | * | H | iPr |
| 3-8-766 | p-F-Ph | H | H | nBu |
| 3-8-767 | p-F-Ph | CH₃ | H | nBu |
| 3-8-768 | p-F-Ph | CH(CH₃)₂ | H | nBu |
| 3-8-769 | p-F-Ph | CH₂CH(CH₃)₂ | H | nBu |
| 3-8-770 | p-F-Ph | CH(CH₃)CH₂CH₃ | H | nBu |
| 3-8-771 | p-F-Ph | CH₂Ph | H | nBu |
| 3-8-772 | p-F-Ph | CH₂-indol-3-yl | H | nBu |
| 3-8-773 | p-F-Ph | CH₂CH₂SCH₃ | H | nBu |
| 3-8-774* | p-F-Ph | * | H | nBu |
| 3-8-775 | p-F-Ph | H | H | Ph |
| 3-8-776 | p-F-Ph | CH₃ | H | Ph |
| 3-8-777 | p-F-Ph | CH(CH₃)₂ | H | Ph |
| 3-8-778 | p-F-Ph | CH₂CH(CH₃)₂ | H | Ph |
| 3-8-779 | p-F-Ph | CH(CH₃)CH₂CH₃ | H | Ph |
| 3-8-780 | p-F-Ph | CH₂Ph | H | Ph |
| 3-8-781 | p-F-Ph | CH₂-indol-3-yl | H | Ph |
| 3-8-782 | p-F-Ph | CH₂CH₂SCH₃ | H | Ph |
| 3-8-783* | p-F-Ph | * | H | Ph |
| 3-8-784 | p-F-Ph | H | H | Bn |
| 3-8-785 | p-F-Ph | CH₃ | H | Bn |
| 3-8-786 | p-F-Ph | CH(CH₃)₂ | H | Bn |
| 3-8-787 | p-F-Ph | CH₂CH(CH₃)₂ | H | Bn |
| 3-8-788 | p-F-Ph | CH(CH₃)CH₂CH₃ | H | Bn |
| 3-8-789 | p-F-Ph | CH₂Ph | H | Bn |
| 3-8-790 | p-F-Ph | CH₂-indol-3-yl | H | Bn |
| 3-8-791 | p-F-Ph | CH₂CH₂SCH₃ | H | Bn |
| 3-8-792* | p-F-Ph | * | H | Bn |
| 3-8-793 | p-F-Ph | H | H | CH₃ |
| 3-8-794 | p-F-Ph | CH₃ | H | CH₃ |
| 3-8-795 | p-F-Ph | CH(CH₃)₂ | H | CH₃ |
| 3-8-796 | p-F-Ph | CH₂CH(CH₃)₂ | H | CH₃ |
| 3-8-797 | p-F-Ph | CH(CH₃)CH₂CH₃ | H | CH₃ |
| 3-8-798 | p-F-Ph | CH₂Ph | H | CH₃ |
| 3-8-799 | p-F-Ph | CH₂-indol-3-yl | H | CH₃ |
| 3-8-800 | p-F-Ph | CH₂CH₂SCH₃ | H | CH₃ |
| 3-8-801* | p-F-Ph | * | H | CH₃ |
| 3-8-802 | p-F-Ph | H | H | Et |
| 3-8-803 | p-F-Ph | CH₃ | H | Et |
| 3-8-804 | p-F-Ph | CH(CH₃)₂ | H | Et |
| 3-8-805 | p-F-Ph | CH₂CH(CH₃)₂ | H | Et |
| 3-8-806 | p-F-Ph | CH(CH₃)CH₂CH₃ | H | Et |
| 3-8-807 | p-F-Ph | CH₂Ph | H | Et |
| 3-8-808 | p-F-Ph | CH₂-indol-3-yl | H | Et |
| 3-8-809 | p-F-Ph | CH₂CH₂SCH₃ | H | Et |
| 3-8-810* | p-F-Ph | * | H | Et |
| 3-8-811 | p-I-Ph | H | H | iPr |
| 3-8-812 | p-I-Ph | CH₃ | H | iPr |
| 3-8-813 | p-I-Ph | CH(CH₃)₂ | H | iPr |
| 3-8-814 | p-I-Ph | CH₂CH(CH₃)₂ | H | iPr |
| 3-8-815 | p-I-Ph | CH(CH₃)CH₂CH₃ | H | iPr |
| 3-8-816 | p-I-Ph | CH₂Ph | H | iPr |
| 3-8-817 | p-I-Ph | CH₂-indol-3-yl | H | iPr |
| 3-8-818 | p-I-Ph | CH₂CH₂SCH₃ | H | iPr |
| 3-8-819* | p-I-Ph | * | H | iPr |
| 3-8-820 | p-I-Ph | H | H | nBu |
| 3-8-821 | p-I-Ph | CH₃ | H | nBu |
| 3-8-822 | p-I-Ph | CH(CH₃)₂ | H | nBu |
| 3-8-823 | p-I-Ph | CH₂CH(CH₃)₂ | H | nBu |
| 3-8-824 | p-I-Ph | CH(CH₃)CH₂CH₃ | H | nBu |
| 3-8-825 | p-I-Ph | CH₂Ph | H | nBu |
| 3-8-826 | p-I-Ph | CH₂-indol-3-yl | H | nBu |
| 3-8-827 | p-I-Ph | CH₂CH₂SCH₃ | H | nBu |
| 3-8-828* | p-I-Ph | * | H | nBu |
| 3-8-829 | p-I-Ph | H | H | Ph |
| 3-8-830 | p-I-Ph | CH₃ | H | Ph |
| 3-8-831 | p-I-Ph | CH(CH₃)₂ | H | Ph |
| 3-8-832 | p-I-Ph | CH₂CH(CH₃)₂ | H | Ph |
| 3-8-833 | p-I-Ph | CH(CH₃)CH₂CH₃ | H | Ph |
| 3-8-834 | p-I-Ph | CH₂Ph | H | Ph |
| 3-8-835 | p-I-Ph | CH₂-indol-3-yl | H | Ph |
| 3-8-836 | p-I-Ph | CH₂CH₂SCH₃ | H | Ph |
| 3-8-837* | p-I-Ph | * | H | Ph |
| 3-8-838 | p-I-Ph | H | H | Bn |
| 3-8-839 | p-I-Ph | CH₃ | H | Bn |
| 3-8-840 | p-I-Ph | CH(CH₃)₂ | H | Bn |
| 3-8-841 | p-I-Ph | CH₂CH(CH₃)₂ | H | Bn |
| 3-8-842 | p-I-Ph | CH(CH₃)CH₂CH₃ | H | Bn |
| 3-8-843 | p-I-Ph | CH₂Ph | H | Bn |
| 3-8-844 | p-I-Ph | CH₂-indol-3-yl | H | Bn |
| 3-8-845 | p-I-Ph | CH₂CH₂SCH₃ | H | Bn |
| 3-8-846* | p-I-Ph | * | H | Bn |
| 3-8-847 | p-I-Ph | H | H | CH₃ |
| 3-8-848 | p-I-Ph | CH₃ | H | CH₃ |
| 3-8-849 | p-I-Ph | CH(CH₃)₂ | H | CH₃ |
| 3-8-850 | p-I-Ph | CH₂CH(CH₃)₂ | H | CH₃ |
| 3-8-851 | p-I-Ph | CH(CH₃)CH₂CH₃ | H | CH₃ |
| 3-8-852 | p-I-Ph | CH₂Ph | H | CH₃ |
| 3-8-853 | p-I-Ph | CH₂-indol-3-yl | H | CH₃ |
| 3-8-854 | p-I-Ph | CH₂CH₂SCH₃ | H | CH₃ |
| 3-8-855* | p-I-Ph | * | H | CH₃ |
| 3-8-856 | p-I-Ph | H | H | Et |
| 3-8-857 | p-I-Ph | CH₃ | H | Et |
| 3-8-858 | p-I-Ph | CH(CH₃)₂ | H | Et |
| 3-8-859 | p-I-Ph | CH₂CH(CH₃)₂ | H | Et |
| 3-8-860 | p-I-Ph | CH(CH₃)CH₂CH₃ | H | Et |
| 3-8-861 | p-I-Ph | CH₂Ph | H | Et |
| 3-8-862 | p-I-Ph | CH₂-indol-3-yl | H | Et |
| 3-8-863 | p-I-Ph | CH₂CH₂SCH₃ | H | Et |
| 3-8-864* | p-I-Ph | * | H | Et |
| 3-8-865 | p-Me-Ph | H | H | iPr |
| 3-8-866 | p-Me-Ph | CH₃ | H | iPr |
| 3-8-867 | p-Me-Ph | CH(CH₃)₂ | H | iPr |
| 3-8-868 | p-Me-Ph | CH₂CH(CH₃)₂ | H | iPr |
| 3-8-869 | p-Me-Ph | CH(CH₃)CH₂CH₃ | H | iPr |
| 3-8-870 | p-Me-Ph | CH₂Ph | H | iPr |
| 3-8-871 | p-Me-Ph | CH₂-indol-3-yl | H | iPr |
| 3-8-872 | p-Me-Ph | CH₂CH₂SCH₃ | H | iPr |
| 3-8-873* | p-Me-Ph | * | H | iPr |
| 3-8-874 | p-Me-Ph | H | H | nBu |
| 3-8-875 | p-Me-Ph | CH₃ | H | nBu |
| 3-8-876 | p-Me-Ph | CH(CH₃)₂ | H | nBu |
| 3-8-877 | p-Me-Ph | CH₂CH(CH₃)₂ | H | nBu |
| 3-8-878 | p-Me-Ph | CH(CH₃)CH₂CH₃ | H | nBu |
| 3-8-879 | p-Me-Ph | CH₂Ph | H | nBu |
| 3-8-880 | p-Me-Ph | CH₂-indol-3-yl | H | nBu |
| 3-8-881 | p-Me-Ph | CH₂CH₂SCH₃ | H | nBu |
| 3-8-882* | p-Me-Ph | * | H | nBu |
| 3-8-883 | p-Me-Ph | H | H | Ph |
| 3-8-884 | p-Me-Ph | CH₃ | H | Ph |
| 3-8-885 | p-Me-Ph | CH(CH₃)₂ | H | Ph |
| 3-8-886 | p-Me-Ph | CH₂CH(CH₃)₂ | H | Ph |
| 3-8-887 | p-Me-Ph | CH(CH₃)CH₂CH₃ | H | Ph |
| 3-8-888 | p-Me-Ph | CH₂Ph | H | Ph |
| 3-8-889 | p-Me-Ph | CH₂-indol-3-yl | H | Ph |
| 3-8-890 | p-Me-Ph | CH₂CH₂SCH₃ | H | Ph |
| 3-8-891* | p-Me-Ph | * | H | Ph |
| 3-8-892 | p-Me-Ph | H | H | Bn |
| 3-8-893 | p-Me-Ph | CH₃ | H | Bn |
| 3-8-894 | p-Me-Ph | CH(CH₃)₂ | H | Bn |
| 3-8-895 | p-Me-Ph | CH₂CH(CH₃)₂ | H | Bn |
| 3-8-896 | p-Me-Ph | CH(CH₃)CH₂CH₃ | H | Bn |
| 3-8-897 | p-Me-Ph | CH₂Ph | H | Bn |
| 3-8-898 | p-Me-Ph | CH₂-indol-3-yl | H | Bn |
| 3-8-899 | p-Me-Ph | CH₂CH₂SCH₃ | H | Bn |
| 3-8-900* | p-Me-Ph | * | H | Bn |
| 3-8-901 | p-Me-Ph | H | H | CH₃ |
| 3-8-902 | p-Me-Ph | CH₃ | H | CH₃ |
| 3-8-903 | p-Me-Ph | CH(CH₃)₂ | H | CH₃ |
| 3-8-904 | p-Me-Ph | CH₂CH(CH₃)₂ | H | CH₃ |
| 3-8-905 | p-Me-Ph | CH(CH₃)CH₂CH₃ | H | CH₃ |
| 3-8-906 | p-Me-Ph | CH₂Ph | H | CH₃ |
| 3-8-907 | p-Me-Ph | CH₂-indol-3-yl | H | CH₃ |
| 3-8-908 | p-Me-Ph | CH₂CH₂SCH₃ | H | CH₃ |
| 3-8-909* | p-Me-Ph | * | H | CH₃ |
| 3-8-910 | p-Me-Ph | H | H | Et |
| 3-8-911 | p-Me-Ph | CH₃ | H | Et |
| 3-8-912 | p-Me-Ph | CH(CH₃)₂ | H | Et |
| 3-8-913 | p-Me-Ph | CH₂CH(CH₃)₂ | H | Et |
| 3-8-914 | p-Me-Ph | CH(CH₃)CH₂CH₃ | H | Et |
| 3-8-915 | p-Me-Ph | CH₂Ph | H | Et |
| 3-8-916 | p-Me-Ph | CH₂-indol-3-yl | H | Et |
| 3-8-917 | p-Me-Ph | CH₂CH₂SCH₃ | H | Et |
| 3-8-918* | p-Me-Ph | * | H | Et |
| 3-8-919 | tBu | H | H | iPr |
| 3-8-920 | tBu | CH₃ | H | iPr |
| 3-8-921 | tBu | CH(CH₃)₂ | H | iPr |
| 3-8-922 | tBu | CH₂CH(CH₃)₂ | H | iPr |
| 3-8-923 | tBu | CH(CH₃)CH₂CH₃ | H | iPr |
| 3-8-924 | tBu | CH₂Ph | H | iPr |
| 3-8-925 | tBu | CH₂-indol-3-yl | H | iPr |
| 3-8-926 | tBu | CH₂CH₂SCH₃ | H | iPr |
| 3-8-927* | tBu | * | H | iPr |
| 3-8-928 | tBu | H | H | nBu |
| 3-8-929 | tBu | CH₃ | H | nBu |
| 3-8-930 | tBu | CH(CH₃)₂ | H | nBu |
| 3-8-931 | tBu | CH₂CH(CH₃)₂ | H | nBu |
| 3-8-932 | tBu | CH(CH₃)CH₂CH₃ | H | nBu |
| 3-8-933 | tBu | CH₂Ph | H | nBu |
| 3-8-934 | tBu | CH₂-indol-3-yl | H | nBu |
| 3-8-935 | tBu | CH₂CH₂SCH₃ | H | nBu |
| 3-8-936* | tBu | * | H | nBu |
| 3-8-937 | tBu | H | H | Ph |
| 3-8-938 | tBu | CH₃ | H | Ph |
| 3-8-939 | tBu | CH(CH₃)₂ | H | Ph |
| 3-8-940 | tBu | CH₂CH(CH₃)₂ | H | Ph |
| 3-8-941 | tBu | CH(CH₃)CH₂CH₃ | H | Ph |
| 3-8-942 | tBu | CH₂Ph | H | Ph |
| 3-8-943 | tBu | CH₂-indol-3-yl | H | Ph |
| 3-8-944 | tBu | CH₂CH₂SCH₃ | H | Ph |
| 3-8-945* | tBu | * | H | Ph |
| 3-8-946 | tBu | H | H | Bn |
| 3-8-947 | tBu | CH₃ | H | Bn |
| 3-8-948 | tBu | CH(CH₃)₂ | H | Bn |
| 3-8-949 | tBu | CH₂CH(CH₃)₂ | H | Bn |
| 3-8-950 | tBu | CH(CH₃)CH₂CH₃ | H | Bn |
| 3-8-951 | tBu | CH₂Ph | H | Bn |
| 3-8-952 | tBu | CH₂-indol-3-yl | H | Bn |
| 3-8-953 | tBu | CH₂CH₂SCH₃ | H | Bn |
| 3-8-954* | tBu | * | H | Bn |
| 3-8-955 | tBu | H | H | CH₃ |
| 3-8-956 | tBu | CH₃ | H | CH₃ |
| 3-8-957 | tBu | CH(CH₃)₂ | H | CH₃ |
| 3-8-958 | tBu | CH₂CH(CH₃)₂ | H | CH₃ |
| 3-8-959 | tBu | CH(CH₃)CH₂CH₃ | H | CH₃ |
| 3-8-960 | tBu | CH₂Ph | H | CH₃ |
| 3-8-961 | tBu | CH₂-indol-3-yl | H | CH₃ |
| 3-8-962 | tBu | CH₂CH₂SCH₃ | H | CH₃ |
| 3-8-963* | tBu | * | H | CH₃ |
| 3-8-964 | tBu | H | H | Et |
| 3-8-965 | tBu | CH₃ | H | Et |
| 3-8-966 | tBu | CH(CH₃)₂ | H | Et |
| 3-8-967 | tBu | CH₂CH(CH₃)₂ | H | Et |
| 3-8-968 | tBu | CH(CH₃)CH₂CH₃ | H | Et |
| 3-8-969 | tBu | CH₂Ph | H | Et |
| 3-8-970 | tBu | CH₂-indol-3-yl | H | Et |
| 3-8-971 | tBu | CH₂CH₂SCH₃ | H | Et |
| 3-8-972* | tBu | * | H | Et |

*R⁶ and R^{7a} (or R⁶ and R^{7b}) are joined together by (CH₂)₃ to form five-membered ring.
Abbreviations used in Table 8:
Ph = phenyl;
iPr = isopropyl;
nBu = n-butyl;
1-Nap = 1-naphthyl;
2-Nap = 2-naphthyl;
Bn = benzyl;
Et = ethyl; and
tBu = tert-butyl.

In one embodiment, the invention is the 5′-deuterated-2′-methyl-2′-fluoro-nucleotide phosphoramidate of the following structure:

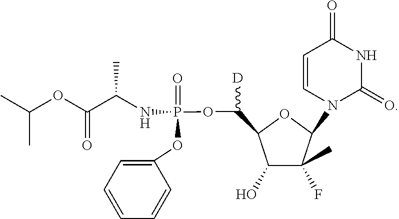 [SMILES: [2H]C(O[P@@](=O)(N[C@@H](C)C(=O)OC(C)C)Oc1ccccc1)[C@H]1O[C@@H](n2ccc(=O)[nH]c2=O)[C@](C)(F)[C@@H]1O]

In one embodiment, the invention is the 5′-deuterated-2′-methyl-2′-fluoro-nucleotide phosphoramidate of the following structure:

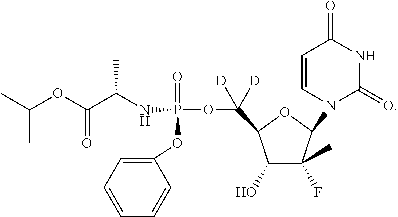 [SMILES: [2H]C([2H])(O[P@@](=O)(N[C@@H](C)C(=O)OC(C)C)Oc1ccccc1)[C@H]1O[C@@H](n2ccc(=O)[nH]c2=O)[C@](C)(F)[C@@H]1O]

In one embodiment, the invention is the 5′-deuterated-2′-methyl-2′-fluoro-nucleotide phosphoramidate of the following structure:

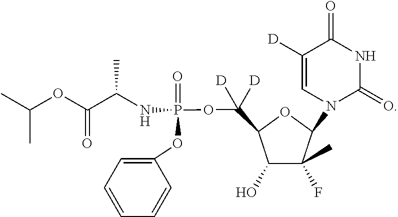 [SMILES: [2H]c1cn([C@@H]2O[C@H](C([2H])([2H])O[P@@](=O)(N[C@@H](C)C(=O)OC(C)C)Oc3ccccc3)[C@@H](O)[C@@]2(C)F)c(=O)[nH]c1=O]

In one embodiment, the invention is the 5′-deuterated 2′-methyl-2′-fluoro-nucleotide phosphoramidate of the following structure:

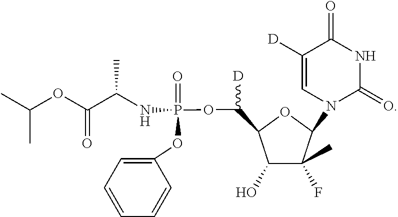 [SMILES: [2H]c1cn([C@@H]2O[C@H](C([2H])O[P@@](=O)(N[C@@H](C)C(=O)OC(C)C)Oc3ccccc3)[C@@H](O)[C@@]2(C)F)c(=O)[nH]c1=O]

In one embodiment, a 5′-deuterated stabilized nucleoside 5′-phosphate lipid conjugate is provided having the formula:

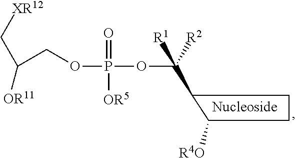

wherein X is S or O; R¹ and R² are each independently hydrogen, deuterium or methyl and at least one of R¹ or R² is deuterium and typically both R¹ and R² are D; R⁴ is hydrogen, acyl or amino acid; R⁵ is hydrogen; R¹¹ and R¹² are each independently C₁-C₂₀ alkyl, C₁-C₂₀ alkenyl, C₁-C₂₀ alkynyl or C₆-C₂₀ acyl; and nucleoside is as described above.

The present disclosure provides a significant improvement over the compounds disclosed in, for example, U.S. Patent Application Publication US 2012/0071434; U.S. Ser. No. 13/36,435), published Mar. 22, 2012 and assigned to Alios BioPharma, Inc. which describes phosphorothioamidate nucleosides for the treatment of viral diseases. It is known that the replacement of oxygen with sulfur in a nucleoside 5′-phosphate stabilizes the phosphate against nucleotidase or other hydrolyzing action, which forms the basis for the use of phosphorothioates and other thiophosphate derivatives in antisense and aptamer stabilization chemistry which was developed in the 1980's. See generally, among other references, Pearson, “Characterization of ectonucleotidases on vascular smooth-muscle cells”, Biochem J. (1985), 230, 503-507. One of the compounds disclosed by Alios is:

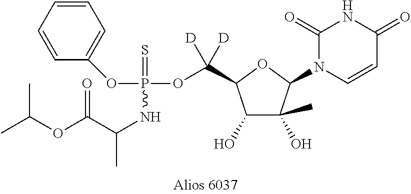 [SMILES: [2H]C([2H])(OP(=S)(NC(C)C(=O)OC(C)C)Oc1ccccc1)[C@H]1O[C@@H](n2ccc(=O)[nH]c2=O)[C@](C)(O)[C@@H]1O]

The monothiophosphate metabolite which would result from the cleavage of the phosphorothioamidate 6037 in vivo is stabilized from further enzymatic breakdown to the free 5′-hydroxyl nucleoside due to the presence of the stabilizing, unnatural sulfur atom. Therefore, the stabilizing effect of deuteration at the 5′-position is masked by the sulfur. Further, it is also known that nucleoside monothiophosphates are to a small extent hydrolyzed to nucleoside monophosphates via the Hint1 enzyme (the enzyme that is also responsible for production of monophosphate Formula V (described further below) as well as monothiophosphates from their respective prodrugs), releasing H₂S, a toxic metabolite, which can cause physiological and pathogenic effects (see Ozga et al. J. Biol. Chem. 2010, 285, 40809).

A significant improvement provided by the present invention is the surprising discovery that 5′-deuteration using a more natural phosphoramidate, i.e., without sulfur, protects the monophosphate from further breakdown to the free hydroxyl group in a manner that minimizes toxicity and more closely mimics natural compounds. The fact that this is a nonobvious invention is dramatically highlighted with a review of U.S. Publication 2011/0251152 (U.S. Ser. No. 13/076,552), assigned to Pharmasset, Inc., the company that developed Sofosbuvir. On page 16 of the publication, this nucleoside-experienced company described the use of deuteration in six different species of Sofosbuvir, but never considered placing the deuterium in the 5′-position, now determined to be the most important position.

Highly Active Nucleotide Phosphoramidate Structures

Further provided herein are 2′-β-methyl-5′-deuterated uridine phosphoramidates of Formula I (provided below), including Formula II, or Formula IIIA or IIIB, or a pharmaceutically acceptable salt thereof, wherein deuterium has an enrichment over protium of at least 90% (i.e., less than 10% 1H hydrogen), and wherein R¹ and R² are independently deuterium, hydrogen, or C(H)ₘ(D)ₙ; and at least one of R¹ and R² is deuterium; R³ is hydrogen, deuterium, halogen (F, Cl, Br, or I), or C(H)ₘ(D)ₙ.

It has been found that 2′-methyl-5′-deuterated uridine phosphoramidates, for example as provided by Formula I, are superior NS5B inhibitors for the treatment of hepatitis C, or any other disorder disclosed herein. In one embodiment, only one of the 5′-substituents of Formula I, II, IIA, or IIIB is deuterium, while the other is hydrogen. R³ is hydrogen, deuterium, halogen (F, Cl, Br, or I), C(H)ₘ(D)ₙ; or, alternatively, alkyne, wherein the R³ alkyne and the C⁴-oxygen of the pyrimidine can combine to form a heterocyclic ring.

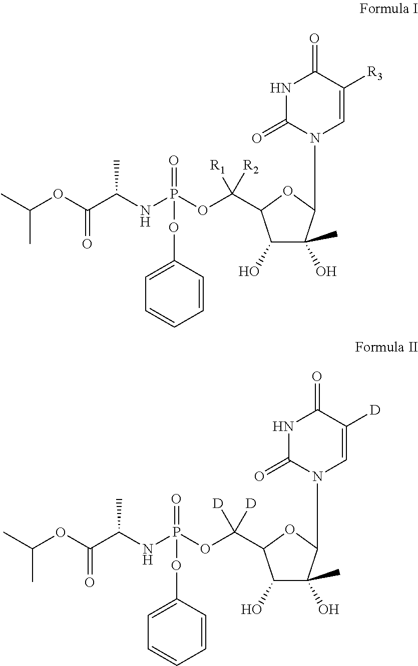

Formula II comprises mixtures of stereoisomers. For example Formula II can include a mixture, including a 50/50 mixture of stereoisomers of Formula II, wherein the mixture comprises:

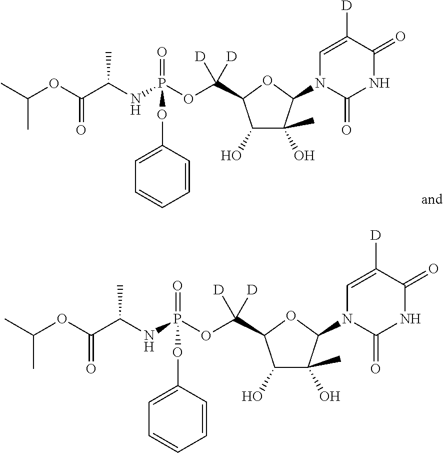 [SMILES: [2H]c1cn([C@@H]2O[C@H](C([2H])([2H])O[P@@](=O)(N[C@@H](C)C(=O)OC(C)C)Oc3ccccc3)[C@@H](O)[C@@]2(C)O)c(=O)[nH]c1=O ; [2H]c1cn([C@@H]2O[C@H](C([2H])([2H])O[P@](=O)(N[C@@H](C)C(=O)OC(C)C)Oc3ccccc3)[C@@H](O)[C@@]2(C)O)c(=O)[nH]c1=O]

Therefore, in one embodiment a method for the treatment of a host infected with hepatitis C or a related or other disorder as described herein is provided that includes the administration of an effective amount of an isolated compound of Formula I or II of at least 90% purity, or a pharmaceutically acceptable salt thereof, optionally in a pharmaceutically acceptable carrier.

In an alternative embodiment, one or both of the 5′-deuterium(s) independently represents at least 50% enrichment. In another embodiment, the enrichment is independently at least 75% or 80%. In another embodiment, one or both of the 5′-deuterium(s) independently represents at least 90%, 95% 96%, 97%, 98% or 99% enrichment. In another embodiment, the deuterium in the 5-position of the pyrimidine represents at least 50% enrichment. In another embodiment, the enrichment is independently at least 75% or 80%. In another embodiment, one, or both or the 5′-deuterium(s) independently represent at least 90%, 95%, 96%, 97%, 98% or 99% enrichment. In the absence of an indication to the contrary, the deuterium is at least 90% at that position.

In another embodiment, the nucleoside derivative of Formula I or II is administered as a phosphorus R or S stereoisomer, wherein the phosphorus stereoisomer is at least in 90% pure form, and typically, 95, 98 or 99% pure form. In an alternative embodiment, the compound is administered as a mixture of phosphorous chiral center stereoisomers, such as a 50/50 mixture of stereoisomers at the phosphorous chiral center.

In another embodiment, an effective amount of a compound of the Formula IIIA or IIIB or its pharmaceutically acceptable salt, optionally in a pharmaceutically acceptable carrier, is provided to a host in need of hepatitis C therapy, or another therapy as disclosed herein.

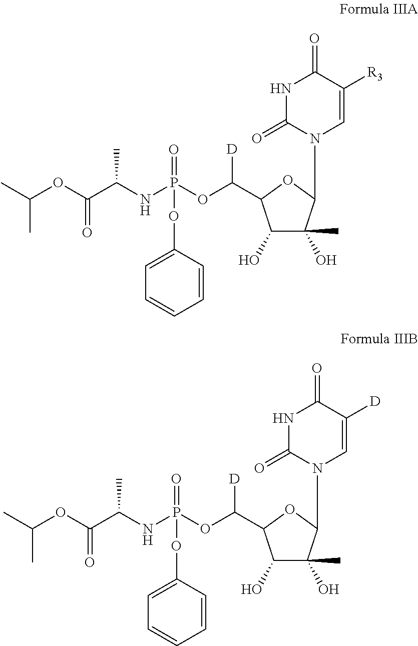

On administration to the host, for example, the phosphoramidate of Formula II is metabolized to the 5′-OH, 5′-D, D-monophosphate (Formula V) via a series of enzymatic steps:

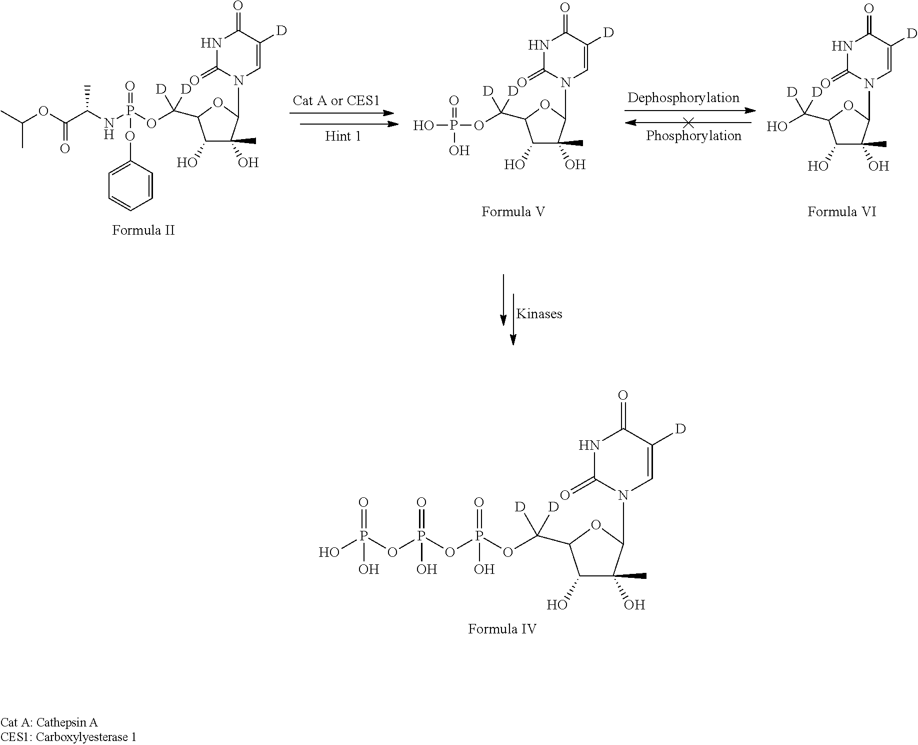 [SMILES: [2H]c1cn(C2OC(C([2H])([2H])OP(=O)(N[C@@H](C)C(=O)OC(C)C)Oc3ccccc3)[C@@H](O)[C@@]2(C)O)c(=O)[nH]c1=O ; [2H]c1cn(C2OC(C([2H])([2H])OP(=O)(O)O)[C@@H](O)[C@@]2(C)O)c(=O)[nH]c1=O ; [2H]c1cn(C2OC(C([2H])([2H])O)[C@@H](O)[C@@]2(C)O)c(=O)[nH]c1=O ; [2H]c1cn(C2OC(C([2H])([2H])OP(=O)(O)OP(=O)(O)OP(=O)(O)O)[C@@H](O)[C@@]2(C)O)c(=O)[nH]c1=O]

Formula II, for example, is converted to its active species, the nucleoside triphosphate (Formula IV), via the nucleoside monophosphate (Formula V). Alternatively, the nucleoside monophosphate (Formula V) can undergo dephosphorylation to 5′-deuterated 2′-C-methyluridine (Formula VI). The 5′-deuterated nucleoside triphosphate (Formula IV) is the pharmacologically active metabolite that inhibits hepatitis C viral replication, whereas the 5′-deuterated 2′-C-methyluridine (Formula VI) shows little activity because it is a poor substrate for nucleoside monophosphate kinase.

5′-Deuterated nucleoside monophosphate (Formula V) if dephosphorylated will produce 5′-deuterated nucleoside (Formula VI). The undeuterated nucleoside monophosphate (Formula VIII) will produce undeuterated 2′-C-methyluridine (Formula IX):

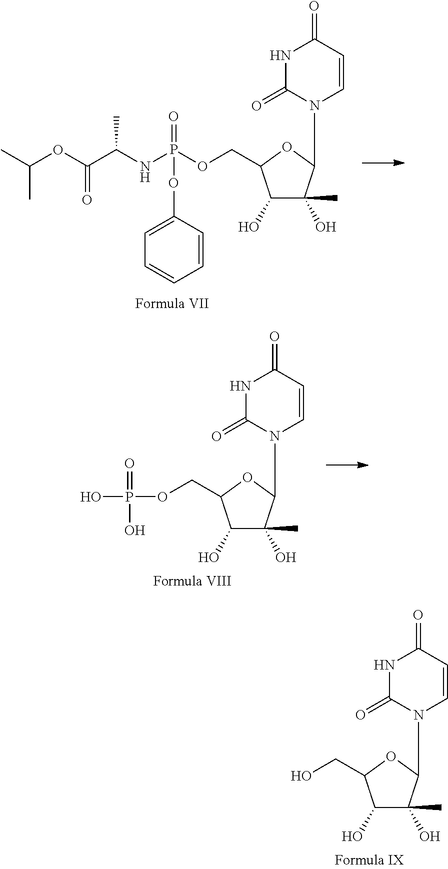 [SMILES: CC(C)OC(=O)[C@H](C)NP(=O)(OCC1OC(n2ccc(=O)[nH]c2=O)[C@](C)(O)[C@@H]1O)Oc1ccccc1 ; C[C@]1(O)C(n2ccc(=O)[nH]c2=O)OC(COP(=O)(O)O)[C@H]1O ; C[C@]1(O)C(n2ccc(=O)[nH]c2=O)OC(CO)[C@H]1O]

Chemical Description and Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in any suitable order unless otherwise indicated herein or otherwise clearly contradicted by context. The use of any and all examples, or exemplary language (e.g., “such as”), is intended merely for illustration and does not pose a limitation on the scope of the invention unless otherwise claimed.

An “active agent” is a compound (including a compound disclosed herein), element, or mixture that when administered to a patient, alone or in combination with another compound, element, or mixture, confers, directly or indirectly, a physiological effect on the patient. The indirect physiological effect may occur via a metabolite or other indirect mechanism.

The term “optionally substituted”, as used herein, means that one or more hydrogens on the designated atom or group can be replaced with any pharmaceutically acceptable non-hydrogen substituent that achieves the intended purpose. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

Suitable groups that may be present at an “optionally substituted” position include, but are not limited to, those below and should be selected according to the bulk tolerance, charge, polarity, molecular weight, lipophilicity and other physical and imparted biological properties at the target position that must be taken into consideration, such as efficacy and safety. Nonlimiting examples to select from include: alkyl groups (including cycloalkyl groups) having the number of carbons useful to achieve the intended purpose; for example, 1 to about 22 carbon atoms or 1 to about 8 carbon atoms, or 1 to about 6 carbon atoms; alkenyl and alkynyl groups including groups having one or more unsaturated linkages and from 3 to about 22 carbon atoms or 2 to about 8 carbon atoms, or 2 to about 6 carbon atoms; halogen; cyano; hydroxyl; nitro; azido; acyl (such as a C₂-C₆ acyl group); carboxamide; alkoxy groups having one or more oxygen linkages and from 1 to about 22 carbon atoms, 1 to about 8 carbon atoms, or from 1 to about 6 carbon atoms; aryloxy such as phenoxy; alkylthio groups including those having one or more thioether linkages and from 1 to about 22 carbon atoms, 1 to about 8 carbon atoms, or from 1 to about 6 carbon atoms; alkylsulfinyl groups including those having one or more sulfinyl linkages and from 1 to about 8 carbon atoms, or from 1 to about 6 carbon atoms; alkylsulfonyl groups including those having one or more sulfonyl linkages and from 1 to about 8 carbon atoms, or from 1 to about 6 carbon atoms; aminoalkyl groups including groups having one or more N atoms and from 1 to about 8, or from 1 to about 6 carbon atoms; aryl having 6 or more carbons and one or more rings, (e.g., phenyl, biphenyl, naphthyl, or the like, each ring either substituted or unsubstituted aryl); arylalkyl having 1 to 3 separate or fused rings and from 6 to about 18 ring carbon atoms, with benzyl being an exemplary arylalkyl- group; arylalkoxy having 1 to 3 separate or fused rings and from 6 to about 18 ring carbon atoms, with benzyloxy being an exemplary arylalkoxy group; or a saturated, unsaturated, or heteroaryl group having 1 to 3 separate or fused rings with 3 to about 8 members per ring and one or more N, O or S atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such heterocyclic groups may be further substituted, e.g. with hydroxy, alkyl, alkoxy, halogen and amino. In certain embodiments “optionally substituted” includes one or more substituents independently chosen from C₁-C₆alkyl, C₂-C₆alkenyl, halogen, hydroxyl, amino, cyano, —CHO, —COOH, —CONH₂, C₁-C₆alkoxy, C₂-C₆acyl, C₁-C₆alkylester, (mono- and di-C₁-C₆alkylamino)C₀-C₂alkyl-, C₁-C₂haloalkyl, and C₁-C₂haloalkoxy. In one embodiment halogen is fluoro, chloro, bromo and iodo. In another embodiment, halogen is fluoro. In another embodiment halogen is chloro, bromo and iodo.

“Deuteration” and “deuterated” means that a hydrogen is replaced by a deuterium such that the deuterium exists over natural abundance and is thus “enriched”. An enrichment of 50% means that rather than hydrogen at the specified position the deuterium content is 50%. For clarity, it is confirmed that the term “enriched” as used herein does not mean percentage enriched over natural abundance. In other embodiments, there will be at least 80%, at least 90%, or at least 95% deuterium enrichment at the specified deuterated position or positions. In other embodiments there will be at least 96%, at least 97%, at least 98% or at least 99% deuterium enrichment at the specified deuterated position or positions. In the absence of indication to the contrary, the enrichment of deuterium in the specified position of the compound described herein is at least 90%.

A dash (“-”) that is not between two letters or symbols is used to indicate a point of attachment for a substituent. For example, —(C═O)NH₂ is attached through carbon of the keto (C═O) group.

“Acyl” refers to a group of the formula —C(O)R″, wherein R″ is a substituent. In one embodiment R″ is a straight, branched, or cyclic alkyl (including C₁-C₃ alkyl), amino acid, aryl including phenyl, alkaryl, aralkyl- including benzyl, alkoxyalkyl- including methoxymethyl, aryloxyalkyl- such as phenoxymethyl; or substituted alkyl (including lower alkyl), aryl including phenyl may be substituted with chloro, bromo, fluoro, iodo, C₁ to C₄ alkyl or C₁ to C₄ alkoxy, sulfonate esters such as alkyl or aralkyl sulphonyl including methanesulfonyl, the mono, di or triphosphate ester, trityl or monomethoxy-trityl, substituted benzyl, alkaryl, aralkyl- including benzyl, alkoxyalkyl- including methoxymethyl, aryloxyalkyl- such as phenoxymethyl. Aryl groups in the esters may comprise a phenyl group. In particular, acyl groups include acetyl, trifluoroacetyl, methylacetyl, cyclopropylacetyl, cyclopropyl carboxy, propionyl, butyryl, pentanoyl, hexanoyl, heptanoyl, octanoyl, neo-heptanoyl, phenylacetyl, 2-acetoxy-2-phenylacetyl, diphenylacetyl, α-methoxy-α-trifluoromethyl-phenylacetyl, bromoacetyl, 2-nitro-benzeneacetyl, 4-chloro-benzeneacetyl, 2-chloro-2,2-diphenylacetyl, 2-chloro-2-phenylacetyl, trimethylacetyl, chlorodifluoroacetyl, perfluoroacetyl, fluoroacetyl, bromodifluoroacetyl, methoxyacetyl, 2-thiopheneacetyl, chlorosulfonylacetyl, 3-methoxypheny (acetyl, phenoxyacetyl, tert-butylacetyl, trichloroacetyl, monochloro-acetyl, dichloroacetyl, 7H-dodecafluoro-heptanoyl, perfluoro-heptanoyl, 7H-dodeca-fluoroheptanoyl, 7-chlorododecafluoro-heptanoyl, 7-chloro-dodecatluoro-heptanoyl, 7H-dodecafluoroheptanoyl, 7H-dodeca-fluoroheptanoyl, nona-fluoro-3,6-dioxa-heptanoyl, nonafluoro-3,6-dioxaheptanoyl, perfluoroheptanoyl, methoxybenzoyl, methyl 3-amino-5-phenylthiophene-2-carboxyl, 3,6-dichloro-2-methoxy-benzoyl, 4(1,1,2,2-tetrafluoro-ethoxy)-benzoyl, 2-bromo-propionyl, omega-aminocapryl, decanoyl, n-pentadecanoyl, stearyl, 3-cyclopentyl-propionyl, I-benzene-carboxyl, O-acetylmandelyl, pivaloyl acetyl, 1-adamantane-carboxyl, cyclohexane-carboxyl, 2,6-pyridinedicarboxyl, cyclopropane-carboxyl, cyclobutane-carboxyl, perfluorocyclohexyl carboxyl, 4-methylbenzoyl, chloromethyl isoxazolyl carbonyl, perfluorocyclohexyl carboxyl, crotonyl-methyl-1H-indazole-3-carbonyl, 2-propenyl, isovaleryl, 1-pyrrolidinecarbonyl, 4-phenylbenzoyl. When the term acyl is used, it is meant to be a specific and independent disclosure of acetyl, trifluoroacetyl, methylacetyl, cyclopropylacetyl, propionyl, butyryl, pentanoyl, hexanoyl, heptanoyl, octanoyl, neo-heptanoyl, phenylacetyl, diphenylacetyl, α-trifluoromethyl-phenylacetyl, bromoacetyl, 4-chloro-benzeneacetyl, 2-chloro-2,2-diphenylacetyl, 2-chloro-2-phenylacetyl, trimethylacetyl, chlorodifluoroacetyl, perfluoroacetyl, fluoroacetyl, bromodifluoroacetyl, 2-thiopheneacetyl, tert-butylacetyl, trichloroacetyl, monochloro-acetyl, dichloroacetyl, methoxybenzoyl, 2-bromo-propionyl, decanoyl, n-pentadecanoyl, stearyl, 3-cyclopentyl-propionyl, 1-benzene-carboxyl, pivaloyl acetyl, 1-adamantane-carboxyl, cyclohexane-carboxyl, 2,6-pyridinedicarboxyl, cyclopropane-carboxyl, cyclobutane-carboxyl, 4-methylbenzoyl, crotonyl, 1-methyl-1H-indazole-3-carbonyl, 2-propenyl, isovaleryl, 4-phenylbenzoyl.

“Alkyl” is a straight or branched chain saturated aliphatic hydrocarbon group, and is selected to achieve the desired goal; i.e, is suitable for the intended use. In certain embodiments, the alkyl is C₁-C₃ or C₁-C₆. In certain embodiments, the alkyl can range from 1 to about 22 carbon atoms. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species. An alkyl range from C₁-C₂₂ intends independently all straight and branched chains 1, 2, 3, 4, 5, 6, 7, 8, 9, 10, 11, 12, 13, 14, 15, 16, 17, 18, 19, 20, 21 or 22 carbon atoms. Other embodiments include alkyl groups having from 1 to 8 carbon atoms, 1 to 4 carbon atoms or 1 or 2 carbon atoms, e.g. C₁-C₈alkyl, C₁-C₄alkyl, and C₁-C₂alkyl. When C₀-Cₙalkyl is used herein in conjunction with another group, for example, (C₃-C₇cycloalkyl)C₀-C₄alkyl-, the indicated group, in this case cycloalkyl, is either directly bound by a single covalent bond (C₀alkyl), or attached by an alkyl chain having the specified number of carbon atoms, in this case 1, 2, 3, or 4 carbon atoms. Examples of alkyl include, but are not limited to, methyl, ethyl, n-propyl, isopropyl, n-butyl, isobutyl, sec-butyl, tert-butyl, n-pentyl, isopentyl, tert-pentyl and neopentyl. In one embodiment, the alkyl group is optionally substituted as described above.

“Alkylaryl” or “alkaryl” refers to an alkyl group with an aryl substituent. The term aralkyl or arylalkyl refers to an aryl group with an alkyl substituent.

“Alkenyl” is an aliphatic hydrocarbon group having one or more double carbon-carbon bonds that may occur at any stable point along the chain, and is selected to achieve the desired goal; i.e, is suitable for the intended use. Nonlimiting examples are C₂-C₆, C₃-C₆, C₂-C₂₂, and C₃-C₂₂. The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described above.

“Alkoxy” is —O-alkyl as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Alkynyl” is an aliphatic hydrocarbon group having one or more triple carbon-carbon bonds that may occur at any stable point along the chain and is selected to achieve the desired goal; i.e, is suitable for the intended use. The specified ranges as used herein indicate an alkenyl group having each member of the range described as an independent species, as described above for the alkyl moiety. Nonlimiting embodiments are C₂-C₆, C₃-C₆, C₂-C₂₂ and C₃-C₂₂. Examples of alkynyl include, but are not limited to, ethynyl and propynyl. In one embodiment, the alkynyl group is optionally substituted as described above.

“Allenyl” is an alkenyl group having two consecutive double bonds, i.e., a group of formula —C═C═CH₂.

“Cycloalkyl” is a saturated hydrocarbon ring and is selected to achieve the desired goal; i.e, is suitable for the intended use. The specified ranges as used herein indicate a cycloalkyl group having each member of the range described as an independent species, as described above for the alkyl moiety. As used herein, cycloalkyl typically refers to a ring having from 3 to about 8 carbon atoms or from 3 to 7 (3, 4, 5, 6, or 7) carbon atoms. Cycloalkyl substituents may be pendant from a substituted nitrogen or carbon atom, or a substituted carbon atom that may have two substituents may have a cycloalkyl group, which is attached as a spiro group. Examples of cycloalkyl groups include cyclopropyl, cyclobutyl, cyclopentyl, and cyclohexyl. In one embodiment, the cycloalkyl group is optionally substituted as described above.

“Halo” or “halogen” indicates any of fluoro, chloro, bromo, and iodo.

“Haloalkyl” refers to an alkyl or cycloalkyl group substituted with 1 or more halogen atoms, typically fluoro, chloro, bromo or iodo, up to the maximum allowable number of halogen atoms. Examples of haloalkyl include, but are not limited to, fluoromethyl, difluoromethyl trifluoromethyl, and 2-fluoroethyl.

“Haloalkoxy” indicates a haloalkyl group as defined herein attached through an oxygen bridge (oxygen of an alcohol radical).

“Aryl”, as used herein, and unless otherwise specified, refers to phenyl, biphenyl, or naphthyl and typically phenyl. In one embodiment, the aryl group is optionally substituted with one or more moieties that do not adversely affect the desired properties of the molecules and for example, can be any of those described above. Possible substituents include any referred to as “optional substituents” above, and for example can be alkyl, alkenyl, alkynyl, halogen (fluoro, chloro, bromo or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “heteroaryl” refers to a monovalent aryl radical of 5-, 6-, or 7-membered rings, and includes fused ring systems (at least one of which is aromatic) of 5-12 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The term “heterocycle,” as used herein refers to a saturated or a partially unsaturated (i.e., having one or more double and/or triple bonds within the ring) carbocyclic radical of 3 to about 12, and more typically 3 to 10 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo[4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. Examples of heterocyclic rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, piperidonyl, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, dihydroisoquinolinyl, tetrahydroisoquinolinyl, pyrazolidinylimidazolinyl, imidazolidinyl, 2-oxa-5-azabicyclo[2.2.2]octane, 3-oxa-8-azabicyclo[3.2.1]octane, 8-oxa-3-azabicyclo[3.2.1]octane, 6-oxa-3-azabicyclo[3.1.1]heptane, 2-oxa-5-azabicyclo[2.2.1]heptane, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro moieties are also included within the scope of this definition. Examples of a heterocyclic group wherein 1 or 2 ring carbon atoms are substituted with oxo (═O) moieties are pyrimidinonyl and 1,1-dioxo-thiomorpholinyl. The heterocycle groups herein are optionally substituted independently with one or more substituents described herein.

A “dosage form” means a unit of administration of an active agent. Non-limiting examples of dosage forms include tablets, capsules, injections, suspensions, liquids, intravenous fluids, emulsions, creams, ointments, suppositories, inhalable forms, transdermal forms, and the like.

The term “5′-deuterated carbon” unless otherwise indicated can refer to a 5′-mono-deuterated or 5′-di-deuterated carbon, and typically means a 5′-dideuterated carbon moiety.

“Pharmaceutical compositions” are compositions comprising at least one active agent, such as a compound or salt of one of the active compounds disclosed herein, and at least one other substance, such as a carrier. Pharmaceutical compositions optionally contain more than one active agents. “Pharmaceutical combinations” or “combination therapy” refers to the administration of at least two active agents, and in one embodiment, three or four or more active agents which may be combined in a single dosage form or provided together in separate dosage forms optionally with instructions that the active agents are to be used together to treat a disorder, such as but not limited to a viral disease such as hepatitis C, or a disorder associated with hepatitis C, or another viral infection as described herein.

“Pharmaceutically acceptable salts” includes derivatives of the disclosed compounds in which the parent compound is modified by making inorganic and organic, suitably non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. The pharmaceutically acceptable salt can be in the form of a pure crystal, or single polymorphic form, or can be used in non-crystalline or amorphic, glassy, or vitreous form, or a mixture thereof. In an alternative embodiment, the active compound can be provided in the form of a solvate.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

The term “carrier” means a diluent, excipient, or vehicle with which an active compound is provided.

A “pharmaceutically acceptable excipient” means an excipient that is useful in preparing a pharmaceutical composition/combination that is generally safe, is sufficiently non-toxic, and neither biologically nor otherwise undesirable. A “pharmaceutically acceptable excipient” as used in the present application includes both one and more than one such excipient.

A “patient” or “host” is a human or non-human animal, including, but not limited to, simian, avian, feline, canine, bovine, equine or porcine in need of medical treatment. Medical treatment can include treatment of an existing condition, such as a disease or disorder, or a prophylactic or diagnostic treatment. In a particular embodiment, the patient or host is a human patient. In an alternative embodiment, the patient such as a host is treated to prevent a disorder or disease described herein.

Methods of Treatment

The disclosure provides a method to treat a host, typically a human, infected with any disorder that can be treated with a nucleoside or nucleotide, including but not limited to, a viral disease, tumor, cancer or other neoplastic or abnormal cellular proliferation, hyperuricaemia, a disorder treated with an immunosuppressive agent, a disorder treatable with an anti-methylating agent or a phosphodiesterase inhibitor, a disorder treated with an epigenetic modulator, or a neural or cardiovascular disease using an effective amount of a 5′deuterated analogue of the active nucleoside or nucleotide, optionally as a pharmaceutically acceptable salt and optionally in a pharmaceutically acceptable carrier. In one embodiment, the 5′-deuterated analogue of the nucleoside or nucleotide for the selected indication is a phosphoramidate, 3,5-cyclic phosphoramidate, phosphate ester, diester, or triester, nucleotide derivative of a monophosphate, diphosphate, or triphosphate, a 3′,5′-cyclic phosphate (including CycloSAL), a phospholipid (including acylphospholipids and etherphospholipids), a HepDirect prodrug, a SATE derivative (S-acyl-2-thioester)s, a DTE (dithiodiethyl) prodrug or a protein conjugate.

In one embodiment the disease is hepatitis C. In one embodiment, the disorder is HIV. In one embodiment, hepatitis C, or another disorder described herein, is treated with an effective amount of a 5′-deuterated nucleos(t)ide compound described herein, optionally as a pharmaceutically acceptable salt and optionally in a pharmaceutically acceptable carrier.

In another embodiment, an effective amount of one of the 5′-deuterated nucleoside phosphate compounds described herein, optionally as a pharmaceutically acceptable salt and optionally in a pharmaceutically acceptable carrier can be used to treat a host, typically a human, with a secondary condition associated with hepatitis C, or another disorder described herein, including but not limited to those disorders described below in (i) through (viii).

This disclosure provides methods of treating a viral infection in a patient, including a hepatitis C infection, by providing an effective amount of a 5′-deuterated nucleotide, 5′-deuterated-nucleoside, a 5′-deuterated nucleotide phosphate (mono, di or triphosphate), a 5′-deuterated stabilized nucleotide prodrug, or 5′-deuterated acyclic nucleoside prodrug described herein, for example, in any of Tables 1-6, or pharmaceutically acceptable salt thereof, to the patient infected with a hepatitis C virus. A 5′-deuterated nucleotide, 5′-deuterated nucleoside, a 5′-deuterated stabilized nucleotide prodrug, a 5′-deuterated nucleotide phosphate (mono, di or triphosphate) or a 5′-deuterated acyclic nucleoside prodrug described herein or salt may be provided as the only active agent or may be provided together with one or more additional active agents. In certain embodiments the compound or salt is administered together with a NS3 protease inhibitor, a NS5A inhibitor, a NS5B inhibitor, or a combination of these.

An effective amount of a pharmaceutical composition/combination of the disclosure may be an amount sufficient to (a) inhibit the progression of hepatitis C or other disorder described herein; (b) cause a regression of the hepatitis C infection or other disorder described herein; or (c) cause a cure of a hepatitis C infection, or other disorder described herein, for example such that HCV virus or HCV antibodies can no longer be detected in a previously infected patient's blood or plasma. An amount of a pharmaceutical composition/combination effective to inhibit the progress or cause a regression of hepatitis C, or other disorder described herein, includes an amount effective to stop the worsening of symptoms of hepatitis C, or other disorder described herein, or reduce the symptoms experienced by a patient infected with the hepatitis C virus, or other disorder described herein. Alternatively a halt in progression or regression of a disorder described herein, for example hepatitis C, may be indicated by any of several markers for the disease. For example, a lack of increase or reduction in the hepatitis C viral load or a lack of increase or reduction in the number of circulating HCV antibodies in a patient's blood are markers of a halt in progression or regression of hepatitis C infection. Other hepatitis C disease markers include aminotransferase levels, particularly levels of the liver enzymes AST and ALT. Normal levels of AST are from 5 to 40 units per liter of serum (the liquid part of the blood) and normal levels of ALT are from 7 to 56 units per liter of serum. These levels will typically be elevated in a HCV infected patient. Disease regression is usually marked by the return of AST and ALT levels to the normal range.

In yet another embodiment, an effective amount of one of the 5′-deuterated nucleotide, 5′-deuterated nucleoside, 5′-deuterated stabilized nucleotide prodrug, or 5′-deuterated acyclic nucleoside prodrug described herein, optionally as a pharmaceutically acceptable salt and optionally in a pharmaceutically acceptable carrier can be used as a prophylaxis to ward off or prevent a host, typically a human, from having a disorder described herein, for example the hepatitis C infection. In an alternative embodiment, an effective amount of one of the 5′-deuterated nucleotide, 5′-deuterated nucleoside, 5′-deuterated nucleotide phosphate (including the mono, di or triphosphate), 5′-deuterated stabilized nucleotide prodrug, or 5′-deuterated acyclic nucleoside prodrug compounds described herein, optionally as a pharmaceutically acceptable salt and optionally in a pharmaceutically acceptable carrier can be used to treat a secondary condition associated with a disorder described herein, for example hepatitis C, including but not limited to those disorders described below in (i) through (viii).

(i) Cryoglobulinemia which is abnormal antibodies (called cryoglobulins) that come from hepatitis C virus stimulation of lymphocytes. These antibodies can deposit in small blood vessels, thereby causing inflammation of the vessels (vasculitis) in tissues throughout the body including the skin, joints and kidneys (glomerulonephritis).

(ii) B-cell non-Hodgkin's lymphoma associated with hepatitis C, which is considered to be caused by excessive stimulation by hepatitis C virus of B-lymphocytes, resulting in abnormal reproduction of the lymphocytes.

(iii) Skin conditions such as lichen planus and porphyria cutanea tarda have been associated with hepatitis C infection.

(iv) Cirrhosis, which is a disease in which normal liver cells are replaced with scar or abnormal tissue. Hepatitis C is one of the most common causes of liver cirrhosis.

(v) Ascites, which is the accumulation of fluid in the abdominal cavity commonly caused by cirrhosis of the liver, which can be caused by hepatitis C infection.

(vi) Hepatocellular carcinoma, of which 50% of the cases in the U.S. are currently caused by chronic hepatitis C infection.

(vii) Hepatitis C related jaundice, which is a yellowish pigmentation caused by increased bilirubin.

(viii) Thrombocytopenia is often found in patients with hepatitis C and may be the result of bone marrow inhibition, decrease in liver thrombopoietin production and/or an autoimmune mechanism. In many patients, as hepatitis C advances, the platelet count decreases and both bone marrow viral inhibition and antiplatelet antibodies increase.

Other symptoms and disorders associated with hepatitis C that may be treated by an effective amount of a pharmaceutical composition/combination of the disclosure include decreased liver function, fatigue, flu-like symptoms: fever, chills, muscle aches, joint pain, and headaches, nausea, aversion to certain foods, unexplained weight loss, psychological disorders including depression, and tenderness in the abdomen.

Certain of the active compounds presented herein can also be used to enhance liver function generally associated with hepatitis C infection, for example, synthetic function including synthesis of proteins such as serum proteins (e.g., albumin, clotting factors, alkaline phosphatase, aminotransferases (e.g., alanine transaminase, aspartate transaminase), 5′-nucleosidase, y glutaminyltranspeptidase, etc.), synthesis of bilirubin, synthesis of cholesterol, and synthesis of bile acids; a liver metabolic function, including carbohydrate metabolism, amino acid and ammonia metabolism, hormone metabolism, and lipid metabolism; detoxification of exogenous drugs; and a hemodynamic function, including splanchnic and portal hemodynamics.

Certain of the compounds and pharmaceutical compositions/combinations disclosed herein are also useful for treating viral infections in patients other than a hepatitis C infection. In an alternative embodiment, the infection may be an RNA viral infection, such as Togaviridae, Picornaviridae, Coronaviridae, or Flaviviridae viral infection. The disclosure includes a method of treating a Togaviridae, Picornaviridae, Coronaviridae, or Flaviviridae viral infection by administering an effective amount of one of the active compounds disclosed herein, to a subject infected with a togavirus, picornavirus, coronavirus, or flavivirus. Flaviviridae viral infections include infections with viruses of the genera Flavivirus, Pestivirus, and Hepacivirus. Flavivirus infections include yellow fever, Dengue fever, West Nile virus, encephalitis, including St. Louis encephalitis, Japanese B encephalitis, California encephalitis, central European encephalitis, Russian spring-summer encephalitis, and Murray Valley encephalitis, Wesselsbron disease, and Powassan disease. Pestivirus infections include primarily livestock diseases, including swine fever in pigs, BVDV (bovine viral diarrhea virus) in cattle, and Border Disease virus infections. Hepacivirus infections includes Hepatitis C and canine Hepacivirus. Togavirus infections include Sindbis virus, Eastern equine encephalitis virus, Western equine encephalitis virus, Venezuelan equine encephalitis virus, Ross River virus, O'nyong'nyong virus, Chikungunya virus, Semliki Forest virus, and Rubella virus. Picornavirus infections include infections with viruses of the genuses Aphthovirus, Aquamavirus, Avihepatovirus, Cardiovirus, Cosavirus, Dicipivirus, Enterovirus, Erbovirus, Hepatovirus, Kobuvirus, Megrivirus, Parechovirus, Salivirus, Sapelovirus, Senecavirus, Teschovirus, and Tremovirus. Coronavirus infections include infections with virus of the genuses Alphacoronavirus, Betacoronavirus (which includes Severe acute respiratory coronavirus (SARS)), Gammacoronavirus, and Deltacoronavirus. The disclosure includes compositions comprising a compound of the present disclosure useful in an effective amount for treating Dengue fever, West Nile fever, yellow fever, or BVDV (bovine viral diarrhea virus) and methods of treating these infections by administering a 5′-deuterated nucleotide phosphate, 5′-deuterated-hydroxyl nucleoside, a 5′-deuterated stabilized nucleotide phosphate prodrug, or a 5′-deuterated acyclic nucleoside prodrug compound described herein to a patient infected with the virus.

Certain of the active compounds and methods described herein are useful for the treatment of cancer or other abnormal proliferative disorders. As contemplated herein, the cancer treated can be a primary tumor or a metastatic tumor. In one aspect, the methods described herein are used to treat a solid tumor, for example, melanoma, lung cancer (including lung adenocarcinoma, basal cell carcinoma, squamous cell carcinoma, large cell carcinoma, bronchioloalveolar carcinoma, bronchiogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcmoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas and leiomyosarcomas, mixed mullerian tumors); glioma, glioblastoma, medullablastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, lymphoma, leukemia, and cancers of the vagina among others.

Combination Therapy

The present disclosure also includes pharmaceutical compositions and combinations comprising a 5′-deuterated nucleotide, 5′-deuterated nucleoside, a 5′-deuterated stabilized nucleotide prodrug, or a 5′-deuterated acyclic nucleoside prodrug compound described herein and at least one additional active agent, to treat any of the disorders described herein. In one embodiment, a methods of treatment are presented comprising administering a compound or composition described herein to a patient infected with hepatitis C, or another disorder described herein. In certain embodiments the additional active agent is an HCV NS3 protease inhibitor or an HCV NS5A or another NS5B inhibitor.

In nonlimiting embodiments, the 5′-deuterated nucleotide, 5′-deuterated nucleoside, 5′-deuterated stabilized nucleotide prodrug, or 5′-deuterated acyclic nucleoside prodrug compounds of the present disclosure can be administered in combination or alternation with one or more of the selected active compound for the selected target indication. In certain embodiments, the second agent is a caspase inhibitor, a cyclophilin inhibitor, a cytochrome P450 monooxygenase inhibitor, an entry inhibitor, a glucocorticoid, a protease inhibitor (including an HIV or HCV inhibitor), a hematopoietin, a homeopathic therapy, an immunomodulatory compound, an immunosuppressant, an interleukin, an interferon or interferon enhancer, an IRES inhibitor, an monoclonal or polyclonal antibody, a nucleoside or nucleotide analogue or prodrug, a non-nucleoside inhibitor, an NS4B inhibitor, an NS5A inhibitor, an NS5B inhibitor, a P7 protein inhibitor, a polymerase inhibitor, an RNAi compound, a therapeutic vaccine, a TNF agonist, a tubulin inhibitor, a sphingosine-1-phosphate receptor modulator, or a TLR agonist.

Nonlimiting examples of active agents in these categories are:

Caspase Inhibitors: IDN-6556 (Idun Pharmaceuticals);

Cyclophilin Inhibitors: for example, NIM811 (Novartis), SCY-635 (Scynexis), and DEBIO-025 (Debiopharm);

Cytochrome P450 monooxygenase inhibitors: ritonavir, ketoconazole, troleandomycin, 4-methyl pyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, and VX-497 (Merimebodib). Preferred CYP inhibitors include ritonavir, ketoconazole, troleandomycin, 4-methyl pyrazole, cyclosporin, and clomethiazole;

Entry Inhibitors: ITX-5061 (iTherX);

Glucocorticoids: hydrocortisone, cortisone, prednisone, prednisolone, methylprednisolone, triamcinolone, paramethasone, betamethasone, and dexamethasone;

HCV Protease Inhibitors: for example Sovaprevir and ACH-2684. ABT-450 (Abbott), ACL-181 and AVL-192 (Avila), BMS-032 (Bristol Myers Squibb), Boceprevir (Merck), danoprevir (Hoffman-La Roche and Genentech), GS-9256 (Gilead), GS-9451 (Gilead), Telaprevir (VX-950, Vertex), VX-985 (Vertex), Simeprevir (TMC435, Tibotec), Fosamprenavir (prodrug of Amprenavir, Glaxo/Vertex), indinavir (Crixivan, Merck), TMC435350 (Tibotec/Medivir), Faldaprevir (BI 201335. Boehringer Ingelheim), PHX-1766 (Phenomix), Vaniprevir (MK-7009, Merck), narlaprevir (SCH900518, Schering), MK-5172 (Merck);

Hematopoietins: hematopoietin-1 and hematopoietin-2. Other members of the hematopoietin superfamily such as the various colony stimulating factors (e.g. G-CSF, GM-CSF, M-CSF), Epo, and SCF (stem cell factor);

Homeopathic Therapies: Milk Thistle, silymarin, ginseng, glycyrrhizin, licorice root, schisandra, vitamin C, vitamin E, beta carotene, and selenium;

Immunomodulatory compounds: thalidomide, IL-2, hematopoietins, IMPDH inhibitors, for example Merimepodib (Vertex Pharmaceuticals Inc.), interferon, including natural interferon (such as OMNIFERON, Viragen and SUMIFERON, Sumitomo, a blend of natural interferons), natural interferon alpha (ALFERON, Hemispherx Biopharma, Inc.), interferon alpha-n1 from lymphblastoid cells (WELLFERON, Glaxo Wellcome), oral alpha interferon, Peg-interferon, Peg-interferon alfa 2a (PEGASYS, Roche), recombinant interferon alfa 2a (ROFERON, Roche), inhaled interferon alpha 2b (AERX, Aradigm), Peg-interferon alpha 2b (ALBUFERON, Human Genome Sciences/Novartis, PEGINTRON, Schering), recombinant interferon alfa 2b (INTRON A, Schering), pegylated interferon alfa 2b (PEG-INTRON, Schering, VIRAFERONPEG, Schering), interferon beta-1a (REBIF, Ares-Serono, Inc. and Pfizer), consensus interferon alpha (INFERGEN, Intermune), interferon gamma-1b (ACTIMMUNE, Intermune, Inc.), un-pegylated interferon alpha, alpha interferon, and its analogs, and synthetic thymosin alpha 1 (ZADAXIN, SciClone Pharmaceuticals Inc.), and lamdba interferon (BMS);

Immunosupressants: sirolimus (RAPAMUNE, Wyeth);

Interleukins: (IL-1, IL-3, IL-4, IL-5, IL-6, IL-10, IL-11, IL-12), LIF, TGF-beta, TNF-alpha) and other low molecular weight factors (e.g. AcSDKP, pEEDCK, thymic hormones, and minicytokines);

Interferon Enhancers: EMZ702 (Transition Therapeutics);

IRES inhibitors: VGX-410C (VGX Pharma);

Monoclonal and Polyclonal antibodies: XTL-6865 (HEPX-C, XTL), HuMax-HepC (Genmab), Hepatitis C Immune Globin (human) (CIVACIR, Nabi Biopharmceuticals), XTL-002 (XTL), Rituximab (RITUXAN, Genentech/IDEC), GS-6624 (Gilead);

Nucleoside analogues: Sofosbuvir (PSI-7977, Pharmasset and Gilead), PSI-7851 (Pharmasset), PSI-7977 (Pharmasset), R7128 (mericitabine, Roche), R7348 (Roche), NM283 (valopicitabine, Idenix), GS-6620 (Gilead), TMC-649 (Tibotec), VX-135 (Vertex, Alios), ALS-2200 (Alios), IDX184 (Idenix), IDX21437 (Idenix), IDX21459 (Idenix), Lamivudine (EPIVIR, 3TC, GlaxoSmithKline), MK-0608 (Merck), zalcitabine (HIVID, Roche US Pharmaceuticals), ribavirin (including COPEGUS (Roche), REBETOL (Schering), VILONA (ICN Pharmaceuticals, and VIRAZOLE (ICN Pharmaceuticals), isatoribine (Anadys Pharmaceuticals), ANA245 (Anadys Pharmaceuticals), and viramidine (ICN), an amidine prodrug of ribavirin. Combinations of nucleoside analogues may also be employed;

Non-nucleoside inhibitors: PSI-6130 (Roche/Pharmasset), ABT-333 and ABT-072 (Abbott), delaviridine (RESCRIPTOR, Pfizer), PF-868554 (Pfizer), GSK-852 (GlaxoSmithKline), Setrobuvir (ANA-598, Anadys), VX-222 (Vertex), BI-127 (Boehringer Ingelheim), and BMS-325 (Bristol Meyers);

NS4B inhibitors: clemizole (Eiger BioPharmaceuticals, Inc.);

NS5A inhibitors: Daclatasvir (BMS-790052, BMS), AZD-729 (Astra Zeneca); PPI-461 (Presidio), PPI-688 (Presidio), samatasvir (IDX719, Idenix), ledipasvir (GS-5885, Gilead), GS-5816 (Gilead), ombitasvir (ABT-267, AbbVie), GSK2336805 (GlaxoSmithKline), and elbasvir (MK-8742, Merck);

NS5B inhibitors: MBX-700 (Microbotix/Merck), RG-9190, VX-222 (Vertex), and BMS-791325 (Bristol Meyers Squibb);

P7 protein inhibitor: amantadine (SYMMETREL, Endo Pharmaceuticals, Inc.);

Polymerase inhibitors: ANA598 (Anadys), Tegobuvir (GS 9190, Gilead);

RNA interference: SIRNA-034 RNAi (Sirna Therapeutics);

Therapeutic Vaccines: IC41 (Intercell), GI 5005 (Globeimmune), Chronvac-C(Tripep/Inovio);

TNF agonists: adalimumab (HUMIRA, Abbott), entanercept (ENBREL, Amgen and Wyeth), infliximab (REMICADE, Centocor, Inc.);

Tubulin inhibitors: Colchicine;

Sphingosine-1 phosphatereceptor modulators: FTY720 (Novartis);

TLR agonists: TLR7 agonist (Anadys Pharmaceuticals), CPG10101 (Coley), and TLR9 agonists including CPG 7909 (Coley); and,

Vaccines: HCV/MF59 (Chiron), IC41 (Intercell).

For example, in some embodiments, the additional active agent is sovaprevir or ACH-2684 (HCV NS3 protease inhibitors) and/or and NS5A inhibitor.

The disclosure includes compositions in which the additional active agent is

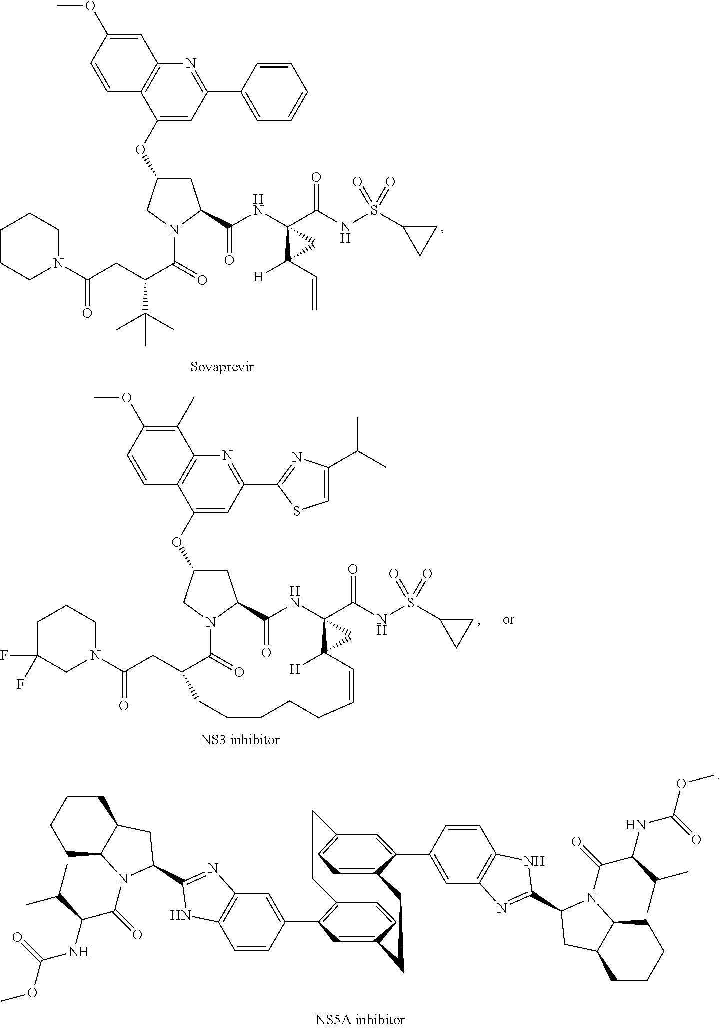

NS3 protease inhibitors, useful in the pharmaceutical compositions and combinations described here have been disclosed previously, for example in U.S. Pat. No. 7,906,619, issued Mar. 15, 2011, is hereby incorporated by reference in its entirety for its teachings regarding 4-amino-4-oxobutanoyl peptides. The '619 patent is particularly incorporated by reference at the Examples section beginning in column 50 and extending to column 85 which discloses compounds useful in compositions/combination with a 5′-deuterated nucleotide, 5′-deuterated nucleoside, 5′-deuterated stabilized nucleotide prodrug, or a 5′-deuterated acyclic nucleoside prodrug compound described here.

U.S. Publication No. 2010/0216725, published Aug. 26, 2010, is hereby incorporated by reference in its entirety for its teachings regarding 4-amino-4-oxobutanoyl peptides. The '725 application is particularly incorporated by reference at the Examples section beginning at page 22 and extending to page 100 which discloses compounds useful in compositions/combination with a 5′-deuterated nucleotide phosphate, 5′-deuterated-hydroxyl nucleoside, a 5′-deuterated stabilized nucleotide phosphate prodrug, or a 5′-deuterated acyclic nucleoside prodrug described herein.

U.S. Publication No. 2010/0152103, published Jun. 17, 2010, is hereby incorporated by reference in its entirety for its teachings regarding 4-amino-4-oxobutanoyl peptide cyclic analogues. The '103 application is particularly incorporated by reference at the Examples section beginning at page 19 and extending to page 60 which discloses compounds useful in compositions/combination with a 5′-deuterated nucleotide, 5′-deuterated nucleoside, a 5′-deuterated stabilized nucleotide prodrug, or a 5′-deuterated acyclic nucleoside prodrug described herein. Particularly the compounds disclosed herein may be used in combination with an NS3 protease inhibitor of the formulae shown below.

NS5A inhibitors, useful in the pharmaceutical compositions and combinations described here have been disclosed previously. U.S. Publication No. 2012/0302528, published Nov. 29, 2012, is hereby incorporated by reference in its entirety for its teachings regarding NS5A Inhibitors.

In certain embodiments the NS3 protease inhibitor is chosen from

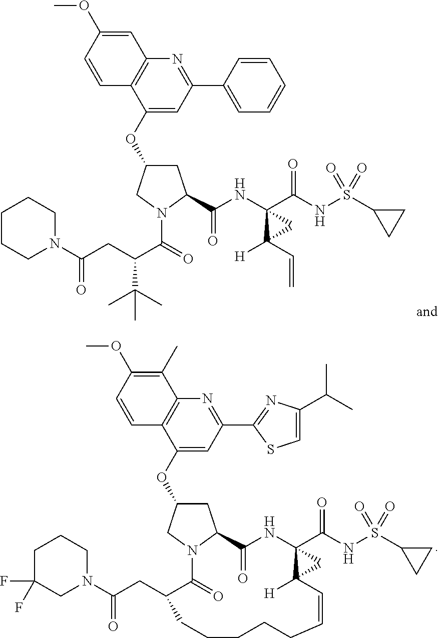

The NS5A inhibitor is chosen from

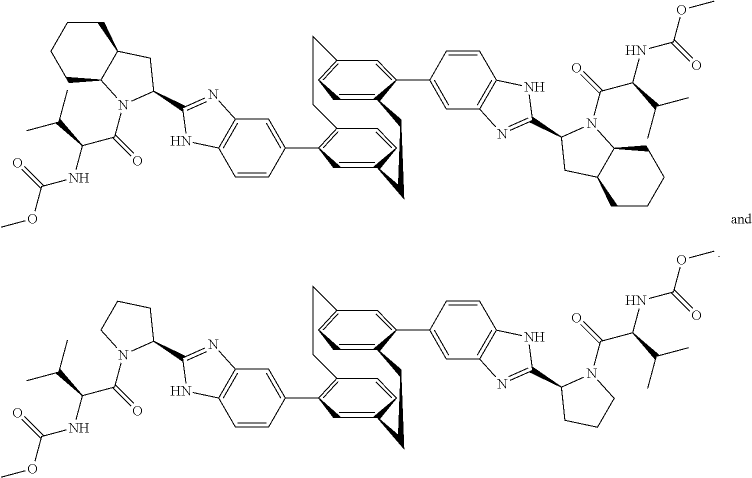

Pharmaceutical Compositions

The selected compound disclosed herein can be administered as the neat chemical, but is preferably administered as a pharmaceutical composition. The disclosure provides pharmaceutical compositions comprising any of the 5′-deuterated nucleotide, a 5′-deuterated nucleotide phosphate (such as a mono, di or triphosphate), 5′-deuterated nucleoside, 5′-deuterated stabilized nucleotide prodrug, or 5′-deuterated acyclic nucleoside prodrug compounds described herein (including any in Tables 1-6), together with at least one pharmaceutically acceptable carrier in an effective amount to treat the target indication. The pharmaceutical composition/combination may contain a compound or salt of any of the active compounds described herein as the only active agent, but in another embodiment may also contain at least one additional active agent. In certain embodiments for the treatment of HCV it is preferred that the additional active agent is an NS3 protease inhibitor or NS5A or NS5B inhibitor.

In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a 5′-deuterated nucleotide, 5′-deuterated nucleoside, or 5′-deuterated stabilized nucleotide prodrug, or 5′-deuterated acyclic nucleoside prodrug described herein and optionally from about 0.1 mg to about 2000 mg, from about 10 mg to about 1000 mg, from about 100 mg to about 800 mg, or from about 200 mg to about 600 mg of an additional active agent in a unit dosage form. In certain embodiments the 5′-deuterated nucleotide, 5′-deuterated nucleoside, 5′-deuterated stabilized nucleotide prodrug, or 5′-deuterated acyclic nucleoside prodrug compound is delivered in an oral dosage form such as a pill, tablet or capsule in an effective amount, which may in some embodiments be at least 10, 25, 50, 100, 150, 200, 250, 300, 350, 400, 450, 500, 550, 600, 650, 700, 750, 800, 850, 900, 950 or 1000 mg, or any dosage falling in between these dosages. The pharmaceutical composition may also include a molar ratio of a 5′-deuterated nucleotide, 5′-deuterated nucleoside, a 5′-deuterated stabilized nucleotide prodrug, or a 5′-deuterated acyclic nucleoside prodrug compound and an additional active agent. For example, for the treatment of HCV, the pharmaceutical composition may contain a molar ratio of about 0.5:1, about 1:1, about 2:1, about 3:1 or from about 1.5:1 to about 4:1, and the other active agent may be, for example, an NS3 protease inhibitor, an NS5A inhibitor or another NS5B inhibitor.

Compounds disclosed herein may be administered by any suitable means, including orally, topically, parenterally, by inhalation or spray, sublingually, transdermally, via buccal or sublingual transmucosal administration, rectally, as an ophthalmic solution or injection, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and should be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present disclosure.

The pharmaceutical compositions/combinations can be formulated for oral administration. These compositions typically contain between 5 or 10 to 99 weight % (wt. %) of any of the selected compounds described herein, for example, a 5′-deuterated nucleotide, 5′-deuterated nucleoside, a 5′-deuterated stabilized nucleotide prodrug, or a 5′-deuterated acyclic nucleoside prodrug compound and usually at least about 5 wt. % of a 5′-deuterated nucleotide, 5′-deuterated nucleoside, a 5′-deuterated stabilized nucleotide prodrug, or a 5′-deuterated acyclic nucleoside prodrug compound. Some embodiments contain from about 25 wt. % to about 50 wt. % or from about 5 wt. % to about 75 wt. % of the active material.

Specifically for HCV, an effective amount of a pharmaceutical composition/combination of the disclosure may be an amount sufficient, for example, to (a) inhibit the progression of hepatitis C or other disorder described herein; (b) cause a regression of the hepatitis C infection or other disorder described herein; (c) cause a cure of a hepatitis C infection, or other disorder described herein, for example such that HCV virus or HCV antibodies can no longer be detected in a previously infected patient's blood or plasma, or (d) treat an HCV-associated disorder. An amount of a pharmaceutical composition/combination effective to inhibit the progress or cause a regression of a disorder described herein, for example hepatitis C, includes an amount effective to stop the worsening of symptoms of the disorder or reduce the symptoms experienced by a patient with the disorder. Alternatively a halt in progression or regression of the disorder may be indicated by any of several markers for the disease. For example, in the case of HCV, a lack of increase or reduction in the hepatitis C viral load or a lack of increase or reduction in the number of circulating HCV antibodies in a patient's blood can be markers of a halt in progression or regression of hepatitis C infection. Other hepatitis C disease markers include aminotransferase levels, particularly levels of the liver enzymes AST and ALT.

The compound or pharmaceutically acceptable salt of any of the compounds described herein, including 5′-deuterated nucleotide, 5′-deuterated nucleoside, 5′-deuterated stabilized nucleotide, or 5′-deuterated acyclic nucleoside prodrug compounds described herein and at least one additional active agent may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the disclosure may comprise administering or delivering the compound or salt of any of the active compounds described herein, and an additional active agent sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

Frequency of dosage may also vary depending on the compound used and the particular disease treated. However, for treatment of most infectious disorders, a dosage regimen of 4 times daily or less is preferred and a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease in the patient undergoing therapy.

The pharmaceutical packaging may include an active compound or salt as described herein in a container together with instructions for using the compound to treat a patient suffering from a disorder described herein, for example Hepatitis C infection, are included herein.

Packaged pharmaceutical compositions/combinations are also included herein. Such packaged combinations include any of the compounds described herein, including 5′-deuterated nucleotide, 5′-deuterated nucleoside, 5′-deuterated stabilized nucleotide prodrug, or 5′-deuterated acyclic nucleoside prodrug compounds described herein in a container together with instructions for using the combination to treat or prevent a viral infection, such as a hepatitis C infection, in a patient.

For HCV, the packaged pharmaceutical composition/combination may include one or more additional active agents. In certain embodiments the additional active agent is an NS3 protease inhibitor, an NS5A or another NS5B inhibitor.

The packaged pharmaceutical combination may include any of the deuterated compounds described herein, including a 5′-deuterated nucleotide, 5′-deuterated nucleoside, a 5′-deuterated stabilized nucleotide prodrug, or a 5′-deuterated acyclic nucleoside prodrug compound described herein or pharmaceutically acceptable salt thereof and the additional active agent provided simultaneously in a single dosage form, concomitantly in separate dosage forms, or provided in separate dosage forms for administration separated by some amount of time that is within the time in which both the compound such as a 5′-deuterated nucleotide, 5′-deuterated nucleoside, a 5′-deuterated stabilized nucleotide prodrug, or a 5′-deuterated acyclic nucleoside prodrug compound described herein and the additional active agent are within the bloodstream of the patient.

The packaged pharmaceutical combination may include a 5′-deuterated nucleotide, 5′-deuterated nucleoside, a 5′-deuterated stabilized nucleotide prodrug, or a 5′-deuterated acyclic nucleoside prodrug compound described herein or pharmaceutically acceptable salt thereof provided in a container with an additional active agent provided in the same or separate container, with instructions for using the combination to treat an HCV infection in a patient.

Chemical Syntheses: Schemes

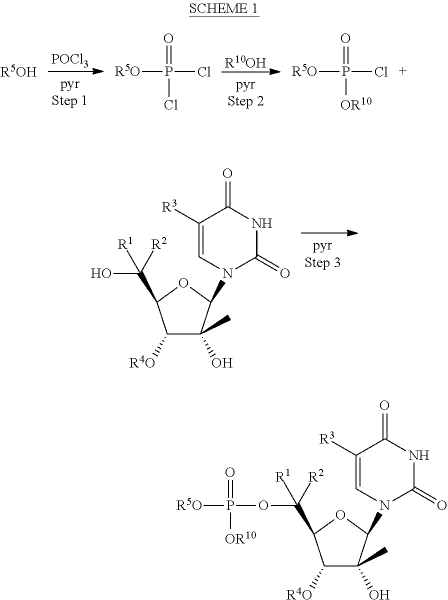

Scheme 1 illustrates the general synthesis of a 5′-deuterated stabilized uridine diphosphate compound.

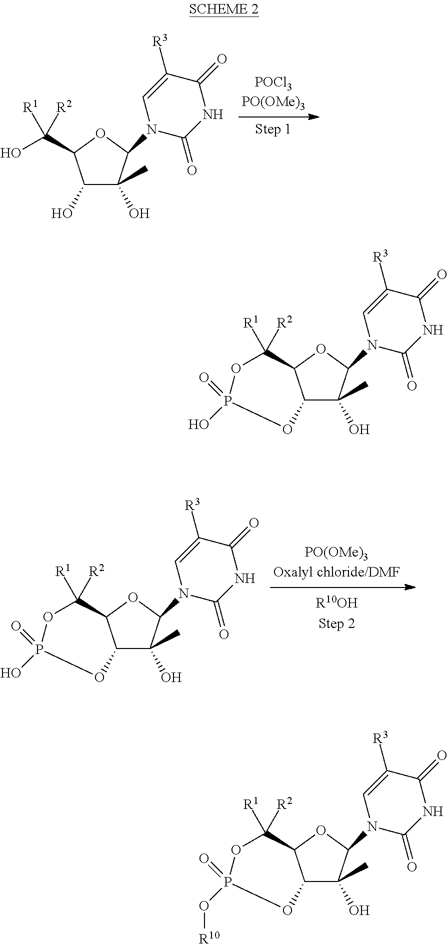

Scheme 2 illustrates the general synthesis of a 3′,5′-deuterated stabilized uridine cyclic phosphate compound.

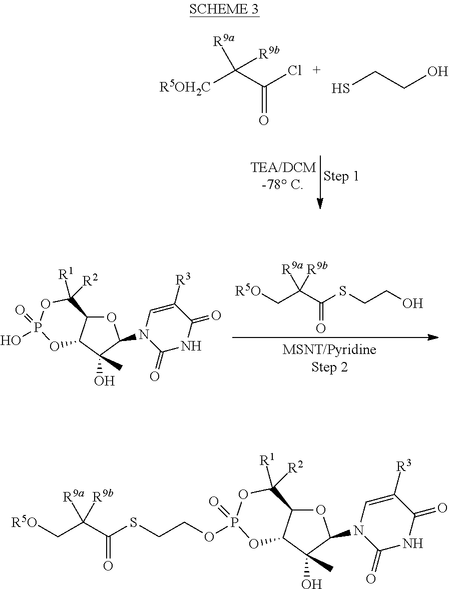

Scheme 3 illustrates the general synthesis of a 3′,5′-deuterated stabilized uridine cyclic phosphate SATE compound.

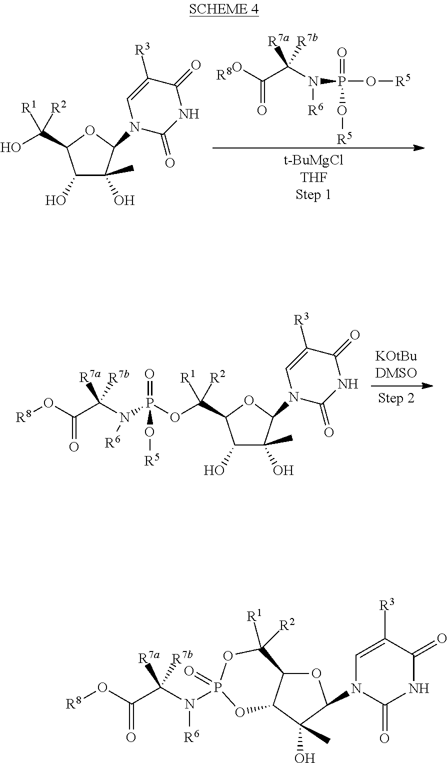

Scheme 4 illustrates the general synthesis of a 3′,5′-deuterated stabilized uridine cyclic phosphoramidate compound.

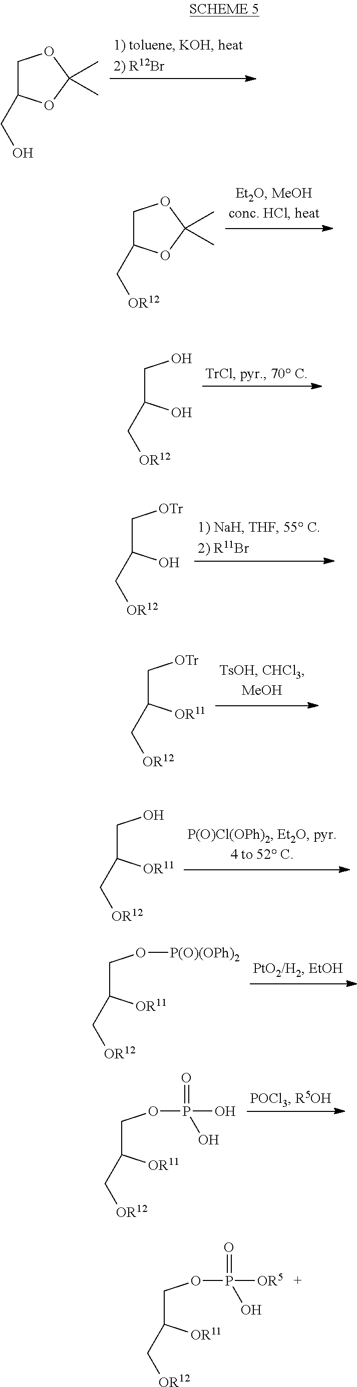

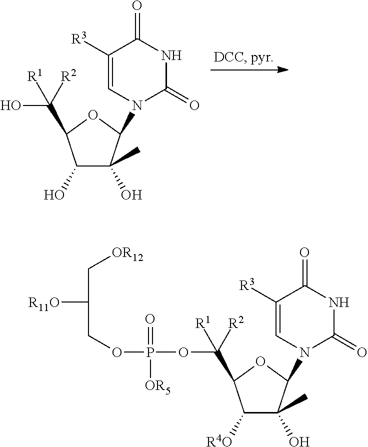

Scheme 5 illustrates the general synthesis of a stabilized lipid conjugate of a 5′-nucleoside phosphate.

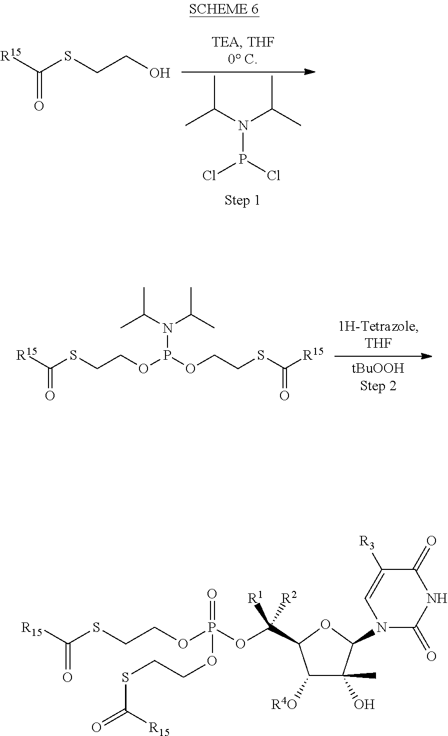

Scheme 6 illustrates the general synthesis of a 5′-deuterated stabilized uridine phosphate bis-SATE compound.

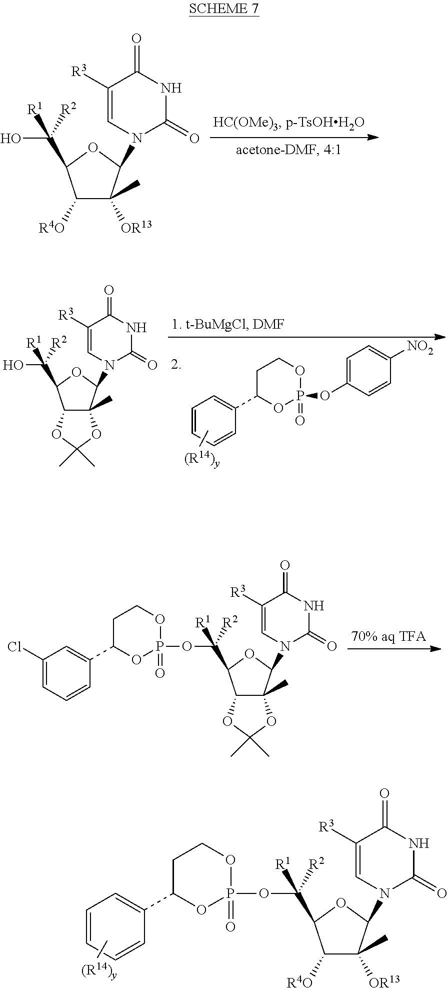

Scheme 7 illustrates the general synthesis of a 5′-deuterated stabilized uridine phosphate HepDirect compound.

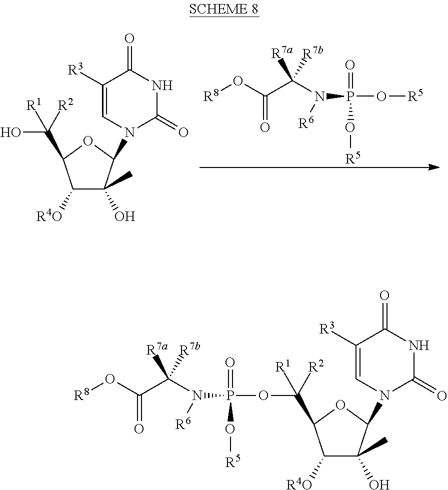

Scheme 8 illustrates the general synthesis of a 5′-deuterated stabilized uridine phosphoramidate compound.

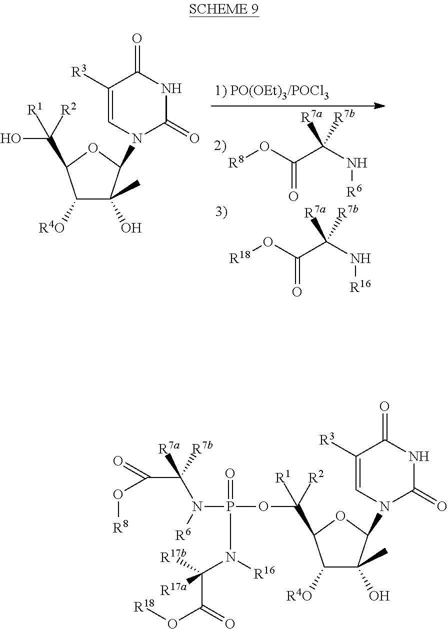

Scheme 9 illustrates the general synthesis of a 5′-deuterated stabilized uridine bisphosphoramidate compound.

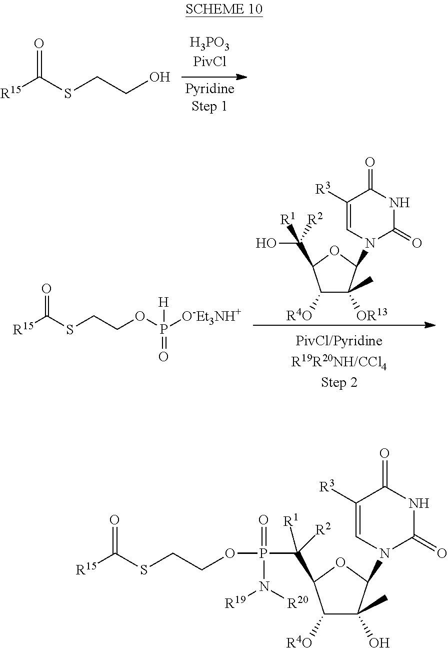

Scheme 10 illustrates the general synthesis of a 5′-deuterated stabilized uridine phosphate SATE amidate compound.

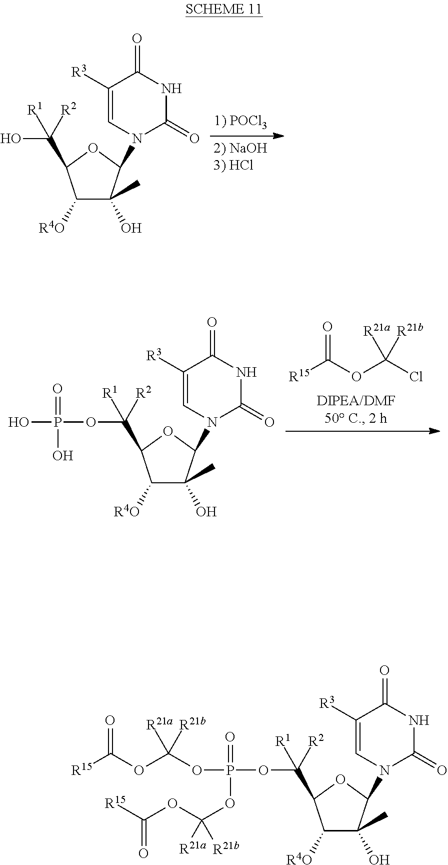

Scheme 11 illustrates the general synthesis of 5′-deuterated stabilized uridine bis-(isopropoxycarbonyloxymethyl)phosphate and bis-(tert-butyloxycarbonyloxymethyl)phosphate derivatives.

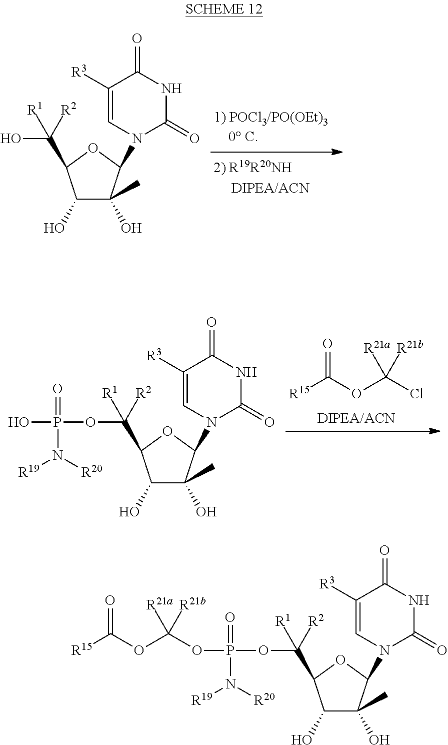

Scheme 12 illustrates the general synthesis of 5′-deuterated stabilized uridine phosphoramidate esters.

### EXAMPLES

### Example 1

### Synthesis of 5′-Deuterated Stabilized Uridine 5′-Phosphate Diesters

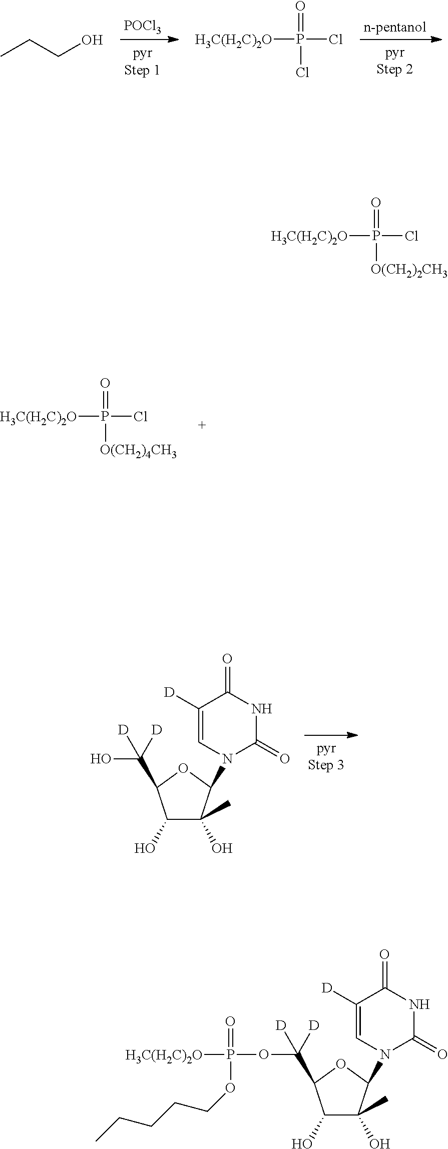

In step 1, phosphorus oxychloride is reacted with n-propanol in pyridine optionally at an elevated temperature. In step 2, the dichloride is reacted with n-pentanol in pyridine optionally at an elevated temperature. In step 3 the phosphoryl chloride is treated with 5-deutero-5′,5′-dideutero-2′-C-methyluridine from Example 11 in pyridine to generate the 5′-phosphodiester. Once the reaction is complete as determined by HPLC, the reaction is concentrated and purified by silica gel column chromatography to afford the product.

### Example 2

### Synthesis of 5′-Deuterated Stabilized Uridine 3′,5′-Cyclic Phosphates

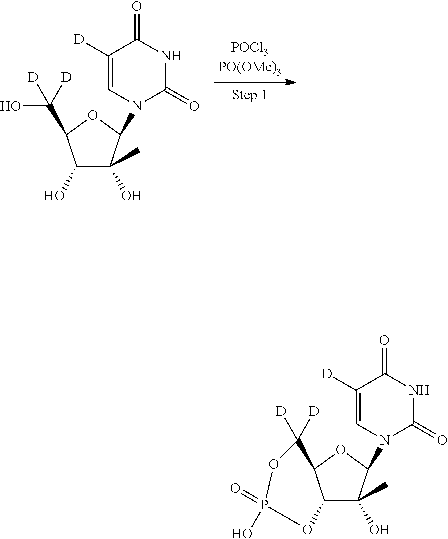

In step 1, 5-deutero-5′,5′-dideutero-2′-C-methyluridine from Example 11 is dissolved in trimethyl phosphate (PO(OMe)₃) and cooled to ˜0° C. To this solution is added excess POCl₃ and the reaction mixture is allowed to warm to room temperature. The reaction mixture is quenched with a solution of base such as potassium hydroxide and the solution is concentrated in vacuo. The residue is purified by HPLC to give the 3′,5′-cyclic phosphate of 5-deutero-5′,5′-dideutero-2′-C-methyluridine.

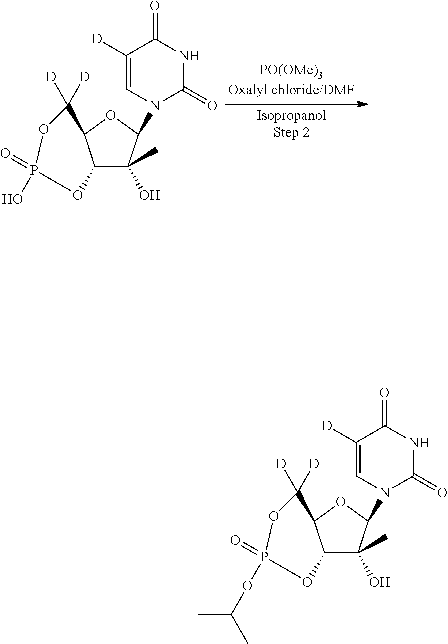

In step 2, a solution of the 3′,5′-cyclic phosphate in DCM and PO(OMe)₃ is cooled to 0° C., oxalyl chloride and a small amount of DMF is added. After stirring for an hour isopropanol is added and the reaction is allowed to warm to room temperature and stirred until the reaction is complete. The solvent is evaporated and the residue purified by HPLC to give the isopropylester.

### Example 3

### Synthesis of 5′-Deuterated Stabilized Uridine 3′,5′-Cyclic Phosphate SATE Compounds

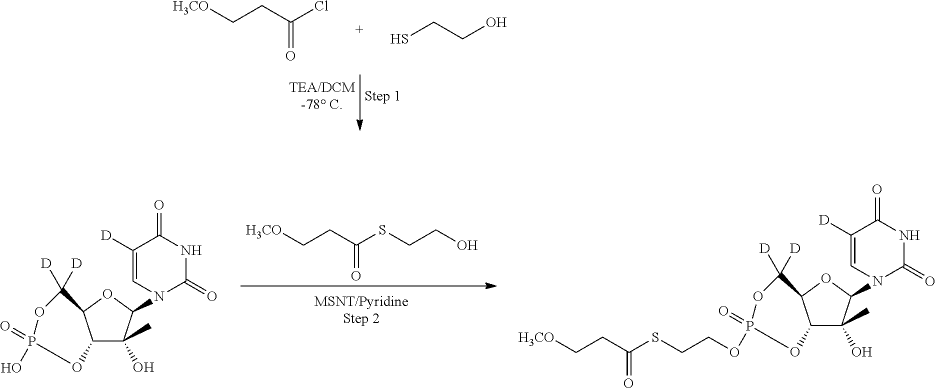

In step 1, 3-methoxypropanoyl chloride is added to a solution containing mercaptoethanol and triethylamine in DCM at −78° C. The reaction is allowed to warm to room temperature after stirring at −78° C. for 1 h. After stirring for an hour the reaction is diluted with water and the DCM layer is separated and evaporated to dryness. The residue is purified by chromatography over silica gel to give the SATE alcohol. In step 2, the cyclic phosphate, from Example 2 and the SATE alcohol are dissolved in pyridine. To this solution is added 1-(2-mesitylene-2-sulfonyl-3-notro)-1,2,4-triazole (MSNT). The mixture is stirred under an inert atmosphere for 3 days protected from light. All volatiles are evaporated and the residue purified by chromatography to give the product.

### Example 4

### Synthesis of 5′-Deuterated Stabilized Uridine 3′,5′-Cyclic Phosphoramidates

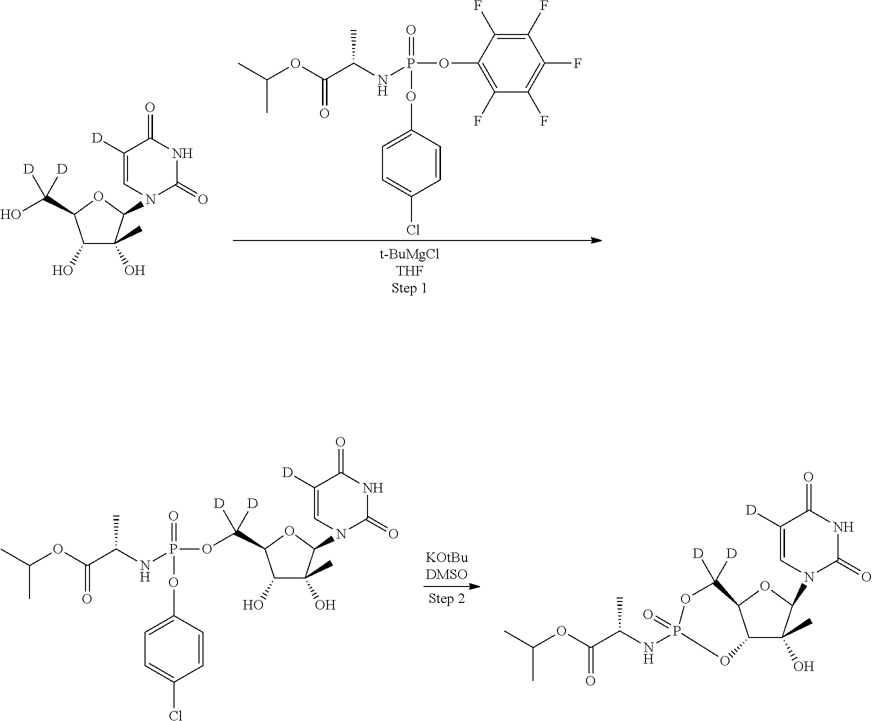

In step 1, 5-deutero-5′,5′-dideutero-2′-C-methyluridine from Example 11 is coupled with the activated phosphate in the presence of t-butylmagnesium chloride in an organic solvent such as tetrahydrofuran according to the method of Ross et al., J. Org. Chem., 76, 8311 (2011). In step 2, the phosphoramidate nucleotide is treated with potassium tert-butoxide in dimethyl sulfoxide. Once the reaction is complete as determined by HPLC, the solution is cooled to 0° C. neutralized with 1N HCl, diluted with water, extracted with DCM and evaporated to dryness. The product is isolated by chromatography.

### Example 5

### Synthesis of 5′-Deuterated Stabilized Uridine-5′-Phosphate Lipid Conjugates

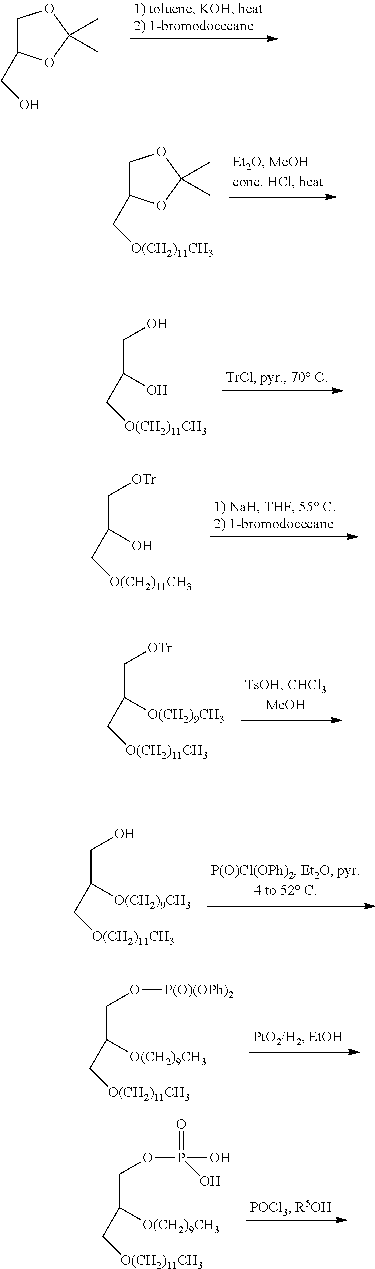

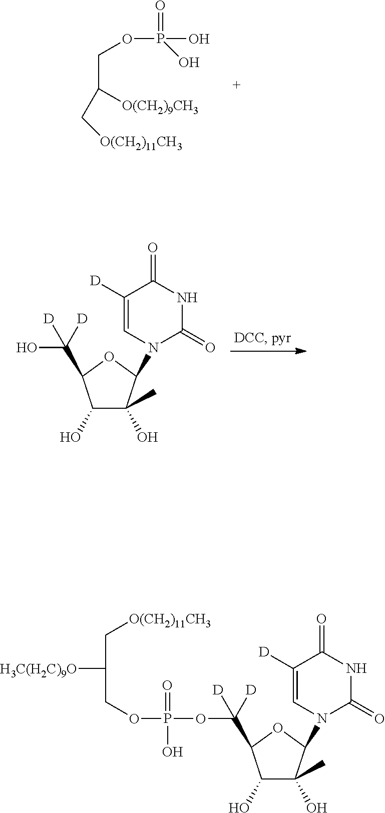

In step 1, solketal is treated with potassium hydroxide in toluene and then treated with 1-bromododecane at an elevated temperature to afford the product. In step 2, the acetal is treated with concentrated hydrochloric acid in a mixture of diethyl ether and methanol at an elevated temperature to afford the diol product. In step 3, the diol is treated with trityl chloride in pyridine at an elevated temperature to generate the trityl derivative. In step 4, the trityl derivative is treated with sodium hydride in tetrahydrofuran and then treated with 1-bromodecane. In step 5, the trityl protecting group is removed by treating the compound with p-toluenesulfonic acid in chloroform and methanol. In step 6, the primary alcohol is treated with diphenylchlorophosphate in diethyl ether and pyridine to afford the corresponding diphenyl phosphate. In step 7, the diphenyl phosphate is treated with platinum oxide in ethanol under a hydrogen atmosphere to afford the phosphate. In step 8, the phosphate is treated with 5-deutero-5′,5′-dideutero-2′-C-methyluridine from Example 11 in pyridine and dicyclohexylcarbodiimide is added. The reaction is monitored by HPLC and concentrated in vacuo upon completion. The crude product is purified using silica gel chromatography to afford the lipid conjugate.

### Example 6

### Synthesis of 5′-Deuterated Stabilized Uridine-5′-Phosphate Lipid Conjugates

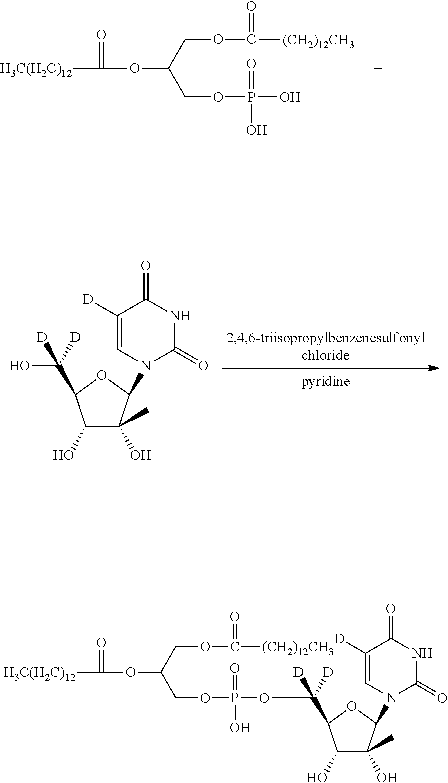

Dimyristoylphosphatic acid is synthesized according to the method of Hostetler, K. Y., et al., J. Biol. Chem., 265, 6112-6117 (1990). Dimyristoylphosphatic acid is lyophilized from cyclohexane. Dimyristoylphosphatic acid, 5-deutero-5′,5′-dideutero-2′-C-methyluridine from Example 11 and 2,4,6-triisopropylbenzenesulfonyl chloride are diluted in pyridine. The reaction is monitored by HPLC and diluted with water when the reaction is complete. The reaction is concentrated in vacuo and the product is purified on silica gel chromatography that is eluted with chloroform:methanol (90:10) (v:v). Fractions containing the product are combined and concentrated.

### Example 7

### Synthesis of 5′-Deuterated Stabilized Uridine-5′-Phosphate Bis SATEs

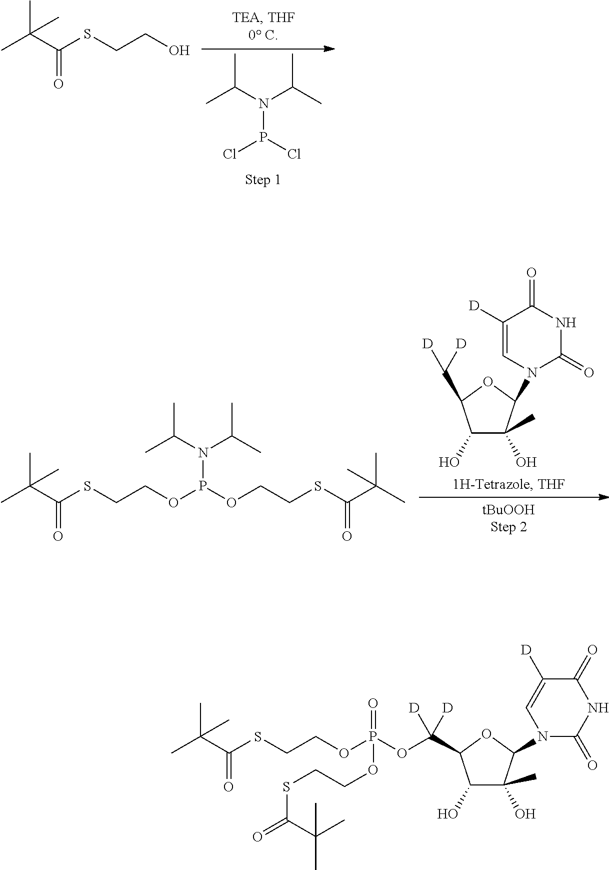

In step 1, the SATE alcohol, hydroxyethyl 2,2-dimethylpropanethioate is dissolved in THF and cooled to 0° C. To this solution is added diisopropylphosphoramidous dichloride and the reaction is stirred for 2 h. Hexane is added and the formed precipitate is filtered. The filtrate is concentrated and the residue purified by chromatography over silica gel to give the phosphoramidite reagent. In step 2, 5-deutero-5′,5′-dideutero-2′-C-methyluridine from Example 11 and 1H-tetrazole is dissolved in THF and the phosphoramidite reagent added and stirred at room temperature for 2 h. The reaction mixture is cooled to −40° C. and tertiarybutylhydroperoxide is added and the reaction is stirred for 2 h at room temperature. All volatiles are evaporated and the residue is dissolved in DCM and washed with 10% sodium bisulfate and concentrated. The residue is purified by chromatography over silica gel to give the product.

### Example 8

### Synthesis of 5′-Deuterated Stabilized Uridine CycloSal-Nucleotide Derivatives

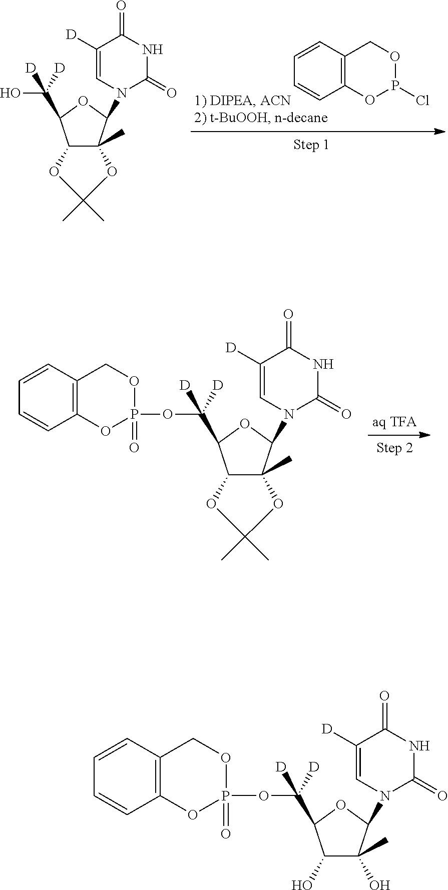

In step 1, the protected nucleoside (from Example 11), in acetonitrile or a mixture of N,N-dimethylformamide and tetrahydrofuran is cooled to −20° C. and treated sequentially with diisopropylethylamine and an acetonitrile solution of 2-chloro-4H-benzo[d][1,3,2]dioxaphosphinine (prepared by the method of Warnecke et al. J. Org. Chem., 74, 3024 (2009)). The reaction mixture is warmed to room temperature and stirred, then cooled to −20° C., and to it is added a tert-butyl hydroperoxide solution in n-decane. The reaction mixture is warmed to room temperature, stirred at this temperature until the progress of the reaction is determined to be satisfactory by HPLC analysis, and evaporated to dryness under reduce pressure. The resulting residue is subjected to column chromatography on silica gel to afford the protected monophosphate derivative. In step 2, the acetonide-protected derivative is treated with a 70% aqueous solution of trifluoroacetic acid at 0° C. The volatiles are removed under reduced pressure and the remaining residue is partitioned between an organic solvent and a saturated aqueous solution of sodium bicarbonate. The organic phase is removed, dried over sodium sulfate, and evaporated under reduced pressure. The crude material is subjected to column chromatography on silica gel to afford the cycloSal-nucleotide (mixture of diastereomers).

### Example 9

### (S)-Isopropyl 2-(((S)-(Perfluorophenoxy)(Phenoxy)Phosphoryl)Amino) Propanoate (Compound 1)

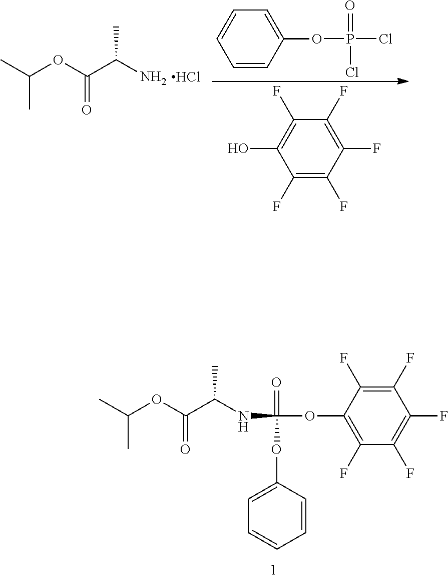

L-Alanine isopropyl ester HCl salt (160 g) is charged in a 5 L four-necked flask equipped with mechanical stirrer, thermometer and dropping funnel To the flask, dichloromethane (1 L) is added and the suspension is cooled to −70° C., followed by addition of triethylamine (200 g, 276 mL) over 45 min. To the mixture is added a solution of phenyl dichlorophosphate (200 g) in dichloromethane (1 L) over 2.5 h. The reaction mixture is stirred at this temperature for an additional 90 min and then allowed to warm up to 0° C. over a period of 2 h and stirred for 2 h at 0° C. To the mixture a solution of 2,3,4,5,6-pentafluorophenol (174.4 g) in 400 mL dichloromethane and a solution of triethylamine (105.4 g) in 200 mL dichloromethane are added dropwise simultaneously over a period of 1.2 h. The mixture is warmed to rt and stirred overnight.* The solid, triethylamine HCl salt, is filtered off and the cake is washed with dichloromethane (3×150 mL). The filtrate is concentrated under reduced pressure and the residue triturated with MTBE (3.0 L). The white solid is removed by filtration. The cake is washed with MTBE (3×150 mL). The filtrate is concentrated and the resulting crude solid triturated with 20% ethyl acetate in hexane (2.0 L). The solid is collected by filtration and washed with 10% NaHCO3 until the aq phase reached pH 7, the solid is then washed with water and dried in a vacuum oven (55° C.) for 28 h. The dried solid is mixed with 500 mL heptane-EtOAc (5:1) and stirred for 1 h. The solid is collected by filtration and washed with heptane-EtOAc (5:1, 2×80 mL) to afford a >99% single isomer. The solid is dried to give compound 1. *In an alternative work-up procedure, the reaction mixture is filtered and the DCM layer is washed with an aq. 0.1 N NaOH solution, followed by water, dried, and evaporated to dryness. The residue is suspended in heptane/EtOAc (5:1) and the solid is filtered. The solid is resuspended in heptane/toluene (85:15) to isolate the pure single isomer.

### Example 10

### Preparation of (S)-isopropyl 2-(((R)-(((2S,3R,4R,5R)-5-(2,4-dioxo-3,4-dihydropyrimidin-1(2H)-yl)-3,4-dihydroxy-4-methyltetrahydrofuran-2-yl)dideuteromethoxy)(phenoxy)phosphoryl)amino)propanoate (Compound 7)

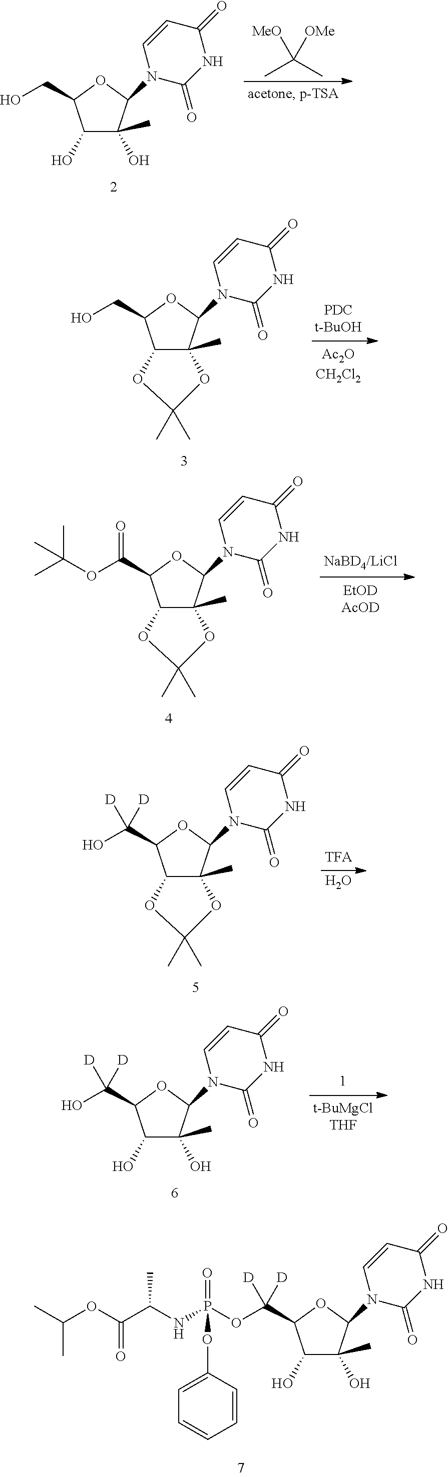

2,2-Dimethylpropane (140 mL) is added to 2′-C-methyluridine 2 (100 g) in acetone (700 mL). The resulting mixture is cooled in an ice bath for 30 min, then p-toluenesulfonic acid (11 g) is added and the reaction mixture is stirred at rt for 24 h. After completion of the reaction (monitored by HPLC), the reaction mixture is cooled in an ice bath for 30 min and neutralized using cold potassium carbonate (12 g in 13 mL water, pH 7-8). The solvent is removed under reduced pressure until dryness. THF (˜500 mL) is added to the residue and the solids are removed by filtration. The filtrate is co-evaporated with silica gel and purified by chromatography over silica gel (5-15% MeOH in CHCl₃) to give compound 3. 1H NMR (400 MHz, DMSO-d6, 300 K): δ 1.22 (s, 3H), 1.34 (s, 3H), 1.49 (s, 3H), 3.63 (dd, J=12.0 Hz, 2.8 Hz, 1H), 3.69 (dd, J=12.0 Hz, 3.1 Hz, 1H), 4.15 (m, 1H), 4.47 (d, J=2.8 Hz, 1H), 5.25 (br s, 1H), 5.63 (dd, J=8.2 Hz, 2.3 Hz), 6.01 (s, 1H), 7.85 (d, J=8.2 Hz, 1H), 11.37 (s, 1H); LC-MS: 299 amu (M+1).

Compound 4 is prepared following the procedure reported by Corey et al. (J. Org. Chem. 1984, 49, 4735) with modifications described below. To acetonide 3 (50 g) in CH₂Cl₂ (1 L) is added PDC (126.1 g) at rt followed by Ac₂O (171 g) and t-BuOH (248 g). The reaction temperature is maintained below 35° C. during the addition of reagents and then stirred at rt for 5 h. The reaction mixture is poured into aq. K₂CO₃ (250 g in 600 mL H₂O) and the organic layer is washed with CuSO₄ (100 g in 1 L H₂O). Activated charcoal (10 g) and silica gel (100 g) are added to the organic layer and stirred for 30 min and filtered. The filtrate is evaporated and residue purified by chromatography over silica gel (0-50% EtOAc in CHCl₃) to afford 4. 1H NMR (400 MHz, DMSO-d6, 300 K): δ 1.25 (s, 3H), 1.41 (s, 3H), 1.46 (s, 9H), 1.48 (s, 3H), 3.31 (s, 1H), 4.61 (s, 1H), 4.79 (s, 1H), 5.70 (dd, J=8.1 Hz, 2.0 Hz, 1H), 5.93 (br s, 1H), 7.97 (d, J=8.1 Hz, 1H), 11.41 (s, 1H); LC-MS: 369 amu (M+1).

Lithium chloride (1.76 g) was stirred with NaBD₄ (1.58 g) in EtOD for 1 h. Compound 4 (2.97 g) was added to this solution and stirred at rt for 3 h and quenched with acetic acid-d, diluted with ethyl acetate, washed with brine, and evaporated to dryness. The residue was purified by chromatography over silica gel to give the 5′-dideuterated compound 5.

Compound 5 (2.1 g) was treated with trifluoroacetic acid in the presence of water to give the 5′-dideuterated nucleoside 6. 1H NMR (400 MHz, CD₃OD, 300 K): δ 1.16 (s, 3H), 3.84 (d, J=9.2 Hz, 1H), 3.91 (d, J=9.2 Hz, 1H), 5.67 (d, J=8.1 Hz, 1H), 5.96 (s, 1H), 8.14 (d, J=8.1 Hz, 1H); 13C NMR (100 MHz, CD3OD, 300 K): δ 20.2, 73.4, 80.0, 83.8, 93.2, 102.3, 142.5, 152.5, 166.0 (ribose C-5′ not observed).

Compound 6 (1.0 g) was converted to the phosphoramidate derivative 7 following the procedure described by Ross et al. (J. Org. Chem. 2011, 76, 8311). 1H NMR (400 MHz, CD₃OD, 300 K): δ 1.15 (s, 3H), 1.21 (2×d, J=6.3 Hz, 6H), 1.35 (dd, J=7.2 Hz, JH, P=0.9 Hz, 3H), 3.79 (d, J=9.2 Hz, 1H), 3.91 (dq, JH, P=10.0 Hz, J=7.2 Hz, 1H), 4.08 (dd, J=9.2 Hz, JH, P=2.2 Hz, 1H), 4.96 (septet, J=6.3 Hz, 1H), 5.60 (d, J=8.1 Hz, 1H), 5.96 (s, 1H), 7.20 (m, 1H), 7.26 (m, 2H), 7.37 (m, 2H), 7.67 (d, J=8.1 Hz, 1H); 31P NMR (162 MHz, CD₃OD, 300 K): δ 3.8; LC-MS: 530 amu (M+1).

### Example 11

### Preparation of (S)-isopropyl 2-(((S)-(((2R,3R,4R,5R)-5-(5-deutero-2,4-dioxo-3,4-dihydropyrimidin-1(2H)-yl)-3,4-dihydroxy-4-methyltetrahydrofuran-2-yl)dideuteromethoxy)(phenoxy)phosphoryl)amino)propanoate (Formula II, Compound 10)

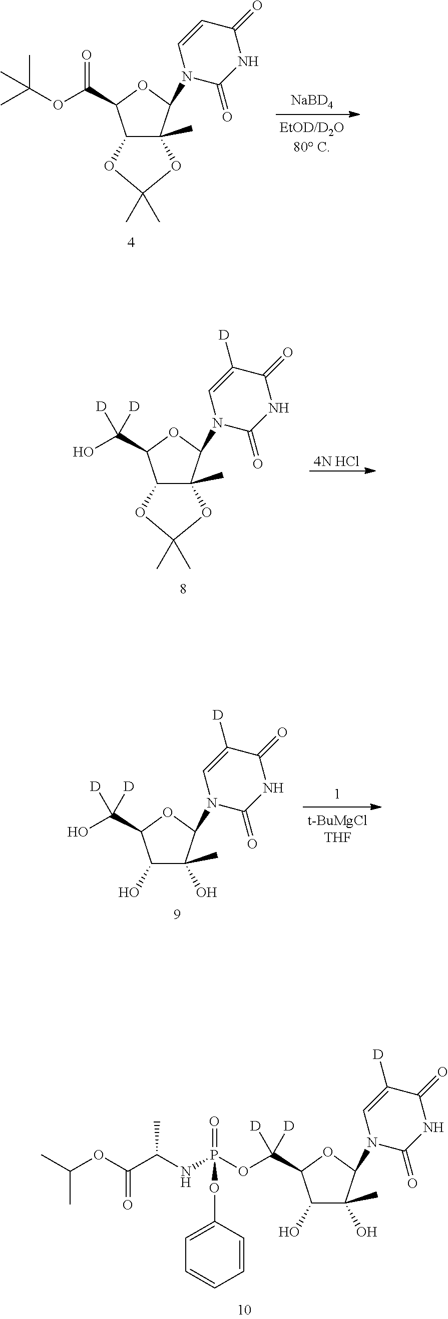

NaBD₄ (7.96 g) is added in portions to a cooled (5° C.) 70:30 v/v mixture of EtOD/D₂O (350 mL, 99% D) in a 1 L flask, followed by the addition of acetonide ester 4 (35 g) in portions (slowly bubbles). The resulting reaction mixture is stirred at rt for 3 h, and then heated at 80° C. for 1 d (1H NMR spectroscopic analysis indicates >85% deuterium incorporation at the 5-uracil position). The reaction mixture is filtered to remove solids and concentrated under reduced pressure to remove EtOD. Additional D₂O is added and the resulting mixture reheated at 95° C. to increase the deuterium incorporation at the 5 position to >98% (D-incorporation monitored by 1H NMR spectroscopy). After completion of the reaction, half the solvent is removed under reduced pressure, the mixture is cooled in an ice bath, AcOD (59 g) is added, and resulting mixture is stirred for 15-20 min. EtOAc (300 mL) and brine (100 mL) are added, the organic layer is separated, and the aq layer is again extracted with EtOAc (150 mL), followed by THF (150 mL). The combined organic layers are concentrated, the resulting residue is dissolved in 10% MeOH and CHCl₃ (300 mL), filtered, concentrated, and purified by chromatography over silica gel (ISCO, eluent DCM/MeOH) to give the deuterated acetonide 8. 1H NMR (400 MHz, DMSO-d6, 300 K): δ 1.22 (s, 3H), 1.36 (s, 3H), 1.49 (s, 3H), 3.31 (s, 2H), 4.14 (d, J=2.8 Hz, 1H), 4.47 (d, J=2.8 Hz, 1H), 5.21 (s, 1H), 6.01 (s, 1H), 7.85 (s, 1H), 11.36 (s, 1H); LC-MS: 302 amu (M+1).

Deuterated acetonide 8 (50 g) is added to a cooled (5° C.) 4 N HCl (250 mL) solution and stirred at rt for 3 h, during which time a white precipitate forms. The solvent is evaporated to dryness and to the residue is added water (100 mL) and stirred. The suspension is cooled to 5° C., stirred for 1 h. and the white precipitate is collected by filtration. The solid is washed with cold water (75 mL) and dried to afford the deuterated nucleoside 9. 1H NMR (400 MHz, CD₃OD, 300 K): δ 1.15 (s, 3H), 3.84 (d, J=9.2 Hz, 1H), 3.91 (d, J=9.2 Hz, 1H), 5.96 (s, 1H), 8.14 (s, 1H); 13C NMR (100 MHz, CD3OD, 300 K): δ 20.2, 73.4, 80.0, 83.8, 93.2, 142.4, 152.5, 166.0 (ribose C-5′ and uracil C-5 not observed); LC-MS: 262 amu (M+1).

Nucleoside 9 (37.3 g) in THF (750 mL) is cooled to −5° C. t-BuMgCl (1 M in THF, 430 mL) is added and the mixture is stirred for 30 min at the same temperature. The reaction mixture is stirred for another 30 minutes at rt., then cooled again to −5° C., and a solution of 1 (129.5 g) in THF (650 mL) is added slowly. The reaction mixture is stirred at rt. for 24 h., cooled to −5° C., and to it added cold 2N HCl (200 mL), followed by stirring for 10 min, and the addition of a saturated aq. solution of NaHCO₃ (˜250 mL, pH ˜8) and solid NaCl (50 g). The resulting mixture is stirred for 1 h. and the organic layer is separated. The aq. layer is extracted with THF (2×150 mL). All organic layers are combined and evaporated to dryness. The residue is purified partially over a short silica gel (500 mL) column (10-20% MeOH in CHCl3), then purified additionally by chromatography over silica gel (ISCO, 4×300 g cartridge, eluted with 0-10% MeOH in CH₂Cl₂) to afford the title compound 10. 1H NMR (400 MHz, CD₃OD, 300 K): δ 1.15 (s, 3H), 1.21 (2×d, J=6.3 Hz, 6H), 1.35 (dd, J=7.2 Hz, JH, P=0.9 Hz, 3H), 3.79 (d, J=9.2 Hz, 1H), 3.91 (dq, JH, P=10.0 Hz, J=7.2 Hz, 1H), 4.08 (dd, J=9.2 Hz, JH, P=2.3 Hz, 1H), 4.96 (septet, J=6.3 Hz, 1H), 5.96 (s, 1H), 7.20 (m, 1H), 7.26 (m, 2H), 7.37 (m, 2H), 7.67 (s, 1H); 31P NMR (162 MHz, CD₃OD, 300 K): δ 3.8; LC-MS: 531 amu (M+1).

### Example 12

### Alternative Preparation of Formula II (Compound 10)

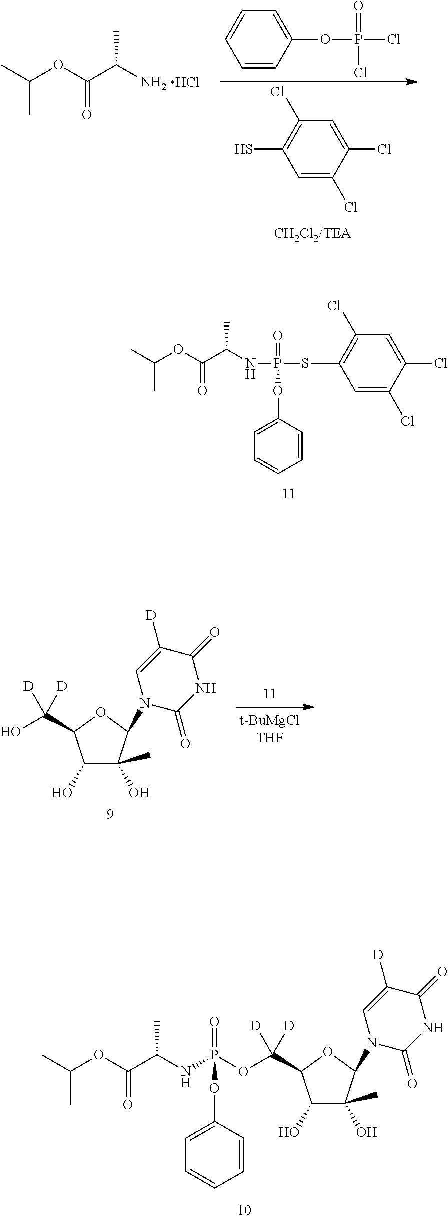

Phenoxydichlorophosphate (12.58 g) is added to a cold (−50° C.) solution of L-alanine isopropyl ester in CH₂Cl₂ (100 mL), followed by the addition of triethylamine (18.3 mL) in CH₂Cl₂ (36 mL) maintained at a temperature below −40° C. The reaction mixture is warmed to room temperature slowly and stirred for 2 h. and again cooled to −50° C. A solution of 2,4,5-trichlorothiophenol (12.74 g) in CH₂Cl₂ (20 mL) containing triethylamine (9.1 mL) is added. The reaction is warmed to rt. and stirred for 15 h. The reaction mixture is washed with water (˜300 mL) followed by saturated aq. NaHCO₃ (˜300 mL). The organic layer is separated, dried over Na₂SO₄, and evaporated to dryness under reduced pressure. The crude material is passed through a short column of silica (CH₂Cl₂/EtOAc 0:1 v/v to ˜1:4 v/v) and the product is collected after evaporation of the solvent. The product is dissolved in 100 mL of 2.5% EtOAc in heptane and the solution seeded with compound 11 (˜10 mg) and stirred for 1 h. at rt. The precipitate is collected by filtration, washed with a small amount of the above EtOAc/heptane solvent mixture, and dried to afford 11 as a single isomer. 1H NMR (400 MHz, CDCl₃, 300 K): δ 1.24 (d, J=6.3 Hz, 3H), 1.26 (d, J=6.3 Hz, 3H), 1.41 (d, J=7.0 Hz, 3H), 3.99-4.21 (m, 2H), 5.02 (septet, J=6.3 Hz, 1H), 7.17-7.24 (m, 3H), 7.34 (m, 2H), 7.52 (s, 1H), 7.73 (d, JH, P=2.2 Hz, 1H); 31P NMR (162 MHz, CDCl3, 300 K): δ 21.1.

A suspension of 9 (1.0 g) in THF is cooled to −20° C. and t-BuMgCl (11.6 mL, 1 M in THF) is added slowly, maintaining the temperature of the mixture below −20° C. The reaction mixture is warmed slowly to rt (˜2 h), stirred for 2 h, and then cooled to −10° C. Compound 11 (3.74 g) is added and the reaction mixture is warmed to rt and stirred. After 15 h, the reaction mixture is cooled to 0° C., 2N aq HCl is added (to pH ˜2), and the solution is stirred for 30 min at 0° C. Aqueous NaHCO₃ is added (to pH ˜8), followed by NaCl (˜3 g), and the mixture is stirred for 30 min. The organic layer is separated, dried, and evaporated under reduced pressure. The crude material is purified by column chromatography on silica gel (5% MeOH in CH₂Cl₂) to afford pure 10.

After compound 11 is isolated by filtration, the filtrate, which is enriched in the other stereoisomer at phosphorus, can be concentrated and purified by chromatographic techniques. This stereoisomer of 11 is treated with nucleoside 9 to give compound 31 described in Example 20.

### Example 13

### Alternative Preparation of Formula II (Compound 10)

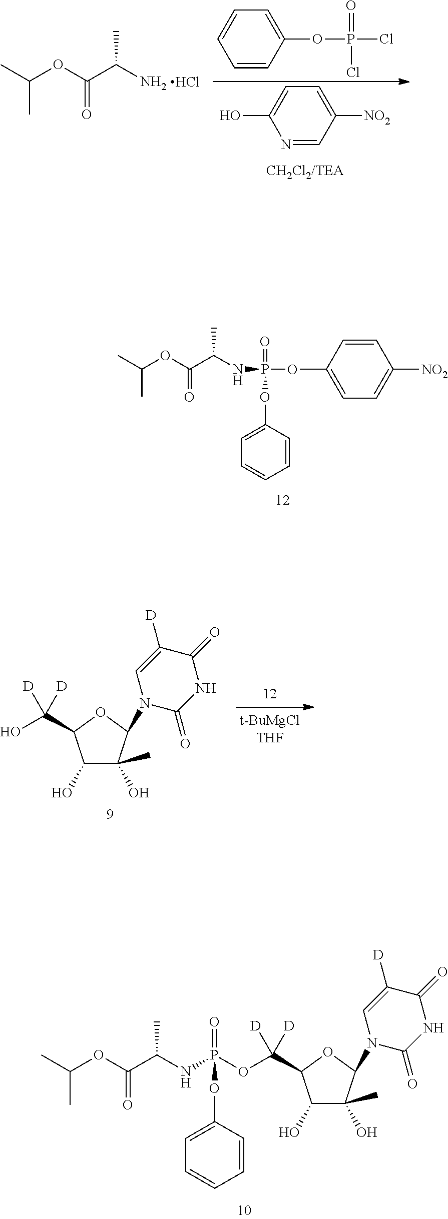

Compound 12 is prepared in a manner analogous to that described above in Example 12 for compound 11. Spectroscopic data for 12: 1H NMR (400 MHz, CDCl3, 300 K): δ 1.19 (d, J=6.3 Hz, 3H), 1.22 (d, J=6.3 Hz, 3H), 1.39 (d, J=6.7 Hz, 3H), 4.25-4.38 (m, 2H), 4.96 (septet, J=6.3 Hz, 1H), 7.10 (d, J=9.0 Hz, 1H), 7.19 (m, 1H), 7.25 (m, 2H), 7.34 (m, 2H), 8.50 (dd, J=9.0 Hz, J=2.9 Hz, 1H), 9.15 (d, J=2.9 Hz, 1H); 31P NMR (162 MHz, CDCl3, 300 K): δ −3.4.

Nucleoside 9 is treated with compound 12 in a manner analogous to that described in Example 11 to give compound 10.

Compound 31 described in Example 20 can be prepared using the other stereoisomer of compound 12 in a manner analogous to that described in Example 12.

### Example 14

### Preparation of (S)-isopropyl 2-(((S)-(((2R,3R,4R,5R)-5-(5-deutero-2,4-dioxo-3,4-dihydropyrimidin-1(2H)-yl)-3,4-dihydroxy-4-methyltetrahydrofuran-2-yl)methoxy)(phenoxy)phosphoryl)amino)propanoate (Compound 17)

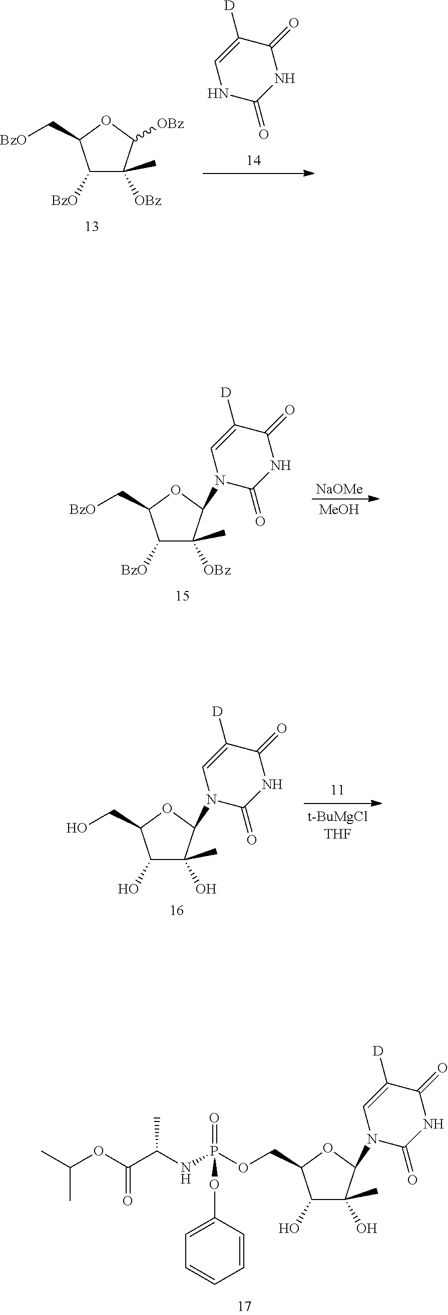

1,2,3,5-Tetra-O-benzoyl-2-C-methyl-β-D-ribofuranose 13 (2.44 g) was treated with uracil-5-dl 14 (1.0 g), following the procedure described in Harry-O'kuru et al. (J. Org. Chem. 1997, 62, 1754) using non-deuterated uracil, to give protected nucleoside 15. Compound 15 was treated with NaOMe in MeOH to give 2′-C-methyluridine-5-dl (16). 1H NMR (400 MHz, CD₃OD, 300 K): δ 1.16 (s, 3H), 3.78 (dd, J=12.5 Hz, 2.6 Hz, 1H), 3.84 (d, J=9.2 Hz, 1H), 3.92 (d of app t, J=9.2 Hz, 2.4 Hz, 1H), 3.98 (dd, J=12.5 Hz, 2.2 Hz, 1H), 5.96 (s, 1H), 8.14 (s, 1H); 13C NMR (100 MHz, CD3OD, 300 K): δ 20.2, 60.5, 73.4, 80.0, 83.9, 93.1, 142.4, 152.5, 166.0 (uracil C-5 not observed).

Compound 16 (0.7 g) was converted to the phosphoramidate derivative 17 in a manner analogous to that described in Example 11. 1H NMR (400 MHz, CD3OD, 300 K): δ 1.15 (s, 3H), 1.21 (2×d, J=6.3 Hz, 6H), 1.35 (dd, J=7.2 Hz, JH, P=0.9 Hz, 3H), 3.79 (d, J=9.2 Hz, 1H), 3.91 (dq, JH, P=10.0 Hz, J=7.2 Hz, 1H), 4.08 (m, 1H), 4.37 (ddd, J=11.8 Hz, JH, P=5.9 Hz, J=3.7 Hz, 1H), 4.50 (ddd, J=11.8 Hz, JH, P=5.9 Hz, J=2.0 Hz, 1H), 4.96 (septet, J=6.3 Hz, 1H), 5.96 (s, 1H), 7.20 (m, 1H), 7.26 (m, 2H), 7.37 (m, 2H), 7.67 (s, 1H); 31P NMR (162 MHz, CD3OD, 300 K): δ 3.8; LC-MS: 529 amu (M+1).

### Example 15

### Preparation of (S)-isopropyl 2-(((S)-(((2R,3R,4R,5R)-5-(6-deutero-2,4-dioxo-3,4-dihydropyrimidin-1(2H)-yl)-3,4-dihydroxy-4-methyltetrahydrofuran-2-yl)methoxy)(phenoxy)phosphoryl)amino)propanoate (Compound 18)

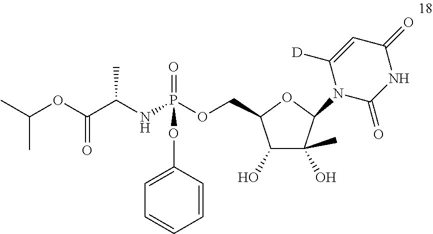 [SMILES: [2H]c1cc(=O)[nH]c(=O)n1[C@@H]1O[C@H](CO[P@@](=O)(N[C@@H](C)C(=O)OC(C)C)Oc2ccccc2)[C@@H](O)[C@@]1(C)O]

Compound 18 was prepared using uracil-6-d1 in a manner analogous to that described for compound 17 in Example 14. 1H NMR (400 MHz, CD₃OD, 300 K): δ 1.15 (s, 3H), 1.21 (2×d, J=6.3 Hz, 6H), 1.35 (dd, J=7.2 Hz, JH, P=0.9 Hz, 3H), 3.79 (d, J=9.2 Hz, 1H), 3.91 (dq, JH, P=10.0 Hz, J=7.1 Hz, 1H), 4.09 (m, 1H), 4.37 (ddd, J=11.8 Hz, JH, P=5.9 Hz, J=3.7 Hz, 1H), 4.50 (ddd, J=11.8 Hz, JH, P=5.9 Hz, J=2.0 Hz, 1H), 4.96 (septet, J=6.3 Hz, 1H), 5.60 (s, 1H), 5.96 (s, 1H), 7.20 (m, 1H), 7.26 (m, 2H), 7.37 (m, 2H); 31P NMR (162 MHz, CD₃OD, 300 K): δ 3.8; LC-MS: 529 amu (M+1).

### Example 16

### Preparation of (S)-isopropyl 2-(((S)-(((2R,3R,4R,5R)-5-(5,6-dideutero-2,4-dioxo-3,4-dihydropyrimidin-1(2H)-yl)-3,4-dihydroxy-4-methyltetrahydrofuran-2-yl)methoxy)(phenoxy)phosphoryl)amino)propanoate (Compound 19)

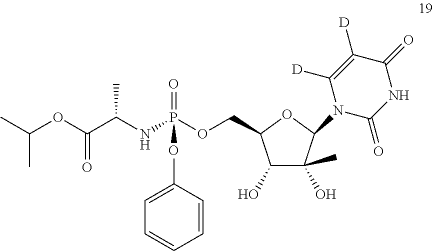 [SMILES: [2H]c1c([2H])n([C@@H]2O[C@H](CO[P@@](=O)(N[C@@H](C)C(=O)OC(C)C)Oc3ccccc3)[C@@H](O)[C@@]2(C)O)c(=O)[nH]c1=O]

Compound 19 was prepared using uracil-5,6-d2 in a manner analogous to that described for compound 17 in Example 14. 1H NMR (400 MHz, CD₃OD, 300 K): δ 1.15 (s, 3H), 1.21 (2×d, J=6.3 Hz, 6H), 1.35 (dd, J=7.2 Hz, JH, P=0.9 Hz, 3H), 3.79 (d, J=9.2 Hz, 1H), 3.91 (dq, JH, P=10.0 Hz, J=7.2 Hz, 1H), 4.09 (m, 1H), 4.37 (ddd, J=11.8 Hz, JH, P=5.9 Hz, J=3.7 Hz, 1H), 4.50 (ddd, J=11.8 Hz, JH, P=5.9 Hz, J=2.0 Hz, 1H), 4.96 (septet, J=6.3 Hz, 1H), 5.96 (s, 1H), 7.20 (m, 1H), 7.26 (m, 2H), 7.37 (m, 2H); 31P NMR (162 MHz, CD₃OD, 300 K): δ 3.8; LC-MS: 530 amu (M+1).

### Example 17

### Preparation of (S)-isopropyl 2-(((S)-(((2R,3R,4R,5R)-5-(5,6-dideutero-2,4-dioxo-3,4-dihydropyrimidin-1(2H)-yl)-3,4-dihydroxy-4-methyltetrahydrofuran-2-yl)dideuteromethoxy)(phenoxy)phosphoryl)amino)propanoate (Compound 22)

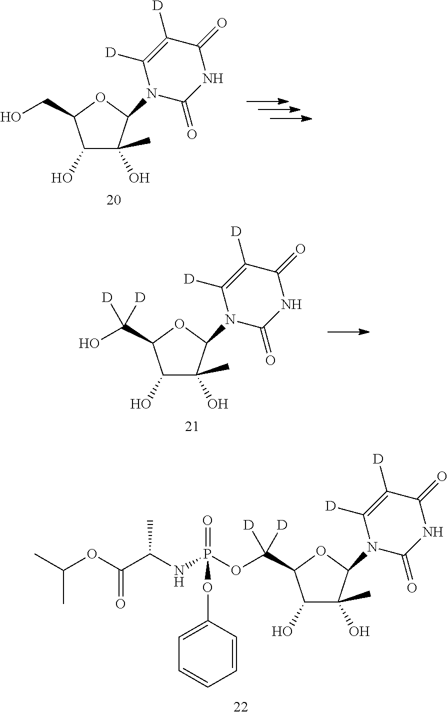 [SMILES: [2H]c1c([2H])n([C@@H]2O[C@H](CO)[C@@H](O)[C@@]2(C)O)c(=O)[nH]c1=O ; [2H]c1c([2H])n([C@@H]2O[C@H](C([2H])([2H])O)[C@@H](O)[C@@]2(C)O)c(=O)[nH]c1=O ; [2H]c1c([2H])n([C@@H]2O[C@H](C([2H])([2H])O[P@@](=O)(N[C@@H](C)C(=O)OC(C)C)Oc3ccccc3)[C@@H](O)[C@@]2(C)O)c(=O)[nH]c1=O]

Nucleoside 20 was prepared via uracil-5,6-d2 in a manner analogous to that described for compound 16 in Example 14. Nucleoside 20 was converted to deuterated nucleoside 21 in a manner analogous to that described for compound 8 in Examples 10 and 11. Spectroscopic data for 21: 1H NMR (400 MHz, CD₃OD, 300 K): δ 1.15 (s, 3H), 3.84 (d, J=9.2 Hz, 1H), 3.91 (d, J=9.2 Hz, 1H), 5.95 (s, 1H); 13C NMR (100 MHz, CD3OD, 300 K): δ 20.2, 73.4, 80.0, 83.8, 93.1, 152.5, 166.0 (ribose C-5′, uracil C-5, and uracil C-6 not observed). Nucleoside 21 was converted to the phosphoramidate derivative 22 in a manner analogous to that described in Example 11. Spectroscopic data for 22: 1H NMR (400 MHz, CD₃OD, 300 K): δ 1.15 (s, 3H), 1.21 (2×d, J=6.3 Hz, 6H), 1.35 (dd, J=7.2 Hz, JH, P=0.9 Hz, 3H), 3.79 (d, J=9.2 Hz, 1H), 3.91 (dq, JH, P=10.0 Hz, J=7.2 Hz, 1H), 4.08 (dd, J=9.2 Hz, JH, P=2.2 Hz, 1H), 4.96 (septet, J=6.3 Hz, 1H), 5.95 (s, 1H), 7.20 (m, 1H), 7.26 (m, 2H), 7.37 (m, 2H); 31P NMR (162 MHz, CD3OD, 300 K): δ 3.8; LC-MS: 532 amu (M+1).

### Example 18

### Preparation of (S)-isopropyl 2-(((S)-(((2R,3R,4R,5R)-5-(2,4-dioxo-3,4-dihydropyrimidin-1(2H)-yl)-3,4-dihydroxy-4-methyltetrahydrofuran-2-yl)deuteromethoxy)(phenoxy)phosphoryl)amino)propanoate (Formula III, Compound 26)

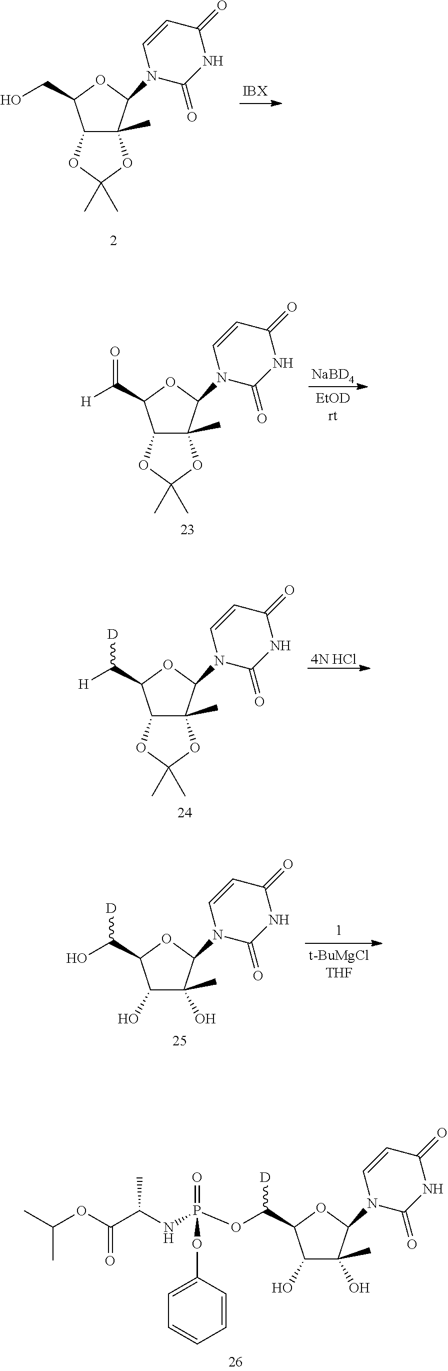

Commercially available 2-iodoxybenzoic acid (IBX, 3.54 g) is washed consecutively with acetonitirle (2×30 mL), acetone (2×20 mL), and Et₂O (10 mL), and then dried thoroughly in vacuo before use. A mixture of 2 (0.894 g) and washed IBX (2.52 g) in anhydrous acetonitrile (90 mL) is refluxed for 2 h. The mixture is cooled and filtered to remove solids. The filtrate is concentrated and treated with CH₂Cl₂. Solids are removed again by filtration and the filtrate is concentrated under reduced pressure to give 23 as a colorless foam.

Compound 23 (1.19 g) is dissolved in EtOD (15 mL), and into this turbid solution is added NaBD₄ (0.168 g) in portions at 0° C. with stirring. The reaction mixture is stirred at rt. for 2 h. and then cooled to 0° C. before adding a saturated aq. solution of NH₄Cl (1 mL) to quench the reaction. Brine (30 mL) is added and the mixture is extracted with EtOAc (5×30 mL). The combined organic extracts are died over anhydrous Na₂SO₄, filtered, evaporated to dryness under reduced pressure. The crude material is purified by column chromatography on silica gel (10% MeOH in CH₂Cl₂ as eluent) to give 24.

Compound 25 is prepared in a manner analogous to that described for compound 8 in Examples 10 and 11. 1H NMR (400 MHz, CD₃OD, 300 K): δ 1.16 (s, 6H), 3.76 (br d, 2.6 Hz, 1H), 3.84 (d, J=9.2 Hz, 2H), 3.92 (d of d, J=9.2 Hz, 2.4 Hz, 2H), 3.96 (br d, J=2.2 Hz, 1H), 5.67 (d, J=8.1 Hz, 2H), 5.96 (s, 2H), 8.14 (2×d, J=8.1 Hz, 2H); 13C NMR (100 MHz, CD₃OD, 300 K): δ 20.2, 60.2 (t, JH, D=21.3 Hz), 73.4, 80.0, 83.8, 93.1, 102.3, 142.5, 152.5, 166.0.

Phosphoramidate 26 is prepared in a manner analogous to that described for compound 10 in Example 11.

### Example 19

### Preparation of (S)-isopropyl 2-(((S)-(((2R,3R,4R,5R)-5-(5-deutero-2,4-dioxo-3,4-dihydropyrimidin-1(2H)-yl)-3,4-dihydroxy-4-methyltetrahydrofuran-2-yl)deuteromethoxy)(phenoxy)phosphoryl)amino)propanoate (Formula III, Compound 29)

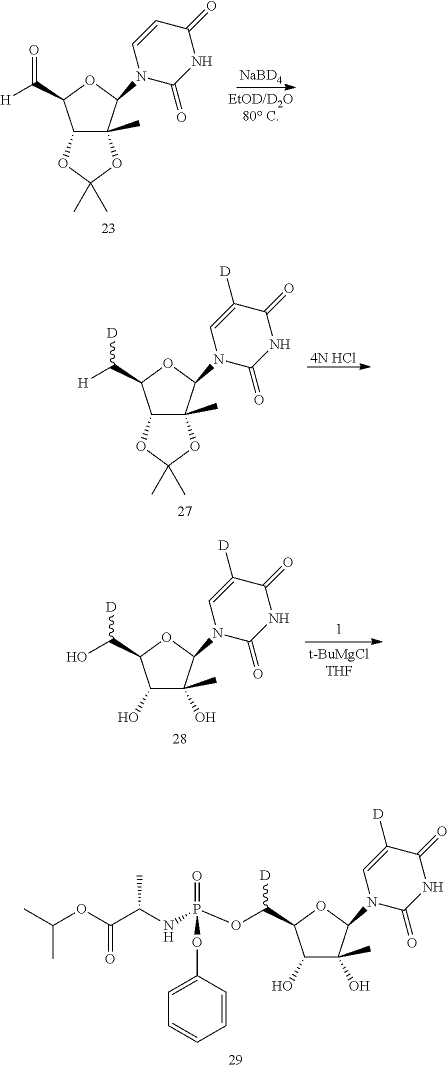

Compounds 27, 28, and 29 are prepared using methods analogous to those described in Example 11. Spectroscopic data for 29: 1H NMR (400 MHz, CD₃OD, 300 K): δ 1.16 (s, 6H), 3.76 (br d, 2.6 Hz, 1H), 3.84 (d, J=9.2 Hz, 2H), 3.92 (d of d, J=9.2 Hz, 2.4 Hz, 2H), 3.96 (br d, J=2.2 Hz, 1H), 5.96 (s, 2H), 8.14 (2×s, 2H); 13C NMR (100 MHz, CD₃OD, 300 K): δ 20.2, 60.2 (t, JH, D=21.4 Hz), 73.4, 80.0, 83.8, 93.1, 142.4, 152.5, 166.0 (uracil C-5 not observed).

### Example 20

### Preparation of (S)-isopropyl 2-(((S)-(((2R,3R,4R,5R)-5-(5-deutero-2,4-dioxo-3,4-dihydropyrimidin-1(2H)-yl)-3,4-dihydroxy-4-methyltetrahydrofuran-2-yl)dideuteromethoxy)(phenoxy)phosphoryl)amino)propanoate (Compound 31)

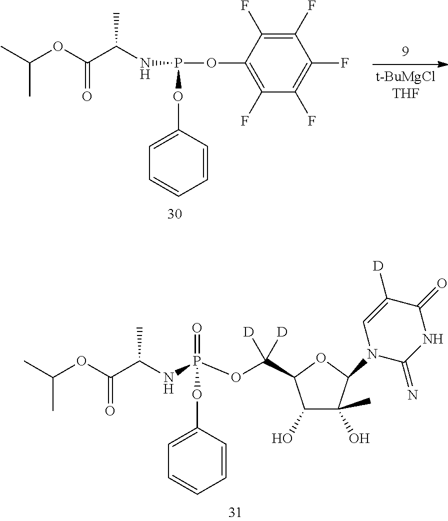 [SMILES: CC(C)OC(=O)[C@H](C)N[P@@](Oc1ccccc1)Oc1c(F)c(F)c(F)c(F)c1F ; [2H]c1cn([C@@H]2O[C@H](C([2H])([2H])O[P@](=O)(N[C@@H](C)C(=O)OC(C)C)Oc3ccccc3)[C@@H](O)[C@@]2(C)O)c(=N)[nH]c1=O]

The combined MTBE and EtOAc/hexanes washings from the synthesis of compound 1 in Example 9 are concentrated under reduce pressure to give a mixture of 30 and 1. Nucleoside 9 is treated with this mixture in a manner analogous to that described in Example 11 to give the phosphoramidate derivatives 31 and 10. Pure 31 is isolated by preparative HPLC. 1H NMR (400 MHz, CD₃OD, 300 K): δ 1.15 (s, 3H), 1.24 (d, J=6.3 Hz, 3H), 1.25 (d, J=6.3 Hz, 3H), 1.34 (dd, J=7.2 Hz, JH, P=1.0 Hz, 3H), 3.80 (d, J=9.2 Hz, 1H), 3.92 (dq, JH, P=9.0 Hz, J=7.2 Hz, 1H), 4.12 (dd, J=9.2 Hz, JH, P=2.7 Hz, 1H), 5.00 (septet, J=6.3 Hz, 1H), 5.99 (s, 1H), 7.22 (m, 1H), 7.26 (m, 2H), 7.39 (m, 2H), 7.72 (s, 1H); 31P NMR (162 MHz, CD₃OD, 300 K): δ 3.9; LC-MS: 531 amu (M+1).

### Example 21

### Synthesis of alpha-2′-fluoro-beta-2′-methyl-3′-deoxy-5′-deuterouridine phosphoramidate compound

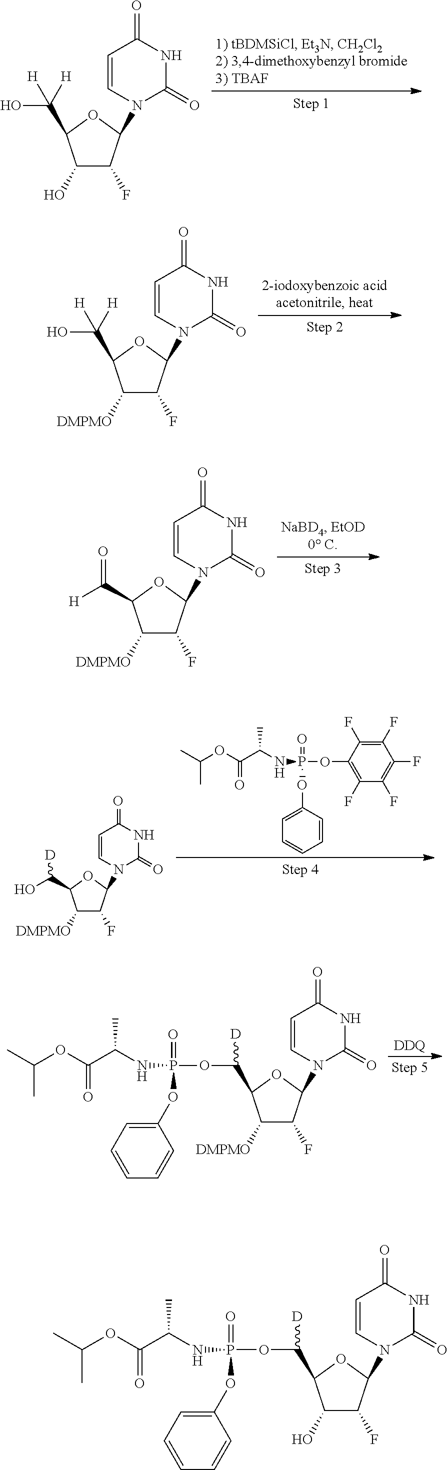

In step 1, the nucleoside is diluted with an organic solvent such as dichloromethane, an organic base such as triethylamine and tert-butyldimethylsilyl chloride. Once the reaction is determined to be complete by HPLC analysis, the reaction is diluted with brine. The organic layer is concentrated in vacuo and the product is purified by column chromatography. The silyl protected nucleoside is reacted with 3,4-dimethoxybenzyl bromide in the presence of boron trifluoride etherate in an organic solvent such as dichloromethane. The di-protected nucleoside is next treated with tetrabutylammonium fluoride in acetonitrile to afford the 5′-deprotected nucleoside. In step 2, the nucleoside is treated with 2-iodoxybenzoic acid in acetonitrile at an elevated temperature. In step 3, the aldehyde is treated with sodium borodeuteride in a deuterated protic solvent such as deuterated ethanol. After the reaction is quenched at a reduced temperature with an aqueous solution of ammonium chloride, the product is purified by column chromatography. In step 4, the nucleoside is coupled with the activated phosphate in the presence of t-butylmagnesium chloride in an organic solvent such as tetrahydrofuran according to the method of Ross et al., J. Org. Chem., 76, 8311 (2011). In step 5, the nucleoside is deprotected with DDQ in a mixture of solvents such as methanol and water.

### Example 22

### Synthesis of alpha-2′-fluoro-beta-2′-methyl-3′-deoxy-5′-deuterouridine phosphoramidate Compounds

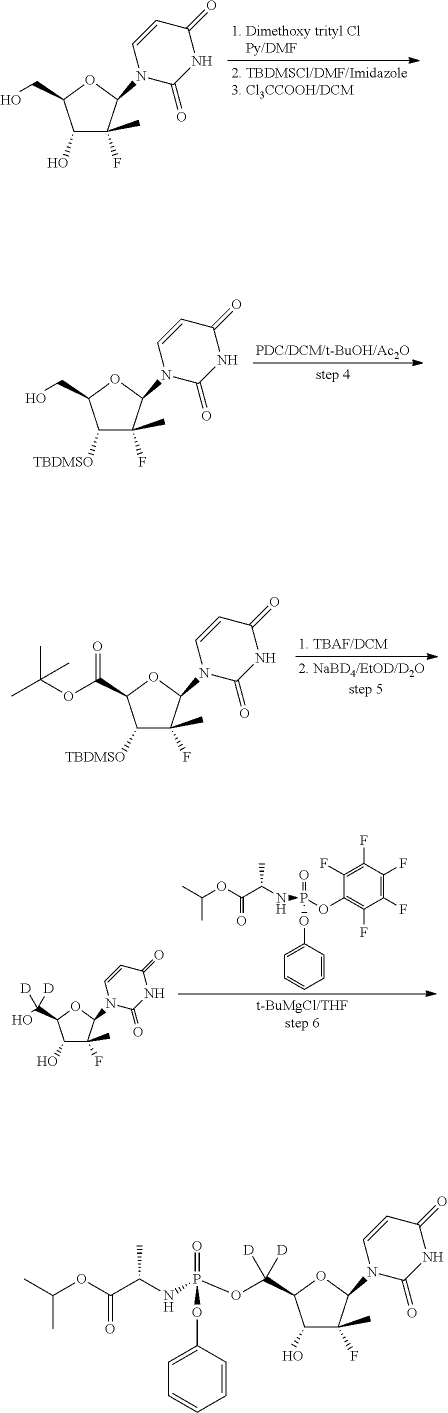 [SMILES: C[C@@]1(F)[C@H](O)[C@@H](CO)O[C@H]1n1ccc(=O)[nH]c1=O ; CC(C)(C)[Si](C)(C)O[C@@H]1[C@@H](CO)O[C@@H](n2ccc(=O)[nH]c2=O)[C@]1(C)F ; CC(C)(C)OC(=O)[C@H]1O[C@@H](n2ccc(=O)[nH]c2=O)[C@](C)(F)[C@@H]1O[Si](C)(C)C(C)(C)C ; [2H]C([2H])(O)[C@H]1O[C@@H](n2ccc(=O)[nH]c2=O)[C@](C)(F)[C@@H]1O ; CC(C)OC(=O)[C@H](C)N[P@](=O)(Oc1ccccc1)Oc1c(F)c(F)c(F)c(F)c1F ; [2H]C([2H])(O[P@@](=O)(N[C@@H](C)C(=O)OC(C)C)Oc1ccccc1)[C@H]1O[C@@H](n2ccc(=O)[nH]c2=O)[C@](C)(F)[C@@H]1O]

In step 1, the nucleoside is treated with dimethoxytrityl chloride in pyridine and DMF. Once the reaction is determined to be complete by HPLC analysis, the volatiles were removed and residue is diluted with aq. NaHCO₃ and extracted with DCM. The organic layer is concentrated in vacuo and the product (trityl protected nucleoside) is used as is in the next step. In step 2, the product (trityl protected nucleoside) is then reacted with TBDMS-chloride in DMF in presence of imidazole to give the silyl protected trityl nucleoside. In step 3, the trityl group is then removed by treating the product with trichloroacetic acid and methanol to give TBDMS-protected nucleoside after evaporation of all volatiles and purifying the product by chromatography over silica gel. In step 4 the TBDMS-protected nucleoside is treated with an oxidizing agent such as pyridinium dichromate in a mixture of organic solvents such as t-butanol and dichloromethane and an anhydride such as acetic anhydride. In step 5, the TBDMS protecting group is removed with TBAF (tetrabutylammonium fluoride) in DCM and the product is purified by chromatography and reduced with sodium borodeuteride in a combination of protic solvents such as D₂O and deuterated ethanol. In step 6, the nucleoside was treated with a phosphate diester to generate the product according to the method of Ross et al., J. Org. Chem., 76, 8311 (2011).

1H NMR (400 MHz, MeOH-d4, 300 K): δ 1.21 (d, 6H), 1.34 (2d, 6H), 3.92 (m, 1H), 4.10 (m, 1H), 4.96 (m, 1H), 5.62 (d, 1H), 6.13 (d, 1H), 7.20 (m, 1H), 7.26 (m, 2H), 7.37 (m, 2H), 7.61 (d, 1H); LC-MS: 532 amu (M+1).

### Example 23

### Synthesis of 5′-deuterated stabilized uridine bisamidates

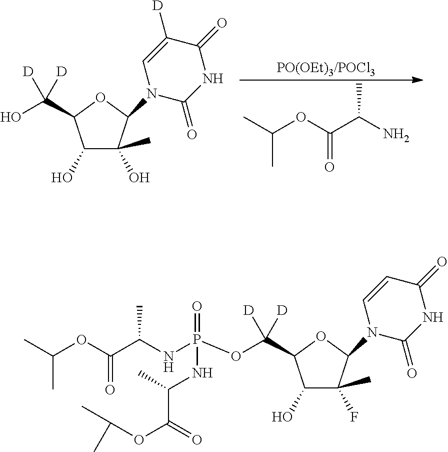

The compound 5-deutero-5′,5′-dideutero-2′-C-methyluridine from Example 11 is dissolved in PO(OEt)₃ and POCl₃ is added at 0° C. The reaction mixture is stirred for 30 min. L-Alanine isopropyl ester hydrochloride, PO(OEt)₃ and triethylamine in acetonitrile are added and the reaction is stirred at 0° C. for 30 min. The solvent is evaporated and the residue is purified by HPLC to afford the product.

### Example 24

### Synthesis of 5′-deuterated stabilized uridine mono-SATE amidate derivatives

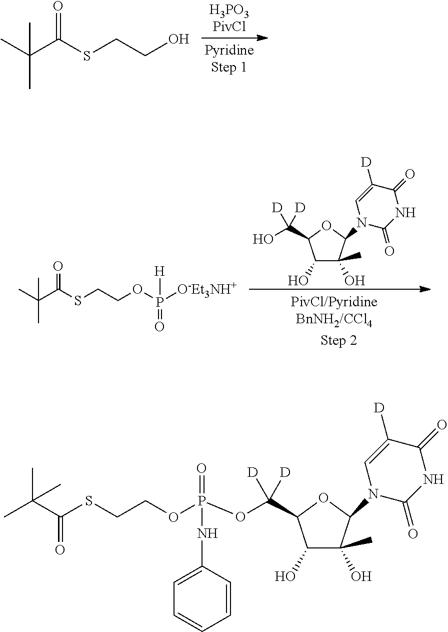

In step 1, phosphorous acid is dissolved in pyridine and added to hydroxyethyl 2,2-dimethylpropanethioate. The reaction mixture is stirred at room temperature and a white precipitate forms. The reaction is cooled to 0° C. and pivalyl chloride added. The reaction is then stirred at room temperature for 4 h and quenched with triethylammonium bicarbonate and extracted with ethyl acetate. The organic layer is concentrated in vacuo and the residue is purified by chromatography to give the intermediate H-phosphonate monoester. In step 2, the H-phosphonate and 5-deutero-5′,5′-dideutero-2′-C-methyluridine from Example 11 are dissolved in pyridine and pivalyl chloride is added at 0-5° C. After stirring for 2 h, the mixture is diluted with DCM and washed with a solution of ammonium chloride. The solvent is evaporated and the residue passed over silica gel and the product is then treated with benzylamine in carbon tetrachloride and stirred for 3 h. All volatiles are evaporated to dryness and residue purified by chromatography over silica gel to afford the product.

### Example 25

### Synthesis of 5′-deuterated stabilized uridine bis-(isopropoxycarbonyloxymethyl)phosphate and bis-(tert-butyloxycarbonyloxymethyl)phosphate derivatives

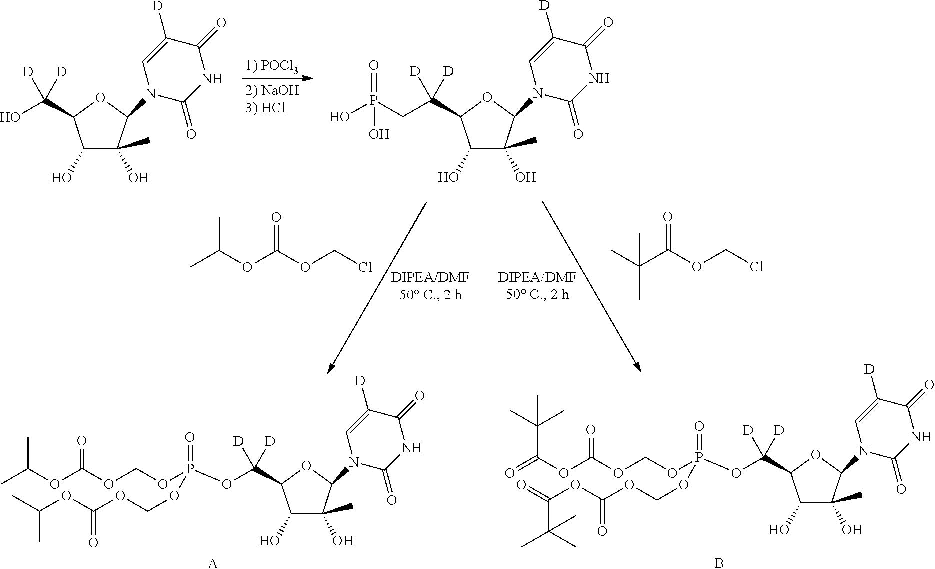

5-deutero-5′,5′-dideuterouridine is phosphorylated with phosphorus oxychlorde, quenched with aqueous sodium hydroxide and acidified. The nucleoside monophosphate is purified by HPLC. The nucleoside monophosphate is treated with isopropyl chloromethyl carbonate and diisopropylethylamine in DMF at 50° C. for 1 h and then stirred at room temperature for 24 h. The reaction mixture is diluted with water and extracted with DCM, dried and evaporated. The residue is purified by HPLC to give the bis-(isopropoxycarbonyloxymethyl)phosphate (A). Similarly the pivalate based prodrug is synthesized using chloromethyl pivalate (B).

### Example 26

### Synthesis of 5′-deuterated stabilized uridine phosphoramidate esters

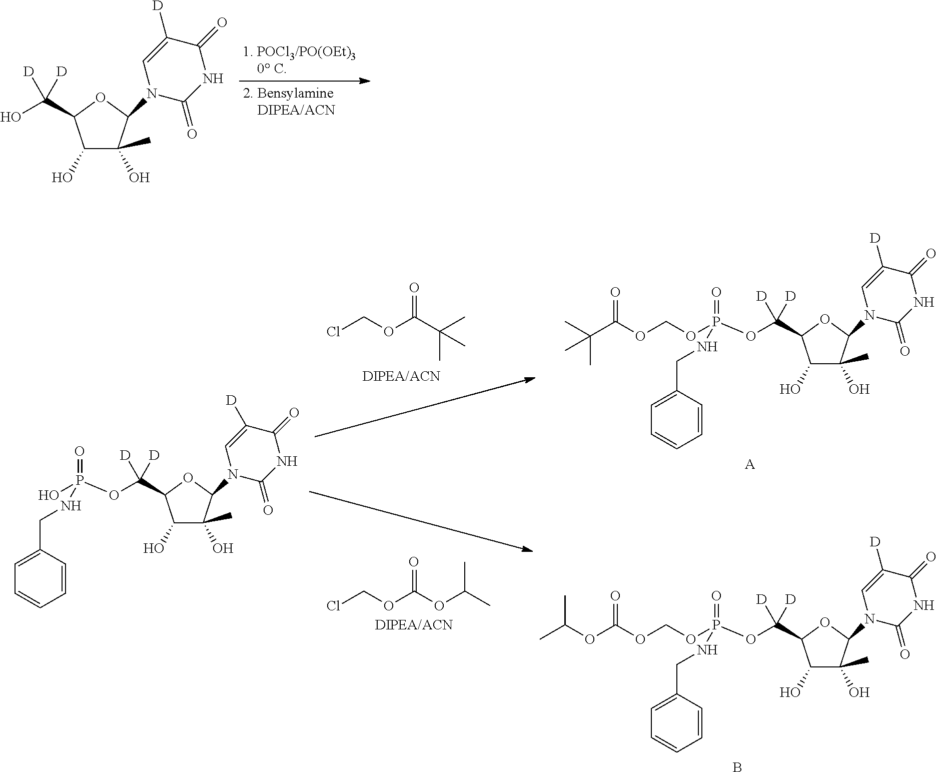

The compound 5′-deutero-5′,5′-dideuterouridine from Example 11 is dissolved in PO(OMe)₃ and POCl₃ is added at 0° C. The reaction mixture is stirred for 30 min. Benzylamine in acetonitrile is added followed by diisopropylethylamine and stirred for 30 min. The reaction is quenched with water and the product is isolated by HPLC. The phosphoramidate is treated with chloromethyl pivalate in DMF in the presence of diisopropylethylamine and stirred at 100° C. The reaction mixture is diluted with ethyl acetate and washed with water and the product is purified by HPLC to give compound A. Compound B is prepared similarly by using isopropylchloromethyl carbonate instead of chloromethyl pivalate.

### Example 27

### Synthesis of Ribofuranosyl Derivative

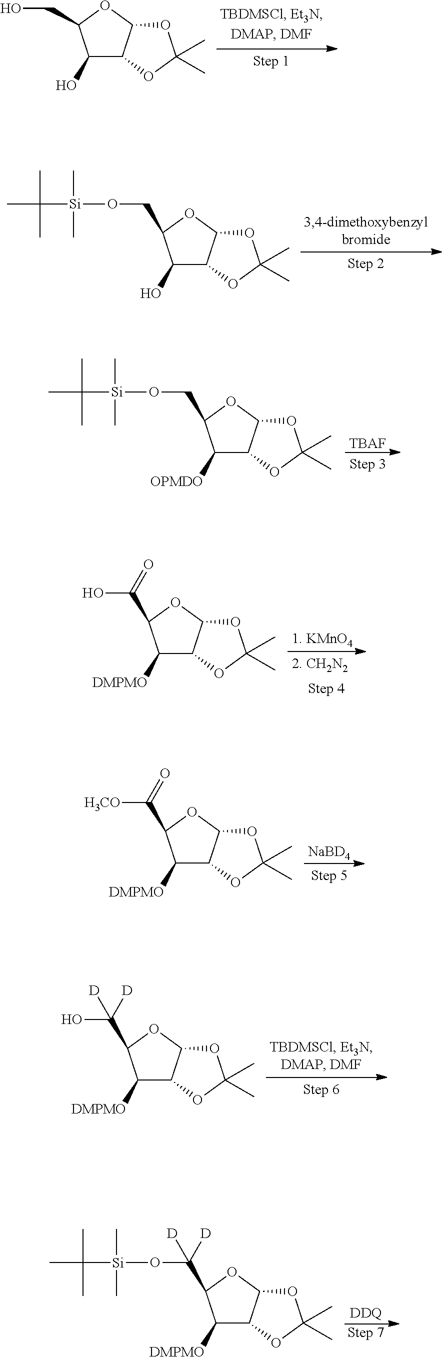

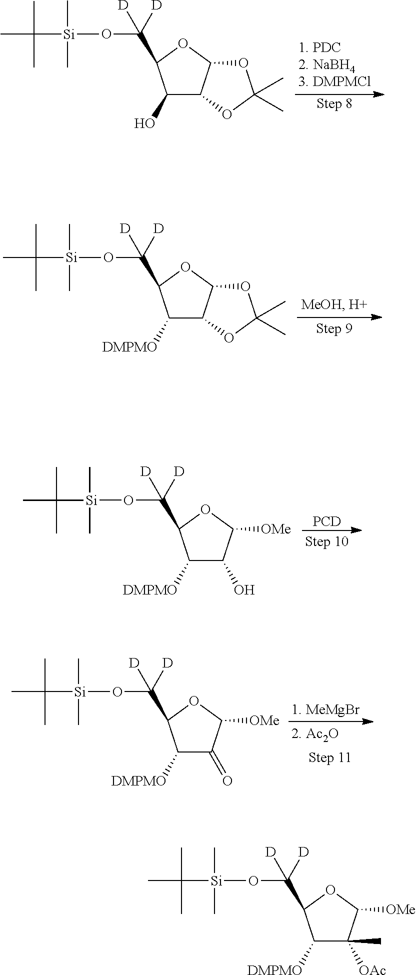

In step 1, 1,2-O-isopropylidene-α-D-xylofuranose is treated with tert-butyldimethylsilyl chloride in an organic solvent such as N,N-dimethylformamide (DMF), and organic bases such as triethylamine and 4-dimethylaminopyridine. In step 2, the alcohol is treated with 3,4-dimethoxybenzyl bromide in the presence of a base such as sodium hydride and an organic solvent such as DMF. In step 3, the furanose derivative is treated with tetra-n-butylammonium fluoride in an organic solvent such as acetonitrile. In step 4, the furanose derivative is treated with an oxidizing agent such as potassium permanganate. The carboxylic acid is treated with diazomethane to afford the methyl ester. In step 5, the methyl ester is treated with sodium borodeuteride in a protic solvent such as deuterated ethanol optionally at a lowered temperature to afford the deuterated alcohol. In step 6, the primary alcohol is treated with tert-butyldimethylsilyl chloride in an organic solvent such as (DMF), and organic bases such as triethylamine and 4-dimethylaminopyridine. In step 7, the 3,4-dimethoxybenzyl protecting group is removed by treating the xylose derivative with 2,3-dichloro-5,5-dicyano-1,4-benzoquinone. In step 8, the alcohol is treated with an oxidizing agent such as pyridinium dichromate (PDC) in an organic solvent such as dichloromethane and an anhydride such as acetic anhydride. The reaction can optionally be carried out at an elevated temperature. The ketone is treated with a reducing agent such as sodium borohydride in a protic solvent such as ethanol optionally at a lowered temperature. The alcohol is treated with 3,4-dimethoxybenzyl bromide in the presence of a base such as sodium hydride and an organic solvent such as DMF. In step 9, the acetonide is treated with an acid such as hydrochloric acid in a protic solvent to afford the 1-O-methyl compound. In step 10, the alcohol is treated with an oxidizing agent such as pyridinium dichromate (PDC) in an organic solvent such as dichloromethane and an anhydride such as acetic anhydride. The reaction can optionally be carried out at an elevated temperature. In step 11, the ketone is treated with methylmagnesium bromide to afford alcohol. The alcohol is treated with an anhydride such as acetic anhydride in an organic solvent such as pyridine to afford the acetate derivative.

### Example 28

### Coupling of ribose derivative to 5-deuterouracil

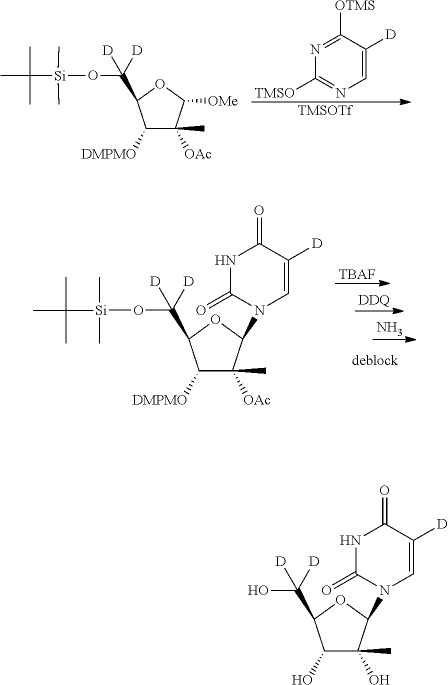

The furanose derivative from Example 27 is treated with 2,4-bis-O-trimethylsilyl-5-deuterouracil in an organic solvent such as acetonitrile in the presence of a catalyst such as trimethylsilyl triflate. The nucleoside is then deblocked. In the first step, the nucleoside is treated with tetrabutylammonium fluoride in an organic solvent such as acetonitrile. In the next step the nucleoside is treated with DDQ. In the final step the nucleoside is treated with 50% ammonia in a protic solvent such as methanol to afford the product.

### Example 29

### Coupling of Ribose Derivative to Uracil

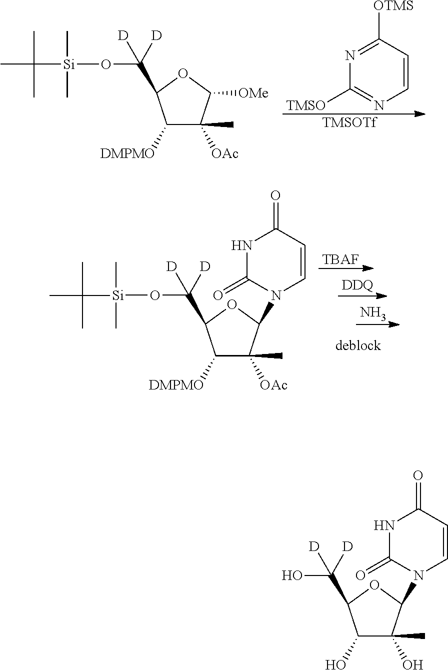

The furanose derivative from Example 27 is treated with 2,4-bis-O-trimethylsilyluracil in an organic solvent such as acetonitrile in the presence of a catalyst such as trimethylsilyl triflate.

The nucleoside is then deblocked. In the first step, the nucleoside is treated with tetrabutylammonium fluoride in an organic solvent such as acetonitrile. In the next step, the nucleoside is treated with DDQ. In the final step, the nucleoside is treated with 50% ammonia in a protic solvent such as methanol to afford the product.

### Example 30

### Synthesis of Phosphoramidate Furanose Compound and Coupling to 5′-Deuterouracil

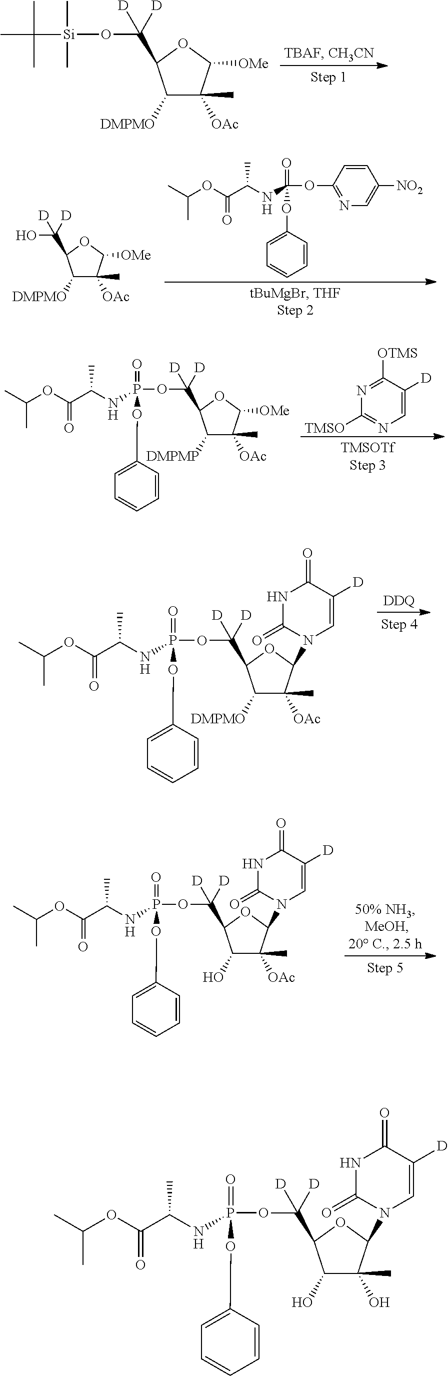

In step 1, the furanose derivative from Example 27 is treated with tetrabutylammonium fluoride in an organic solvent such as acetonitrile. In step 2, the furanose derivative is treated with the phosphoramidate reagent from Example 13, a Grignard reagent such as tert-butylmagnesium bromide in an organic solvent such as tetrahydrofuran optionally at a lowered temperature. In step 3, the phosphoramidate is treated with 2,4-bis-O-trimethylsilyl-5-deuterouracil in the presence of a catalyst such as trimethylsilyl triflate and an organic solvent such as acetonitrile optionally at an elevated temperature. In step 4, the nucleoside is treated with DDQ. In step 5, the nucleoside is treated with 50% NH₃ in a protic solvent such as methanol to afford the product.

### Example 31

### Synthesis of Phosphoramidate Furanose Compound and Coupling to Uracil

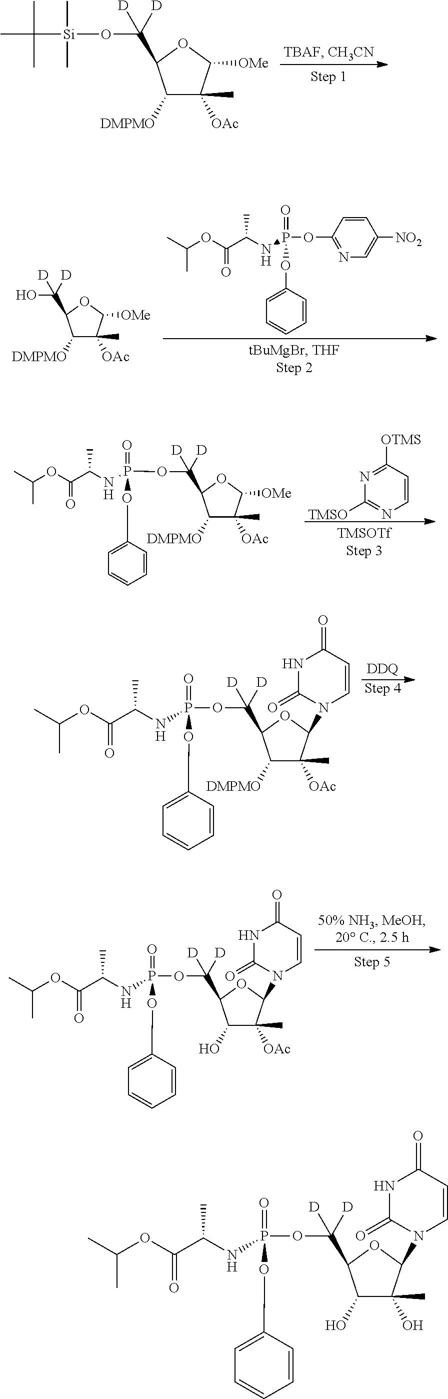

In step 1, the furanose derivative from Example 27 is treated with tetrabutylammonium fluoride in an organic solvent such as acetonitrile. In step 2, the furanose derivative is treated with the phosphoramidate reagent from Example 13, a Grignard reagent such as tert-butylmagnesium bromide in an organic solvent such as tetrahydrofuran optionally at a lowered temperature. In step 3, the phosphoramidate is treated with 2,4-bis-O-trimethylsilyluracil in the presence of a catalyst such as trimethylsilyl triflate and an organic solvent such as acetonitrile optionally at an elevated temperature. In step 4, the nucleoside is treated with DDQ. In step 5, the nucleoside is treated with 50% NH₃ in a protic solvent such as methanol to afford the product.

### Example 32

### Synthesis of Formula II Via an Acetal Protected Nucleoside

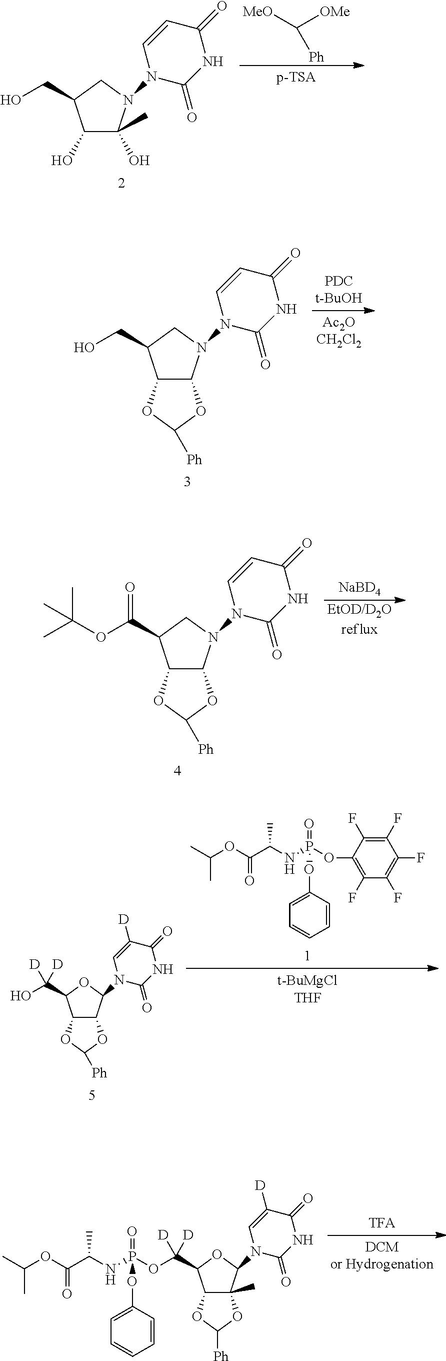

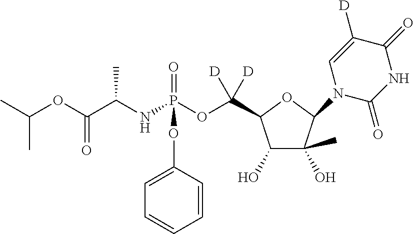 [SMILES: [2H]c1cn([C@@H]2O[C@H](C([2H])([2H])O[P@@](=O)(N[C@@H](C)C(=O)OC(C)C)Oc3ccccc3)[C@@H](O)[C@@]2(C)O)c(=O)[nH]c1=O]

The compound 2′-C-methyluridine is treated with an acetal such as benzaldehyde dimethyl acetal, and an acid catalyst such as p-toluenesulfonic acid to generate acetal 3. Compound 3 is oxidized with an oxidizing agent such as pyridinium dichromate in the presence of an alcohol such as tert-butanol, an anhydride such as acetic anhydride and an organic solvent such as dichloromethane. The ester is reduced with a reducing agent such as sodium borodeuteride in a combination of protic solvents such as EtOD and D₂O optionally at an elevated temperature. Compound 5 is treated with the phosphoramidate from Example 20, a Grignard reagent such as tert-butylmagnesium bromide in an organic solvent such as tetrahydrofuran optionally at a reduced temperature. The acetal is treated with an acid such as trifluoroacetic acid in an organic solvent such as dichloromethane to afford the product. The acetal can also be treated with a catalyst such as Pd(OH)₂ in an organic solvent such as cyclohexene to afford the product.

Phosphate prodrugs such as the compounds illustrated in Table 2, Table 3, Table 4, Table 5, and Table 6 can be prepared using the chemistry illustrated in Examples 1-32. It will be appreciated by those skilled in the art that protecting group modifications can be made to carry out the chemistry. For a review on protecting groups see, Green, T. W. and Wuts, P. G. M., Protective Groups in Organic Synthesis, 4^{th} edition, John Wiley & Sons, New York, 2006.

### Example 33

### Determination of Nucleoside Concentrations in Human Hepatocytes

For ease of reference, the following Formulas are referred to in this example:

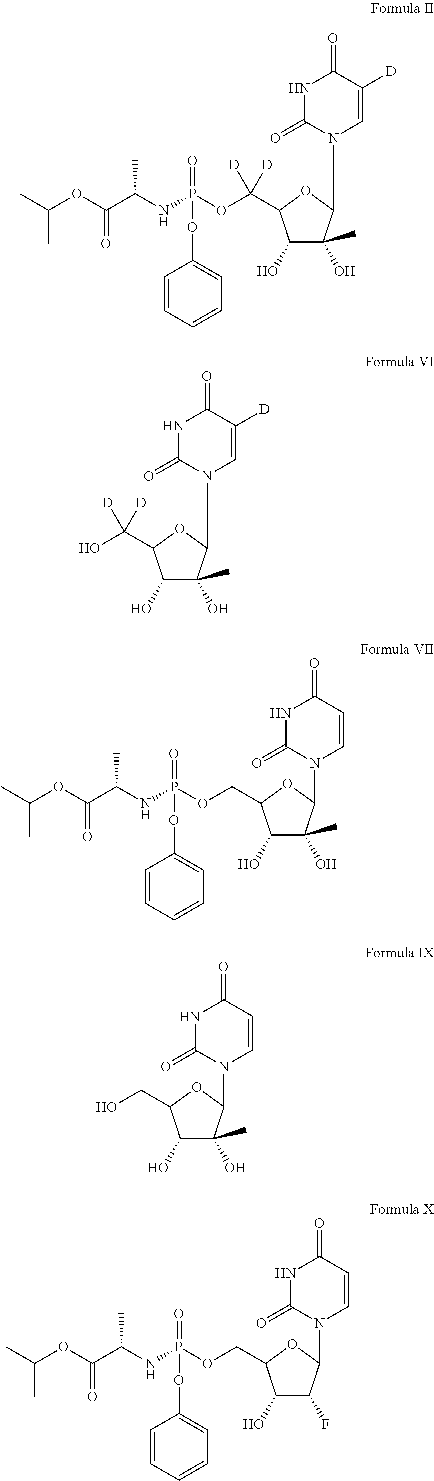 [SMILES: [2H]c1cn(C2OC(C([2H])([2H])O[P@@](=O)(N[C@@H](C)C(=O)OC(C)C)Oc3ccccc3)[C@@H](O)[C@@]2(C)O)c(=O)[nH]c1=O ; [2H]c1cn(C2OC(C([2H])([2H])O)[C@@H](O)[C@@]2(C)O)c(=O)[nH]c1=O ; CC(C)OC(=O)[C@H](C)N[P@](=O)(OCC1OC(n2ccc(=O)[nH]c2=O)[C@](C)(O)[C@@H]1O)Oc1ccccc1 ; C[C@]1(O)C(n2ccc(=O)[nH]c2=O)OC(CO)[C@H]1O ; CC(C)OC(=O)[C@H](C)N[P@](=O)(OCC1OC(n2ccc(=O)[nH]c2=O)[C@H](F)[C@@H]1O)Oc1ccccc1]

### Preparation of Hepatocyte Cells

Fresh liver hepatocytes were received plated in a 12-well or 6-well format (Life Technologies, Catalogue #HMFN12 and #HMNF06). Upon receipt, shipping media was removed immediately and replaced with 1 mL or 2 mL pre-warmed culture medium (Supplemented modified Chee's Media; Xenotech LLC, Catalogue# K2300) for 12- and 6-well formats, respectively. Cells were acclimated overnight at 37° C. with 5% CO₂ atmosphere. Media was aspirated from 12- and 6-well plates and replaced with 1 mL or 2 mL respectively of fresh media containing either 20 μM Formula II, 20 μM Formula VII, or solvent control (0.05% DMSO). Samples were incubated at 37° C., 5% CO₂ atmosphere, in duplicate for Formula II and in singlet for Formula VII in each well format. Stability of the compounds in the absence of cells was also assayed. At 24 hours, media was removed and frozen. Cells were washed twice with cold PBS. 70% cold Methanol (0.75 mL or 1.5 mL, for 12- and 6-well formats, respectively) containing Formula X as an internal standard was added to each well and cells were gently removed from the plate by scraping. The recovered cells suspended in the methanol solution were aspirated into a vial and frozen at −80° C.

### Extraction and LC-MS/MS Analysis of Hepatocyte Cells

Cell solutions were extracted overnight at −80° C. in 70% methanol, removed from the freezer, defrosted and vortexed. Tubes were centrifuged at 3000 rpm for 15 minutes at 4° C. Supernatants were removed and analyzed by LC-MS/MS. Six concentrations of Formula II, Formula VII, Formula VI or Formula IX were prepared by 3-fold serial dilution in DMSO. Aliquots of the compounds at the specified concentrations were spiked into 70% methanol containing internal standard. Two concentrations were also spiked into cell solutions from the experiment incubated in the absence of compound. Samples were frozen at −80° C. overnight, then defrosted and vortexed. Samples were centrifuged at 3000 rpm for 15 minutes. Supernatants were removed and analyzed by LC-MS/MS. The calibration concentrations were 5, 1.67, 0.556, 0.185, 0.0617 and 0.0206 μM. The analytes were quantified using linear regression of calibration standard values with instrument response. The acceptance criteria used and calibration standard concentrations was ±30% of nominal concentration. Calibration standards that did not meet the specified criteria were not used in the calibration curve. Sample values were accepted when at least 66% of the standard concentrations during the run were within 30% of nominal. The “r” value required for acceptance of the run was >0.98. Cell samples were analyzed without internal standard due to only 81% extraction efficiency of internal standard from cells while calibration was conducted without cells, this gave more accurate determination of concentrations.

### Extraction and LC-MS/MS Analysis of Hepatocyte Media

Hepatocyte media incubates were removed from the freezer, defrosted and vortexed. 2 parts hepatocyte media incubated to 1 part acetonitrile-containing internal standard were mixed and then centrifuged at 3000 rpm for 15 minutes at 4° C. Supernatants were removed and analyzed by LC-MS/MS. Controls were six concentrations of Formula II, Formula VII, Formula VI or Formula IX prepared by 3-fold serial dilution in DMSO. Aliquots of the compounds were spiked into fresh hepatocyte media to afford 5, 1.67, 0.556, 0.185, 0.0617 and 0.0206 μM concentrations of calibration media. 2 parts calibration media mixed with 1 part acetonitrile-containing internal standard samples were centrifuged at 3000 rpm for 15 minutes at 4° C. Supernatants were removed and analyzed by LC-MS/MS. Analyte concentrations in the samples were quantified using linear regression of calibration standard values with instrument response. The acceptance criteria used and calibration standard concentrations was ±30% of nominal concentration. Calibration standards that did not meet the specified criteria were not used in the calibration curve. Sample values were accepted when at least 66% of the standard concentrations during the run were within 30% of nominal. The “r” value required for acceptance of the run was >0.98.

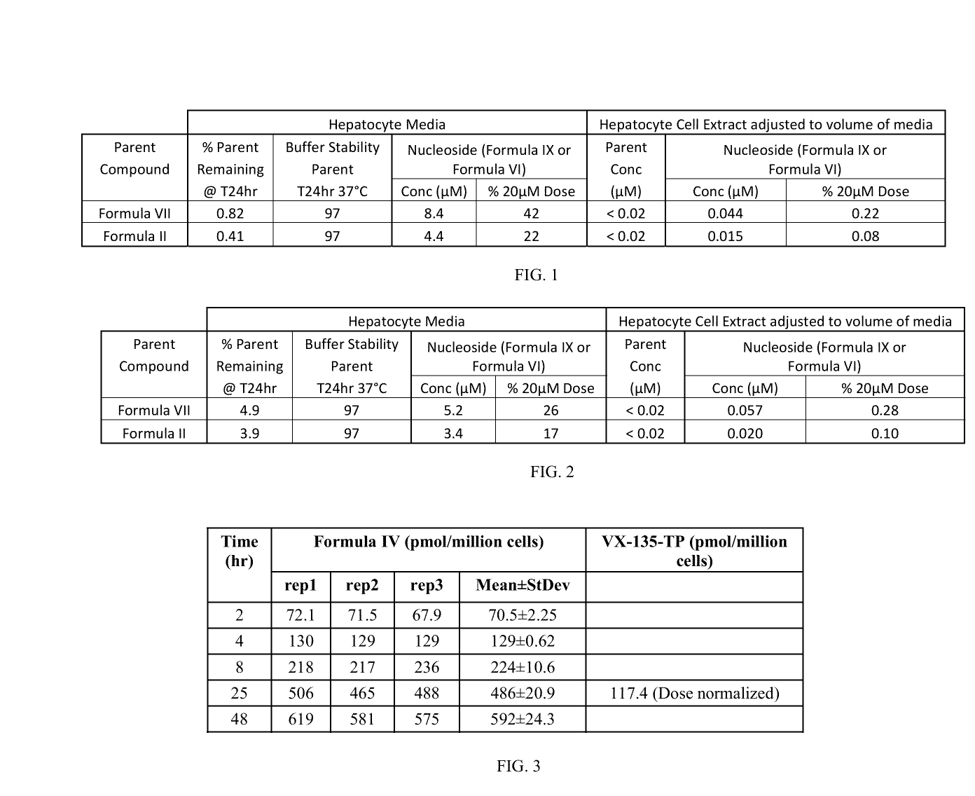

As the data shows in FIG. 1 and FIG. 2, there is more dephosphorylated nucleoside (i.e., undesired 5′-OH nucleoside) in the samples incubated with the undeuterated phosphoramidate than with the 5′-deuterated phosphoramidate. Specifically, using 20 μM Formula II or its undeuterated Formula VII counterpart (12 well plate (1 ml) with hepatocytes seeded at 0.67 million cells per well for 24 hours) results in a 1.9 fold (media, i.e., extracellular concentration) and 2.9 fold (cell extract, i.e., intracellular) higher concentration of undeuterated dephosphorylated 2′-methyl uridine (Formula IX) compared to that resulting from the 5′-deuterated form (Formula VI). Results of incubation of 20 μM Formula II or its undeuterated counterpart (6 well plate (2 ml) with hepatocytes seeded at 1.7 million cells per well for 24 hours) indicate a 1.5 fold (cell extract, i.e., intracellular) and 2.8 fold (cell extract, i.e., intracellular) higher concentration in higher concentration of undeuterated dephosphorylated 2′-methyl uridine (Formula IV) compared to that resulting from the 5′-deuterated form (Formula VI). Thus, on average, the hepatocyte nucleotidase activity leads to about twice as much 5′-OH-nucleoside produced when the 5′-position is not deuterated. This difference in 5′-monophosphate pool available for activation to the triphosphate when 5′-deuterated nucleoside derivative is used can have a significant effect on efficacy, dosage, toxicity and/or pharmacokinetics of the drug.

### Example 34

### Triphosphate Levels (Formula IV) in Comparison to Triphosphate Levels of VX-135-TP

The results of three experiments comparing the triphosphate levels of Formula IV to the level of VX-135 triphosphate are described in this Example. Human hepatocytes were used according to the general methods described in Example 33. The concentrations of Formula IV produced in human liver hepatocytes (pmol Formula IV/million cells) were determined at 2, 4, 8, 25, or 48 hours of incubation with 5 μM Formula II.

As a comparison to a clinical trial candidate as further described in FIG. 3, a poster presented by Alios (EASL 2013) indicates that the level of VX-135 triphosphate measured in human hepatocytes after 24 hours incubation with 50 μM VX-135 was 1174 pmol/million cells. In contrast, the level of Formula IV after 25 hours of incubation of human hepatocytes with 5 μM of Formula II, i.e., a ten times lower concentration, is 486 pmol/million cells. Therefore, the amount of triphosphate produced by incubation of Formula II is 4-fold greater (does-normalized) than the amount of triphosphate produced by VX-135. While the precise structure of VX-135 is not currently known, it is a uridine nucleotide analog prodrug NS5B inhibitor. Because the structure has not been disclosed, the inhibitory effect (IC₅₀) of the triphosphate produced by VX-135 on the RNA-dependent RNA polymerase (RdRp) activity of NS5B could not be compared with Formula IV.

### Example 35

### Determination of Formula IV Concentration from Formula II Dosing

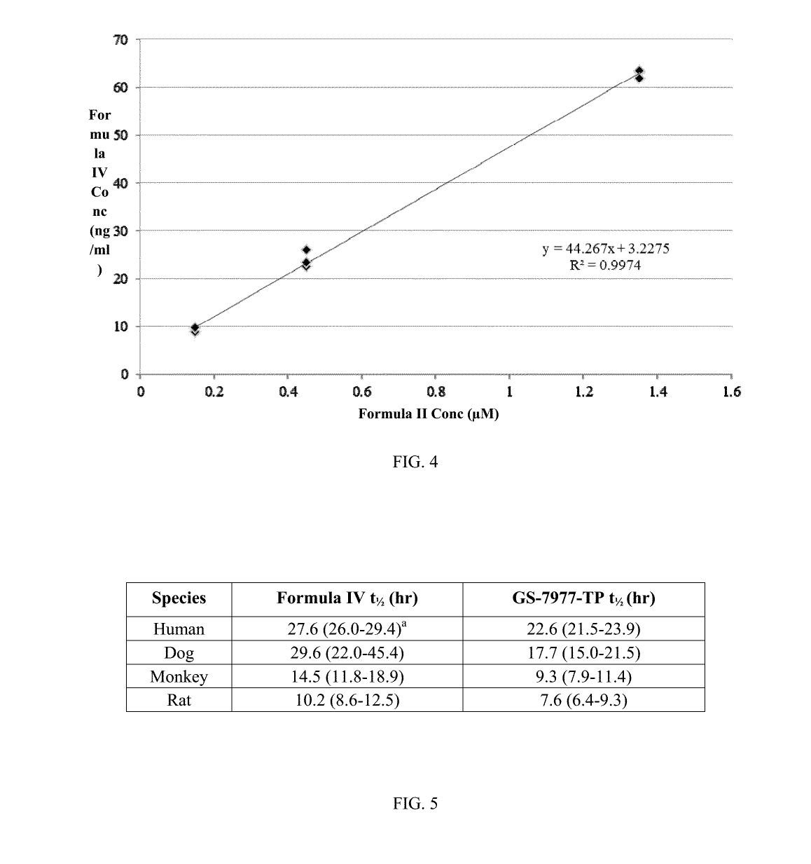

The relationship of the concentration of Formula IV (ng/ml) as a result of Formula II (μM) concentration in human liver hepatocytes was determined. The general methods of Example 33 were used to determine compound concentrations. The Formula IV concentrations in human hepatocytes were determined after 24 hour incubations with 0.15, 0.45, and 1.35 μM Formula II. The results were plotted and the linear regression was calculated using Microsoft Excel. As shown in FIG. 4, there is a linear relationship at the concentrations tested for dosing of Formula II, and the resulting concentration of the active triphosphate compound (Formula IV).

Additionally, after incubation of Formula II at 50 nM in primary hepatocytes for 24 hours, the level of Formula IV ranged from 9.2-16.2 pmol/million cells. These concentrations are 5- to 8-fold higher than that obtained when the Huh-luc/neo cells were incubated with 50 nM Formula II. Since Formula IV is the active species which inhibits HCV replicon replication in Huh-luc/neo cells, the predicted EC₅₀ of Formula II in primary human hepatocytes would be 6.25-10 nM (against a putative HCV in primary hepatocytes) presuming the linear relationship obtained in FIG. 4 between Formula II and Formula IV continues at lower concentration.

### Example 36

### Determination of the Half-Lives of Formula IV and GS-7977-TP

In this Example, the general methods of Example 33 were used to determine the half-lives of the active triphosphate (Formula IV or GS-7977-TP) in human, dog, monkey, and rat hepatocytes. Briefly, Formula II or GS-7977 (Sofosbuvir) were added at selected concentrations to hepatocytes (human, dog, monkey and rat) and incubated at 37° C. Supernatant cell extracts of Formula IV or GS-7977-TP (the active triphosphate metabolites) were measured by high performance liquid chromatography with tandem mass spectrometric detection (LC-MS/MS). Human hepatocyte cells used for half-life determinations were human liver hepatocyte 12-well format cells and were seeded at 0.67 million cells per well. Canine hepatocyte cells used for half-life determinations were beagle dog liver hepatocyte 12-well format cells and were seeded at 0.67 million cells per well. Monkey hepatocyte cells used for half-life determinations were Cynomolgus monkey liver hepatocyte 12-well format cells, and were seeded at 0.9 million cells per well. Rat hepatocyte cells for half-life determinations were Sprague-Dawley (SD) rat liver hepatocyte 12-well format cells, and were seeded at 0.67 million cells per well. All cells were obtained from Life Technologies.

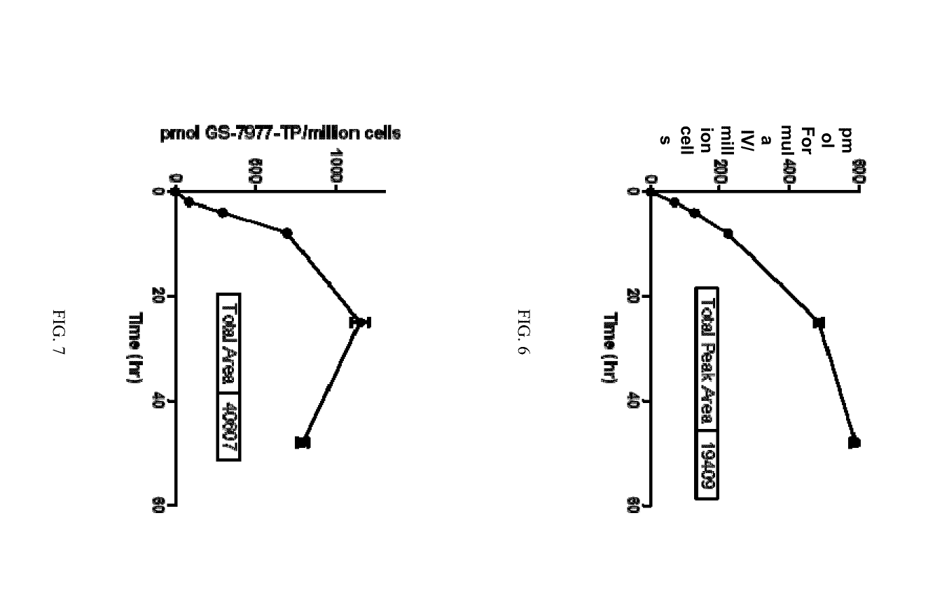

As shown in FIG. 5, the half-life of Formula IV is greater than the half-life of the triphosphate of Sofosbuvir in hepatocytes from all four species. The longest half-life was in human hepatocytes, followed by dog, then monkey and then rat. The half-lives range from 10-30 hours for Formula IV and 8-23 hours for the triphosphate of Sofosbuvir.

### Example 37

### Triphosphate Levels (Formula IV and GS-7977-TP) in Human Hepatocytes

In this Example, the triphosphate levels of Formula IV and GS-7977-TP were determined using the general methods as described in Example 33. Results for the three experiments determining the triphosphate levels of Formula IV as described in the Table shown in FIG. 3, are plotted graphically and shown here in FIG. 6. Levels of the corresponding triphosphate of Sofosbuvir (GS-79777) were also determined for comparison and are shown in FIG. 7. Briefly, the concentrations of Formula IV or GS-7977-TP produced in human liver hepatocytes (pmol/million cells) were determined at 2, 4, 8, 25, or 48 hours of incubation with 5 μM Formula II or GS7977 (Sofosbuvir), respectively.

Further, as described in FIGS. 6 and 7, over a 48 hour period, while the intracellular conversion to the corresponding triphosphate of Sofosbuvir (GS-7977) as measured in human hepatocytes is 2-fold greater than that of the triphosphate derived from Formula II (i.e., Formula IV), the concentration of Formula IV is still increasing at 48 hours, while the concentration of the triphosphate metabolite of Sofosbuvir decreases from 24 to 48 hours. The increasing concentration of Formula IV, combined with its half-life of >24 h, suggest accumulation of Formula IV (the triphosphate of Formula II) levels in hepatocytes on repeat dosing. This trend, after an initial in vivo initial dosing ramp up acclimation, can lead to a higher steady state concentration of Formula IV in vivo for the triphosphate derived from Formula II than the triphosphate of Sofosbuvir. In addition, the intrinsic potency (the inhibitory effect on the RdRp activity of NS5B) of Formula IV (the triphosphate of Formula II) is 1.5 fold better than the intrinsic potency of the triphosphate of Sofosbuvir (see Table 9 in Example 38), which may allow for a lower maintenance dosage regime over time.

### Example 38

### NS5B RNA Polymerase IC₅₀ Determination

The NS5B RNA polymerase reaction was monitored via incorporation of [α-³²P]-CTP into nascent RNA synthesized from a negative-strand RNA template derived from the HCV 5′ nontranslated region (NTR) and including the internal ribosomal entry site (IRES).

To generate the negative-strand IRES RNA template, duplex DNA (NTR bases 1-341) was amplified from the HCV pFK-I341PI-Luc/NS3-3′/ET plasmid using the primers

5′-NTR-1-21
(GCCAGCCCCCTGATGGGGGCGACACTCCAC)
and
T7-5NTR-341-317
(GAAATTAATACGACTCACTATAGGGGGTGC
ACGGTCTACGAGACCTCC,

T7 promoter sequence underlined). Negative-strand RNA was transcribed from this duplex DNA using T7 RNA polymerase (MEGAscript T7 Transcription Kit, Life Technologies). RNA was purified from reaction components (MEGAclear, Life Technologies) and its yield and purity assessed by agarose gel electrophoresis and optical absorption.

NS5B RNA polymerase reactions for IC₅₀ determination were performed in 96-well microtiter plates in 20 μL reactions containing assay buffer (50 mM Na⁺ HEPES, 1 mM MgCl₂, 0.75 mM MnCl₂, 2 mM DTT, pH 7.5), 1 U/μL SUPERase•In (Life Technologies), 20 ng/μL IRES RNA template, 1 μM each ATP, CTP, GTP, and UTP (Life Technologies) including [α-³²P]-CTP at a final specific activity of 50 Ci/mmol (PerkinElmer), test compounds in 10-point half-log dilution series, and NS5B polymerase. Reactions were incubated at 27° C. for 60 minutes and terminated by dilution to 100 μL in 1× stop solution (final concentrations 144 mM Na⁺ citrate, 1.44 M NaCl, 10 mM EDTA, pH 7.0). 80 μL stopped samples were applied by vacuum to nylon membrane filtermats (PerkinElmer), washed 4× in 60 mM Na⁺ citrate, 600 mM NaCl (pH 7.0), rinsed sequentially in H₂O and EtOH, dried, and counted in a MicroBeta2 counter with scintillation cassette (PerkinElmer). NS5B polymerase activity was shown in parallel reactions to be within linear range. Compound activity was expressed as the concentration that reduced radiolabel incorporation by 50% (IC₅₀) as determined by sigmoidal curve-fitting using non-linear regression analysis (Prism Software, GraphPad, La Jolla, Calif.).

The inhibitory activity of nucleoside triphosphate compounds (Formula IV and GS-7977-triphosphate) against wild-type NS5B polymerase are shown in Table 9. The IC₅₀ values are presents as mean±standard deviation from N independent experiments for compounds against wild-type (WT) NS5B polymerase.

TABLE 9

| | GT-1b WT a | |
|---|---|---|
| Compound | IC₅₀ (μM) | N |
| Formula IV | 1.4 ± 0.1 | 4 |
| GS-7977-TP | 2.1 ± 0.3 | 4 |

This specification has been described with reference to embodiments, which are illustrated by the accompanying Examples. The invention can, however, be embodied in different forms and should not be construed as limited to the embodiments set forth herein. Given the teaching herein, one of ordinary skill in the art will be able to modify the invention for a desired purpose and such variations are considered within the scope of the disclosure.

We claim:

1. A compound selected from:
5′-Deuterated Stabilized Uridine Phosphate Structures 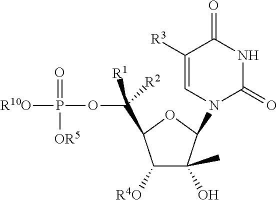 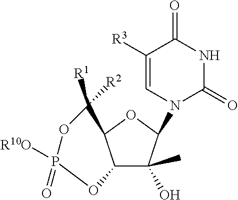 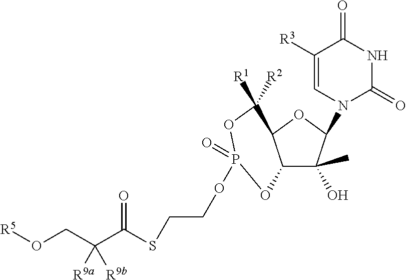 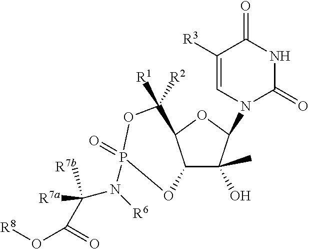 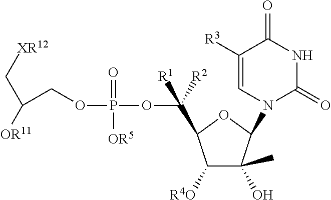 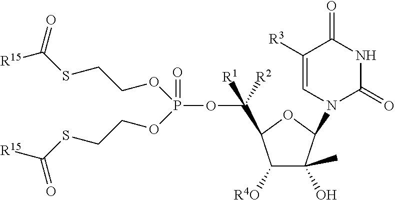 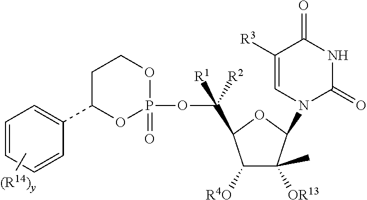 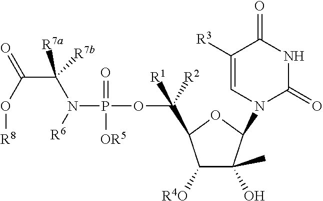 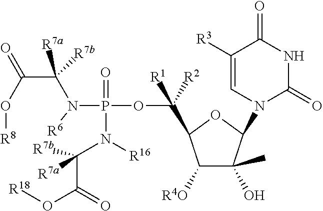 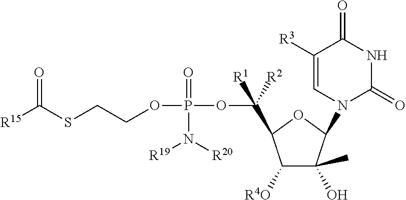 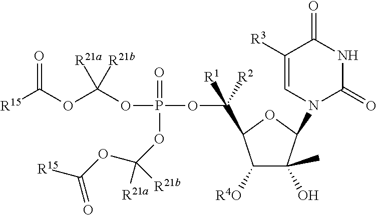 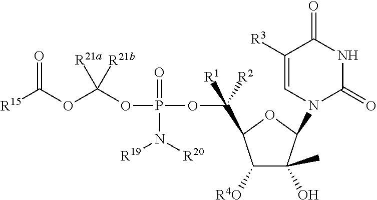
wherein:
R¹ and R² are independently deuterium, hydrogen, or C(H)ₘ(D)ₙ; and at least one of R¹ or R² is deuterium;
R³ is hydrogen, deuterium, halogen (F, Cl, Br, or I), C(H)ₘ(D)ₙ; or alkyne; wherein the R³ alkyne and the C⁴-oxygen of the pyrimidine can form a heterocyclic ring;
R⁴ is hydrogen, deuterium, C(H)ₘ(D)ₙ, acyl or phosphate;
R⁵ is hydrogen, deuterium, alkyl, cycloalkyl, aryl, heteroaryl, heterocyclic, -alkyl(heterocycle), -alkyl(heteroaryl), or -alkylaryl;
R⁶ is hydrogen, deuterium, C₁₋₃alkyl or C₃₋₅cycloalkyl;
R^{7a} and R^{7b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{7a} and R^{7b} form an exo-double bond; or
R^{7a} and R^{7b} form a cycloalkyl or heterocyclic group; or
R⁶ and R^{7a} or R^{7b} form a cycloalkyl group;
R⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R^{9a} and R^{9b} are each independently hydrogen, deuterium, C₁-C₃alkyl, C₃-C₄ cycloalkyl; or
R^{9a} and R^{9b} form a C₃-C₅cycloalkyl group;
R¹⁰ is hydrogen, deuterium, alkyl, cycloalkyl, aryl, heteroaryl, heterocyclic, -alkyl(heterocycle), -alkyl(heteroaryl or -alkylaryl;
R¹¹ is C₁-C₂₂alkyl, cycloalkyl, C₃-C₂₂alkenyl or C₃-C₂₂alkynyl or R¹¹ is —C(O)—C₆-C₂₂alkyl, —C(O)—C₆-C₂₂alkenyl or —C(O)—C₆-C₂₂alkynyl;
R¹² is C₁-C₂₂alkyl, C₃-C₂₂alkenyl or C₃-C₂₂alkynyl or R¹² is —C(O)—C₆-C₂₂alkyl, —C(O)—C₆-C₂₂alkenyl or —C(O)—C₆-C₂₂alkynyl;
R¹³ is hydrogen, deuterium, C(H)ₘ(D)ₙ, acyl or phosphate;
R¹⁴ is independently deuterium, halogen, C₁-C₂haloalkyl, C₁-C₂haloalkoxy, C₁-C₆alkyl, cycloalkyl, allenyl, C₂-C₆alkenyl, C₂-C₆alkynyl, C₁-C₆alkoxy, (C₃-C₆cycloalkyl)C₀-C₄alkyl-, or (aryl)C₀-C₂alkyl-;
R¹⁵ is C₁-C₆alkyl, C₃-C₆ cycloalkyl, —C₁-C₃alkyl-O—C₁-C₅alkoxy, or —C(R^{9a})(R^{9b})CH₂OR⁵;
R¹⁶ is hydrogen, deuterium, C₁₋₃alkyl or C₃₋₅cycloalkyl;
R^{17a} and R^{17b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{17a} and R^{17b} form an exo-double bond; or
R^{17a} and R^{17b} form a cycloalkyl or heterocyclic group; or
R¹⁶ and R^{17a} or R^{17b} form a cycloalkyl group;
R¹⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R¹⁹ and R²⁰ are independently hydrogen, deuterium, alkyl, cycloalkyl, aryl, heteroaryl, heterocyclic, -alkylaryl or a suitable side chain of an amino acid ester; or
R¹⁹ and R²⁰ form a cycloalkyl or heterocyclic group;
R^{21a} and R^{21b} are independently hydrogen, deuterium, C₁₋₆alkyl, C₃-C₆ cycloalkyl; or
R^{21a} and R^{21b} form a 3 to 6 membered ring;
m is 0, 1, 2, or 3;
n is 0, 1, 2, or 3;
m+n=3;
X is S or O;
y is 0, 1, 2, 3, 4, or 5; and
wherein each deuterium is at least 90% enriched;
or a pharmaceutically acceptable salt thereof.

2. A compound selected from:
5′-Deuterated Stabilized Cytidine Phosphate Structures 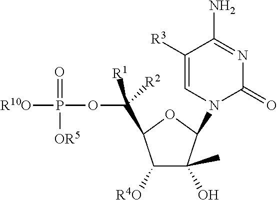 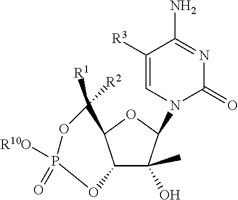 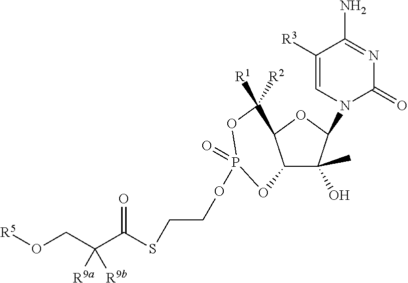 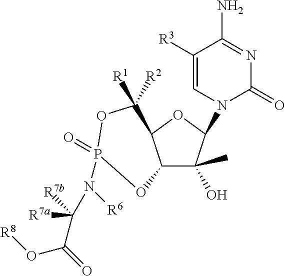 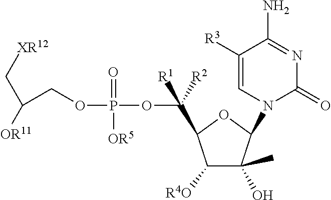 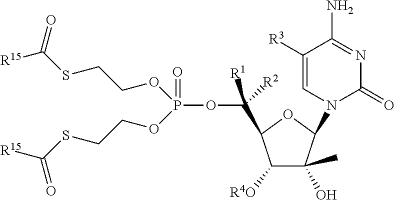 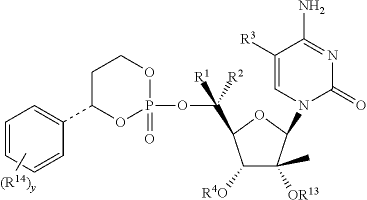 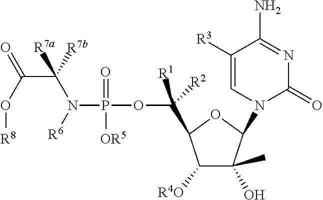 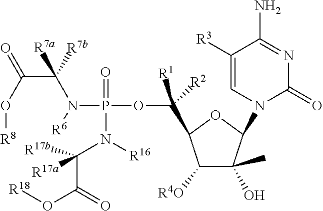 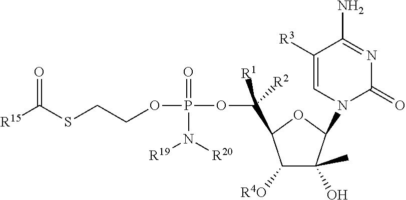 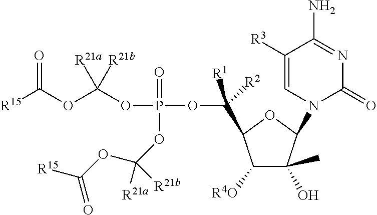 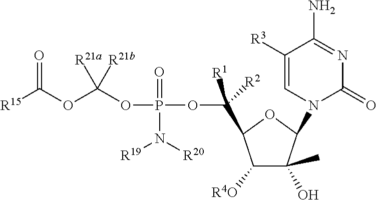
wherein:
R¹ and R² are independently deuterium, hydrogen, or C(H)ₘ(D)ₙ; and at least one of R¹ or R² is deuterium;
R³ is hydrogen, deuterium, halogen (F, Cl, Br, or I), C(H)ₘ(D)ₙ; or alkyne; wherein the R³ alkyne and the C⁴-oxygen of the pyrimidine can form a heterocyclic ring;
R⁴ is hydrogen, deuterium, C(H)ₘ(D)ₙ, acyl or phosphate;
R⁵ is hydrogen, deuterium, alkyl, cycloalkyl, aryl, heteroaryl, heterocyclic, -alkyl(heterocycle), -alkyl(heteroaryl), or -alkylaryl;
R⁶ is hydrogen, deuterium, C₁₋₃alkyl or C₃₋₅cycloalkyl;
R^{7a} and R^{7b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{7a} and R^{7b} form an exo-double bond; or
R^{7a} and R^{7b} form a cycloalkyl or heterocyclic group; or
R⁶ and R^{7a} or R^{7b} form a cycloalkyl group;
R⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R^{9a} and R^{9b} are each independently hydrogen, deuterium, C₁-C₃alkyl, C₃-C₄ cycloalkyl; or
R^{9a} and R^{9b} form a C₃-C₅cycloalkyl group;
R¹⁰ is hydrogen, deuterium, alkyl, cycloalkyl, aryl, heteroaryl, heterocyclic, -alkyl(heterocycle), -alkyl(heteroaryl or -alkylaryl;
R¹¹ is C₁-C₂₂alkyl, cycloalkyl, C₃-C₂₂alkenyl or C₃-C₂₂alkynyl or R¹¹ is —C(O)—C₆-C₂₂alkyl, —C(O)—C₆-C₂₂alkenyl or —C(O)—C₆-C₂₂alkynyl;
R¹² is C₁-C₂₂alkyl, C₃-C₂₂alkenyl or C₃-C₂₂alkynyl or R¹² is —C(O)—C₆-C₂₂alkyl, —C(O)—C₆-C₂₂alkenyl or —C(O)—C₆-C₂₂alkynyl;
R¹³ is hydrogen, deuterium, C(H)ₘ(D)ₙ, acyl or phosphate;
R¹⁴ is independently deuterium, halogen, C₁-C₂haloalkyl, C₁-C₂haloalkoxy, C₁-C₆alkyl, cycloalkyl, allenyl, C₂-C₆alkenyl, C₂-C₆alkynyl, C₁-C₆alkoxy, (C₃-C₆cycloalkyl)C₀-C₄alkyl-, or (aryl)C₀-C₂alkyl-;
R¹⁵ is C₁-C₆alkyl, C₃-C₆ cycloalkyl, —C₁-C₃alkyl-O—C₁-C₅alkoxy, or —C(R^{9a})(R^{9b})CH₂OR⁵;
R¹⁶ is hydrogen, deuterium, C₁₋₃alkyl or C₃₋₅cycloalkyl;
R^{17a} and R^{17b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{17a} and R^{17b} form an exo-double bond; or
R^{17a} and R^{17b} form a cycloalkyl or heterocyclic group; or
R¹⁶ and R^{17a} or R^{17b} form a cycloalkyl group;
R¹⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R¹⁹ and R²⁰ are independently hydrogen, deuterium, alkyl, cycloalkyl, aryl, heteroaryl, heterocyclic, -alkylaryl or a suitable side chain of an amino acid ester; or
R¹⁹ and R²⁰ form a cycloalkyl or heterocyclic group;
R^{21a} and R^{21b} are independently hydrogen, deuterium, C₁₋₆alkyl, C₃-C₆ cycloalkyl; or
R^{21a} and R^{21b} form a 3 to 6 membered ring;
m is 0, 1, 2, or 3;
n is 0, 1, 2, or 3;
m+n=3;
X is S or O;
y is 0, 1, 2, 3, 4, or 5; and
wherein each deuterium is at least 90% enriched;
or a pharmaceutically acceptable salt thereof.

3. A compound selected from:
5′-Deuterated Stabilized Uridine Phosphate Structures 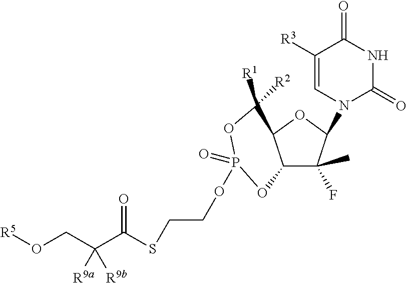 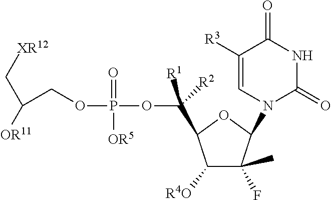 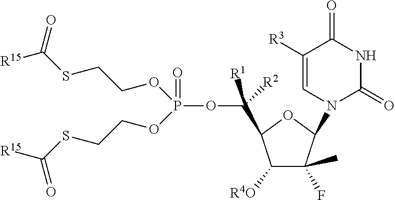 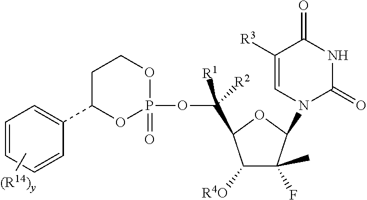 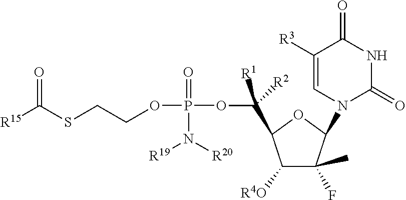 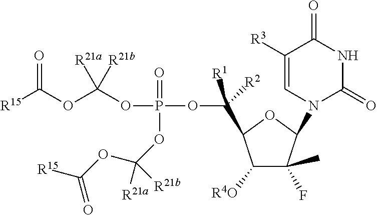 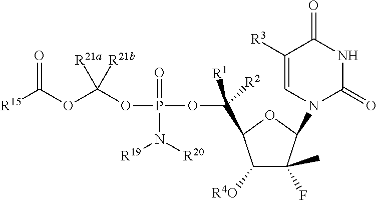
wherein:
R¹ and R² are independently deuterium, hydrogen, or C(H)ₘ(D)ₙ; and at least one of R¹ or R² is deuterium;
R³ is hydrogen, deuterium, halogen (F, Cl, Br, or I), C(H)ₘ(D)ₙ; or alkyne; wherein the R³ alkyne and the C⁴-oxygen of the pyrimidine can form a heterocyclic ring;
R⁴ is hydrogen, deuterium, C(H)ₘ(D)ₙ, acyl or phosphate;
R⁵ is hydrogen, deuterium, alkyl, cycloalkyl, aryl, heteroaryl, heterocyclic, -alkyl(heterocycle), -alkyl(heteroaryl), or -alkylaryl;
R⁶ is hydrogen, deuterium, C₁₋₃alkyl or C₃₋₅cycloalkyl;
R^{7a} and R^{7b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{7a} and R^{7b} form an exo-double bond; or
R^{7a} and R^{7b} form a cycloalkyl or heterocyclic group; or
R⁶ and R^{7a} or R^{7b} form a cycloalkyl group;
R⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R^{9a} and R^{9b} are each independently hydrogen, deuterium, C₁-C₃alkyl, C₃-C₄ cycloalkyl; or
R^{9a} and R^{9b} form a C₃-C₅cycloalkyl group;
R¹⁰ is hydrogen, deuterium, alkyl, cycloalkyl, aryl, heteroaryl, heterocyclic, -alkyl(heterocycle), -alkyl(heteroaryl or -alkylaryl;
R¹¹ is C₁-C₂₂alkyl, cycloalkyl, C₃-C₂₂alkenyl or C₃-C₂₂alkynyl or R¹¹ is —C(O)—C₆-C₂₂alkyl, —C(O)—C₆-C₂₂alkenyl or —C(O)—C₆-C₂₂alkynyl;
R¹² is C₁-C₂₂alkyl, C₃-C₂₂alkenyl or C₃-C₂₂alkynyl or R¹² is —C(O)—C₆-C₂₂alkyl, —C(O)—C₆-C₂₂alkenyl or —C(O)—C₆-C₂₂alkynyl;
R¹³ is hydrogen, deuterium, C(H)ₘ(D)ₙ, acyl or phosphate;
R¹⁴ is independently deuterium, halogen, C₁-C₂haloalkyl, C₁-C₂haloalkoxy, C₁-C₆alkyl, cycloalkyl, allenyl, C₂-C₆alkenyl, C₂-C₆alkynyl, C₁-C₆alkoxy, (C₃-C₆cycloalkyl)C₀-C₄alkyl-, or (aryl)C₀-C₂alkyl-;
R¹⁵ is C₁-C₆alkyl, C₃-C₆ cycloalkyl, —C₁-C₃alkyl-O—C₁-C₅alkoxy, or —C(R^{9a})(R^{9b})CH₂OR⁵;
R¹⁶ is hydrogen, deuterium, C₁₋₃alkyl or C₃₋₅cycloalkyl;
R^{17a} and R^{17b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{17a} and R^{17b} form an exo-double bond; or
R^{17a} and R^{17b} form a cycloalkyl or heterocyclic group; or
R¹⁶ and R^{17a} or R^{17b} form a cycloalkyl group;
R¹⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R¹⁹ and R²⁰ are independently hydrogen, deuterium, alkyl, cycloalkyl, aryl, heteroaryl, heterocyclic, -alkylaryl or a suitable side chain of an amino acid ester; or
R¹⁹ and R²⁰ form a cycloalkyl or heterocyclic group;
R^{21a} and R^{21b} are independently hydrogen, deuterium, C₁₋₆alkyl, C₃-C₆ cycloalkyl; or
R^{21a} and R^{21b} form a 3 to 6 membered ring;
m is 0, 1, 2, or 3;
n is 0, 1, 2, or 3;
m+n=3;
X is S or O;
y is 0, 1, 2, 3, 4, or 5; and
wherein each deuterium is at least 90% enriched;
or a pharmaceutically acceptable salt thereof.

4. A compound selected from:
5′-Deuterated Ribavirin Structures 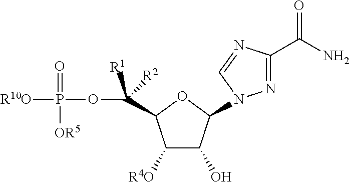 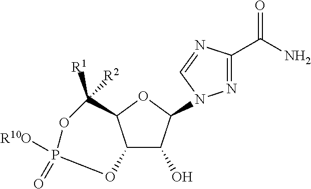 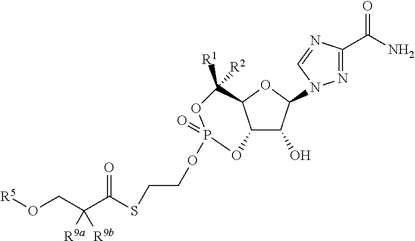 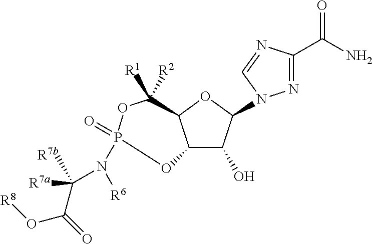 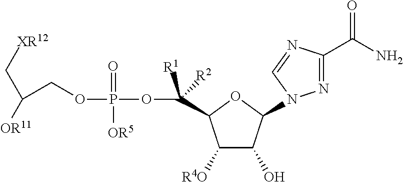 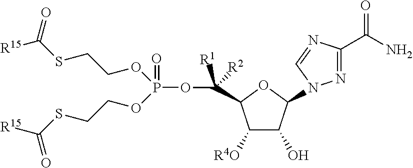 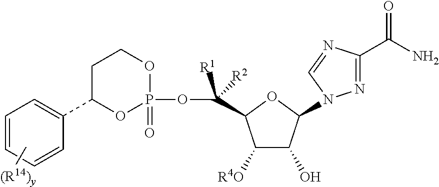 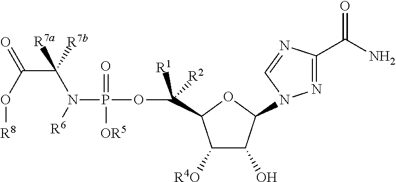 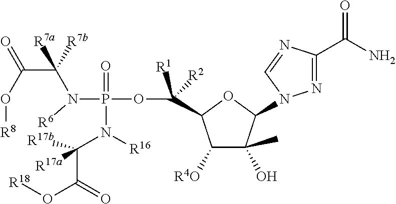 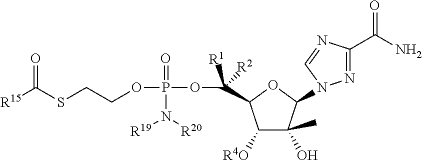 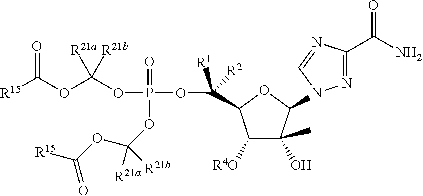 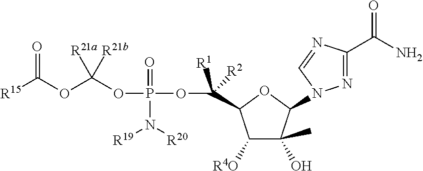
wherein:
R¹ and R² are independently deuterium, hydrogen, or C(H)ₘ(D)ₙ; and at least one of R¹ or R² is deuterium;
R³ is hydrogen, deuterium, halogen (F, Cl, Br, or I), C(H)ₘ(D)ₙ; or alkyne; wherein the R³ alkyne and the C⁴-oxygen of the pyrimidine can form a heterocyclic ring;
R⁴ is hydrogen, deuterium, C(H)ₘ(D)ₙ, acyl or phosphate;
R⁵ is hydrogen, deuterium, alkyl, cycloalkyl, aryl, heteroaryl, heterocyclic, -alkyl(heterocycle), -alkyl(heteroaryl), or -alkylaryl;
R⁶ is hydrogen, deuterium, C₁₋₃alkyl or C₃₋₅cycloalkyl;
R^{7a} and R^{7b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{7a} and R^{7b} form an exo-double bond; or
R^{7a} and R^{7b} form a cycloalkyl or heterocyclic group; or
R⁶ and R^{7a} or R^{7b} form a cycloalkyl group;
R⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R^{9a} and R^{9b} are each independently hydrogen, deuterium, C₁-C₃alkyl, C₃-C₄ cycloalkyl; or
R^{9a} and R^{9b} form a C₃-C₅cycloalkyl group;
R¹⁰ is hydrogen, deuterium, alkyl, cycloalkyl, aryl, heteroaryl, heterocyclic, -alkyl(heterocycle), -alkyl(heteroaryl or -alkylaryl;
R¹¹ is C₁-C₂₂alkyl, cycloalkyl, C₃-C₂₂alkenyl or C₃-C₂₂alkynyl or R¹¹ is —C(O)—C₆-C₂₂alkyl, —C(O)—C₆-C₂₂alkenyl or —C(O)—C₆-C₂₂alkynyl;
R¹² is C₁-C₂₂alkyl, C₃-C₂₂alkenyl or C₃-C₂₂alkynyl or R¹² is —C(O)—C₆-C₂₂alkyl, —C(O)—C₆-C₂₂alkenyl or —C(O)—C₆-C₂₂alkynyl;
R¹³ is hydrogen, deuterium, C(H)ₘ(D)ₙ, acyl or phosphate;
R¹⁴ is independently deuterium, halogen, C₁-C₂haloalkyl, C₁-C₂haloalkoxy, C₁-C₆alkyl, cycloalkyl, allenyl, C₂-C₆alkenyl, C₂-C₆alkynyl, C₁-C₆alkoxy, (C₃-C₆cycloalkyl)C₀-C₄alkyl-, or (aryl)C₀-C₂alkyl-;
R¹⁵ is C₁-C₆alkyl, C₃-C₆ cycloalkyl, —C₁-C₃alkyl-O—C₁-C₅alkoxy, or —C(R^{9a})(R^{9b})CH₂OR⁵;
R¹⁶ is hydrogen, deuterium, C₁₋₃alkyl or C₃₋₅cycloalkyl;
R^{17a} and R^{17b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{17a} and R^{17b} form an exo-double bond; or
R^{17a} and R^{17b} form a cycloalkyl or heterocyclic group; or
R¹⁶ and R^{17a} or R^{17b} form a cycloalkyl group;
R¹⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R¹⁹ and R²⁰ are independently hydrogen, deuterium, alkyl, cycloalkyl, aryl, heteroaryl, heterocyclic, -alkylaryl or a suitable side chain of an amino acid ester; or
R¹⁹ and R²⁰ form a cycloalkyl or heterocyclic group;
R^{21a} and R^{21b} are independently hydrogen, deuterium, C₁₋₆alkyl, C₃-C₆ cycloalkyl; or
R^{21a} and R^{21b} form a 3 to 6 membered ring;
m is 0, 1, 2, or 3;
n is 0, 1, 2, or 3;
m+n=3;
X is S or O;
y is 0, 1, 2, 3, 4, or 5; and
wherein each deuterium is at least 90% enriched;
or a pharmaceutically acceptable salt thereof.

5. A compound selected from: 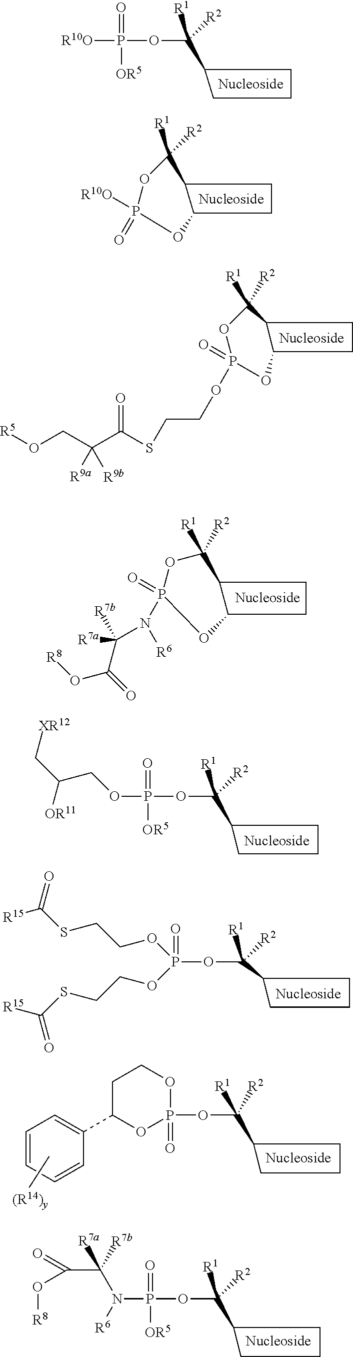 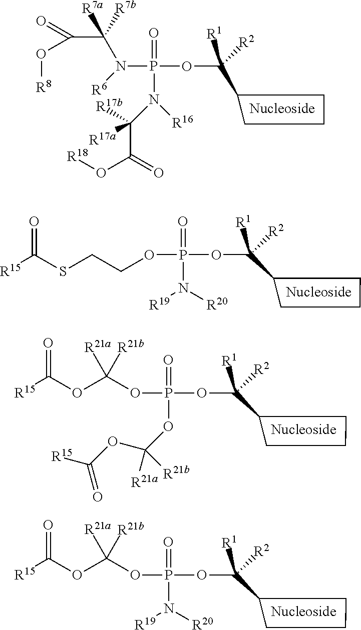
wherein:
R¹ and R² are independently deuterium, hydrogen, or C(H)ₘ(D)ₙ; and at least one of R¹ or R² is deuterium;
R³ is hydrogen, deuterium, halogen (F, Cl, Br, or I), C(H)ₘ(D)ₙ; or alkyne; wherein the R³ alkyne and the C⁴-oxygen of the pyrimidine can form a heterocyclic ring;
R⁴ is hydrogen, deuterium, C(H)ₘ(D)ₙ, acyl or phosphate;
R⁵ is hydrogen, deuterium, alkyl, cycloalkyl, aryl, heteroaryl, heterocyclic, -alkyl(heterocycle), -alkyl(heteroaryl), or -alkylaryl;
R⁶ is hydrogen, deuterium, C₁₋₃alkyl or C₃₋₅cycloalkyl;
R^{7a} and R^{7b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{7a} and R^{7b} form an exo-double bond; or
R^{7a} and R^{7b} form a cycloalkyl or heterocyclic group; or
R⁶ and R^{7a} or R^{7b} form a cycloalkyl group;
R⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R^{9a} and R^{9b} are each independently hydrogen, deuterium, C₁-C₃alkyl, C₃-C₄ cycloalkyl; or
R^{9a} and R^{9b} form a C₃-C₅cycloalkyl group;
R¹⁰ is hydrogen, deuterium, alkyl, cycloalkyl, aryl, heteroaryl, heterocyclic, -alkyl(heterocycle), -alkyl(heteroaryl or -alkylaryl;
R¹¹ is C₁-C₂₂alkyl, cycloalkyl, C₃-C₂₂alkenyl or C₃-C₂₂alkynyl or R¹¹ is —C(O)—C₆-C₂₂alkyl, —C(O)—C₆-C₂₂alkenyl or —C(O)—C₆-C₂₂alkynyl;
R¹² is C₁-C₂₂alkyl, C₃-C₂₂alkenyl or C₃-C₂₂alkynyl or R¹² is —C(O)—C₆-C₂₂alkyl, —C(O)-C₆C₂₂alkenyl or —C(O)—C₆-C₂₂alkynyl;
R¹³ is hydrogen, deuterium, C(H)ₘ(D)ₙ, acyl or phosphate;
R¹⁴ is independently deuterium, halogen, C₁-C₂haloalkyl, C₁-C₂haloalkoxy, C₁-C₆alkyl, cycloalkyl, allenyl, C₂-C₆alkenyl, C₂-C₆alkynyl, C₁-C₆alkoxy, (C₃-C₆cycloalkyl)C₀-C₄alkyl-, or (aryl)C₀-C₂alkyl-;
R¹⁵ is C₁-C₆alkyl, C₃-C₆ cycloalkyl, —C₁-C₃alkyl-O—C₁-C₅alkoxy, or —C(R^{9a})(R^{9b})CH₂OR⁵;
R¹⁶ is hydrogen, deuterium, C₁₋₃alkyl or C₃₋₅cycloalkyl;
R^{17a} and R^{17b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{17a} and R^{17b} form an exo-double bond; or
R^{17a} and R^{17b} form a cycloalkyl or heterocyclic group; or
R¹⁶ and R^{17a} or R^{17b} form a cycloalkyl group;
R¹⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R¹⁹ and R²⁰ are independently hydrogen, deuterium, alkyl, cycloalkyl, aryl, heteroaryl, heterocyclic, -alkylaryl or a suitable side chain of an amino acid ester; or
R¹⁹ and R²⁰ form a cycloalkyl or heterocyclic group;
R^{21a} and R^{21b} are independently hydrogen, deuterium, C₁₋₆alkyl, C₃-C₆ cycloalkyl; or
R^{21a} and R^{21b} form a 3 to 6 membered ring;
m is 0, 1, 2, or 3;
n is 0, 1, 2, or 3;
m+n=3;
X is S or O;
y is 0, 1, 2, 3, 4, or 5; and
wherein each deuterium is at least 90% enriched; and
the nucleoside is: 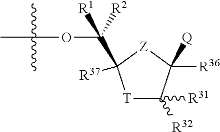
wherein;
Z is O, S or CH═CH₂;
T is O, S or CR³³R³⁴;
R¹ and R² are as defined above;
R³¹ is H, OH, C₂₋₄ alkenyl, C₂₋₄ alkynyl, C₁₋₄ alkyl-O—, C₁₋₄ alkyl, C₁₋₄ haloalkyl, or C₁₋₄ alkyl substituted with from 1 to 4 substituents each of which is independently OH or amino;
R³² is H, OH, amino, cyano, azido, C₁₋₄ alkyl-O—, C₁₋₄ alkyl, C₁₋₄ haloalkyl, or C₁₋₄ alkyl substituted with from 1 to 4 substituents each of which is independently OH or amino;
R³³ is OR⁴, H, OH, cyano, azido, halogen, amino, C₁₋₄ alkyl-O—, C₂₋₄ alkenyl, C₂₋₄ alkynyl, C₁₋₄ alkyl, C₁₋₄ haloalkyl, or C₁₋₄ alkyl substituted with from 1 to 4 substituents each of which is independently OH or amino;
R³⁴ is H, OH, cyano, azido, halogen, amino, C₁₋₄ alkyl-O—, C₂₋₄ alkenyl, C₂₋₄ alkynyl, C₁₋₄ alkyl, C₁₋₄ haloalkyl, or C₁₋₄ alkyl substituted with from 1 to 4 substituents each of which is independently OH or amino;
R³⁶ is H, methyl, hydroxymethyl, or fluoromethyl;
R³⁷ is H, halogen, azido, heteroaryl or cyano;
Q is: 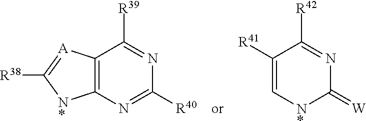
wherein:
* denotes the point of attachment of Q to the C-1 carbon of the furanose ring;
A is N or C—R^{w};
W is O or S;
R³⁸ is H, C₁₋₄ alkyl, C₂₋₄ alkenyl, C₂₋₄ alkynyl, halogen, cyano, carboxy, C₁₋₄ alkyloxycarbonyl, azido, amino, C₁₋₄alkylamino, di(C₁₋₄ alkyl)amino, OH, C₁₋₆ alkyl-O—, C₁₋₄ alkyl-S—, C₁₋₆ alkyl-SO₂—, aminomethyl, or (C₁₋₄alkyl)₁₋₂aminomethyl;
R³⁹ and R⁴² are each independently H, OH, mercapto, halogen, C₁₋₄ alkyl-O—, C₁₋₄ alkyl-S—, amino, C₁₋₄ alkylamino, di(C₁₋₄ alkyl)amino, C₃₋₆ cycloalkylamino, di(C₃₋₆ cycloalkyl)amino, or an amino acyl residue of formula: 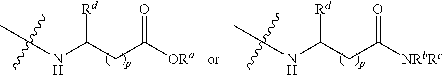
wherein p is an integer equal to zero, 1, 2, 3 or 4;
R⁴⁰ is H, OH, mercapto, halogen, C₁₋₄ alkyl-O—, C₁₋₄ alkyl-S—, amino, C₁₋₄ alkylamino, di(C₁₋₄ alkyl)amino, C₃₋₆ cycloalkylamino, di(C₃₋₆ cycloalkyl)amino, phenyl-C₁₋₂ alkylamino, or C₁₋₄ alkylC(═O)NH—;
R⁴¹ is H, C₁₋₆ alkyl, C₂₋₆ alkenyl optionally substituted with halogen, C₂₋₆ alkynyl, CF₃, or halogen;
R^{a}, R^{b}, and R^{e} are each independently H or C₁₋₆ alkyl;
R^{d} is H, C₁₋₄ alkyl, phenyl-C₁₋₂ alkyl, or phenyl;
R^{w} is H, cyano, nitro, NHC(═O)NH₂, C(═O)NR^{x}R^{x}, CSNR^{x}R^{x}, C(═O)OR^{x}, C(═NH)NH₂, OH, C₁₋₃ alkoxy, amino, C₁₋₄ alkylamino, di(C₁₋₄ alkyl)amino, halogen, C₂-C₆ alkenyl, C₂-C₆ alkynyl, C₁₋₃ alkyl, or C₁₋₃ alkyl substituted with from one to three groups independently selected from aryl, halogen, amino, OH, carboxy, and C₁₋₃ alkyl-O—; and each R^{x} is independently H or C₁₋₆ alkyl;
or a pharmaceutically acceptable salt thereof.

6. A compound selected from: 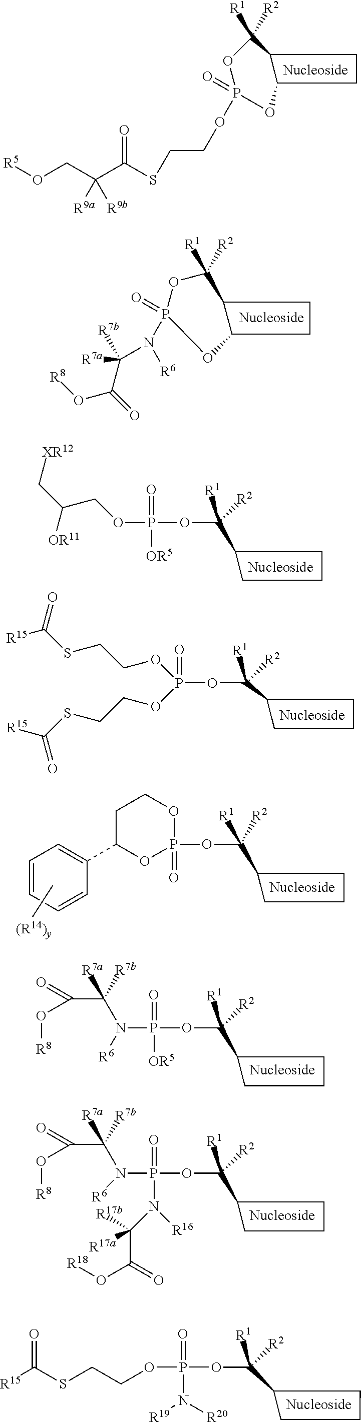 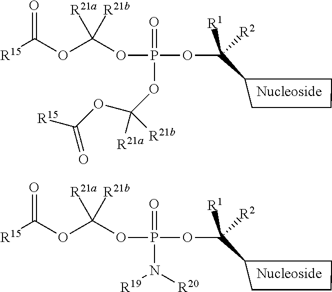
wherein:
R¹ and R² are independently deuterium, hydrogen, or C(H)ₘ(D)ₙ; and at least one of R¹ or R² is deuterium;
R³ is hydrogen, deuterium, halogen (F, Cl, Br, or I), C(H)ₘ(D)ₙ; or alkyne; wherein the R³ alkyne and the C⁴-oxygen of the pyrimidine can form a heterocyclic ring;
R⁴ is hydrogen, deuterium, C(H)ₘ(D)ₙ, acyl or phosphate;
R⁵ is hydrogen, deuterium, alkyl, cycloalkyl, aryl, heteroaryl, heterocyclic, -alkyl(heterocycle), -alkyl(heteroaryl), or -alkylaryl;
R⁶ is hydrogen, deuterium, C₁₋₃alkyl or C₃₋₅cycloalkyl;
R^{7a} and R^{7b} are independently hydrogen, deuterium, C₂-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{7a} and R^{7b} form an exo-double bond; or
R^{7a} and R^{7b} form a cycloalkyl or heterocyclic group; or
R⁶ and R^{7a} or R^{7b} form a cycloalkyl group;
R⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R^{9a} and R^{9b} are each independently hydrogen, deuterium, C₁-C₃alkyl, C₃-C₄ cycloalkyl; or
R^{9a} and R^{9b} form a C₃-C₅cycloalkyl group;
R¹⁰ is hydrogen, deuterium, alkyl, cycloalkyl, aryl, heteroaryl, heterocyclic, -alkyl(heterocycle), -alkyl(heteroaryl or -alkylaryl;
R¹¹ is C₁-C₂₂alkyl, cycloalkyl, C₃-C₂₂alkenyl or C₃-C₂₂alkynyl or R¹¹ is —C(O)—C₆-C₂₂alkyl, —C(O)—C₆-C₂₂alkenyl or —C(O)—C₆-C₂₂alkynyl;
R¹² is C₁-C₂₂alkyl, C₃-C₂₂alkenyl or C₃-C₂₂alkynyl or R¹² is —C(O)—C₆-C₂₂alkenyl or —C(O)—C₆-C₂₂alkenyl or —C(O)—C₆-C₂₂alkynyl;
R¹³ is hydrogen, deuterium, C(H)ₘ(D)ₙ, acyl or phosphate;
R¹⁴ is independently deuterium, halogen, C₁-C₂haloalkyl, C₁-C₂haloalkoxy, C₁-C₆alkyl, cycloalkyl, allenyl, C₂-C₆alkenyl, C₂-C₆alkynyl, C₁-C₆alkoxy, (C₃-C₆cycloalkyl)C₀-C₄alkyl-, or (aryl)C₀-C₂alkyl-;
R¹⁵ is C₁-C₆alkyl, C₃-C₆ cycloalkyl, —C₁-C₃alkyl-O—C₁-C₅alkoxy, or —C(R^{9a})(R^{9b})CH₂OR⁵;
R¹⁶ is hydrogen, deuterium, C₁₋₃alkyl or C₃₋₅cycloalkyl;
R^{17a} and R^{17b} are independently hydrogen, deuterium, C₂-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{17a} and R^{17b} form an exo-double bond; or
R^{17a} and R^{17b} form a cycloalkyl or heterocyclic group; or
R¹⁶ and R^{17a} or R^{17b} form a cycloalkyl group;
R¹⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R¹⁹ and R²⁰ are independently hydrogen, deuterium, alkyl, cycloalkyl, aryl, heteroaryl, heterocyclic, -alkylaryl or a suitable side chain of an amino acid ester; or
R¹⁹ and R²⁰ form a cycloalkyl or heterocyclic group;
R^{21a} and R^{21b} are independently hydrogen, deuterium, C₁₋₆alkyl, C₃-C₆ cycloalkyl; or
R^{21a} and R^{21b} form a 3 to 6 membered ring;
m is 0, 1, 2, or 3;
n is 0, 1, 2, or 3;
m+n=3;
X is S or O;
y is 0, 1, 2, 3, 4, or 5; and
wherein each deuterium is at least 90% enriched;
or a pharmaceutically acceptable salt thereof.

7. A compound selected from:
5′-Deuterated Stabilized Acyclic Nucleoside Phosphate Prodrug
Structures 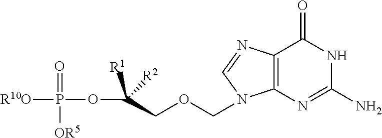 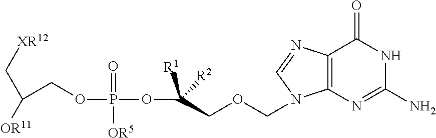 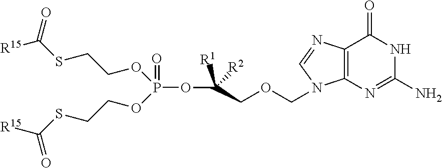 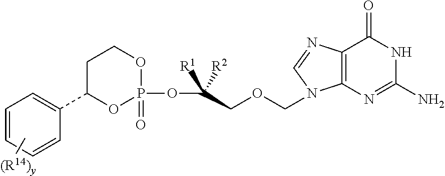 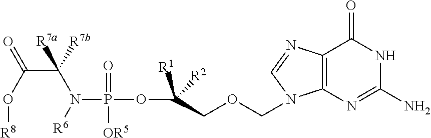 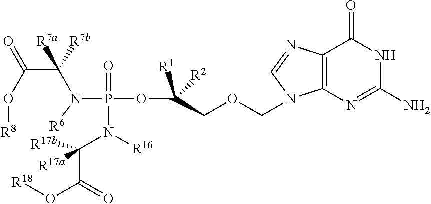 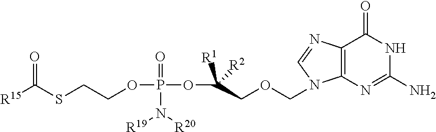 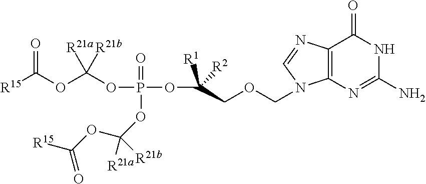 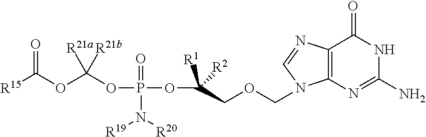
wherein:
R¹ and R² are independently deuterium, hydrogen, or C(H)ₘ(D)ₙ; and at least one of R¹ or R² is deuterium;
R³ is hydrogen, deuterium, halogen (F, Cl, Br, or I), C(H)ₘ(D)ₙ; or alkyne; wherein the R³ alkyne and the C⁴-oxygen of the pyrimidine can form a heterocyclic ring;
R⁴ is hydrogen, deuterium, C(H)ₘ(D)ₙ, acyl or phosphate;
R⁵ is hydrogen, deuterium, alkyl, cycloalkyl, aryl, heteroaryl, heterocyclic, -alkyl(heterocycle), -alkyl(heteroaryl), or -alkylaryl;
R⁶ is hydrogen, deuterium, C₁₋₃alkyl or C₃₋₅cycloalkyl;
R^{7a} and R^{7b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{7a} and R^{7b} form an exo-double bond; or
R^{7a} and R^{7b} form a cycloalkyl or heterocyclic group; or
R⁶ and R^{7a} or R^{7b} form a cycloalkyl group;
R⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R^{9a} and R^{9b} are each independently hydrogen, deuterium, C₁-C₃alkyl, C₃-C₄ cycloalkyl; or
R^{9a} and R^{9b} form a C₃-C₅cycloalkyl group;
R¹⁰ is hydrogen, deuterium, alkyl, cycloalkyl, aryl, heteroaryl, heterocyclic, -alkyl(heterocycle), -alkyl(heteroaryl or -alkylaryl;
R¹¹ is C₁-C₂₂alkyl, cycloalkyl, C₃-C₂₂alkenyl or C₃-C₂₂alkynyl or R¹¹ is —C(O)—C₆-C₂₂alkyl, —C(O)—C₆-C₂₂alkenyl or —C(O)—C₆-C₂₂alkynyl;
R¹² is C₁-C₂₂alkyl, C₃-C₂₂alkenyl or C₃-C₂₂alkynyl or R¹² is —C(O)—C₆-C₂₂alkyl, —C(O)—C₆-C₂₂alkenyl or —C(O)—C₆-C₂₂alkynyl;
R¹³ is hydrogen, deuterium, C(H)ₘ(D)ₙ, acyl or phosphate;
R¹⁴ is independently deuterium, halogen, C₁-C₂haloalkyl, C₁-C₂haloalkoxy, C₁-C₆alkyl, cycloalkyl, allenyl, C₂-C₆alkenyl, C₂-C₆alkynyl, C₁-C₆alkoxy, (C₃-C₆cycloalkyl)C₀-C₄alkyl-, or (aryl)C₀-C₂alkyl-;
R¹⁵ is C₁-C₆alkyl, C₃-C₆ cycloalkyl, —C₁-C₃alkyl-O—C₁-C₅alkoxy, or —C(R^{9a})(R^{9b})CH₂OR⁵;
R¹⁶ is hydrogen, deuterium, C₁₋₃alkyl or C₃₋₅cycloalkyl;
R^{17a} and R^{17b} are independently hydrogen, deuterium, C₁-C₆alkyl, halogen, C₃-C₆cycloalkyl, C₂-C₆alkenyl, C₂-C₆alkynyl, C₁-C₆alkoxy, (C₃-C₆cycloalkyl)C₀-C₄alkyl-, (aryl)C₀-C₂alkyl-, or a side chain of an amino acid; or
R^{17a} and R^{17b} form an exo-double bond; or
R^{17a} and R^{17b} form a cycloalkyl or heterocyclic group; or
R¹⁶ and R^{17a} or R^{17b} form a cycloalkyl group;
R¹⁸ is C₁-C₆alkyl, C₃-C₆cycloalkyl, aryl, C₂-C₆alkenyl, C₂-C₆alkynyl, (C₃-C₇cycloalkyl)C₀-C₄alkyl-, (aryl)C₀-C₄alkyl-, (C₃-C₆heterocycloalkyl)C₀-C₄alkyl-, or (heteroaryl)C₀-C₄alkyl-;
R¹⁹ and R²⁰ are independently hydrogen, deuterium, alkyl, cycloalkyl, aryl, heteroaryl, heterocyclic, -alkylaryl or a suitable side chain of an amino acid ester; or
R¹⁹ and R²⁰ form a cycloalkyl or heterocyclic group;
R^{21a} and R^{21b} are independently hydrogen, deuterium, C₁₋₆alkyl, C₃-C₆ cycloalkyl; or
R^{21a} and R^{21b} form a 3 to 6 membered ring;
m is 0, 1, 2, or 3;
n is 0, 1, 2, or 3;
m+n=3;
X is S or O;
y is 0, 1, 2, 3, 4, or 5; and
wherein each deuterium is at least 90% enriched;
or a pharmaceutically acceptable salt thereof.

8. A pharmaceutical composition comprising a compound or salt of the compound of any one of claims 1 to 7 together with a pharmaceutically acceptable carrier.

9. The pharmaceutical composition of claim 8, comprising at least one additional active agent.

10. A method for treating a hepatitis C virus infection in a host comprising administering an effective amount of a compound of claim 1 to treat the hepatitis C virus infection optionally in a pharmaceutically acceptable carrier.

11. A method for treating a hepatitis C virus infection in a host comprising administering an effective amount of a compound of claim 2 to treat the hepatitis C virus infection optionally in a pharmaceutically acceptable carrier.

12. A method for treating a hepatitis C virus infection in a host comprising administering an effective amount of a compound of claim 3 to treat the hepatitis C virus infection optionally in a pharmaceutically acceptable carrier.

13. A method for treating hepatitis C virus infection or RSV in a host comprising administering an effective amount of a compound of claim 4 to treat the hepatitis C virus infection optionally in a pharmaceutically acceptable carrier.

14. A method for treating a viral infection in a host comprising administering an effective amount of a compound of claim 5 to treat the hepatitis C virus infection optionally in a pharmaceutically acceptable carrier.

15. A method for treating a respiratory syncytial virus (RSV) infection in a host comprising: administering an effective amount of a compound of claim 4 to treat the respiratory syncytial virus infection optionally in a pharmaceutically acceptable carrier.

16. A method for treating hepatitis C infection in a host comprising: administering an effective amount of a compound of claim 6 to treat the Hepatitis C infection optionally in a pharmaceutically acceptable carrier.

17. A method for treating HSV infection in a host comprising: administering an effective amount of a compound of claim 7 to treat HSV C infection optionally in a pharmaceutically acceptable carrier.

18. A compound of claim 6 having the nucleoside structure; 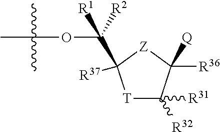
wherein:
Z is O, S or CH═CH₂;
T is O, S or CR³³R³⁴;
R¹ and R² are as defined above;
R³¹ is H, OH, C₂₋₄ alkenyl, C₂₋₄ alkynyl, C₁₋₄ alkyl-O—, C₁₋₄ alkyl, C₁₋₄ haloalkyl, or C₁₋₄ alkyl substituted with from 1 to 4 substituents each of which is independently OH or amino;
R³² is H, OH, amino, cyano, azido, halogen, C₁₋₄ alkyl-O—, C₁₋₄ alkyl, C₁₋₄ haloalkyl, or C₁₋₄ alkyl substituted with from 1 to 4 substituents each of which is independently OH or amino;
R³³ is OR⁴, H, OH, cyano, azido, halogen, amino, C₁₋₄ alkyl-O—, C₂₋₄ alkenyl, C₂₋₄ alkynyl, C₁₋₄ alkyl, C₁₋₄ haloalkyl, or C₁₋₄ alkyl substituted with from 1 to 4 substituents each of which is independently OH or amino;
R³⁴ is H, OH, cyano, azido, halogen, amino, C₁₋₄ alkyl-O—, C₂₋₄ alkenyl, C₂₋₄ alkynyl, C₁₋₄ alkyl, C₁₋₄ haloalkyl, or C₁₋₄ alkyl substituted with from 1 to 4 substituents each of which is independently OH or amino;
R³⁶ is H, methyl, hydroxymethyl, or fluoromethyl;
R³⁷ is H, halogen, azido, heteroaryl or cyano;
Q is: 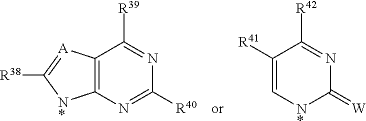
wherein:
* denotes the point of attachment of Q to the C-1 carbon of the furanose ring;
A is N or C—R^{w};
W is O or S;
R³⁸ is H, C₁₋₄ alkyl, C₂₋₄ alkenyl, C₂₋₄ alkynyl, halogen, cyano, carboxy, C₁₋₄ alkyloxycarbonyl, azido, amino, C₁₋₄alkylamino, di(C₁₋₄ alkyl)amino, OH, C₁₋₆ alkyl-O—, C₁₋₄ alkyl-S—, C₁₋₆ alkyl-SO₂—, aminomethyl, or (C₁₋₄alkyl)₁₋₂aminomethyl;
R³⁹ and R⁴² are each independently H, OH, mercapto, halogen, C₁₋₄ alkyl-O—, C₁₋₄ alkyl-S—, amino, C₁₋₄ alkylamino, di(C₁₋₄ alkyl)amino, C₃₋₆ cycloalkylamino, di(C₃₋₆ cycloalkyl)amino, or an amino acyl residue of formula: 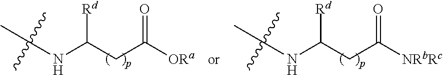
wherein p is an integer equal to zero, 1, 2, 3 or 4;
R⁴⁰ is H, OH, mercapto, halogen, C₁₋₄ alkyl-O—, C₁₋₄ alkyl-S—, amino, C₁₋₄ alkylamino, di(C₁₋₄ alkyl)amino, C₃₋₆ cycloalkylamino, di(C₃₋₆ cycloalkyl)amino, phenyl-C₁₋₂ alkylamino, or C₁₋₄ alkylC(═O)NH—;
R⁴¹ is H, C₁₋₆ alkyl, C₂₋₆ alkenyl optionally substituted with halogen, C₂₋₆ alkynyl, CF₃, or halogen;
R^{a}, R^{b}, and R^{e} are each independently H or C₁₋₆ alkyl;
R^{d} is H, C₁₋₄ alkyl, phenyl-C₁₋₂ alkyl, or phenyl;
R^{w} is H, cyano, nitro, NHC(═O)NH₂, C(═O)NR^{x}R^{x}, CSNR^{x}R^{x}, C(═O)OR^{x}, C(═NH)NH₂, OH, C₁₋₃ alkoxy, amino, C₁₋₄ alkylamino, di(C₁₋₄ alkyl)amino, halogen, C₂-C₆ alkenyl, C₂-C₆ alkynyl, C₁₋₃ alkyl, or C₁₋₃ alkyl substituted with from one to three groups independently selected from aryl, halogen, amino, OH, carboxy, and C₁₋₃ alkyl-O—; and
R^{x} is each independently H or C₁₋₆ alkyl.

19. The compound of claim 1, wherein both R¹ and R² are deuterium.

20. The compound of claim 2, wherein both R¹ and R² are deuterium.

21. The compound of claim 3, wherein both R¹ and R² are deuterium.

22. The compound of claim 4, wherein both R¹ and R² are deuterium.

23. The compound of claim 5, wherein both R¹ and R² are deuterium.

24. The compound of claim 6, wherein both R¹ and R² are deuterium.

25. The compound of claim 7, wherein both R¹ and R² are deuterium.

26. The methods of claims 10-17, wherein the host is a human.


SEQUENCE LISTING (2 sequences)

SEQ ID NO: 1 - DNA, 30 bp, Hepatitis C virus
gccagccccctgatgggggcgacactccac

SEQ ID NO: 2 - DNA, 48 bp, Hepatitis C virus
gaaattaatacgactcactatagggggtgcacggtctacgagacctcc